(19) United States
(12) Patent Application Publication
(10) Pub. No.: US 2025/0388592 A1
(43) Pub. Date: Dec. 25, 2025

(54) (AZA)SPIROHEPTANE DERIVATIVES FOR THE TREATMENT OF NEURODEGENERATIVE DISORDERS

(71) Applicant: REMYND N.V., Leuven (Heverlee) (BE)

(72) Inventors: Johan Gerard GRIFFIOEN, Kessel-Lo (BE); Katrien PRINCEN, Heverlee (BE); Marieke VOETS, Herent (BE); Caterina BISSANTZ, Freiburg (DE); Giuseppe CECERE, Basel (CH); Matthias NETTEKOVEN, Grenzach-Wyhlen (DE); Hasane RATNI, Habsheim (FR); Mark ROGER-EVANS, Bottmingen (CH); Gydo VAN DER HEIJDEN, Nijmegen (NL); Rutger FOLMER, Wijchen (NL); Tim BERKENBOSCH, Zutphen (NL)

(73) Assignee: REMYND N.V., Leuven (Heverlee) (BE)

(21) Appl. No.: 18/881,472

(22) PCT Filed: Aug. 10, 2023
(86) PCT No.: PCT/EP2023/072180

§ 371 Date: Jan. 6, 2025

(30) Foreign Application Priority Data
Aug. 11, 2022 (EP) 22189963.6

(51) Int. Cl. C07D 487/10; A61K 31/397; A61K 31/4155; A61K 31/4178; A61K 31/4184; A61K 31/422; A61K 31/427; A61K 31/437; A61K 31/4439; A61K 31/444; A61K 31/454; A61K 31/4545; A61K 31/496; A61K 31/497; A61K 31/506; A61P 25/28; C07D 519/00

(52) U.S. Cl. CPC C07D 487/10; A61K 31/397; A61K 31/4155; A61K 31/4178; A61K 31/4184; A61K 31/422; A61K 31/427; A61K 31/437; A61K 31/4439; A61K 31/444; A61K 31/454; A61K 31/4545; A61K 31/496; A61K 31/497; A61K 31/506; A61P 25/28; C07D 519/00

(57) ABSTRACT

The application relates to compounds of formula (B1A), (B1B), (B1C) or a salt, a solvate, a hydrate, a polymorph, a tautomer, a racemate or a stereoisomer thereof, to pharmaceutical compositions comprising such a compound, and to the compounds for use as a medicine, in particular for use in the treatment of neurodegenerative disorders such as Alzheimer's disease.

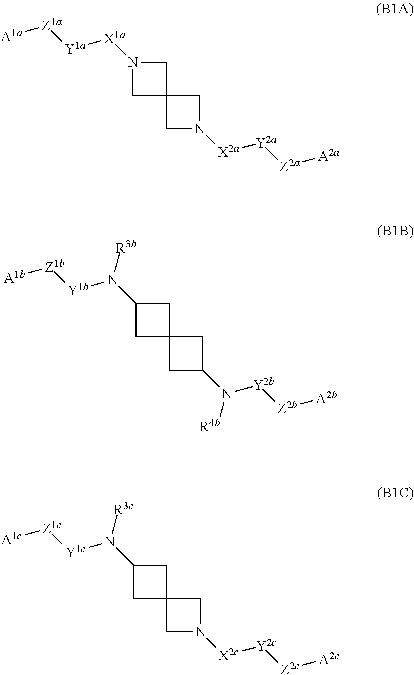

### FIELD OF THE INVENTION

The present invention relates to (aza)spiroheptane derivatives useful in the treatment of certain neurodegenerative disorders, such as neurodegenerative disorders characterized by cytotoxic TAU misfolding and/or aggregation and/or aggregation of amyloid beta.

### BACKGROUND OF THE INVENTION

TAU is a protein with the ability to bind- and consequently stabilise and define-microtubule structure and function in neurons. The binding of TAU to microtubules is regulated by phosphorylation of TAU; several TAU phosphorylation sites and their corresponding kinases have been identified which control phosphorylation status of TAU and consequently modulate the affinity of TAU-binding to microtubules.

Tauopathies are characterised by insoluble aggregates or polymers of hyperphosphorylated TAU which are formed by self-polymerisation of TAU monomers.

An important aspect of the TAU aggregation is its associated cytotoxicity, which reduces neuronal integrity and functionality and ultimately resulting in disease symptoms. A direct role of TAU in disease onset has been established unequivocally by the elucidation of familial mutations in TAU, which appear to be responsible for a very early and sometimes aggressive form of tauopathy. Such mutations comprise changes in the amino acid sequence of TAU that -directly or indirectly promote neurotoxic aggregation.

Alzheimer's disease is the best known of these, where TAU protein is deposited within neurons in the form of neurofibrillary tangles (NFTs). They were first described by the eponymous Alois Alzheimer in one of his patients suffering from the disorder.

The term “Alzheimer's disease” as used herein, refers to a chronic progressive nervous disease characterised by neurodegeneration with as most important (early) symptom being memory loss. As the disease advances, symptoms may include confusion, irritability and aggression, mood swings, language breakdown, long-term memory loss, and the general withdrawal of the sufferer as their senses decline. Tangles are formed by hyperphosphorylation of a microtubule-associated protein known as tau, causing it to aggregate in an insoluble form. (These aggregations of hyperphosphorylated tau protein are also referred to as PHF, or “paired helical filaments”). The precise mechanism of tangle formation is not completely understood, and it is still controversial whether tangles are a primary causative factor in the disease or play a more peripheral role. AD is also classified as an amyloidosis because of the presence of senile plaques.

Other conditions in which neurofibrillary tangles are commonly observed include: Progressive supranuclear palsy, dementia pugilistica (chronic traumatic encephalopathy), frontotemporal dementia and parkinsonism linked to chromosome 17, Lytico-Bodig disease (Parkinson-dementia complex of Guam), tangle-predominant dementia with NFTs, similar to AD, but without plaques, ganglioglioma and gangliocytoma, meningioangiomatosis, subacute sclerosing panencephalitis, tuberous sclerosis, Hallervorden-Spatz disease, and lipofuscinosis.

The non-Alzheimer's tauopathies are sometimes grouped together as “Pick's complex”. In Pick's disease and corticobasal degeneration tau proteins are deposited in the form of inclusion bodies within swollen or “ballooned” neurons. Argyrophilic grain disease (AGD), another type of dementia, is marked by the presence of abundant argyrophilic grains and coiled bodies on microscopic examination of brain tissue.

Currently used treatments for tauopathies, including Alzheimer's disease, offer only symptomatic benefit without impacting the underlying neurodegeneration. Treatments aimed to suppress cytotoxic TAU misfolding and/or aggregation in order to delay or halt the progression of disease are presently not available.

Thus, there is a need for new treatments that target the underlying molecular mechanism of noxious TAU misfolding and/or aggregation in order to reduce neuronal cell death and/or degeneration in patients suffering from tauopathies such as Alzheimer's disease (AD).

### SUMMARY OF THE INVENTION

To at least partially overcome the problems stated above, the present invention provides new compounds as defined herein.

Objects of the present invention are compounds of formula (B1A), (1l B), (1C), their use for the treatment of diseases related to the biological function of dysfunction of TAU protein, which diseases comprise Alzheimer's disease, Pick's disease, corticobasal degeneration, progressive supranuclear palsy, frontotemporal dementia and parkinsonism (linked to chromosome 17, FTDP-17), their manufacture and medicaments based on a compound in accordance with the invention in the control or prevention of illnesses.

In a first aspect, the present invention provides a compound of formula (B1A), (1l B), (B1C) or a salt, a solvate, a hydrate, a polymorph, a tautomer, a racemate or a stereoisomer thereof, or a prodrug thereof; wherein

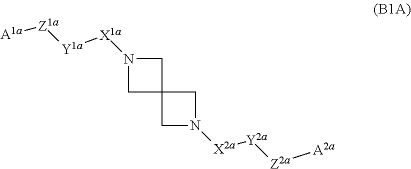

A^{1a} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R^{1a}, wherein each R^{1a} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-10 membered heteroaryl, or a 5-10 membered saturated or partially saturated heterocyclyl;
X^{1a} is selected from —CO—, —SO₂—, —C(R^{3a})₂—, or 5-membered heteroarylene;
Y^{1a} is a single bond, or is selected from —C(R^{3a})₂—, —NR^{5a}— or —O—;
Z^{1a} is a single bond, or is selected from —C(R^{3a})₂—, —NR^{5a}—, or —O—;
each R^{3a} is independently selected from hydrogen, halo or C₁₋₆alkyl;
each R^{5a} is independently selected from hydrogen, or C₁₋₆alkyl;
X^{2a} is a single bond or —CO—;
Y^{2a} is a single bond, or —C(R^{4a})₂—,
Z^{2a} is a single bond, or is selected from —(C(R^{4a})₂)ₙ—, or —CO—; wherein n is an integer selected from 1 or 2;
each R^{4a} is independently selected from hydrogen, halo or C₁₋₆alkyl; or two R^{4a} together with the atom(s) to which they are attached can form a C₃₋₆cycloalkyl;
A^{2a} is selected from the group comprising C₆₋₁₀aryl, 5-10 membered heteroaryl containing at least one N, O and/or S, 5-10 membered saturated or partially saturated heterocyclyl, C₃₋₆cycloalkyl, and C₁₋₆alkyl; wherein each of said group can be unsubstituted or substituted with one or more R^{2a}; wherein each R^{2a} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;

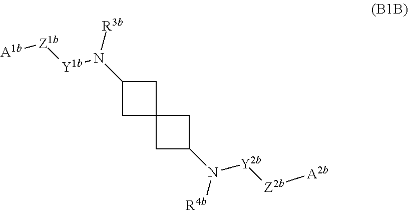

A^{1b} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R^{1b}, wherein each R^{1b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, and C₁₋₆alkylcarbonylamino; and/or two R^{1b} together with the atom(s) to which they are attached can form a C₆₋₁₀aryl, a 5-10 membered heteroaryl, or a 5-10 membered saturated or partially saturated heterocyclyl;
R^{3b} is selected from hydrogen, or C₁₋₆alkyl;
Y^{1b} is selected from —CO—, —C(R^{5b})₂—, or 5-membered heteroarylene;
Z^{1b} is —C(R^{5b})₂—;
R^{4b} is selected from hydrogen, C₁₋₆alkyl, or C₁₋₆alkoxycarbonyl;
each R^{5b} is independently selected from hydrogen, halo or C₁₋₆alkyl;
each R^{6b} is independently selected from hydrogen, or C₁₋₆alkyl;
Y^{2b} is —C(R^{5b})₂—, or a single bond;
Z^{2b} is a single bond, or is selected from —(C(R^{5b})₂)ₘ—, or —CO—; wherein m is an integer selected from 1 or 2;
A^{2b} is selected from the group comprising C₆₋₁₀aryl, 5-10 membered heteroaryl containing at least one N, O and/or S, 5-10 membered saturated or partially saturated heterocyclyl, C₃₋₆cycloalkyl, and C₁₋₆alkyl; wherein each of said group can be unsubstituted or substituted with one or more R^{2b}, wherein each R^{2b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;

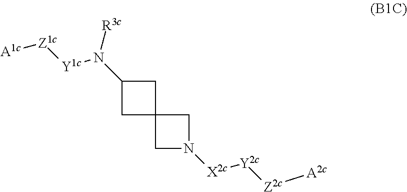

A^{1c} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R¹c, wherein each R^{1c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl;
R^{3c} is selected from hydrogen, or C₁₋₆alkyl;
Y^{1c} is a single bond, or is selected from —C(R^{4c})₂—, —NR^{6c}—, —O—, or a 5 membered heteroarylene;
Z^{1c} is a single bond, or is selected from —C(R^{4c})₂—, or —O—;
each R^{4c} is independently selected from hydrogen, halo or C₁₋₆alkyl;
each R^{6c} is independently selected from hydrogen, or C₁₋₆alkyl;
X^{2c} is a single bond or —CO—;
Y^{2c} is a single bond, or —C(R^{5c})₂—,
Z^{2c} is a single bond, or is selected from —(C(R^{5c})₂)ₚ—, or —CO—; wherein p is an integer selected from 1 or 2;
each R^{5c} is independently selected from hydrogen, halo or C₁₋₆alkyl; or two R^{5c} together with the atom(s) to which they are attached can form a C₃₋₆cycloalkyl;
A^{2c} is selected from the group comprising C₆₋₁₀aryl, 5-10 membered heteroaryl containing at least one N, O and/or S, 5-10 membered saturated or partially saturated heterocyclyl containing at least one N, O and/or S, C₃₋₆cycloalkyl, hydrogen, —N(R^{6c})₂, and C₁₋₆alkyl; wherein each of said group can be unsubstituted or substituted with one or more R^{2c}; wherein each R^{2c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.

Preferably, the present invention provides a compound of formula (B1A), (1l B), (B1C) or a salt, a solvate, a hydrate, a polymorph, a tautomer, a racemate or a stereoisomer thereof, or a prodrug thereof; wherein

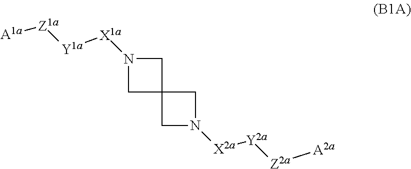

A^{1a} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R^{1a}, wherein each R^{1a} is independently selected from the group comprising halo, hydrogen, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-10 membered heteroaryl, or a 5-10 membered saturated or partially saturated heterocyclyl;
X^{1a} is selected from —CO—, —SO₂—, —C(R^{3a})₂—, or 5-membered heteroarylene;
Y^{1a} is selected from —C(R^{3a})₂—, —NR^{5a}— or —O—; or Y^{1a} is a single bond;
Z^{1a} is a single bond, or is selected from —C(R^{3a})₂—, —NR^{5a}—, or —O—;
each R^{3a} is independently selected from hydrogen, halo or C₁₋₆alkyl;
each R^{5a} is independently selected from hydrogen, or C₁₋₆alkyl;
X^{2a} is a single bond or —CO—;
Y^{2a} is —C(R^{4a})₂— or Y^{2a} is a single bond,
Z^{2a} is a single bond, or is selected from —(C(R^{4a})₂)ₙ—, or —CO—; wherein n is an integer selected from 1 or 2;
each R^{4a} is independently selected from hydrogen, halo or C₁₋₆alkyl; or two R^{4a} together with the atom(s) to which they are attached can form a C₃₋₆cycloalkyl;
A^{2a} is selected from the group comprising C₆₋₁₀aryl, 5-10 membered heteroaryl containing at least one N, O and/or S, 5-10 membered saturated or partially saturated heterocyclyl, and C₃₋₆cycloalkyl; wherein each of said group can be unsubstituted or substituted with one or more R^{2a}; wherein each R^{2a} is independently selected from the group comprising halo, hydrogen, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
with the proviso that:
A^{1a} is not:

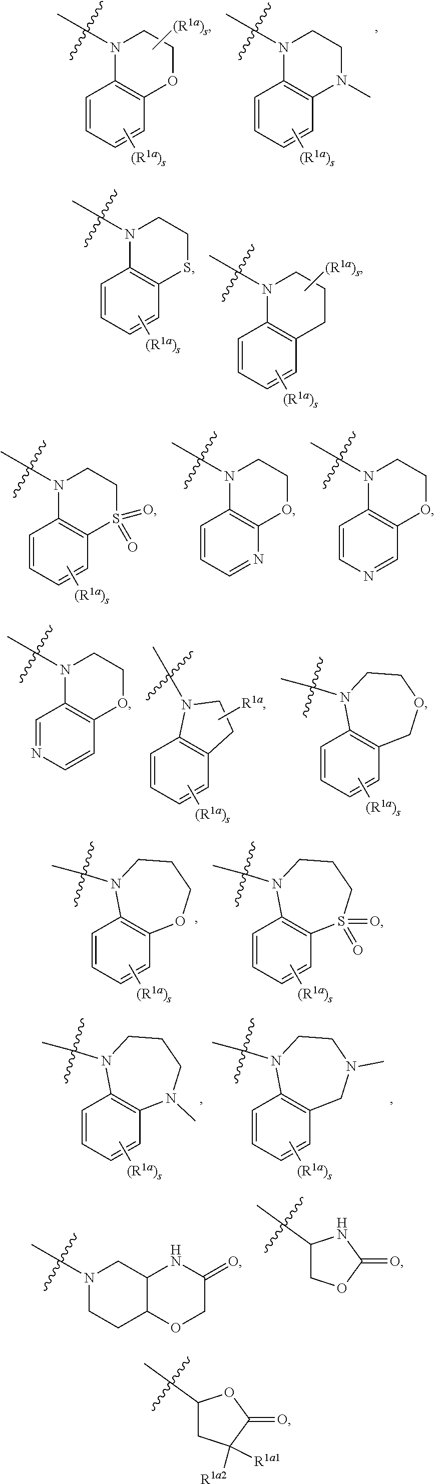

wherein R^{1a1} and R^{1a2} are each independently selected from C₁₋₂alkyl, or R^{1a1} and R^{1a2} together with the carbon atom to which they are attached form a C₃₋₆cycloalkyl, wherein R^{1a} has the same meaning as that defined herein and s is an integer selected from 0, 1 or 2;
A^{2a} is not:

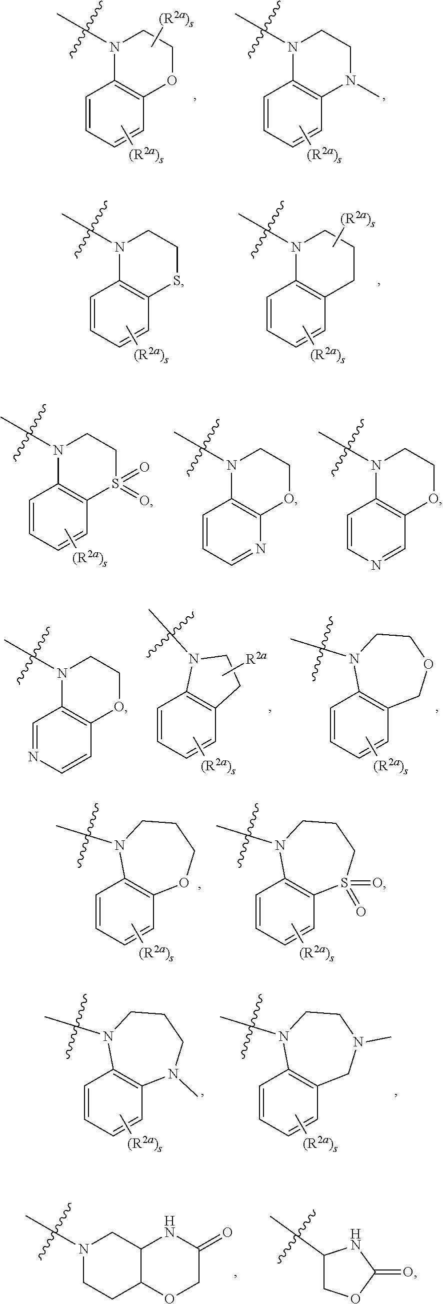

wherein R^{2a} has the same meaning as that defined herein and s is an integer selected from 0, 1 or 2;
with the proviso that said compound is not:
2-Benzyl-6-(toluene-4-sulfonyl)-2,6-diazaspiro[3.3]heptane;
2-(1,3-benzodioxol-5-ylmethyl)-6-[(4-methylphenyl)sulfonyl-2,6-Diazaspiro[3.3]heptane;
2-(1,3-Benzodioxol-5-ylmethyl)-6-(phenylmethyl)-2,6-diazaspiro[3.3]heptane;
2-Benzyl-6-(tosyl)-2,6-diazaspiro[3.3]heptane;
6-benzyl-2,6-diazaspiro[3.3]heptan-2-yl)(3,3,5-trimethyl-2,3-dihydro-1H-pyrrolo[3,2-b]pyridin-1-yl)methanone;
(5-cyclohexyl-1H-pyrazol-3-yl)(6-(cyclopropanecarbonyl)-2,6-diazaspiro[3.3]heptan-2-yl)methanone;
(6-(cyclopropanecarbonyl)-2,6-diazaspiro[3.3]heptan-2-yl)(1-isopropyl-1H-imidazol-4-yl)methanone;
(5-isopropyl-1H-pyrazol-3-yl)(6-(1-methylcyclopropane-1-carbonyl)-2,6-diazaspiro[3.3]heptan-2-yl)methanone;
(5-isopropyl-1H-pyrazol-3-yl)(6-(thiophene-2-carbonyl)-2,6-diazaspiro[3.3]heptan-2-yl)methanone;
[2-(4-fluoro-1-methyl-pyrazole-3-carbonyl)-2,6-diazaspiro[3.3]heptan-6-yl]-(5-isopropyl-1H-pyrazol-3-yl)methanone;

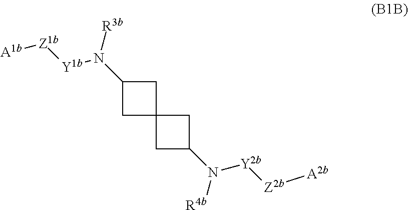

A^{1b} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more Rib, wherein each R^{1b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, and C₁₋₆alkylcarbonylamino; and/or two R^{1b} together with the atom(s) to which they are attached can form a C₆₋₁₀aryl, a 5-10 membered heteroaryl, or a 5-10 membered saturated or partially saturated heterocyclyl;
R^{3b} is selected from hydrogen, or C₁₋₆alkyl;
Y^{1b} is selected from —CO—, —C(R^{5b})₂—, or 5-membered heteroarylene;
Z^{1b} is —C(R^{5b})₂—;
R^{4b} is selected from hydrogen, C₁₋₆alkyl, or C₁₋₆alkoxycarbonyl;
each R^{5b} is independently selected from hydrogen, halo or C₁₋₆alkyl;
each R^{6b} is independently selected from hydrogen, or C₁₋₆alkyl;
Y^{2b} is —C(R^{5b})₂—, or a single bond;
Z^{2b} is a single bond, or is selected from —(C(R^{5b})₂)ₘ—, or —CO—; wherein m is an integer selected from 1 or 2;
A^{2b} is selected from the group comprising C₆₋₁₀aryl, 5-10 membered heteroaryl containing at least one N, O and/or S, 5-10 membered saturated or partially saturated heterocyclyl, C₃₋₆cycloalkyl, and C₁₋₆alkyl; wherein each of said group can be unsubstituted or substituted with one or more R^{2b}, wherein each R^{2b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{5b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;

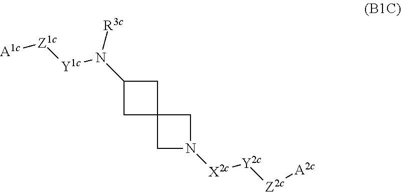

A^{1c} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R¹c, wherein each R¹c is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl;
R^{3c} is selected from hydrogen, or C₁₋₆alkyl;
Y^{1c} is a single bond, or is selected from —C(R^{4c})₂—, —NR^{6c}—, —O—, or a 5 membered heteroarylene;
Z^{1c} is a single bond, or is selected from —C(R^{4c})₂—, or —O—;
each R^{4c} is independently selected from hydrogen, halo or C₁₋₆alkyl;
each R^{6c} is independently selected from hydrogen, or C₁₋₆alkyl;
X^{2c} is a single bond or —CO—;
Y^{2c} is a single bond, or —C(R^{5c})₂—,
Z^{2c} is a single bond, or is selected from —(C(R^{5c})₂)ₚ—, or —CO—; wherein p is an integer selected from 1 or 2;
each R^{5c} is independently selected from hydrogen, halo or C₁₋₆alkyl; or two R^{5c} together with the atom(s) to which they are attached can form a C₃₋₆cycloalkyl;
A^{2c} is selected from the group comprising C₆₋₁₀aryl, 5-10 membered heteroaryl containing at least one N, O and/or S, 5-10 membered saturated or partially saturated heterocyclyl containing at least one N, O and/or S, C₃₋₆cycloalkyl, and —N(R^{6c})₂; wherein each of said group can be unsubstituted or substituted with one or more R^{2c}; wherein each R^{2c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
with the proviso that:
A^{1c} is not

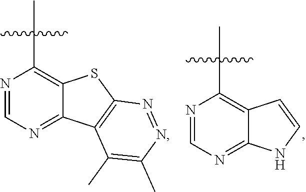

A^{2c} is not

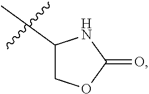 [SMILES: *C1COC(=O)N1]

with the proviso that said compound is not
N-benzyl-2-(6-(2,2,2-trifluoroethyl)thieno[2,3-d]pyrimidin-4-yl)-2-azaspiro[3.3]heptan-6-amine;
N-(2-(2-fluorophenyl)-2-azaspiro[3.3]heptan-6-yl)-6,7-dimethoxy-2,3-dihydro-1H-cyclopenta[b]quinolin-9-amine;
6-((2-((benzyloxy)carbonyl)-2-azaspiro[3.3]heptan-6-yl)amino)-2-chloropyrimidine-4-carboxylic acid;
benzyl 6-((2-chloro-6-(methoxycarbonyl)pyrimidin-4-yl)amino)-2-azaspiro[3.3]heptane-2-carboxylate.

The present invention also relates, in a second aspect, to a pharmaceutical composition comprising a compound according to the first aspect of the invention, and at least one pharmaceutical acceptable carrier.

The present invention also relates to a compound according to the first aspect of the invention, or a pharmaceutical composition according to the second aspect for use as a medicine.

The present compounds and compositions are useful for treating certain neurodegenerative disorders, such as neurodegenerative disorders characterized by suppress cytotoxic TAU misfolding and/or aggregation in order to delay or halt the progression of such diseases. Preferably, the neurodegenerative disorder is selected from the group consisting of Parkinson's disease, Alzheimer's disease, diffuse Lewy body disease, amyotrophic lateral sclerosis, Niemann Pick disease, Hallervorden Spatz syndrome, Down syndrome, Pick's disease, progressive supranuclear palsy, vascular dementia, neuroaxonal dystrophy, Huntington's disease, frontotemporal lobar degeneration (FTLD), multiple system atrophy and Creutzfeld-Jacob's disease, preferably said neurodegenerative disorder is Alzheimer's disease.

The above and other characteristics, features, and advantages of the present invention will become apparent from the following detailed description, which illustrate, by way of example, the principles of the invention.

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. When describing the compounds, processes, articles, and uses of the invention, the terms used are to be construed in accordance with the following definitions, unless the context dictates otherwise.

As used herein, the singular forms “a”, “an”, and “the” include both singular and plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g., from 1.0 to 5.0 includes both 1.0 and 5.0). Any numerical range recited herein is intended to include all sub-ranges subsumed therein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims and statements, any of the embodiments can be used in any combination.

The term “leaving group” or “LG” as used herein means a chemical group which is susceptible to be displaced by a nucleophile or cleaved off or hydrolyzed in basic or acidic conditions. In a particular embodiment, a leaving group is selected from a halogen atom (e.g., Cl, Br, I) or a sulfonate (e.g., mesylate, tosylate, triflate).

The term “protecting group” or “PG” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e., a compound that is sufficiently robust to survive isolation from a reaction mixture.

The term “halo” or “halogen” as a group or part of a group is generic for fluoro, chloro, bromo, iodo.

The term “cyano” as used herein refers to the group —CN.

The term “hydroxyl” or “hydroxy” as used herein refers to the group —OH.

The term “oxo” as used herein refers to the group ═O.

The term “alkyl” as a group or part of a group, refers to a hydrocarbyl group of formula CₙH₂ₙ₊₁ wherein n is a number greater than or equal to 1, with no site of unsaturation. Alkyl groups may be linear or branched and may be substituted as indicated herein. Generally, alkyl groups of this invention comprise from 1 to 18 carbon atoms, preferably from 1 to 10 carbon atoms, more preferably from 1 to 6 carbon atoms, more preferably from 1 to 4 carbon atoms. When a subscript is used herein following a carbon atom, the subscript refers to the number of carbon atoms that the named group may contain. For example, the term “C₁₋₆alkyl”, as a group or part of a group, refers to a hydrocarbyl group of formula CₙH₂ₙ₊₁ wherein n is a number ranging from 1 to 6. Thus, for example, “C₁₋₆alkyl” includes all linear or branched alkyl groups with between 1 and 6 carbon atoms, and thus includes methyl, ethyl, n-propyl, i-propyl, butyl, and its isomers (e.g., n-butyl, i-butyl, and t-butyl); pentyl and its isomers, hexyl, and its isomers, etc. For example, C₁₋₄alkyl includes all linear or branched alkyl groups having 1 to 4 carbon atoms, and thus includes for example methyl, ethyl, n-propyl, i-propyl, 2-methyl-ethyl, butyl, and its isomers (e.g., n-butyl, i-butyl, and t-butyl), and the like. In particular embodiments, the term alkyl refers to C₁₋₁₂alkyl (C₁₋₁₂ hydrocarbons), yet more in particular to C₁₋₆alkyl (C₁₋₉ hydrocarbons), yet more in particular to C₁₋₆alkyl (C₁₋₆ hydrocarbons) as further defined herein. Non-limiting examples of alkyl include methyl, ethyl, 1-propyl (n-propyl), 2-propyl (iPr), 1-butyl, 2-methyl-1-propyl(r-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-icosyl.

When the suffix “ene” is used in conjunction with an alkyl group, i.e., “alkylene”, this is intended to mean the alkyl group as defined herein having two single bonds as points of attachment to other groups. As used herein, the term “alkylene” also referred as “alkanediyl”, by itself or as part of another substituent, refers to alkyl groups that are divalent, i.e., having two monovalent group centers derived by the removal of two hydrogen atoms from the same or two different carbon atoms of a parent alkane, i.e., with two single bonds for attachment to two other groups. Alkylene groups may be linear or branched and may be substituted as indicated herein. Non-limiting examples of alkylene groups include methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

The term “hydrocarbyl” group is used herein in accordance with the definition specified by IUPAC as follows: a univalent group formed by removing a hydrogen atom from a hydrocarbon (that is, a group containing only carbon and hydrogen).

The term “alkenyl” as a group or part of a group, refers to an unsaturated hydrocarbyl group which may be linear, or branched, comprising one or more with at least one site (usually 1 to 3, preferably 1) of unsaturation, namely at least one sp² carbon-sp² carbon double bond. Generally, alkenyl groups of this invention comprise from 2 to 20 carbon atoms, preferably from 2 to 10 carbon atoms, preferably from 2 to 8 carbon atoms, more preferably 2 to 6 carbon atoms. When a subscript is used herein following a carbon atom, the subscript refers to the number of carbon atoms that the named group may contain. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like. The double bond may be in the cis or trans configuration.

When the suffix “ene” is used in conjunction with an alkenyl group, i.e., “alkenylene”, this is intended to mean the alkenyl group as defined herein having two single bonds as points of attachment to other groups. As used herein, the term “alkenylene” by itself or as part of another substituent, refers to alkenyl groups that are divalent, i.e., having two monovalent centers derived by the removal of two hydrogen atoms from the same or two different carbon atoms of a parent alkene, i.e., with two single bonds for attachment to two other groups. Alkenylene groups may be linear or branched and may be substituted as indicated herein. Non-limiting examples of alkenylene groups include —CH═CH—, —C(CH₃)═CH—, —C(CH₃)═C(CH₃)—, —CH═CH—CH₂—, —CH₂—C(CH₃)═CH—, —CH₂—CH═C(CH₃)—, —CH₂—CH₂—CH═CH—, and the like.

The term “alkynyl” as a group or part of a group, refers to a branched or straight chain hydrocarbon comprising at least one site (usually 1 to 3, preferably 1) of unsaturation, namely a sp¹ carbon-sp¹ carbon triple bond. In particular embodiments, the term alkynyl refers to C₂₋₁₂ alkynyl (C₂₋₁₂ hydrocarbons), preferably to C₂₋₉ alkynyl (C₂₋₉ hydrocarbons) yet more preferably to C₂₋₆ alkynyl (C₂₋₆ hydrocarbons) as further defined herein above with at least one site (usually 1 to 3, preferably 1) of unsaturation, namely at least one sp¹ carbon-sp¹ carbon triple bond. Examples of alkynyl include but are not limited to: ethynyl (—C⁰CH), 3-ethyl-cyclohept-1-ynylene, and 1-propynyl (propargyl, —CH₂C⁰CH).

When the suffix “ene” is used in conjunction with an alkynyl group, i.e., “alkynylene”, this is intended to mean the alkynyl group as defined herein having two single bonds as points of attachment to other groups. As used herein, the term “alkynylene” by itself or as part of another substituent, refers to alkynyl groups that are divalent, i.e., with two single bonds for attachment to two other groups. Alkynylene groups may be linear or branched and may be substituted as indicated herein. Non-limiting examples of alkynylene groups include —C⁰C—, —CH₂—C⁰C—, —C⁰C—CH₂—, —CH₂—CH₂—C⁰C—, and the like.

The term “cycloalkyl”, as a group or part of a group, refers to a cyclic alkyl group, that is a monovalent, saturated, hydrocarbyl group having 1 or more cyclic structure, and comprising from 3 to 20 carbon atoms, more preferably from 3 to 10 carbon atoms, more preferably from 3 to 8 carbon atoms; more preferably from 3 to 6 carbon atoms. Cycloalkyl includes all saturated hydrocarbon groups containing 1 or more rings, including monocyclic, bicyclic groups or tricyclic. For example, cycloalkyl comprises a C₃₋₁₀ monocyclic or C₇₋₁₈ polycyclic saturated hydrocarbon, such as for instance cyclopropyl, cyclobutyl, cyclopentyl, cyclopropylethylene, methylcyclopropylene, cyclohexyl, cycloheptyl, cyclooctyl, cyclooctylmethylene, norbornyl, fenchyl, trimethyltricycloheptyl, decalinyl, adamantyl and the like. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₁₀cycloalkyl”, refers to a cyclic alkyl group comprising from 3 to 10 carbon atoms. For example, the term “C₃₋₈cycloalkyl”, refers to a cyclic alkyl group comprising from 3 to 8 carbon atoms. For example, the term “C₃₋₆cycloalkyl”, refers to a cyclic alkyl group comprising from 3 to 6 carbon atoms. For the avoidance of doubt, fused systems of a cycloalkyl ring with a heterocyclic ring are considered as heterocycle irrespective of the ring that is bound to the core structure. Fused systems of a cycloalkyl ring with an aryl ring are considered as aryl irrespective of the ring that is bound to the core structure. Fused systems of a cycloalkyl ring with a heteroaryl ring are considered as heteroaryl irrespective of the ring that is bound to the core structure.

The term “alkoxy” or “alkyloxy”, as a group or part of a group, refers to a group of formula —OR^{b} wherein R^{b} is alkyl as defined herein. Non-limiting examples of suitable C₁₋₆alkoxy include methoxy, ethoxy, propoxy, isopropoxy, butoxy, isobutoxy, sec-butoxy, tert-butoxy, pentyloxy, and hexyloxy.

The term “aryl”, as a group or part of a group, refers to a polyunsaturated, aromatic hydrocarbyl group having a single ring (i.e., phenyl) or multiple aromatic rings fused together (e.g., naphthyl), or linked covalently, typically containing 6 to 20 atoms; preferably 6 to 10, wherein at least one ring is aromatic. Typical aryl groups include, but are not limited to 1 ring, or 2 or 3 rings fused together, derived from benzene, naphthalene, anthracene, biphenyl, and the like. The aromatic ring may optionally include one to two additional rings. Fused systems of an aryl ring with a cycloalkyl ring, or a cycloalkenyl ring, or a cycloalkynyl ring, are considered as aryl irrespective of the ring that is bound to the core structure. Fused systems of an aryl ring with a heterocycle are considered as heterocycle irrespective of the ring that is bound to the core structure. Fused systems of an aryl ring with a heteroaryl are considered as heteroaryl irrespective of the ring that is bound to the core structure. Examples of suitable aryl include C₆₋₂₀aryl, preferably C₆₋₁₀aryl, more preferably C₆₋₉aryl. Non-limiting examples of aryl comprise phenyl, biphenylyl, biphenylenyl, or 1- or 2-naphthanelyl; 1-, 2-, 3-, 4-, 5- or 6-tetralinyl (also known as “1,2,3,4-tetrahydronaphtalene); 1-, 2-, 3-, 4-, 5-, 6-, 7- or 8-azulenyl, 4-, 5-, 6 or 7-indenyl; 4- or 5-indanyl; 5-, 6-, 7- or 8-tetrahydronaphthyl; 1,2,3,4-tetrahydronaphthyl; and 1,4-dihydronaphthyl; 1-, 2-, 3-, 4- or 5-pyrenyl. Preferred aryl is phenyl.

When the suffix “ene” is used in conjunction with an aryl group, i.e., “arylene”, this is intended to mean the aryl group as defined herein having two single bonds as points of attachment to other groups. As used herein, the term “arylene” by itself or as part of another substituent, refers to aryl groups that are divalent, i.e., having two monovalent centres derived by the removal of two hydrogen atoms from the same or two different carbon atoms of a parent aryl, i.e., with two single bonds for attachment to two other groups. Arylene groups may be substituted as indicated herein. The term “arylalkyl”, as a group or part of a group, refers to an alkyl as defined herein, wherein at least one hydrogen atom is replaced by at least one aryl as defined herein. Non-limiting examples of arylalkyl group include benzyl, phenethyl, dibenzylmethyl, benzyl, 2-phenylethan-1-yl, 2-phenylethen-1-yl, naphthylmethyl, 2-naphthylethyl, and the like. The term “C₆₋₁₀arylC₁₋₆alkyl” means that the alkyl moiety of the arylalkyl group can comprises 1 to 6 carbon atoms and the aryl moiety is 6 to 10 carbon atoms.

The term “aryloxy”, as a group or part of a group, refers to a group of formula —O—R^{f} wherein R^{f} is aryl as defined herein.

The term “haloalkyl”, as a group or part of a group, refers to an alkyl group having the meaning as defined herein, wherein one or more hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl and the like.

The term “haloalkoxy”, as a group or part of a group, refers to a group of formula —O—R^{e}, wherein R^{e} is haloalkyl as defined herein. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy, trichloromethoxy, 2-bromoethoxy, pentafluoroethyl, 3,3,3-trichloropropoxy, 4,4,4-trichlorobutoxy.

The term “heterocyclyl” as used herein refer to non-aromatic, fully saturated or partially unsaturated ring system comprising from 3 to 18 atoms including at least one N, O, S, or P, preferably 3 to 14 atoms (3-14 membered heterocyclyl) (for example, 3 to 7 member monocyclic, 7 to 14 member bicyclic, preferably comprising a total of 3 to 10 ring atoms (3-10 membered heterocyclyl), more preferably 4 to 10 atoms (4-10 membered heterocyclyl), yet more preferably 5 to 10 atoms (5-10 membered heterocyclyl). Each ring of the heterocycle or heterocyclyl may have 1, 2, 3 or 4 heteroatoms selected from N, O, P and/or S, where the N and S heteroatoms may optionally be oxidized, and the N heteroatoms may optionally be quaternized; and wherein at least one carbon atom of heterocyclyl can be oxidized to form at least one C═O. The heterocyclyl may be attached at any heteroatom or carbon atom of the ring or ring system, where valence allows. The rings of multi-ring heterocyclyls or heterocycles may be fused, bridged and/or joined through one or more spiro atoms. Fused systems of a heterocycle or heterocyclyl with an aryl ring are considered as heterocycle or heterocyclyl irrespective of the ring that is bound to the core structure. Fused systems of a heterocycle or heterocyclyl with a heteroaryl ring are considered as heteroaryl irrespective of the ring that is bound to the core structure.

Non limiting exemplary heterocycles or heterocyclic groups include piperidinyl, piperazinyl, homopiperazinyl, morpholinyl, tetrahydropyranyl, tetrahydrothiopyranyl (tetrahydro-2H-thiopyranyl), tetrahydrofuranyl, pyrrolidinyl, aziridinyl, oxiranyl, thiiranyl, azetidinyl, oxetanyl, thietanyl, imidazolinyl, pyrazolidinyl imidazolidinyl, oxazolinyl, isoxazolinyl, oxazolidinyl, isoxazolidinyl, thiazolidinyl, isothiazolidinyl, succinimidyl, indolinyl, isoindolinyl, chromanyl (also known as 3,4-dihydrobenzo[b]pyranyl), 2H-pyrrolyl, pyrrolinyl (such as 1-pyrrolinyl, 2-pyrrolinyl, 3-pyrrolinyl), 4H-quinolizinyl, 2-oxopiperazinyl, pyrazolinyl (such as 2-pyrazolinyl, 3-pyrazolinyl), tetrahydro-2H-pyranyl, tetrahydro-2H-thiopyranyl-1-oxide, tetrahydro-2H-thiopyranyl-1,1-dioxide, 2H-pyranyl, 4H-pyranyl, dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formyl-piperazinyl, thiomorpholinyl, dihydrofuranyl, dihydrothienyl, tetrahydrothienyl, dihydropyrazolyl, dihydroimidazolyl, isothiazolinyl, thiazolinyl, triazolinyl, triazolidinyl, oxadiazolinyl, oxadiazolidinyl, thiadiazolinyl, thiadiazolidinyl, tetrazolinyl, tetrazolidinyl, dihydro-pyridinyl, tetrahydro-pyridinyl, 1,2,3,6-tetrahydropyridinyl, hexahydro-pyridinyl, dihydro-pyrimidinyl, tetrahydro-pyrimidinyl, 1,4,5,6-tetrahydropyrimidinyl, dihydro-pyrazinyl, tetrahydro-pyrazinyl, dihydro-pyridazinyl, tetrahydro-pyridazinyl, dihydro-triazinyl, tetrahydro-triazinyl, hexahydro-triazinyl, 1,4-diazepanyl, dihydro-indolyl, indolinyl, tetrahydro-indolyl, dihydro-indazolyl, tetrahydro-indazolyl, dihydro-isoindolyl, dihydro-benzofuranyl, tetrahydro-benzofuranyl, dihydro-benzothienyl, tetrahydro-benzothienyl, dihydro-benzimidazolyl, tetrahydro-benzimidazolyl, dihydro-benzooxazolyl, 2,3-dihydrobenzo[d]oxazolyl, tetrahydro-benzooxazolyl, dihydro-benzooxazinyl, 3,4-dihydro-2H-benzo[b][1,4]oxazinyl, tetrahydro-benzooxazinyl, benzo[1,3]dioxolyl, benzo[1,4]dioxanyl, dihydro-purinyl, tetrahydro-purinyl, dihydro-quinolinyl, 1,2,3,4-tetrahydroquinolinyl, dihydro-isoquinolinyl, 3,4-dihydroisoquinolin-(1H)-yl, tetrahydro-isoquinolinyl, 1,2,3,4-tetrahydroisoquinolinyl, dihydro-quinazolinyl, tetrahydro-quinazolinyl, dihydro-quinoxalinyl, tetrahydro-quinoxalinyl, 1,2,3,4-tetrahydroquinoxalinyl, 2,5-dihydro-1H-pyrrolyl, 4,5-dihydro-1H-imidazolyl, hexahydropyrrolo[3,4-b][1,4]oxazin-(2H)-yl, 3,4-dihydro-2H-pyrido[3,2-b][1,4]oxazinyl, (cis)-octahydrocyclopenta[c]pyrrolyl, hexahydropyrrolo[3,4-b]pyrrol-(1H)-yl, 5H-pyrrolo[3,4-b]pyridin-(7H)-yl, 5,7-dihydro-6H-pyrrolo[3,4-b]pyridinyl, tetrahydro-1H-pyrrolo[3,4-b]pyridin-(2H,7H,7aH)-yl, hexahydro-1H-pyrrolo[3,4-b]pyridin-(2H)-yl, (octahydro-6H-pyrrolo[3,4-b]pyridinyl, hexahydropyrrolo[1,2-a]pyrazin-(1H)-yl, 3,4,6,7,8,8a-hexahydro-1H-pyrrolo[1,2-a]pyrazinyl, 2,3,4,9-tetrahydro-1H-carbazolyl, 1,2,3,4-tetrahydropyrazino[1,2-a]indolyl, 2,3-dihydro-1H-pyrrolo[1,2-a]indolyl, 1,3-dihydro-2H-isoindolyl, octahydro-2H-isoindolyl, 2,5-diazabicyclo[2.2.1]heptanyl, 2-azabicyclo[2.2.1]heptenyl, 3-azabicyclo[3.1.0]hexanyl, 3,6-diazabicyclo[3.1.0]hexanyl, 5-azaspiro[2.4]heptanyl, 4,7-diazaspiro[2.5]octanyl, 2,6-diazaspiro[3.3]heptanyl, 2,5-diazaspiro[3.4]octanyl, 2,6-diazaspiro[3.4]octanyl, 2,7-diazaspiro[3.5]nonanyl, 2,7-diazaspiro[4.4]nonanyl, 2-azaspiro[4.5]decanyl, 2,8-diazaspiro[4.5]decanyl, 3,6-diazabicyclo[3.2.1]octyl, 1,4-dihydroindeno[1,2-c]pyrazolyl, dihydropyranyl, dihydropyridinyl, dihydroquinolinyl, 8H-indeno[1,2-d]thiazolyl, tetrahydroimidazo[1,2-a]pyridinyl, pyridin-2(1H)-one, 8-azabicyclo[3.2.1]oct-2-enyl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “tetrahydrothiopyranyl” also known as “thianyl” or “tetrahydro-2H-thiopyranyl”, as used herein includes tetrahydrothiopyran-2-yl, tetrahydrothiopyran-3-yl and tetrahydrothiopyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxan-5-yl, 1,2,4-trioxan-3-yl, 1,2,4-trioxan-5-yl, 1,2,4-trioxan-6-yl and 1,3,4-trioxan-2-yl. The term “azepanyl” as used herein includes azepan-1-yl, azepan-2-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “chromanyl” as used herein includes chroman-2-yl, chroman-3-yl, chroman-4-yl, chroman-5-yl, chroman-6-yl, chroman-7-yl and chroman-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

When the suffix “ene” is used in conjunction with a heterocyclyl group, i.e., “heterocyclylene”, this is intended to mean the heterocyclyl group as defined herein having two single bonds as points of attachment to other groups. As used herein, the term “heterocyclylene” by itself or as part of another substituent, refers to heterocyclyl groups that are divalent, i.e., having two monovalent centres derived by the removal of two hydrogen atoms from the same or two different carbon atoms of a parent heterocyclyl, i.e., with two single bonds for attachment to two other groups. Heterocyclylene groups may be substituted as indicated herein.

The term “heteroaryl” refers to an aromatic ring system comprising from 5 to 18 atoms including at least one N, O, S, or P, containing 1 or 2 rings which can be fused together or linked covalently, preferably 5 to 14 atoms (5-14 membered heteroaryl), yet more preferably 5 to 10 atoms (5-10 membered heteroaryl), each ring typically containing 5 to 6 atoms; at least one of said rings is aromatic, where the N and S heteroatoms may optionally be oxidized and the N heteroatoms may optionally be quaternized, and wherein at least one carbon atom of said heteroaryl can be oxidized to form at least one C═O. Fused systems of a heteroaryl ring with a cycloalkyl ring, or a cycloalkenyl ring, or a cycloalkynyl ring, are considered as heteroaryl irrespective of the ring that is bound to the core structure. Fused systems of a heteroaryl ring with a heterocycle are considered as heteroaryl irrespective of the ring that is bound to the core structure. Fused systems of a hetero aryl ring with an aryl ring are considered as heteroaryl irrespective of the ring that is bound to the core structure. Non-limiting examples of such heteroaryl, include: pyridinyl, pyrrolyl, thiophenyl (also referred as thienyl), furanyl, thiazolyl, isothiazolyl, thiadiazolyl, triazol-2-yl, 1H-pyrazol-5-yl, pyrazolyl, imidazolyl, oxazolyl, isoxazolyl, triazolyl, oxadiazolyl, tetrazolyl, oxatriazolyl, thiatriazolyl, pyrimidinyl, pyrazinyl, pyridazinyl, oxazinyl, dioxinyl, thiazinyl, triazinyl, pyranyl, thiopyranyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, benzooxazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, benzo[c][1,2,5]oxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, 9H-purinyl, imidazo[1,2-a]pyridinyl, imidazo[1,2-a]pyrazinyl, imidazo[5,1-a]isoquinolinyl, imidazo[1,5-a]pyridinyl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; acridinyl, phthalazinyl, 1,4-dihydroindeno[1,2-c]-1H-pyrazolyl, 2,3-dihydro-1H-inden-1-one, 2,3-dihydro-1H-indenyl, 3,4-dihydroquinolin-2(1H)-one, 5,6-dihydroimidazo[5,1-a]isoquinolinyl, 8H-indeno[1,2-d]thiazolyl, benzo[d]oxazol-2(3H)-one, quinolin-2(1H)-one, quinazolin-4(1H)-one, quinazoline-2,4(1H,3H)-dione, benzo-[d]oxazolyl, and pyrazolo[1,5-a]pyridinyl.

In some preferred embodiment the term “5 or 6-membered heteroaryl” denotes an aromatic ring system, containing one or more heteroatoms, selected from N, S or O, wherein in some embodiment, the heteroaryl can be for example selected from the group comprising pyridinyl, thiadiazolyl, thiazolyl, imidazolyl, isoxazolyl, pyrimidinyl, thienyl or furanyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl or 1H-pyrazol-5-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes triazol-2-yl, 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazol-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl, indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

When the suffix “ene” is used in conjunction with a heteroaryl group, i.e., “heteroarylene”, this is intended to mean the heteroaryl group as defined herein having two single bonds as points of attachment to other groups. As used herein, the term “heteroarylene” by itself or as part of another substituent, refers to heteroaryl groups that are divalent, i.e., having two monovalent centres derived by the removal of two hydrogen atoms from the same or two different carbon atoms of a parent heteroaryl, i.e., with two single bonds for attachment to two other groups. Heteroarylene groups may be substituted as indicated herein.

Heteroaryl and heterocycle or heterocyclyl as used herein includes by way of example and not limitation these groups described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

The term “carbonyl” as a group or part of a group, refers to the group —C(═O)—, also written as —CO—.

The term “carboxy”, “carboxyl” or “hydroxycarbonyl”, as a group or part of a group, refers to the group —C(═O)—OH.

The term “alkoxycarbonyl” or “alkyloxycarbonyl”, as a group or part of a group, refers to a group of formula —C(═O)—O—R^{b}, wherein R^{b} is alkyl as defined herein.

The term “amino” as a group or part of a group, refers to the —NH₂ group.

The term “mono- or di-alkylamino”, as a group or part of a group, refers to a group of formula —N(R^{l})(R^{b}), wherein R^{l} is hydrogen or alkyl, R^{b} is alkyl as defined herein. Thus, such term includes mono-alkyl amino group (e.g., mono-alkylamino group such as methylamino and ethylamino), and di-alkylamino group (e.g., di-alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-1-propylamino, n-butylmethylamino, i-butylmethylamino, t-butylmethylamino, ethyl-n-propylamino, ethyl-1-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “alkylcarbonylamino”, as a group or part of a group, refers to a group of formula —N(R^{l})—C(═O)—R^{b}, wherein R^{l} is hydrogen or alkyl, R^{b} is alkyl as defined herein.

The term “single bond” as used herein for a linking group i.e., in a way that a certain linking group is selected from a single bond, etc. in the formulas herein, refers to a molecule wherein the linking group is not present and therefore refers to compounds with a direct linkage via a single bond between the two moieties being linked by the linking group.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

Any reference to a “compound according to the invention”, or “compound of formula (I)” also includes isomers such as stereoisomers and tautomers, salts such as pharmaceutically and/or physiologically acceptable salts, hydrates, solvates, polymorphs of such compounds unless expressly indicated otherwise.

As used herein and unless otherwise stated, the term “solvate” includes any combination which may be formed by a derivative of this invention with a suitable inorganic solvent (e.g., hydrates) or organic solvent, such as but not limited to alcohols, ketones, esters, ethers, nitriles, and the like.

Preferred statements (features) and embodiments of the methods, compositions, and uses of this invention are set herein below. Each statement and embodiment of the invention so defined may be combined with any other statement and/or embodiment, unless clearly indicated to the contrary. In particular, any feature indicated as being preferred or advantageous may be combined with any other features or statements indicated as being preferred or advantageous. Hereto, the present invention is in particular captured by any one or any combination of one or more of the below numbered statements and embodiments, with any other aspect and/or embodiment.
1. A compound of formula (B1A), (B1B) or (B1C), or a salt, a solvate, a hydrate, a polymorph, a tautomer, a racemate or a stereoisomer thereof; or a prodrug thereof, wherein

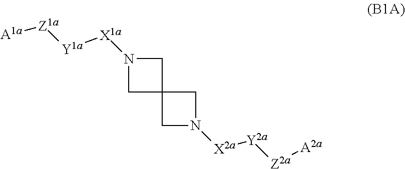

A^{1a} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; preferably A^{1a} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1a} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1a} is selected from the group comprising C₆₋₁₀aryl, C₅₋₆cycloalkyl, 5-6 membered heteroaryl containing at least one N, O and/or S;
   wherein each of said group can be unsubstituted or substituted with one or more R^{1a} wherein each R^{1a} is independently selected from the group comprising hydrogen, halo, C₁₋₆alkyl, C₂₋₆alkenyl, C₂₋₆alkynyl, C₁₋₆-alkoxy, haloC₁₋₆-alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-10 membered heteroaryl, or a 5-10 membered saturated or partially saturated heterocyclyl;
X^{1a} is selected from —CO—, —SO₂—, —C(R^{3a})₂—, or 5-membered heteroarylene;
Y^{1a} is a single bond, or is selected from —C(R^{3a})₂—, —NR^{5a}— or —O—;
Z^{1a} is a single bond, or is selected from —C(R^{3a})₂—, —NR^{5a}—, or —O—;
each R^{3a} is independently selected from hydrogen, halo or C₁₋₆alkyl;
each R^{5a} is independently selected from hydrogen, or C₁₋₆alkyl;
X^{2a} is a single bond or —CO—;
Y^{2a} is a single bond, or —C(R^{4a})₂—,
Z^{2a} is a single bond, or is selected from —(C(R^{4a})₂)ₙ—, or —CO—; wherein n is an integer selected from 1 or 2;
each R^{4a} is independently selected from hydrogen, halo or C₁₋₆alkyl; or two R^{4a} together with the atom(s) to which they are attached can form a C₃₋₆cycloalkyl;
A^{2a} is selected from the group comprising C₆₋₁₀aryl, 5-10 membered heteroaryl containing at least one N, O and/or S, 5-10 membered saturated or partially saturated heterocyclyl, C₃₋₆cycloalkyl, and C₁₋₆alkyl; preferably A^{2a} is selected from the group comprising C₆₋₁₀aryl, 5-9 membered heteroaryl containing at least one N, O and/or S, C₃₋₆cycloalkyl, and C₁₋₄alkyl; preferably A^{2a} is selected from the group comprising C₆₋₁₀aryl, 5-6 membered heteroaryl containing at least one N, O and/or S, and C₄₋₆cycloalkyl; preferably A^{2a} is selected from the group comprising phenyl, 5-6 membered heteroaryl containing at least one N, O and/or S and C₅₋₆cycloalkyl;
   wherein each of said group can be unsubstituted or substituted with one or more R^{2a} wherein each R^{2a} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;

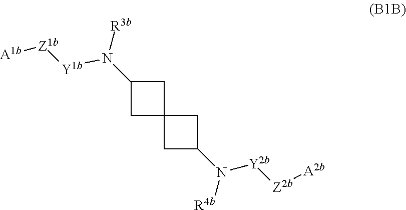

A^{1b} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; preferably A^{1b} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1b} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1b} is selected from the group comprising C₆₋₁₀aryl, C₅₋₆cycloalkyl, 5-6 membered heteroaryl containing at least one N, O and/or S; preferably A^{1b} is phenyl;
   wherein each of said group can be unsubstituted or substituted with one or more Rib wherein each R^{1b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, and C₁₋₆alkylcarbonylamino; and/or two R^{1b} together with the atom(s) to which they are attached can form a C₆₋₁₀aryl, a 5-10 membered heteroaryl, or a 5-10 membered saturated or partially saturated heterocyclyl;
R^{3b} is selected from hydrogen, or C₁₋₆alkyl;
Y^{1b} is selected from —CO—, —C(R^{5b})₂—, or 5-membered heteroarylene;
Z^{1b} is —C(R^{5b})₂—;
R^{4b} is selected from hydrogen, C₁₋₆alkyl, or C₁₋₆alkoxycarbonyl;
each R^{5b} is independently selected from hydrogen, halo or C₁₋₆alkyl;
each R^{6b} is independently selected from hydrogen, or C₁₋₆alkyl;
Y^{2b} is —C(R^{5b})₂—, or a single bond;
Z^{2b} is a single bond, or is selected from —(C(R^{5b})₂)ₘ—, or —CO—; wherein m is an integer selected from 1 or 2;
A^{2b} is selected from the group comprising C₆₋₁₀aryl, 5-10 membered heteroaryl containing at least one N, O and/or S, 5-10 membered saturated or partially saturated heterocyclyl, C₃₋₆cycloalkyl, and C₁₋₆alkyl; preferably A^{2b} is selected from the group comprising C₆₋₁₀aryl, 5-9 membered heteroaryl containing at least one N, O and/or S, C₃₋₆cycloalkyl, and C₁₋₄alkyl; preferably A^{2b} is selected from the group comprising C₆₋₁₀aryl, 5-6 membered heteroaryl containing at least one N, O and/or S, and C₄₋₆cycloalkyl; preferably A^{2b} is selected from the group comprising phenyl, 5-6 membered heteroaryl containing at least one N, O and/or S and C₅₋₆cycloalkyl; preferably A^{2b} is phenyl;
   wherein each of said group can be unsubstituted or substituted with one or more R^{2b} wherein each R^{2b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;

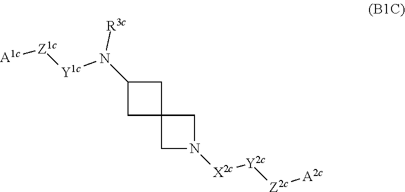

A^{1c} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; preferably A^{1c} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1c} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1c} is selected from the group comprising C₆₋₁₀aryl, C₅₋₆cycloalkyl, 5-6 membered heteroaryl containing at least one N, O and/or S;
   wherein each of said group can be unsubstituted or substituted with one or more R^{1c} wherein each R^{1c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl;
R^{3c} is selected from hydrogen, or C₁₋₆alkyl;
Y^{1c} is a single bond, or is selected from —C(R^{4c})₂—, —NR^{6c}—, —O—, or a 5 membered heteroarylene;
Z^{1c} is a single bond, or is selected from —C(R^{4c})₂—, or —O—;
each R^{4c} is independently selected from hydrogen, halo or C₁₋₆alkyl;
each R^{6c} is independently selected from hydrogen, or C₁₋₆alkyl;
X^{2c} is a single bond or —CO—;
Y^{2c} is a single bond, or —C(R^{5c})₂—,
Z^{2c} is a single bond, or is selected from —(C(R^{5c})₂)ₚ—, or —CO—; wherein p is an integer selected from 1 or 2;
each R^{5c} is independently selected from hydrogen, halo or C₁₋₆alkyl; or two R^{5c} together with the atom(s) to which they are attached can form a C₃₋₆cycloalkyl;
A^{2c} is selected from the group comprising C₆₋₁₀aryl, 5-10 membered heteroaryl containing at least one N, O and/or S, 5-10 membered saturated or partially saturated heterocyclyl containing at least one N, O and/or S, C₃₋₆cycloalkyl, hydrogen, —N(R^{6c})₂, and C₁₋₆alkyl; preferably A^{2c} is selected from the group comprising C₆₋₁₀aryl, 5-9 membered heteroaryl containing at least one N, O and/or S, C₃₋₆cycloalkyl, and C₁₋₄alkyl; preferably A^{2c} is selected from the group comprising C₆₋₁₀aryl, 5-6 membered heteroaryl containing at least one N, O and/or S, and C₄₋₆cycloalkyl; preferably A^{2c} is selected from the group comprising phenyl, 5-6 membered heteroaryl containing at least one N, O and/or S and C₅₋₆cycloalkyl;
   wherein each of said group can be unsubstituted or substituted with one or more R^{2c} wherein each R^{2c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
2. A compound of formula (B1A), (B1B) or (B1C), or a salt, a solvate, a hydrate, a polymorph, a tautomer, a racemate or a stereoisomer thereof; or a prodrug thereof, wherein

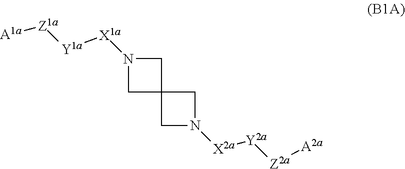

A^{1a} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R^{1a}, wherein each R^{1a} is independently selected from the group comprising halo, hydrogen, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-10 membered heteroaryl, or a 5-10 membered saturated or partially saturated heterocyclyl;
X^{1a} is selected from —CO—, —SO₂—, —C(R^{3a})₂—, or 5-membered heteroarylene;
Y^{1a} is selected from —C(R^{3a})₂—, —NR^{5a}— or —O—; or Y^{1a} is a single bond;
Z^{1a} is a single bond, or is selected from —C(R^{3a})₂—, —NR^{5a}—, or —O—;
each R^{3a} is independently selected from hydrogen, halo or C₁₋₆alkyl;
each R^{5a} is independently selected from hydrogen, or C₁₋₆alkyl;
X^{2a} is a single bond or —CO—;
Y^{2a} is —C(R^{4a})₂— or Y^{2a} is a single bond,
Z^{2a} is a single bond, or is selected from —(C(R^{4a})₂)ₙ—, or —CO—; wherein n is an integer selected from 1 or 2;
each R^{4a} is independently selected from hydrogen, halo or C₁₋₆alkyl; or two R^{4a} together with the atom(s) to which they are attached can form a C₃₋₆cycloalkyl;
A^{2a} is selected from the group comprising C₆₋₁₀aryl, 5-10 membered heteroaryl containing at least one N, O and/or S, 5-10 membered saturated or partially saturated heterocyclyl, and C₃₋₆cycloalkyl; wherein each of said group can be unsubstituted or substituted with one or more R^{2a}; wherein each R^{2a} is independently selected from the group comprising halo, hydrogen, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
with the proviso that:
A^{1a} is not:

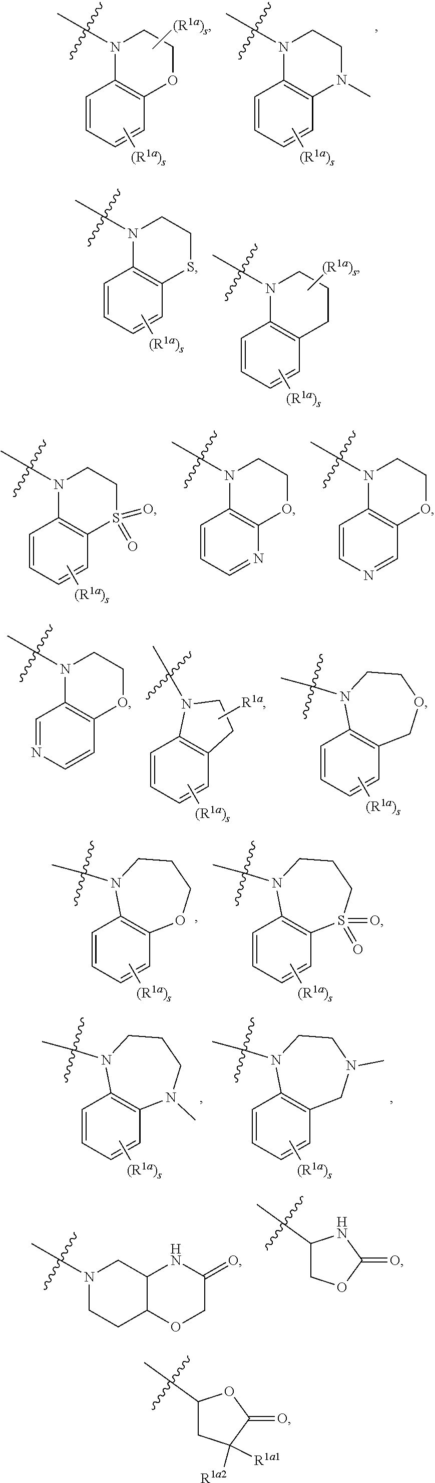

wherein R^{1a1} and R^{1a2} are each independently selected from C₁₋₂alkyl, or R^{1a1} and R^{1a2} together with the carbon atom to which they are attached form a C₃₋₆cycloalkyl, wherein R^{1a} has the same meaning as that defined herein and s is an integer selected from 0, 1 or 2;
A^{2a} is not:

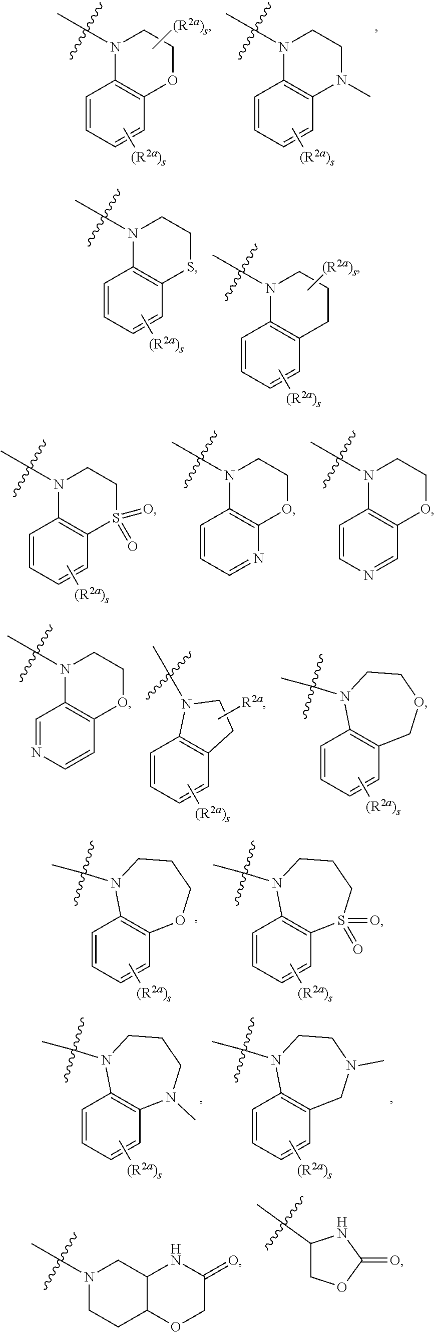

wherein R^{2a} has the same meaning as that defined herein and s is an integer selected from 0, 1 or 2;
with the proviso that said compound is not:
2-Benzyl-6-(toluene-4-sulfonyl)-2,6-diazaspiro[3.3]heptane;
2-(1,3-benzodioxol-5-ylmethyl)-6-[(4-methylphenyl)sulfonyl-2,6-Diazaspiro[3.3]heptane;
2-(1,3-Benzodioxol-5-ylmethyl)-6-(phenylmethyl)-2,6-diazaspiro[3.3]heptane;
2-Benzyl-6-(tosyl)-2,6-diazaspiro[3.3]heptane;
6-benzyl-2,6-diazaspiro[3.3]heptan-2-yl)(3,3,5-trimethyl-2,3-dihydro-1H-pyrrolo[3,2-b]pyridin-1-yl)methanone;
(5-cyclohexyl-1H-pyrazol-3-yl)(6-(cyclopropanecarbonyl)-2,6-diazaspiro[3.3]heptan-2-yl)methanone;
(6-(cyclopropanecarbonyl)-2,6-diazaspiro[3.3]heptan-2-yl)(1-isopropyl-1H-imidazol-4-yl)methanone;
(5-isopropyl-1H-pyrazol-3-yl)(6-(1-methylcyclopropane-1-carbonyl)-2,6-diazaspiro[3.3]heptan-2-yl)methanone;
(5-isopropyl-1H-pyrazol-3-yl)(6-(thiophene-2-carbonyl)-2,6-diazaspiro[3.3]heptan-2-yl)methanone;
[2-(4-fluoro-1-methyl-pyrazole-3-carbonyl)-2,6-diazaspiro[3.3]heptan-6-yl]-(5-isopropyl-1H-pyrazol-3-yl)methanone;

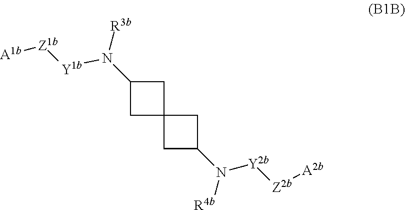

A^{1b} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R^{1b}, wherein each R^{1b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, and C₁₋₆alkylcarbonylamino; and/or two R^{1b} together with the atom(s) to which they are attached can form a C₆₋₁₀aryl, a 5-10 membered heteroaryl, or a 5-10 membered saturated or partially saturated heterocyclyl;
R^{3b} is selected from hydrogen, or C₁₋₆alkyl;
Y^{1b} is selected from —CO—, —C(R^{5b})₂—, or 5-membered heteroarylene;
Z^{1b} is —C(R^{5b})₂—;
R^{4b} is selected from hydrogen, C₁₋₆alkyl, or C₁₋₆alkoxycarbonyl;
each R^{5b} is independently selected from hydrogen, halo or C₁₋₆alkyl;
each R^{5b} is independently selected from hydrogen, or C₁₋₆alkyl;
Y^{2b} is —C(R^{5b})₂—, or a single bond;
Z^{2b} is a single bond, or is selected from —(C(R^{5b})₂)ₘ—, or —CO—; wherein m is an integer selected from 1 or 2;
A^{2b} is selected from the group comprising C₆₋₁₀aryl, 5-10 membered heteroaryl containing at least one N, O and/or S, 5-10 membered saturated or partially saturated heterocyclyl, C₃₋₆cycloalkyl, and C₁₋₆alkyl; wherein each of said group can be unsubstituted or substituted with one or more R^{2b}, wherein each R^{2b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;

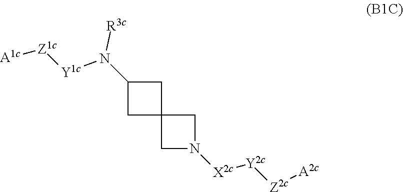

A^{1c} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; wherein each of said group can be unsubstituted or substituted with one or more R^{1c}, wherein each R^{1c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl;
R^{3c} is selected from hydrogen, or C₁₋₆alkyl;
Y^{1c} is a single bond, or is selected from —C(R^{4c})₂—, —NR^{6c}—, —O—, or a 5 membered heteroarylene;
Z^{1c} is a single bond, or is selected from —C(R^{4c})₂—, or —O—;
each R^{4c} is independently selected from hydrogen, halo or C₁₋₆alkyl;
each R^{6c} is independently selected from hydrogen, or C₁₋₆alkyl;
X^{2c} is a single bond or —CO—;
Y^{2c} is a single bond, or —C(R^{5c})₂—,
Z^{2c} is a single bond, or is selected from —(C(R^{5c})₂)ₚ—, or —CO—; wherein p is an integer selected from 1 or 2;
each R^{5c} is independently selected from hydrogen, halo or C₁₋₆alkyl; or two R^{5c} together with the atom(s) to which they are attached can form a C₃₋₆cycloalkyl;
A^{2c} is selected from the group comprising C₆₋₁₀aryl, 5-10 membered heteroaryl containing at least one N, O and/or S, 5-10 membered saturated or partially saturated heterocyclyl containing at least one N, O and/or S, C₃₋₆cycloalkyl, and —N(R^{6c})₂; wherein each of said group can be unsubstituted or substituted with one or more R^{2c}; wherein each R^{2c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
with the proviso that: A^{1c} is not

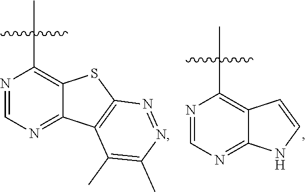

A^{2c} is not

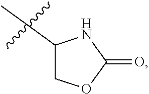 [SMILES: *C1COC(=O)N1]

with the proviso that said compound is not
N-benzyl-2-(6-(2,2,2-trifluoroethyl)thieno[2,3-d]pyrimidin-4-yl)-2-azaspiro[3.3]heptan-6-amine;
N-(2-(2-fluorophenyl)-2-azaspiro[3.3]heptan-6-yl)-6,7-dimethoxy-2,3-dihydro-1H-cyclopenta[b]quinolin-9-amine;
6-((2-((benzyloxy)carbonyl)-2-azaspiro[3.3]heptan-6-yl)amino)-2-chloropyrimidine-4-carboxylic acid;
benzyl 6-((2-chloro-6-(methoxycarbonyl)pyrimidin-4-yl)amino)-2-azaspiro[3.3]heptane-2-carboxylate.
   3. The compound according to statement 1 or 2; wherein
   A^{1a} is selected from the group comprising C₆₋₁₀aryl, C₄₋₆cycloalkyl, 5-9 membered heteroaryl containing at least one N, O and/or S, and 5-9 membered saturated or partially saturated heterocyclyl containing at least one N, O and/or S; preferably A^{1a} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1a} is selected from the group comprising C₆₋₁₀aryl, C₅₋₆cycloalkyl, 5-6 membered heteroaryl containing at least one N, O and/or S;
      wherein each of said group can be unsubstituted or substituted with one or more R^{1a}, wherein each R^{1a} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-10 membered heteroaryl, or a 5-10 membered saturated or partially saturated heterocyclyl; preferably each of said group can be unsubstituted or substituted with one, or more R^{1a}, for example 1, 2, 3 or 4 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, 2, or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, 2 or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, or 2 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, and cyano; and/or two Ria together with the atom(s) to which they are attached can form a C₆₋₁₀aryl, a C₃₋₆cycloalkyl, a 5-9 membered heteroaryl, or a 5-9 membered saturated or partially saturated heterocyclyl;
   X^{1a} is selected from —CO—, —SO₂—, —C(R^{3a})₂—, or 5-membered heteroarylene; preferably X^{1a} is selected from —CO—, —SO₂—, —C(R^{3a})₂—, or 1,2,4-thiadiazolyl; preferably X^{1a} is selected from —CO—, —SO₂—, or —C(R^{3a})₂—;
   Y^{1a} is a single bond, or is selected from —C(R^{3a})₂—, —NR^{5a}— or —O—;
   Z^{1a} is a single bond, or is selected from —C(R^{3a})₂—, —NR^{5a}—, or —O—;
   each R^{3a} is independently selected from hydrogen, halo or C₁₋₆alkyl; preferably each R^{3a} is hydrogen, halo or C₁₋₄alkyl; preferably each R^{3a} is hydrogen, halo or C₁₋₂alkyl;
   each R^{5a} is independently selected from hydrogen, or C₁₋₆alkyl; preferably each R^{5a} is hydrogen, or C₁₋₄alkyl; preferably each R^{5a} is hydrogen, or C₁₋₂alkyl;
   X^{2a} is a single bond or —CO—;
   Y^{2a} is a single bond, or —C(R^{4a})₂—,
   Z^{2a} is a single bond, or is selected from —(C(R^{4a})₂)ₙ—, or —CO—; wherein n is an integer selected from 1 or 2;
   each R^{4a} is independently selected from hydrogen, halo or C₁₋₆alkyl; or two R^{4a} together with the atom(s) to which they are attached can form a C₃₋₆cycloalkyl; preferably each R^{4a} is independently selected from hydrogen, halo or C₁₋₄alkyl; or two R^{4a} together with the atom(s) to which they are attached can form a C₃₋₅cycloalkyl; preferably each R^{4a} is independently selected from hydrogen, halo or C₁₋₂alkyl; or two R^{4a} together with the atom(s) to which they are attached can form a C₃₋₄cycloalkyl;
   A^{2a} is selected from the group comprising C₆₋₁₀aryl, 5-9 membered heteroaryl containing at least one N, O and/or S, and C₃₋₆cycloalkyl; preferably A^{2a} is selected from the group comprising C₆₋₁₀aryl, 5-6 membered heteroaryl containing at least one N, O and/or S, and C₄₋₆cycloalkyl; preferably A^{2a} is selected from the group comprising phenyl, 5-6 membered heteroaryl containing at least one N, O and/or S and C₅₋₆cycloalkyl;
      wherein each of said group can be unsubstituted or substituted with one or more R^{2a} wherein each R^{2a} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2a}, for example 1, 2, 3 or 4 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
   A^{1b} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; preferably A^{1b} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1b} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1b} is selected from the group comprising C₆₋₁₀aryl, C₅₋₆cycloalkyl, 5-6 membered heteroaryl containing at least one N, O and/or S; preferably A^{1b} is phenyl;
      wherein each of said group can be unsubstituted or substituted with one or more R^{1b} wherein each R^{1b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, and C₁₋₆alkylcarbonylamino; and/or two R^{1b} together with the atom(s) to which they are attached can form a C₆₋₁₀aryl, a 5-10 membered heteroaryl, or a 5-10 membered saturated or partially saturated heterocyclyl; preferably each of said group can be unsubstituted or substituted with one, or more R^{1b}, for example 1, 2, 3 or 4 R^{1b}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, 2, or 3 R^{1b}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, 2 or 3 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, or 2 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, and cyano; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl;
   R^{3b} is selected from hydrogen, or C₁₋₆alkyl; preferably each R^{3b} is hydrogen, or C₁₋₄alkyl; preferably each R^{3b} is hydrogen, or C₁₋₂alkyl;
   Y^{1b} is selected from —CO—, —C(R^{5b})₂—, or 5-membered heteroarylene; preferably Y^{1b} is selected from —CO—, —C(R^{5b})₂—, or 1,2,4-thiadiazolyl;
   Z^{1b} is —C(R^{5b})₂—;
   R^{4b} is selected from hydrogen, C₁₋₆alkyl, or C₁₋₆alkoxycarbonyl; preferably R^{4b} is selected from hydrogen, C₁₋₄alkyl, or C₁₋₄alkoxycarbonyl; preferably R^{4b} is selected from hydrogen, C₁₋₂ alkyl, or C₁₋₂alkoxycarbonyl;
   each R^{5b} is independently selected from hydrogen, halo or C₁₋₆alkyl; preferably each R^{5b} is independently selected from hydrogen, halo or C₁₋₄alkyl; preferably each R^{5b} is independently selected from hydrogen, halo or C₁₋₂alkyl; preferably each R^{5b} is independently selected from hydrogen, or C₁₋₂alkyl;
   each R^{5b} is independently selected from hydrogen, or C₁₋₆alkyl; preferably each R^{5b} is independently selected from hydrogen, or C₁₋₄alkyl; preferably each R^{5b} is independently selected from hydrogen, or C₁₋₂alkyl;
   Y^{2b} is —C(R^{5b})₂—, or a single bond;
   Z^{2b} is a single bond, or is selected from —(C(R^{5b})₂)ₘ—, or —CO—; wherein m is an integer selected from 1 or 2;
   A^{2b} is selected from the group comprising C₆₋₁₀aryl, 5-10 membered heteroaryl containing at least one N, O and/or S, 5-10 membered saturated or partially saturated heterocyclyl, C₃₋₆cycloalkyl, and C₁₋₆alkyl; preferably A^{2b} is selected from the group comprising C₆₋₁₀aryl, 5-9 membered heteroaryl containing at least one N, O and/or S, C₃₋₆cycloalkyl, and C₁₋₄alkyl; preferably A^{2b} is selected from the group comprising C₆₋₁₀aryl, 5-6 membered heteroaryl containing at least one N, O and/or S, and C₄₋₆cycloalkyl; preferably A^{2b} is selected from the group comprising phenyl, 5-6 membered heteroaryl containing at least one N, O and/or S and C₅₋₆cycloalkyl; preferably A^{2b} is phenyl;
      wherein each of said group can be unsubstituted or substituted with one or more R^{2b} wherein each R^{2b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, 2, 3, or 4 R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{5b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, 2, or 3, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, or 2, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;
   A^{1c} is selected from the group comprising C₆₋₁₀aryl, C₃₋₆cycloalkyl, 5-10 membered heteroaryl containing at least one N, O and/or S, and 5-10 membered saturated or partially saturated heterocyclyl containing at least one N, O and/or S; preferably A^{1c} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1c} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1c} is selected from the group comprising C₆₋₁₀aryl, C₅₋₆cycloalkyl, 5-6 membered heteroaryl containing at least one N, O and/or S;
      wherein each of said group can be unsubstituted or substituted with one or more R^{1c}, wherein each R^{1c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, 2, 3, or 4 R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, 2, or 3, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, and C₃₋₆cycloalkyl; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, or 2, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, and cyano; and/or two R^{1c} together with the atom(s) to which they are attached can form a C₆₋₁₀aryl, a 5-10 membered heteroaryl, or a 5-10 membered saturated or partially saturated heterocyclyl;
   R^{3c} is selected from hydrogen, or C₁₋₆alkyl; preferably each R^{3c} is hydrogen, or C₁₋₄alkyl; preferably each R^{3c} is hydrogen, or C₁₋₂alkyl;
   Y^{1c} is a single bond, or is selected from —C(R^{4c})₂—, —NR^{6c}—, —O—, or a 5 membered heteroarylene; preferably Y^{1c} is a single bond, or is selected from —C(R^{4c})₂—, —NR^{6c}—, —O—, or 1,2,4-thiadiazolyl;
   Z^{1c} is a single bond, or is selected from —C(R^{4c})₂—, or —O—;
   each R^{4c} is independently selected from hydrogen, halo or C₁₋₆alkyl; preferably each R^{4c} is hydrogen, halo or C₁₋₄alkyl; preferably each R^{4c} is hydrogen, halo or C₁₋₂alkyl;
   each R^{6c} is independently selected from hydrogen, or C₁₋₆alkyl; preferably each R^{6c} is hydrogen, or C₁₋₄alkyl; preferably each R^{6c} is hydrogen, or C₁₋₂alkyl;
   X^{2c} is a single bond or —CO—;
   Y^{2c} is a single bond, or —C(R^{5c})₂,
   Z^{2c} is a single bond, or is selected from —(C(R^{5c})₂)ₚ—, or —CO—; wherein p is an integer selected from 1 or 2;
   each R^{5c} is independently selected from hydrogen, halo or C₁₋₆alkyl; or two R^{5c} together with the atom(s) to which they are attached can form a C₃₋₆cycloalkyl; preferably each R^{5c} is independently selected from hydrogen, halo or C₁₋₄alkyl; or two R^{5c} together with the atom(s) to which they are attached can form a C₃₋₅cycloalkyl; preferably each R^{5c} is independently selected from hydrogen, halo or C₁₋₂alkyl; or two R^{5c} together with the atom(s) to which they are attached can form a C₃₋₄cycloalkyl;
   A^{2c} is selected from the group comprising C₆₋₁₀aryl, 5-10 membered heteroaryl containing at least one N, O and/or S, 5-10 membered saturated or partially saturated heterocyclyl containing at least one N, O and/or S, C₃₋₆cycloalkyl, and —N(R^{6c})₂; preferably A^{2c} is selected from the group comprising C₆₋₁₀aryl, 5-9 membered heteroaryl containing at least one N, O and/or S, and C₃₋₆cycloalkyl; preferably A^{2c} is selected from the group comprising C₆₋₁₀aryl, 5-6 membered heteroaryl containing at least one N, O and/or S, and C₄₋₆cycloalkyl; preferably A^{2c} is selected from the group comprising phenyl, 5-6 membered heteroaryl containing at least one N, O and/or S and C₅₋₆cycloalkyl;
      wherein each of said group can be unsubstituted or substituted with one or more R^{2c} wherein each R^{2c} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2c}, for example 1, 2, 3 or 4 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2c}, for example 1, 2, or 3 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{6c})₂N-carbonyl, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
   4. The compound according to any one of statements 1-3, wherein
   A^{1a} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1a} is selected from the group comprising C₆₋₁₀aryl, C₅₋₆cycloalkyl, 5-6 membered heteroaryl containing at least one N, O and/or S;
      each of said group can be unsubstituted or substituted with one, or more R^{1a}, for example 1, 2, 3 or 4 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, 2, or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, 2 or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, or 2 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, and cyano; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl;
   X^{1a} is selected from —CO—, —SO₂—, —C(R^{3a})₂—, or 1,2,4-thiadiazolyl; preferably X^{1a} is selected from —CO—, —SO₂—, or —C(R^{3a})₂—;
   Y^{1a} is a single bond, or is selected from —C(R^{3a})₂—, —NR^{5a}— or —O—;
   Z^{1a} is a single bond, or is selected from —C(R^{3a})₂—, —NR^{5a}—, or —O—;
   each R^{3a} is hydrogen, halo or C₁₋₄alkyl; preferably each R^{3a} is hydrogen, halo or C₁₋₂alkyl;
   each R^{5a} is hydrogen, or C₁₋₄alkyl; preferably each R^{5a} is hydrogen, or C₁₋₂alkyl;
   X^{2a} is a single bond or —CO—;
   Y^{2a} is a single bond, or —C(R^{4a})₂—,
   Z^{2a} is a single bond, or is selected from —(C(R^{4a})₂)ₙ—, or —CO—; wherein n is an integer selected from 1 or 2;
   each R^{4a} is independently selected from hydrogen, halo or C₁₋₄alkyl; or two R^{4a} together with the atom(s) to which they are attached can form a C₃₋₅cycloalkyl; preferably each R^{4a} is independently selected from hydrogen, halo or C₁₋₂alkyl; or two R^{4a} together with the atom(s) to which they are attached can form a C₃₋₄cycloalkyl;
   A^{2a} is selected from the group comprising C₆₋₁₀aryl, 5-6 membered heteroaryl containing at least one N, O and/or S, and C₄₋₆cycloalkyl; preferably A^{2a} is selected from the group comprising phenyl, 5-6 membered heteroaryl containing at least one N, O and/or S and C₅₋₆cycloalkyl;
      wherein each of said group can be unsubstituted or substituted with one or more R^{2a}, for example 1, 2, 3 or 4 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
   A^{1b} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1b} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1b} is selected from the group comprising C₆₋₁₀aryl, C₅₋₆cycloalkyl, 5-6 membered heteroaryl containing at least one N, O and/or S; preferably Alb is phenyl;
      each of said group can be unsubstituted or substituted with one, or more R^{1b}, for example 1, 2, 3 or 4 R^{1b}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, 2, or 3 R^{1b}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, 2 or 3 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, or 2 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, and cyano; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl;
   each R^{3b} is hydrogen, or C₁₋₄alkyl; preferably each R^{3b} is hydrogen, or C₁₋₂alkyl;
   Y^{1b} is selected from —CO—, —C(R^{5b})₂—, or 1,2,4-thiadiazolyl;
   Z^{1b} is —C(R^{5b})₂—;
   R^{4b} is selected from hydrogen, C₁₋₄alkyl, or C₁₋₄alkoxycarbonyl; preferably R^{4b} is selected from hydrogen, C₁₋₂alkyl, or C₁₋₂alkoxycarbonyl;
   each R^{5b} is independently selected from hydrogen, halo or C₁₋₄alkyl; preferably each R^{5b} is independently selected from hydrogen, halo or C₁₋₂alkyl; preferably each R^{5b} is independently selected from hydrogen, or C₁₋₂alkyl;
   each R^{6b} is independently selected from hydrogen, or C₁₋₄alkyl; preferably each R^{6b} is independently selected from hydrogen, or C₁₋₂alkyl;
   Y^{2b} is —C(R^{5b})₂—, or a single bond;
   Z^{2b} is a single bond, or is selected from —(C(R^{5b})₂)ₘ—, or —CO—; wherein m is an integer selected from 1 or 2;
   A^{2b} is selected from the group comprising C₆₋₁₀aryl, 5-9 membered heteroaryl containing at least one N, O and/or S, C₃₋₆cycloalkyl, and C₁₋₄alkyl; preferably A^{2b} is selected from the group comprising C₆₋₁₀aryl, 5-6 membered heteroaryl containing at least one N, O and/or S, and C₄₋₆cycloalkyl; preferably A^{2b} is selected from the group comprising phenyl, 5-6 membered heteroaryl containing at least one N, O and/or S and C₅₋₆cycloalkyl; preferably A^{2b} is phenyl;
      wherein each of said group can be unsubstituted or substituted with one or more R^{2b}, for example 1, 2, 3, or 4 R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, 2, or 3, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, or 2, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;
   A^{1c} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1c} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1c} is selected from the group comprising C₆₋₁₀aryl, C₅₋₆cycloalkyl, 5-6 membered heteroaryl containing at least one N, O and/or S;
      wherein each of said group can be unsubstituted or substituted with one or more R^{1c}, for example 1, 2, 3, or 4 R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, 2, or 3, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, and C₃₋₆cycloalkyl; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, or 2, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, and cyano; and/or two R^{1c} together with the atom(s) to which they are attached can form a C₆₋₁₀aryl, a 5-10 membered heteroaryl, or a 5-10 membered saturated or partially saturated heterocyclyl;
   each R^{3c} is hydrogen, or C₁₋₄alkyl; preferably each R^{3c} is hydrogen, or C₁₋₂alkyl;
   Y^{1c} is a single bond, or is selected from —C(R^{4c})₂—, —NR^{6c}—, —O—, or 1,2,4-thiadiazolyl;
   Z^{1c} is a single bond, or is selected from —C(R^{4c})₂—, or —O—;
   each R^{4c} is hydrogen, halo or C₁₋₄alkyl; preferably each R^{4c} is hydrogen, halo or C₁₋₂alkyl;
   each R^{6c} is hydrogen, or C₁₋₄alkyl; preferably each R^{6c} is hydrogen, or C₁₋₂alkyl;
   X^{2c} is a single bond or —CO—;
   Y^{2c} is a single bond, or —C(R^{5c})₂—,
   Z^{2c} is a single bond, or is selected from —(C(R^{5c})₂)ₚ—, or —CO—; wherein p is an integer selected from 1 or 2;
   each R^{5c} is independently selected from hydrogen, halo or C₁₋₄alkyl; or two R^{5c} together with the atom(s) to which they are attached can form a C₃₋₅cycloalkyl; preferably each R^{5c} is independently selected from hydrogen, halo or C₁₋₂alkyl; or two R^{5c} together with the atom(s) to which they are attached can form a C₃₋₄cycloalkyl;
   A^{2c} is selected from the group comprising C₆₋₁₀aryl, 5-9 membered heteroaryl containing at least one N, O and/or S, and C₃₋₆cycloalkyl; preferably A^{2c} is selected from the group comprising C₆₋₁₀aryl, 5-6 membered heteroaryl containing at least one N, O and/or S, and C₄₋₆cycloalkyl; preferably A^{2c} is selected from the group comprising phenyl, 5-6 membered heteroaryl containing at least one N, O and/or S and C₅₋₆cycloalkyl;
      wherein each of said group can be unsubstituted or substituted with one or more R^{2c}, for example 1, 2, 3 or 4 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2c}, for example 1, 2, or 3 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{6c})₂N-carbonyl, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
   5. The compound according to any one of statements 1-4, wherein
   A^{1a} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1a} is selected from the group comprising C₆₋₁₀aryl, C₅₋₆cycloalkyl, 5-6 membered heteroaryl containing at least one N, O and/or S;
      each of said group can be unsubstituted or substituted with one, or more R^{1a}, for example 1, 2, 3 or 4 R¹a, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, 2, or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R¹a, for example 1, 2 or 3 R¹a, wherein each R^{1a} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, or 2 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, and cyano; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl;
   A^{2a} is selected from the group comprising C₆₋₁₀aryl, 5-6 membered heteroaryl containing at least one N, O and/or S, and C₄₋₆cycloalkyl; preferably A^{2a} is selected from the group comprising phenyl, 5-6 membered heteroaryl containing at least one N, O and/or S and C₅₋₆cycloalkyl;
      wherein each of said group can be unsubstituted or substituted with one or more R^{2a}, for example 1, 2, 3 or 4 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
   A^{1b} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1b} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1b} is selected from the group comprising C₆₋₁₀aryl, C₅₋₆cycloalkyl, 5-6 membered heteroaryl containing at least one N, O and/or S; preferably Alb is phenyl;
      each of said group can be unsubstituted or substituted with one, or more R^{1b}, for example 1, 2, 3 or 4 R^{1b}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, C₃₋₆cycloalkyl, (R^{5a})₂N-carbonyl, and C₁₋₆alkylcarbonylamino; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, 2, or 3 R^{1b}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, 2 or 3 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, or 2 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, and cyano; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl;
   A^{2b} is selected from the group comprising C₆₋₁₀aryl, 5-9 membered heteroaryl containing at least one N, O and/or S, C₃₋₆cycloalkyl, and C₁₋₄alkyl; preferably A^{2b} is selected from the group comprising C₆₋₁₀aryl, 5-6 membered heteroaryl containing at least one N, O and/or S, and C₄₋₆cycloalkyl; preferably A^{2b} is selected from the group comprising phenyl, 5-6 membered heteroaryl containing at least one N, O and/or S and C₅₋₆cycloalkyl; preferably A^{2b} is phenyl;
      wherein each of said group can be unsubstituted or substituted with one or more R^{2b}, for example 1, 2, 3, or 4 R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, 2, or 3, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{5b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, or 2, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;
   A^{1c} is selected from the group comprising C₆₋₁₀aryl, C₃₋₆cycloalkyl, 5-9 membered heteroaryl containing at least one N, O and/or S, and 5-9 membered saturated or partially saturated heterocyclyl containing at least one N, O and/or S; preferably A^{1c} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1c} is selected from the group comprising C₆₋₁₀aryl, C₅₋₆cycloalkyl, 5-6 membered heteroaryl containing at least one N, O and/or S;
      wherein each of said group can be unsubstituted or substituted with one or more R^{1c}, for example 1, 2, 3, or 4 R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, 2, or 3, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, and C₃₋₆cycloalkyl; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, or 2, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, and cyano; and/or two R^{1c} together with the atom(s) to which they are attached can form a C₆₋₁₀aryl, a 5-10 membered heteroaryl, or a 5-10 membered saturated or partially saturated heterocyclyl;
   A^{2c} is selected from the group comprising C₆₋₁₀aryl, 5-9 membered heteroaryl containing at least one N, O and/or S, and C₃₋₆cycloalkyl; preferably A^{2c} is selected from the group comprising C₆₋₁₀aryl, 5-6 membered heteroaryl containing at least one N, O and/or S, and C₄₋₆cycloalkyl; preferably A^{2c} is selected from the group comprising phenyl, 5-6 membered heteroaryl containing at least one N, O and/or S and C₅₋₆cycloalkyl;
      wherein each of said group can be unsubstituted or substituted with one or more R^{2c}, for example 1, 2, 3 or 4 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2c}, for example 1, 2, or 3 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{6c})₂N-carbonyl, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
   6. The compound according to any one of statements 1-5, wherein
   X^{1a} is selected from —CO—, —SO₂—, —C(R^{3a})₂—, or 1,2,4-thiadiazolyl;
   Y^{1a} is a single bond, or is selected from —C(R^{3a})₂—, —NR^{5a}— or —O—;
   Z^{1a} is a single bond, or is selected from —C(R^{3a})₂—, —NR^{5a}—, or —O—;
   each R^{3a} is hydrogen, halo or C₁₋₂alkyl;
   each R^{5a} is hydrogen, or C₁₋₂alkyl;
   X^{2a} is a single bond or —CO—;
   Y^{2a} is a single bond, or —C(R^{4a})₂—,
   Z^{2a} is a single bond, or is selected from —(C(R^{4a})₂)ₙ—, or —CO—; wherein n is an integer selected from 1 or 2;
   each R^{4a} is independently selected from hydrogen, halo or C₁₋₂alkyl; or two R^{4a} together with the atom(s) to which they are attached can form a C₃₋₄cycloalkyl.
   7. The compound according to any one of statements 1-5; wherein
   each R^{3b} is hydrogen, or C₁₋₂alkyl;
   Y^{1b} is selected from —CO—, —C(R^{5b})₂—, or 1,2,4-thiadiazolyl;
   Z^{1b} is —C(R^{5b})₂—;
   R^{4b} is selected from hydrogen, C₁₋₂alkyl, or C₁₋₂alkoxycarbonyl;
   each R^{5b} is independently selected from hydrogen, halo or C₁₋₂alkyl; preferably each R^{5b} is independently selected from hydrogen, or C₁₋₂alkyl;
   each R^{6b} is independently selected from hydrogen, or C₁₋₂alkyl;
   Y^{2b} is —C(R^{5b})₂—, or a single bond;
   Z^{2b} is a single bond, or is selected from —(C(R^{5b})₂)ₘ—, or —CO—; wherein m is an integer selected from 1 or 2.
   8. The compound according to any one of statements 1-5, wherein
   each R^{3c} is hydrogen, or C₁₋₂alkyl;
   Y^{1c} is a single bond, or is selected from —C(R^{4c})₂—, —NR^{6c}—, —O—, or a 1,2,4-thiadiazolyl;
   Z^{1c} is a single bond, or is selected from —C(R^{4c})₂—, or —O—;
   each R^{4c} is hydrogen, halo or C₁₋₂alkyl;
   each R^{6c} is hydrogen, or C₁₋₂alkyl;
   X^{2c} is a single bond or —CO—;
   Y^{2c} is a single bond, or —C(R^{5c})₂—,
   Z^{2c} is a single bond, or is selected from —(C(R^{5c})₂)ₚ—, or —CO—; wherein p is an integer selected from 1 or 2;
   ach R^{5c} is independently selected from hydrogen, halo or C₁₋₂alkyl; or two R^{5c} together with the atom(s) to which they are attached can form a C₃₋₄cycloalkyl.
   9. The compound according to any one of statements 1-6, wherein
   X^{1a} is selected from —CO—, —SO₂—, or —C(R^{3a})₂—;
   Y^{1a} is a single bond, or is selected from —C(R^{3a})₂—, —NR^{5a}— or —O—;
   Z^{1a} is a single bond, or is selected from —C(R^{3a})₂—, —NR^{5a}—, or —O—;
   each R^{3a} is hydrogen; each R^{5a} is hydrogen;
   X^{2a} is a single bond or —CO—;
   Y^{2a} is a single bond, or —C(R^{4a})₂—,
   Z^{2a} is a single bond, or is selected from —(C(R^{4a})₂)ₙ—, or —CO—; wherein n is an integer selected from 1 or 2;
   each R^{4a} is independently selected from hydrogen.
   10. The compound according to any one of statements 1-5, 7, wherein
   each R^{3b} is hydrogen;
   Y^{1b} is selected from —CO—, —C(R^{5b})₂—;
   Z^{1b} is —C(R^{5b})₂—;
   R^{4b} is selected from hydrogen;
   each R^{5b} is hydrogen;
   each R^{6b} is hydrogen;
   Y^{2b} is —C(R^{5b})₂—, or a single bond;
   Z^{2b} is a single bond, or is selected from —(C(R^{5b})₂)ₘ—, or —CO—; wherein m is an integer selected from 1 or 2.
   11. The compound according to any one of statements 1-5, 8, wherein
   each R^{3c} is hydrogen;
   Y^{1c} is a single bond, or is selected from —C(R^{4c})₂—, —NR^{6c}, or —O—;
   Z^{1c} is a single bond, or is selected from —C(R^{4c})₂—, or —O—;
   each R^{4c} is hydrogen;
   each R^{6c} is hydrogen;
   X^{2c} is a single bond or —CO—;
   Y^{2c} is a single bond, or —C(R^{5c})₂—,
   Z^{2c} is a single bond, or is selected from —(C(R^{5c})₂)ₚ—, or —CO—; wherein p is an integer selected from 1 or 2;
   ach R^{5c} is hydrogen.
   12. The compound according to any one of statements 1-11, having structural formula (B1A1), (B1A2), (B1A3), (B1A4), (B1A5), (B1A6), (B1B1), (B1B2),

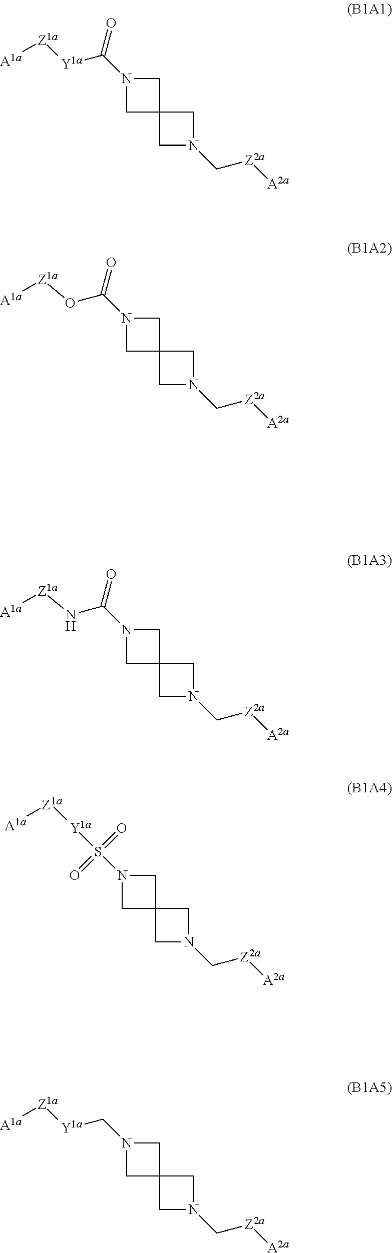

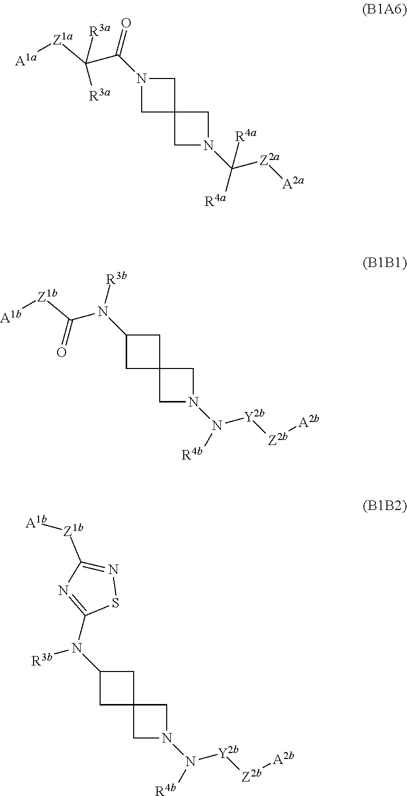

wherein A^{1a}, Y^{1a}, Z^{1a}, R^{2a}, R^{3a}, R^{4a}, Y^{2a}, Z^{2a}, A^{1b}, Y^{1b}, Z^{1b}, R^{2b}, R^{3b}, R^{4b}, Y2b Z^{2b}, A^{2b}, A^{1c}, Y^{1c}, Z^{1c}, R^{2c}, R^{3c}, R^{4c}, X^{2c}, yY^{2c}, Z^{2c} and A^{2c} have the same meaning as that defined in any one of statement 1-11, and na is an integer selected from 1, 2, 3 or 4; nb is an integer selected from 1, 2, 3 or 4; and each nc and mc is an integer selected from 1, 2, 3 or 4.
13. The compound according to any one of statements 1-12, wherein
A^{1a} is selected from the group comprising C₆₋₁₀aryl, C₅₋₆cycloalkyl, 5-6 membered heteroaryl containing at least one N, O and/or S;
   each of said group can be unsubstituted or substituted with one, or more R^{1a}, for example 1, 2, or 3 R¹a, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, 2 or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, or 2 R¹a, wherein each R^{1a} is independently selected from the group comprising hydrogen, halo, C₁₋₆alkyl, C₁₋₆alkoxy, haloC₁₋₆alkyl, haloC₁₋₆alkoxy, and cyano; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl;
A^{2a} is selected from the group comprising phenyl, 5-6 membered heteroaryl containing at least one N, O and/or S and C₅₋₆cycloalkyl;
   wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
A^{1b} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1b} is selected from the group comprising C₆₋₁₀aryl, C₅₋₆cycloalkyl, 5-6 membered heteroaryl containing at least one N, O and/or S; preferably A^{1b} is phenyl;
   each of said group can be unsubstituted or substituted with one, or more R^{1b}, for example 1, 2, or 3 R^{1b}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, 2 or 3 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, or 2 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, and cyano; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl;
A^{2b} is selected from the group comprising C₆₋₁₀aryl, 5-6 membered heteroaryl containing at least one N, O and/or S, and C₄₋₆cycloalkyl; preferably A^{2b} is selected from the group comprising phenyl, 5-6 membered heteroaryl containing at least one N, O and/or S and C₅₋₆cycloalkyl; preferably A^{2b} is phenyl;
   wherein each of said group can be unsubstituted or substituted with one or more R^{2b}, for example 1, 2, or 3, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, or 2, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;
A^{1c} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1c} is selected from the group comprising C₆₋₁₀aryl, C₅₋₆cycloalkyl, 5-6 membered heteroaryl containing at least one N, O and/or S;
   wherein each of said group can be unsubstituted or substituted with one or more R^{1c}, for example 1, 2, or 3, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, and C₃₋₆cycloalkyl; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, or 2, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, and cyano; and/or two R^{1c} together with the atom(s) to which they are attached can form a C₆₋₁₀aryl, a 5-10 membered heteroaryl, or a 5-10 membered saturated or partially saturated heterocyclyl;
A^{2c} is selected from the group comprising C₆₋₁₀aryl, 5-6 membered heteroaryl containing at least one N, O and/or S, and C₄₋₆cycloalkyl; preferably A^{2c} is selected from the group comprising phenyl, 5-6 membered heteroaryl containing at least one N, O and/or S and C₅₋₆cycloalkyl;
   wherein each of said group can be unsubstituted or substituted with one or more R^{2c}, for example 1, 2, or 3 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{6c})₂N-carbonyl, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
14. The compound according to any one of statements 1-13, having structural formula (B1A8), (B1A9), (B1A10), (B1A11), (B1A12), (B1A13),

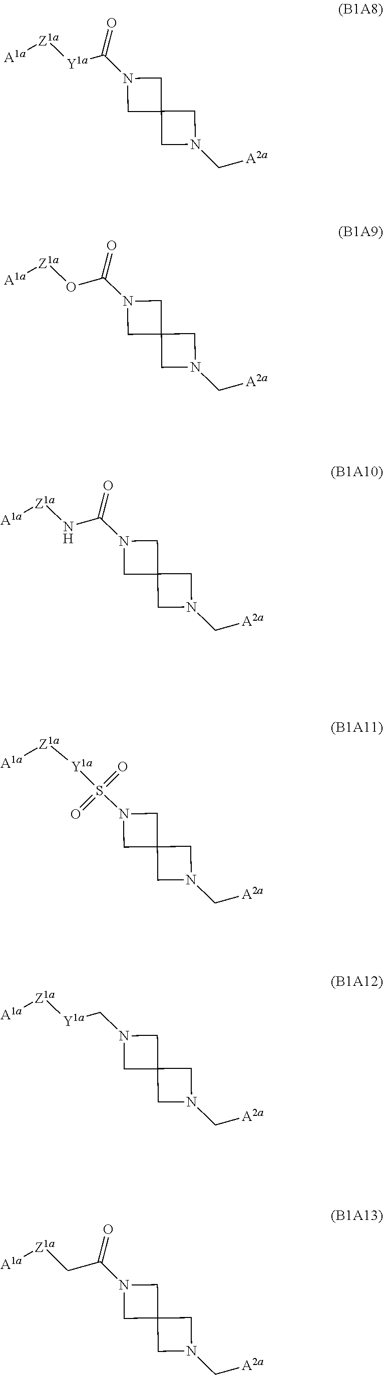

wherein A^{1a}, Y^{1a}, Z^{1a}, and A^{2a} have the same meaning as that defined in any one of statements 1-13.
15. The compound according to any one of statements 1-14, having structural formula (B31A14), (B1A15), (B1A16), (1A17), (B1A18), (B1A19), (B1A20), (B1A21), (B1A22), (B1A23), (B1A24),

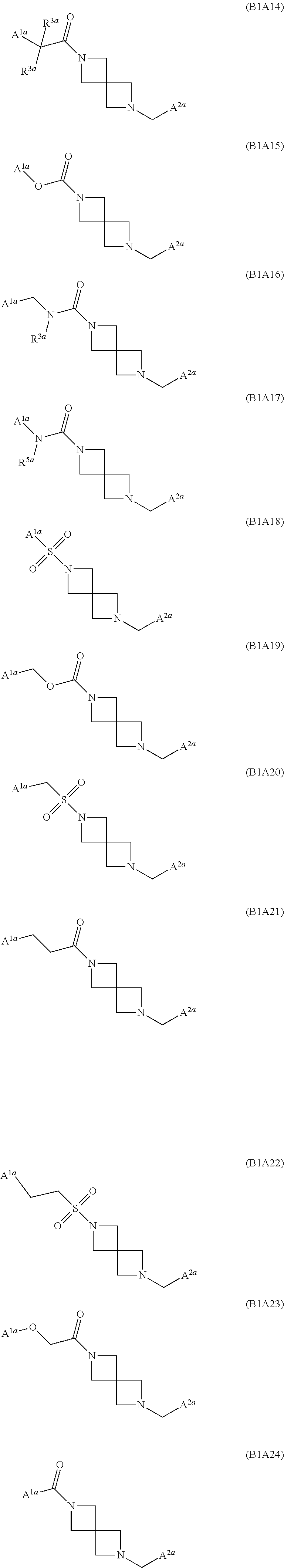

wherein A^{1a}, and A^{2a} have the same meaning as that defined in any one of statements 1-14.
16. The compound according to any one of statements 1-15, wherein
A^{1a} is selected from the group comprising C₆₋₁₀aryl, C₅₋₆cycloalkyl, 5-6 membered heteroaryl containing at least one N, O and/or S;
   each of said group can be unsubstituted or substituted with one, or more R^{1a}, for example 1, 2, or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, 2 or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, or 2 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, and cyano; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl;
A^{2a} is selected from the group comprising C₆₋₁₀aryl, 5-6 membered heteroaryl containing at least one N, O and/or S and C₅₋₆cycloalkyl;
   wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
A^{1b} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1b} is selected from the group comprising C₆₋₁₀aryl, C₅₋₆cycloalkyl, 5-6 membered heteroaryl containing at least one N, O and/or S; preferably A^{1b} is phenyl;
   each of said group can be unsubstituted or substituted with one, or more R^{1b}, for example 1, 2 or 3 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, or 2 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, and cyano; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl;
A^{2b} is selected from the group comprising C₆₋₁₀aryl, 5-6 membered heteroaryl containing at least one N, O and/or S, and C₄₋₆cycloalkyl; preferably A^{2b} is selected from the group comprising phenyl, 5-6 membered heteroaryl containing at least one N, O and/or S and C₅₋₆cycloalkyl; preferably A^{2b} is phenyl;
   wherein each of said group can be unsubstituted or substituted with one or more R^{2b}, for example 1, 2, or 3, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, or 2, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{5b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;
A^{1c} is selected from the group comprising C₆₋₁₀aryl, C₄₋₆cycloalkyl, 5-6 membered heteroaryl containing at least one N, O and/or S, and 5-6 membered saturated or partially saturated heterocyclyl containing at least one N, O and/or S; preferably A^{1c} is selected from the group comprising C₆₋₁₀aryl, C₅₋₆cycloalkyl, 5-6 membered heteroaryl containing at least one N, O and/or S;
   wherein each of said group can be unsubstituted or substituted with one or more R^{1c}, for example 1, 2, or 3, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, and C₃₋₆cycloalkyl; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, or 2, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, and cyano; and/or two R^{1c} together with the atom(s) to which they are attached can form a C₆₋₁₀aryl, a 5-10 membered heteroaryl, or a 5-10 membered saturated or partially saturated heterocyclyl;
A^{2c} is selected from the group comprising C₆₋₁₀aryl, 5-6 membered heteroaryl containing at least one N, O and/or S, and C₄₋₆cycloalkyl; preferably A^{2c} is selected from the group comprising phenyl, 5-6 membered heteroaryl containing at least one N, O and/or S and C₅₋₆cycloalkyl;
   wherein each of said group can be unsubstituted or substituted with one or more R^{2c}, for example 1, 2, or 3 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{6c})₂N-carbonyl, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
17. The compound according to any one of statements 1-16, wherein
A^{1a} is selected from the group comprising phenyl, C₅₋₆cycloalkyl, 5-6 membered heteroaryl containing at least one N;
   each of said group can be unsubstituted or substituted with one, or more R^{1a}, for example 1, 2 or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1a}, for example 1, or 2 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, and cyano; and/or two R^{1a} together with the atom(s) to which they are attached can form a C₆₋₁₀aryl, a C₃₋₆cycloalkyl, a 5-9 membered heteroaryl, or a 5-9 membered saturated or partially saturated heterocyclyl;
A^{2a} is selected from the group comprising phenyl, 5-6 membered heteroaryl containing at least one N, O and/or S and C₅₋₆cycloalkyl;
   wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;
A^{1b} is selected from the group comprising phenyl, C₅₋₆cycloalkyl, 5-6 membered heteroaryl containing at least one N, O and/or S; preferably A^{1b} is phenyl;
   each of said group can be unsubstituted or substituted with one, or more R^{1b}, for example 1, 2 or 3 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, cyano, and hydroxy; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl; preferably each of said group can be unsubstituted or substituted with one, or more R^{1b}, for example 1, or 2 R^{1b}, wherein each R^{1b} is independently selected from the group comprising hydrogen, halo, C₁₋₆alkyl, C₁₋₆alkoxy, haloC₁₋₆alkyl, haloC₁₋₆alkoxy, and cyano; and/or two R^{1b} together with the atom(s) to which they are attached can form a C₆₋₁₀aryl, a C₃₋₆cycloalkyl, a 5-9 membered heteroaryl, or a 5-9 membered saturated or partially saturated heterocyclyl;
A^{2b} is selected from the group comprising phenyl, 5-6 membered heteroaryl containing at least one N, O and/or S and C₅₋₆cycloalkyl; preferably A^{2b} is phenyl;
   wherein each of said group can be unsubstituted or substituted with one or more R^{2b}, for example 1, 2, or 3, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{2b}, for example 1, or 2, R^{2b}, wherein each R^{2b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl;
A^{1c} is selected from the group comprising phenyl, C₄₋₆cycloalkyl, 5-6 membered heteroaryl containing at least one N, O and/or S, and 5-6 membered saturated or partially saturated heterocyclyl containing at least one N, O and/or S; preferably A^{1c} is selected from the group comprising C₆₋₁₀aryl, C₅₋₆cycloalkyl, 5-6 membered heteroaryl containing at least one N, O and/or S;
   wherein each of said group can be unsubstituted or substituted with one or more R^{1c}, for example 1, 2, or 3, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, and C₃₋₆cycloalkyl; and/or two R^{1c} together with the atom(s) to which they are attached can form a C₆₋₁₀aryl, a 5-10 membered heteroaryl, or a 5-10 membered saturated or partially saturated heterocyclyl; preferably wherein each of said group can be unsubstituted or substituted with one or more R^{1c}, for example 1, or 2, R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, and cyano; and/or two R^{1c} together with the atom(s) to which they are attached can form a C₆₋₁₀aryl, a 5-10 membered heteroaryl, or a 5-10 membered saturated or partially saturated heterocyclyl;
A^{2c} is selected from the group comprising phenyl, 5-6 membered heteroaryl containing at least one N, O and/or S, and C₄₋₆cycloalkyl; preferably A^{2c} is selected from the group comprising phenyl, 5-6 membered heteroaryl containing at least one N, O and/or S and C₅₋₆cycloalkyl;
   wherein each of said group can be unsubstituted or substituted with one or more R^{2c}, for example 1, 2, or 3 R^{2c}; wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{6c})₂N-carbonyl, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.
18. The compound according to any one of statements 1-17, having structural formula (B1A25),

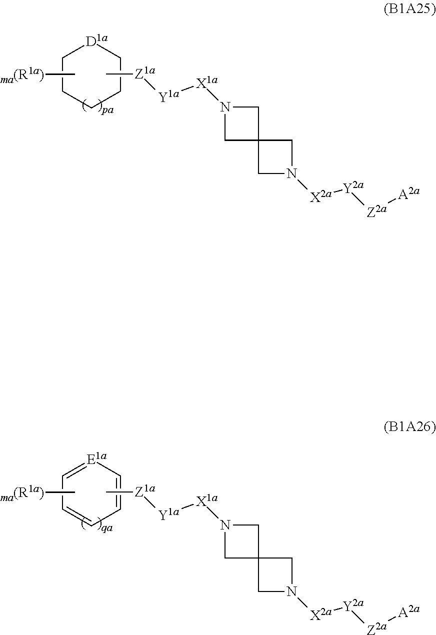

wherein R^{a}, Y^{1a}, Z^{1a}, X^{2a}, X^{a}, Y^{2a}, Z^{2a} and A^{2a} have the same meaning as that defined in any one of statements 1-17, and ma is an integer selected from 1, 2, 3 or 4, pa is an integer selected from 0 or 1, qa is an integer selected from 0 or 1, D^{1a} is selected from CH₂, O or NH, and E^{1a} is selected from CH, N, O or S.
19. The compound according to any one of statements 1-18, having structural formula (B1A27), (B1A28),

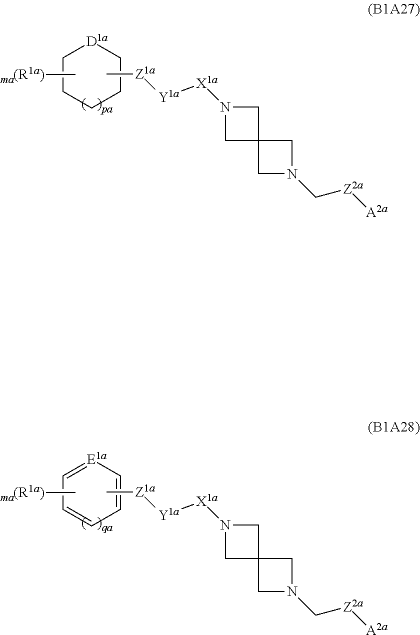

wherein R^{1a}, Y^{1a}, Z^{1a}, X^{1a}, Y^{2a}, Z^{2a} and A^{2a} have the same meaning as that defined in any one of statements 1-18, and ma is an integer selected from 1, 2, 3 or 4, pa is an integer selected from 0 or 1, qa is an integer selected from 0 or 1, D^{1a} is selected from CH₂, O or NH, and E^{1a} is selected from CH, N, O or S.
20. The compound according to any one of statements 1-19, having structural formula (B1A29), (B1A30), (B1A31), (B1A32),

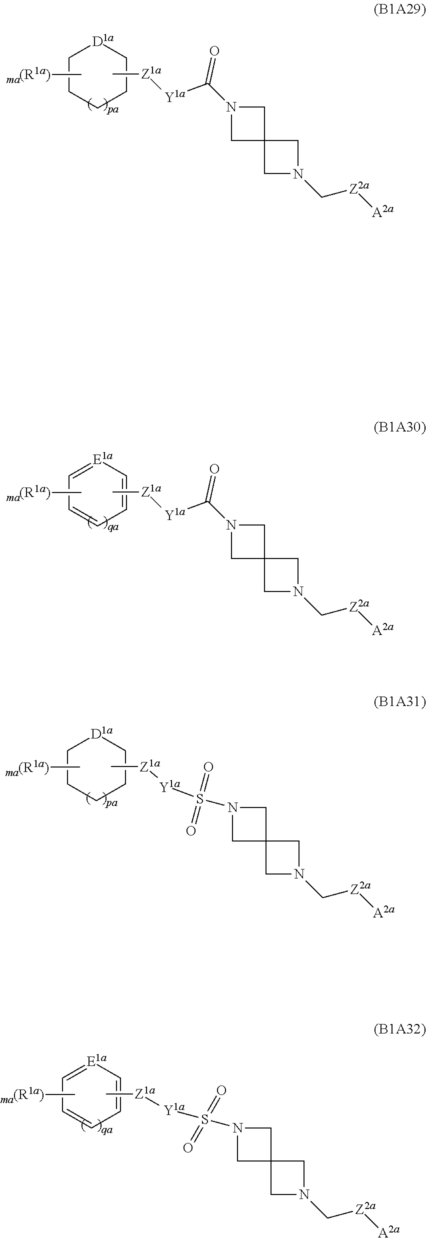

wherein R^{1a}, Y^{1a}, Z^{1a}, X^{1a}, Y^{2a}, Z^{2a} and A^{2a} have the same meaning as that defined in any one of statements 1-19, and ma is an integer selected from 1, 2, 3 or 4, pa is an integer selected from 0 or 1, qa is an integer selected from 0 or 1, D^{1a} is selected from CH₂, O or NH, and E^{1a} is selected from CH, N, O or S.
21. The compound according to any one of statements 1-20, having structural formula (B1A33), (B1A34), (B1A35), (B1A36), (B1A37), (B1A38), (B1A39), (B1A40), (B1A41), (B1A42), (B1A43),

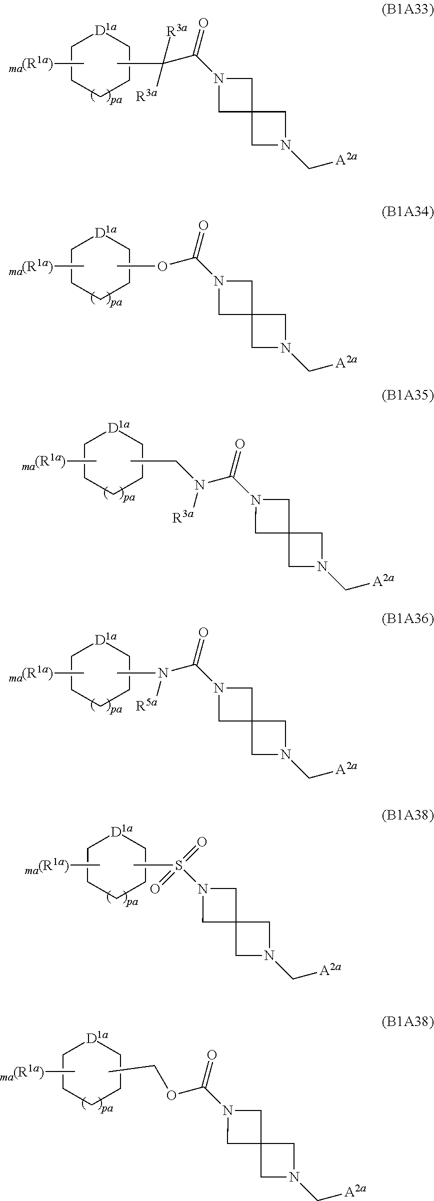

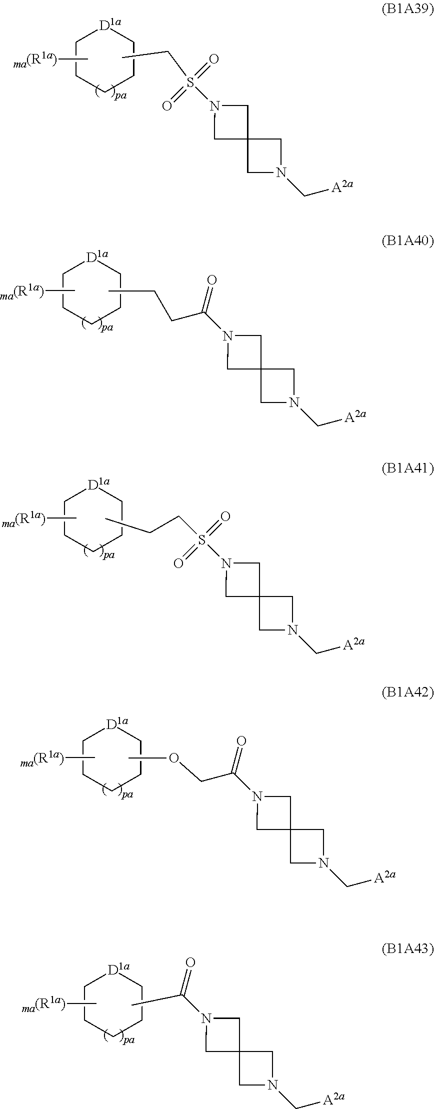

wherein R^{1a}, and A^{2a} have the same meaning as that defined in any one of statements 1-20, and ma is an integer selected from 1, 2, 3 or 4, pa is an integer selected from 0 or 1, D^{1a} is selected from CH₂, O or NH.
22. The compound according to any one of statements 1-21, having structural formula (B1A44), (B1A45), (B1A46), (B1A47), (B1A48), (B1A49), (B1A50), (B1A51), (B1A52), (B1A53), (B1A54),

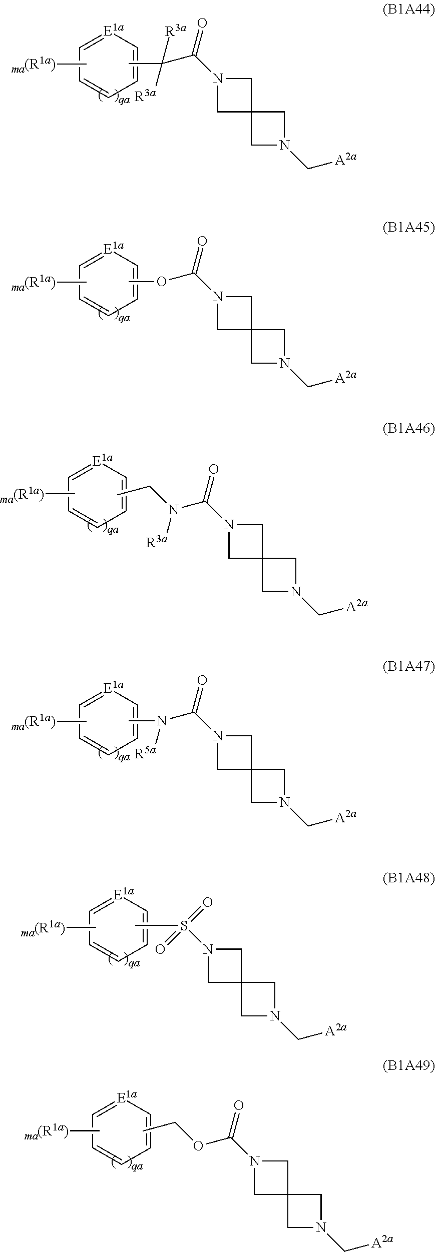

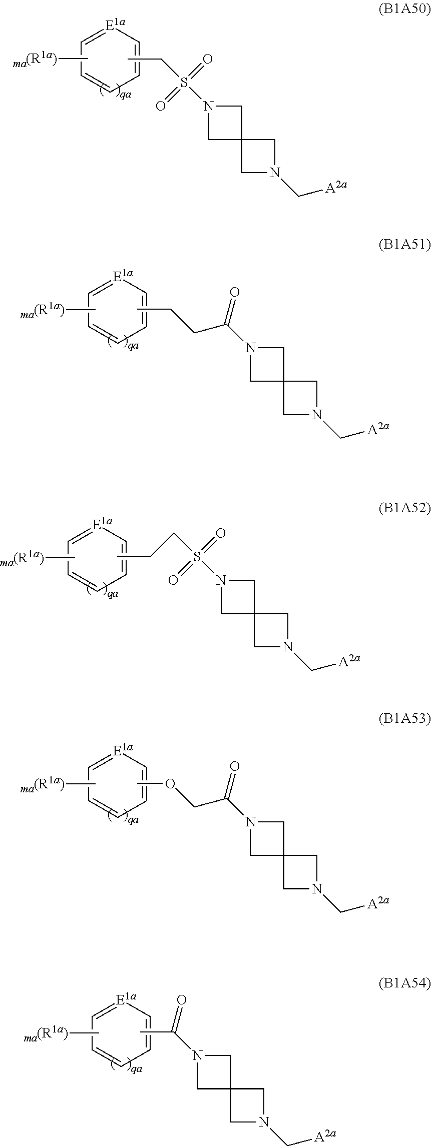

wherein R^{1a}, and A^{2a} have the same meaning as that defined in any one of statements 1-21, and ma is an integer selected from 1, 2, 3 or 4, qa is an integer selected from 0 or 1, E^{1a} is selected from CH, O or N.
23. The compound according to any one of statements 1-22, having structural formula (B1A55), (B1A56), (B1A57), (B1A58), (B1A59), (B1A60), (B1A61), (B1A62), (B1A63), (B1A64), (B1A65),

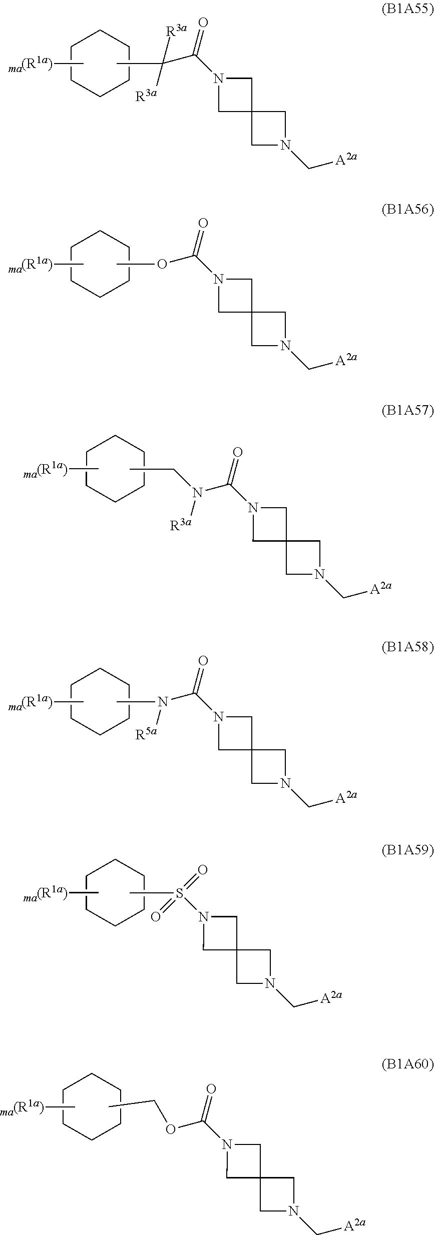

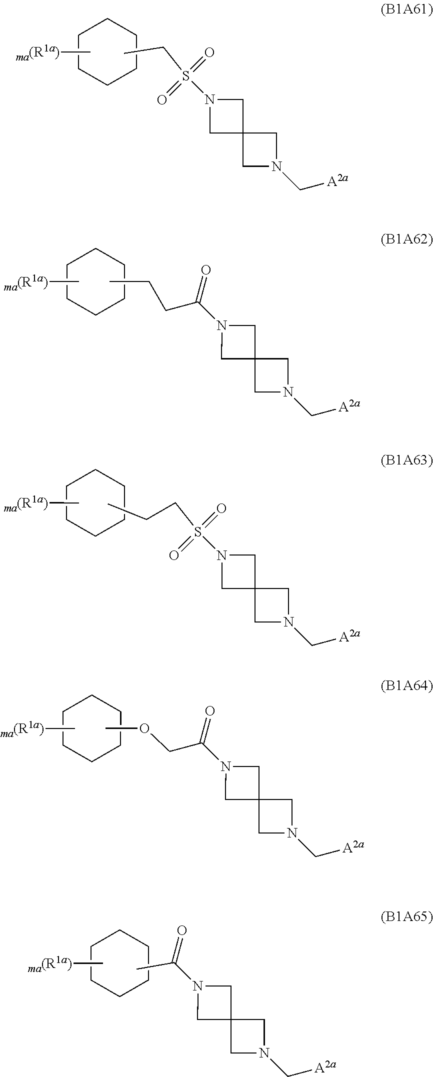

wherein R^{1a}, and A^{2a} have the same meaning as that defined in any one of statements 1-22, and ma is an integer selected from 1, 2, 3 or 4.
24. The compound according to any one of statements 1-23, having structural formula (B1A66), (B1A67), (B1A68), (B1A69), (B1A70), (B1A71), (B1A72), (B1A73), (B1A74), (B1A75), (B1A76),

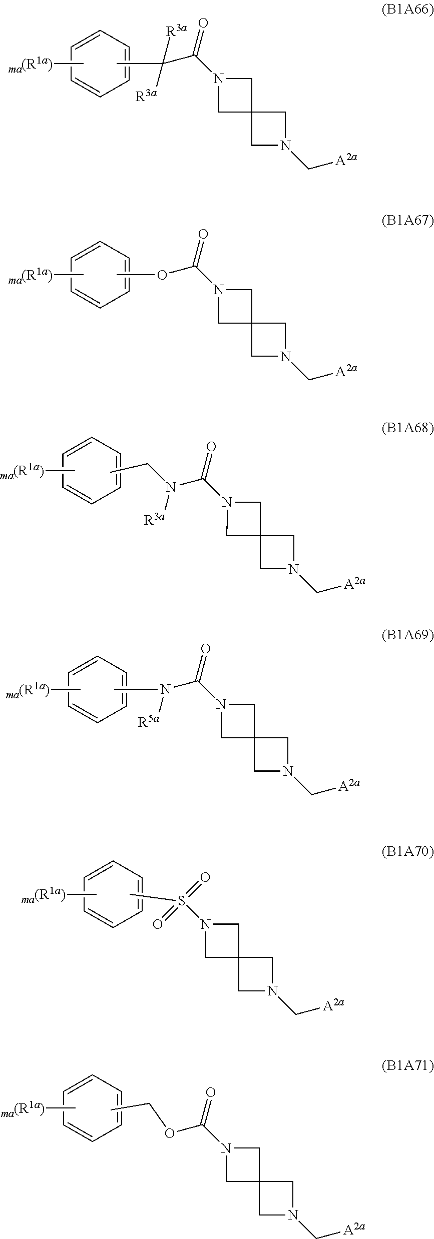

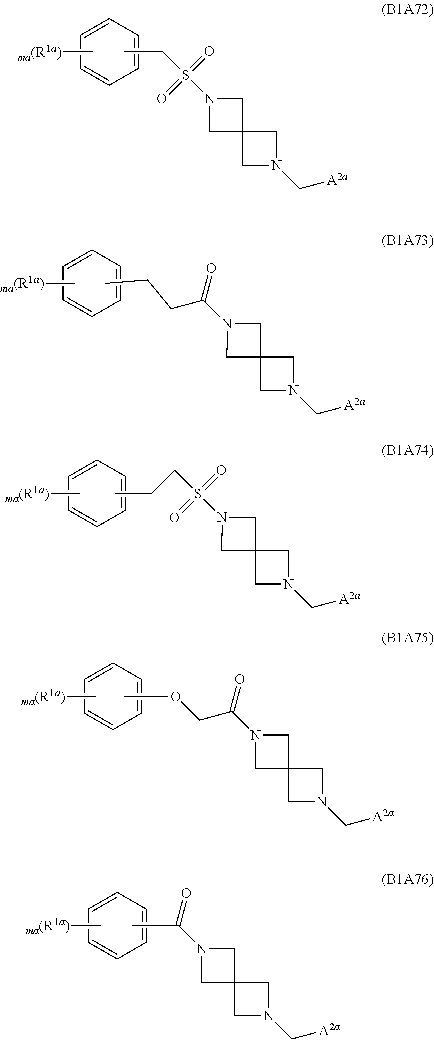

wherein R^{1a}, and A^{2a} have the same meaning as that defined in any one of statements 1-23, and ma is an integer selected from 1, 2, 3 or 4.
25. The compound according to any one of statements 1-24, having structural formula (B1A77), (B1A78)

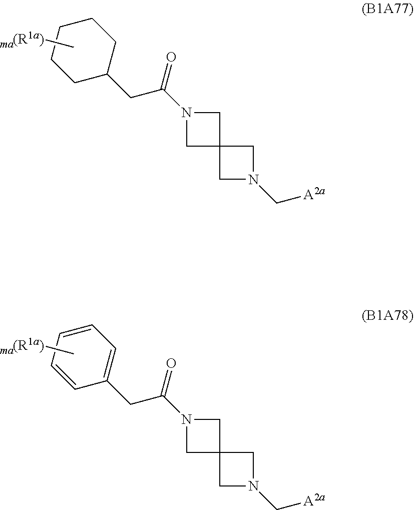

wherein R^{1a}, and A^{2a} have the same meaning as that defined in any one of statements 1-24, and ma is an integer selected from 1, 2, 3 or 4.
26. The compound according to any one of statements 1-25, having structural formula (B1A79), (B1A80),

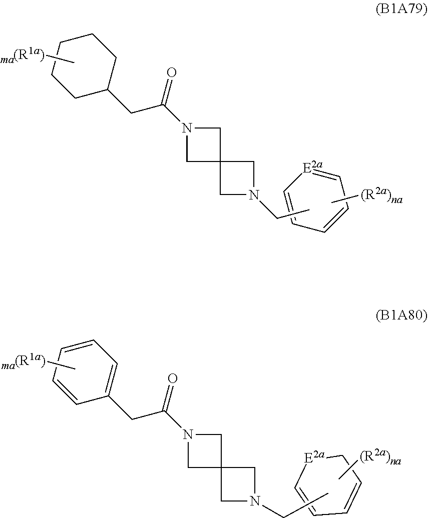

wherein R^{1a}, R^{2a} and A^{2a} have the same meaning as that defined any one of statements 1-25, and ma is an integer selected from 1, 2, 3 or 4, na is an integer selected from 1, 2, 3 or 4, E^{1a} is selected from CH, O or N.
27. A compound according to any one of statements 1-26, wherein said compound is selected from the group comprising the compounds listed in Table 1 B.
28. A pharmaceutical composition comprising a compound according to any one of statements 1-27, and at least one pharmaceutical acceptable carrier.
29. A compound according to any one of statements 1-27, or a pharmaceutical composition according to statement 28 for use as a medicine or medicament.
30. A compound according to any one of statements 1-27 for use as therapeutically active substance.
31. A compound according to any one of statements 1-27, or a pharmaceutical composition according to statement 28, for use in the prevention and/or treatment of neurodegenerative disorders, such as neurodegenerative disorders characterized by cytotoxic TAU misfolding and/or aggregation, in order to delay or halt the progression of such diseases.
32. The compound of the pharmaceutical composition for use according to statement 31, wherein said disease is selected from the group consisting of Parkinson's disease, Alzheimer's disease, diffuse Lewy body disease, amyotrophic lateral sclerosis, Niemann Pick disease, Hallervorden Spatz syndrome, Down syndrome, Pick's disease, progressive supranuclear palsy, vascular dementia, neuroaxonal dystrophy, Huntington's disease, frontotemporal lobar degeneration (FTLD), multiple system atrophy and Creutzfeld-Jacob's disease, preferably said neurodegenerative disorder is Alzheimer's disease.
33. A compound according to any one of statements 1-27, or a pharmaceutical composition according to statement 28, in the prevention and/or treatment of a disease selected from the group consisting of Parkinson's disease, Alzheimer's disease, diffuse Lewy body disease, amyotrophic lateral sclerosis, Niemann Pick disease, Hallervorden Spatz syndrome, Down syndrome, Pick's disease, progressive supranuclear palsy, vascular dementia, neuroaxonal dystrophy, Huntington's disease, frontotemporal lobar degeneration (FTLD), multiple system atrophy and Creutzfeld-Jacob's disease, preferably said neurodegenerative disorder is Alzheimer's disease.
34. A method for the prevention and/or treatment of neurodegenerative disorders, such as neurodegenerative disorders characterized by cytotoxic TAU misfolding and/or aggregation in order to delay or halt the progression of such diseases, which method comprises administering an effective amount of a compound according to any one of statements 1-27, to a subject in need thereof.
35. The method according to statement 34, wherein the disease is selected from the group consisting of Parkinson's disease, Alzheimer's disease, diffuse Lewy body disease, amyotrophic lateral sclerosis, Niemann Pick disease, Hallervorden Spatz syndrome, Down syndrome, Pick's disease, progressive supranuclear palsy, vascular dementia, neuroaxonal dystrophy, Huntington's disease, frontotemporal lobar degeneration (FTLD), multiple system atrophy and Creutzfeld-Jacob's disease, preferably said neurodegenerative disorder is Alzheimer's disease.

The invention provides new compound of formula (B1A), (B1B) or (B1C) as defined herein (including all embodiments thereof as described herein).

In some embodiments of compound of formula (B1A) as defined herein (including all embodiments thereof as described herein),
A^{1a} is selected from the group comprising phenyl, C₅₋₆cycloalkyl, 5-6 membered heteroaryl containing at least one N, O and/or S;
   wherein each of said group can be unsubstituted or substituted with one, two or three R^{1a} wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₃₋₆cycloalkyl; and/or two R^{1a} together with the atom(s) to which they are attached can form a C₆₋₁₀aryl, a C₃₋₆cycloalkyl, a 5-10 membered heteroaryl, or a 5-10 membered saturated or partially saturated heterocyclyl;
X^{1a} is selected from —CO—, —SO₂—, —C(R^{3a})₂—, or 1,2,4-thiadiazolyl;
Y^{1a} is a single bond, or is selected from —C(R^{3a})₂—, —NR^{5a}— or —O—;
Z^{1a} is a single bond, or is selected from —C(R^{3a})₂—, —NR^{5a}—, or —O—;
each R^{3a} is hydrogen;
each R^{5a} hydrogen;
X^{2a} is a single bond or —CO—;
Y^{2a} is a single bond, or —C(R^{4a})₂—,
Z^{2a} is a single bond, or is selected from —(C(R^{4a})₂)ₙ—, or —CO—; wherein n is an integer selected from 1 or 2;
each R^{4a} is hydrogen;
A^{2a} is selected from the group comprising phenyl, or 5-6 membered heteroaryl containing at least one N, O and/or S and C₅₋₆cycloalkyl;
   wherein each of said group can be unsubstituted or substituted with one, two or three R^{2a} wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a C₆₋₁₀aryl, a 5-10 membered heteroaryl, or a 5-10 membered saturated or partially saturated heterocyclyl;

In some embodiments of compound of formula (B1B) as defined herein (including all embodiments thereof as described herein),
A^{1b} is selected from the group comprising phenyl, C₅₋₆cycloalkyl, 5-6 membered heteroaryl containing at least one N, O and/or S; preferably A^{1b} is phenyl;
   wherein each of said group can be unsubstituted or substituted with one, two, or three Rib wherein each R^{1b} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, and C₁₋₆alkylcarbonylamino; and/or two R^{1b} together with the atom(s) to which they are attached can form a C₆₋₁₀aryl, a 5-10 membered heteroaryl, or a 5-10 membered saturated or partially saturated heterocyclyl;
R^{3b} is hydrogen;
Y^{1b} is selected from —CO—, —C(R^{5b})₂—, or 1,2,4-thiadiazolyl;
Z^{1b} is —C(R^{5b})₂—;
R^{4b} is hydrogen; each R^{5b} is hydrogen; each R^{5b} is hydrogen;
Y^{2b} is —C(R^{5b})₂—, or a single bond;
Z^{2b} is a single bond, or is selected from —(C(R^{5b})₂)ₘ—, or —CO—; wherein m is an integer selected from 1 or 2:
A^{2b} is selected from the group comprising phenyl, 5-6 membered heteroaryl containing at least one N, O and/or S and C₅₋₆cycloalkyl; preferably A^{2b} is phenyl;
   wherein each of said group can be unsubstituted or substituted with one, two or three R^{2b} wherein each R^{2b} is independently selected from the group comprising hydrogen, halo, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6b})₂N-carbonyl, —SO₂—R^{6b}, and C₁₋₆alkylcarbonylamino; and/or two R^{2b} together with the atom(s) to which they are attached can form a C₆₋₁₀aryl, a 5-10 membered heteroaryl, or a 5-10 membered saturated or partially saturated heterocyclyl.

In some embodiments of compound of formula (B1C) as defined herein (including all embodiments thereof as described herein),
A^{1c} is selected from the group comprising phenyl, C₅₋₆cycloalkyl, 5-6 membered heteroaryl containing at least one N, O and/or S;
   wherein each of said group can be unsubstituted or substituted with one, two or three R^{1c}, wherein each R^{1c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, and C₁₋₆alkylcarbonylamino; and/or two R^{1c} together with the atom(s) to which they are attached can form a C₆₋₁₀aryl, a 5-10 membered heteroaryl, or a 5-10 membered saturated or partially saturated heterocyclyl;
R^{3c} is hydrogen;
Y^{1c} is a single bond, or is selected from —C(R^{4c})₂—, —NR^{6c}—, —O—, or a 1,2,4-thiadiazolyl;
Z^{1c} is a single bond, or is selected from —C(R^{4c})₂—, or —O—;
each R^{4c} is hydrogen; each R^{6c} is hydrogen;
X^{2c} is a single bond or —CO—;
Y^{2c} is a single bond, or —C(R^{5c})₂—,
Z^{2c} is a single bond, or is selected from —(C(R^{5c})₂)ₚ—, or —CO—; wherein p is an integer selected from 1 or 2;
each R^{5c} is hydrogen;
A^{2c} is selected from the group comprising phenyl, 5-6 membered heteroaryl containing at least one N, O and/or S and C₅₋₆cycloalkyl;
   wherein each of said group can be unsubstituted or substituted with one, two or three R^{2c} wherein each R^{2c} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, C₃₋₆cycloalkyl, (R^{6c})₂N-carbonyl, —SO₂—R^{6c}, and C₁₋₆alkylcarbonylamino; and/or two R^{2c} together with the atom(s) to which they are attached can form a C₆₋₁₀aryl, a 5-10 membered heteroaryl, or a 5-10 membered saturated or partially saturated heterocyclyl.

In a preferred embodiment of the invention, the compound is selected from the group of compounds listed in Table 1 B below, or a salt, a solvate, a hydrate, a polymorph, a tautomer, a racemate, a stereoisomer, or a prodrug thereof.

Any reference to a compound of formula (B1A), (B1B) or (B1C) as defined herein (including all embodiments thereof as described herein) also includes isomers such as stereoisomers and tautomers, salts such as pharmaceutically and/or physiologically acceptable salts, hydrates, solvates, polymorphs, and prodrugs of such compounds unless expressly indicated otherwise.

The term “isomers” as used herein means all possible isomeric forms, including tautomeric and stereochemical forms, which the compounds of formulae herein may possess, but not including position isomers. Typically, the structures shown herein exemplify one tautomeric or resonance form of the compounds, but the corresponding alternative configurations are contemplated as well.

Depending on its substitution pattern, the compounds of the present invention as defined herein (including all embodiments thereof as described herein) may or may not have one or more optical stereocenters and may or may not exist as different enantiomers or diastereomers. Any such enantiomers, diastereomers or other optical isomers are encompassed by the scope of the invention. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration. The terms R- or S-configuration are used herein in accordance with Chemical Abstracts nomenclature. The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

The term “pharmaceutically acceptable salts” relates to any salts that the compounds may form, and which are suitable for administration to subjects, in particular human subjects, according to the present invention. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, ethanolamine, and the like. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g., hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e., ethanedioic), malonic, succinic (i.e., butanedioic acid), maleic, fumaric, malic, tartaric, citric, methanesulfonic, ethanesulfonic, benzenesulfonic, p-toluenesulfonic, cyclohexanesulfamic, salicylic (i.e., 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention as defined herein (including all embodiments thereof as described herein) also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of a hydrogen atom or an amino group include salts of organic carboxylic acids such as acetic, benzoic, lactic, fumaric, tartaric, maleic, malonic, malic, isethionic, lactobionic, and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

Non-limiting examples of suitable such salts include but are not limited to acid addition salts, formed either with inorganic acids such as hydrochloric acid, hydrobromic acid, sulfuric acid, nitric acid, phosphoric acid, and the like, or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. Other salts include 2,2-dichloroacetate, adipate, alginate, ascorbate, aspartate, 2-acetamidobenzoate, caproate, caprate, camphorate, cyclamate, laurylsulfate, edisilate, esylate, isethionate, formate, galactarate, gentisate, gluceptate, glucuronate, oxoglutarate, hippurate, lactobionate, napadisilate, xinafoate, nicotinate, oleate, orotate, oxalate, palmitate, embonate, pidolate, p-aminosalicylate, sebacate, tannate, rhodanide, undecylenate, and the like; or salts formed when an acidic proton present in the parent compound is replaced, such as with ammonia, arginine, benethamine, benzathine, calcium, choline, deanol, diethanolamine, diethylamine, ethanolamine, ethylendiamine, meglumine, glycine, hydrabamine, imidazole, lysine, magnesium, hydroxyethylmorpholine, piperazine, potassium, epolamine, sodium, trolamine, tromethamine, or zinc.

The present invention includes within its scope solvates of the compounds as defined herein (including all embodiments thereof as described herein). The term “solvates” refers to crystals formed by an active compound and a second component (solvent) which, in isolated form, is liquid at room temperature. Such solvates may be formed with common organic solvents, e.g., hydrocarbon solvents such as benzene or toluene; chlorinated solvents such as chloroform or dichloromethane; alcoholic solvents such as methanol, ethanol, or isopropanol; ethereal solvents such as diethyl ether or tetrahydrofuran; or ester solvents such as ethyl acetate. Alternatively, the solvates of the compounds herein may be formed with water, in which case they will be hydrates.

Another embodiment of this invention relates to various precursor or “prodrug” forms of the compounds of the present invention. It may be desirable to formulate the compounds of the present invention in the form of a chemical species which itself is not significantly biologically-active, but which when delivered to the animal, mammal or human will undergo a chemical reaction catalysed by the normal function of the body of the fish, inter alia, enzymes present in the stomach or in blood serum, said chemical reaction having the effect of releasing a compound as defined herein. In general, such prodrugs will be functional derivatives of the compounds described herein which are readily convertible in vivo, e.g., by endogenous enzymes in the gut or the blood, into the required GPR17 modulating compounds described herein. The term “prodrug” thus relates to these species which are converted in vivo into the active pharmaceutical ingredient.

The prodrugs of the compounds of the present invention as defined herein (including all embodiments thereof as described herein) can have any form suitable to the formulator, for example, esters are non-limiting common prodrug forms. In the present case, however, the prodrug may necessarily exist in a form wherein a covalent bond is cleaved by the action of an enzyme present at the target locus. For example, a C—C covalent bond may be selectively cleaved by one or more enzymes at said target locus and, therefore, a prodrug in a form other than an easily hydrolysable precursor, inter alia an ester, an amide, and the like, may be used. The counterpart of the active pharmaceutical ingredient in the prodrug can have different structures such as an amino acid or peptide structure, alkyl chains, sugar moieties and others as known in the art.

For the purpose of the present invention the term “therapeutically suitable prodrug” can be defined herein as a compound modified in such a way as to be transformed in vivo to the therapeutically active form, whether by way of a single or by multiple biological transformations, when in contact with the tissues of the animal, mammal, or human to which the prodrug has been administered, and without undue toxicity, irritation, or allergic response, and achieving the intended therapeutic outcome.

More specifically the term “prodrug”, as used herein, relates to an inactive or significantly less active derivative of a compound such as represented by the structural formulae herein described, which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

The compound of the present invention as defined herein (including all embodiments thereof as described herein) may also exist in different crystal forms, i.e., as polymorphs and mixtures thereof, all of which are encompassed by the present invention.

The term “polymorph” refers to a particular crystalline form of a chemical compound that can crystallize in different crystalline forms, these forms having different arrangements and/or conformations of the molecules in the crystal lattice. Different crystalline forms usually have different X-ray diffraction patterns, infrared spectra, melting points, density, hardness, crystal shape, optical and electrical properties, stability, and solubility. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound described herein can exhibit beneficial effects (e.g., suitability for preparation of useful formulations, improved biological performance) relative to another polymorph or a mixture of polymorphs of the same compound. Preparation and isolation of a particular polymorph of a compound can be achieved by methods known to those skilled in the art including, for example, crystallization using selected solvents and temperatures. Recrystallization solvent, rate of crystallization, storage temperature, and other factors may cause one crystal form to dominate. Various polymorphs of a compound can be prepared by crystallization under different conditions. For a comprehensive discussion of polymorphism see Rolf Hilfiker, Ed., Polymorphism in the Pharmaceutical Industry, Wiley-VCH, Weinheim, 2006.

The present invention also compasses pharmaceutical compositions comprising at least one compound according to the invention as defined herein (including all embodiments thereof as described herein), and at least one pharmaceutically acceptable carrier.

The term “pharmaceutically acceptable carrier^{1′} refers to a diluent, adjuvant, excipient, or carrier, or other ingredient with which a compound of the invention is administered and which a person of skilled in the art would understand to be pharmaceutically acceptable.

Tablets will contain excipients, glidants, fillers, binders, and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid, and the like.

Subsequently, the term “pharmaceutically acceptable carrier” as used herein means any material or substance with which the active ingredient is formulated in order to facilitate its application or dissemination to the locus to be treated, for instance by dissolving, dispersing, or diffusing the said composition, and/or to facilitate its storage, transport, or handling without impairing its effectiveness. The pharmaceutically acceptable carrier may be a solid or a liquid or a gas which has been compressed to form a liquid, e.g., the compositions of this invention can suitably be used as concentrates, emulsions, solutions, granulates, dusts, sprays, aerosols, suspensions, ointments, creams, tablets, pellets, or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, e.g., carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronization, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g., the sodium or potassium salts of oleic or stearic acid, or of natural fatty acid mixtures obtainable from coconut oil or tallow oil. Synthetic surfactants include sodium or calcium salts of polyacrylic acids; fatty sulfonates and sulfates; sulfonated benzimidazole derivatives and alkylarylsulfonates. Fatty sulfonates or sulfates are usually in the form of alkaline or alkaline-earth metal salts, unsubstituted ammonium salts or ammonium salts substituted with an alkyl or acyl group having from 8 to 22 carbon atoms, e.g., the sodium or calcium salt of lignosulfonic acid or dodecylsulfonic acid or a mixture of fatty alcohol sulfates obtained from natural fatty acids, alkaline or alkaline-earth metal salts of sulfuric or sulfonic acid esters (such as sodium lauryl sulfate) and sulfonic acids of fatty alcohol/ethylene oxide adducts. Suitable sulfonated benzimidazole derivatives preferably contain 8 to 22 carbon atoms. Examples of alkylarylsulfonates are the sodium, calcium or alcoholamine salts of dodecylbenzene sulfonic acid or dibutyl-naphthalenesulfonic acid or a naphthalene-sulfonic acid/formaldehyde condensation product. Also suitable are the corresponding phosphates, e.g., salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g., phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulfonates and dialkylsulfosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol, and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon groups optionally substituted with halogen, phenyl, substituted phenyl or hydroxy; for instance, quaternary ammonium salts containing as N-substituent at least one C₈₋₂₂alkyl (e.g., cetyl, lauryl, palmityl, myristyl, oleyl, and the like) and, as further substituents, unsubstituted or halogenated lower alkyl, benzyl and/or hydroxy-lower alkyl.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbucw’, 2 d ed. (Hanser Verlag, Vienna, 1981) and “Encyclopaedia of Surfactants, (Chemical Publishing Co., New York, 1981).

Compounds of the invention as defined herein (including all embodiments thereof as described herein) and their pharmaceutically acceptable salts (hereafter collectively referred to as the active ingredients) may be administered by any route appropriate to the condition to be treated, suitable routes including oral, rectal, nasal, topical (including ocular, buccal, and sublingual), vaginal and parenteral (including subcutaneous, intramuscular, intravenous, intradermal, intrathecal, and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal, and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets, or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, a polyhydric alcohol, e.g., an alcohol having two or more hydroxyl groups such as propylene glycol, butane 1,3-diol, mannitol, sorbitol, glycerol, and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus, the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 μm (including particle sizes in a range between 20 and 500 μm in increments of 5 μm such as 30 μm, 35 μm, etc.), which is administered in the manner in which snuff is taken, e.g., by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foam, or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention as defined herein (including all embodiments thereof as described herein) can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g., microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g., one of them may be in the form of an oral or parenteral formulation whereas the other is in the form of an ampoule for intravenous injection or an aerosol.

The compounds of the present invention as defined herein (including all embodiments thereof as described herein) are useful in the prevention and/or treatment of neurodegenerative diseases or disorders, such as neurodegenerative diseases or disorders characterized by cytotoxic TAU misfolding and/or aggregation and/or aggregation of amyloid beta, but not limited thereto, in order to delay or halt the progression of such diseases or disorders in subjects such as animals, in particular in humans, as described herein.

The term “preventing” or “prevention” as used herein refers to a reduction in risk of acquiring a disease or disorder (i.e., causing at least one of the clinical symptoms of the disease not to develop in a subject, in particular a human subject, that may be exposed to or predisposed to the disease but does not yet experience or display symptoms of the disease).

The term “treating” or “treatment” of any disease or disorder includes, in one embodiment, to improve the disease or disorder (i.e., arresting or reducing the development of the disease or at least reducing one of the clinical symptoms of the disease). In another embodiment “treating” or “treatment” refers to improve at least one physical parameter, which may or may not be discernible by the subject, in particular a human subject, but which is based on or associated with the disease or disorder to be treated. In yet another embodiment, “treating” or “treatment” refers to modulating or alleviating the disease or disorder, either physically (e. g. stabilization of a discernible on non-discernible symptom), physiologically (e. g. stabilization of a physiological parameter), or both. In yet another embodiment, “treating” or “treatment” refers to delaying the onset or progression of the disease or disorder. Accordingly, “treating” or “treatment” includes any causal treatment of the underlying disease or disorder (i.e., disease modification), as well as any treatment of signs and symptoms of the disease or disorder (whether with or without disease modification), as well as any alleviation or amelioration of the disease or disorder, or its signs and symptoms. The terms “disease(s)” and “disorders)” are used largely interchangeably herein.

The term “diagnosis”, “diagnoses” or “diagnosing” of a disease or disorder, as used herein, include, in one embodiment, the identification and measurement of signs and symptoms which are associated with said disease.

The term “subject” refers to an animal preferably a mammalian patient in need of such treatment, such as a human. The term also refers to an animal, preferably a mammal, most preferably a human, who has been the object of treatment, observation, or experiment. The terms “human”, “patient” and “human subject” are typically used interchangeably herein, unless clearly indicated.

The invention also relates to methods of treating an animal disease or disorder, as described in more detail herein, in particular a human disease or disorder, which includes the administration of the compounds of the present invention as defined herein (including all embodiments thereof as described herein) in therapeutically effective amounts.

The term “therapeutically effective amount” as used herein, means that amount of active compound or pharmaceutical agent that, when administered to a subject, elicits the biological or medicinal response in a tissue system, or a subject that is being sought by a researcher, veterinarian, medical doctor, or other clinician, which includes alleviation or partial alleviation of the symptoms of the disease or disorder being treated. The therapeutically effective amount can vary depending on the compound, the disease and its severity, and the condition, age, weight, gender etc. of the subject, in particular a human subject, to be treated.

The compounds of the invention as defined herein (including all embodiments thereof as described herein) are useful for treating or preventing a neurodegenerative disorder

The term “neurodegenerative disorders” as used herein, unless otherwise stated, refers to diseases, disorders or conditions characterized by a degeneration (e.g. including loss of function and eventually cell death) of neurons in either the brain and/or the nervous system of an individual. The progressive damage that occurs to the nerve cells and nervous system connections may have a great impact on mobility, coordination, strength, sensation, and/or cognition.

In particular embodiments, the neurodegenerative disorder may include or may be characterized by the occurrence of cognitive dysfunction in the subject.

In particular embodiments, the neurodegenerative disorder may include or may be characterized by dementia or the symptoms of dementia. Dementia may be diagnosed by any methods known in the art, such as by cognitive and neuropsychological tests (e.g. memory, language skills). Neurodegenerative dementias are progressive and irreversible due to deterioration of nerve cells and their interconnections. Neurodegenerative dementias include Alzheimer's dementia, Lewy body dementia, vascular dementia and frontotemporal lobar dementia, but are not limited thereto.

Accordingly, the neurodegenerative disorder may be, may include or may be characterized by Alzheimer's dementia, Lewy body dementia, vascular dementia and/or frontotemporal lobar dementia (FTD).

In particular embodiments, the neurodegenerative disorder, such as the neurodegenerative disorder which includes or is characterized by dementia as one of its main symptoms, may include or be characterized by protein aggregation. In particular embodiments, the neurodegenerative disorder may include or be characterized by aggregation of tau or amyloid β (Aβ). In further particular embodiments, the neurodegenerative disorder may include or be characterized by tau aggregation and/or beta-amyloid aggregation (i.e. formation of beta-amyloid plaques). In other words, in particular embodiments, the neurodegenerative disorder may be an amyloid β (Aβ)- and/or tau-driven neurodegenerative disorder. In further particular embodiments, the neurodegenerative disorder may include or be characterized by Tau- and/or Aβ-driven neurotoxicity. In further particular embodiments, the neurodegenerative disorder may be a tauopathy (e.g. Alzheimer's disease, Pick's disease, frontotemporal lobar degeneration (FTLD)) or a beta-amyloidopathy (e.g. Alzheimer's disease, Down syndrome).

In particular embodiments, the neurodegenerative disorder may be a tauopathy and/or α-synucleopathy, and thereby includes, but is not limited to Alzheimer's disease, Pick's disease, corticobasal degeneration, progressive supranuclear palsy, frontotemporal dementia, parkinsonism (linked to chromosome 17, FTDP-17), Parkinson's disease, diffuse Lewy body disease, traumatic brain injury, amyotrophic lateral sclerosis (ALS), Niemann-Pick disease, Hallervorden-Spatz syndrome, Down syndrome, neuroaxonal dystrophy, and multiple system atrophy.

In further particular embodiments, the neurodegenerative disorder may be a tauopathy, a beta-amyloidopathy and/or α-synucleopathy.

The term “Tauopathy” as used herein, unless otherwise stated, refers to a disease characterised by dysfunctioning of the TAU protein, for instance manifested by insoluble aggregates or polymers of said protein. The precise molecular mechanisms involved in TAU aggregation are not precisely known, but may involve a partial denaturation or misfolding of TAU in conformations which have a high propensity to self-organise into higher order structures. The misfolding and aggregation may be triggered by hyperphosphorylation of TAU, although at present, it cannot be excluded that such aberrant phosphorylation is a consequence rather than the cause of aggregation.

The compounds of the invention (including all embodiments thereof as described herein) are particularly useful for treating certain neurodegenerative disorders including or being characterized by cytotoxic TAU misfolding and/or aggregation in order to delay or halt the progression of such diseases. Such diseases may be summarized under the term tauopathy, as described elsewhere in the present specification.

TAU is a protein with the ability to bind- and consequently stabilise and define-microtubule structure and function in neurons. The binding of TAU to microtubules is regulated by phosphorylation of TAU; several TAU phosphorylation sites and their corresponding kinases have been identified which control phosphorylation status of TAU and consequently modulate the affinity of TAU-binding to microtubules.

An important aspect of the TAU aggregation is its associated cytotoxicity, which reduces neuronal integrity and functionality and ultimately resulting in disease symptoms. A direct role of TAU in disease onset has been established unequivocally by the elucidation of familial mutations in TAU, which appear to be responsible for a very early and sometimes aggressive form of tauopathy. Such mutations comprise changes in the amino acid sequence of TAU that -directly or indirectly promote neurotoxic aggregation.

Alzheimer's disease is the best known of these, where TAU protein is deposited within neurons in the form of neurofibrillary tangles (NFTs). They were first described by the eponymous Alois Alzheimer in one of his patients suffering from the disorder. The term “Alzheimer's disease” as used herein, refers to a chronic progressive nervous disease characterised by neurodegeneration with as most important (early) symptom being memory loss. As the disease advances, symptoms may include confusion, irritability and aggression, mood swings, language breakdown, long-term memory loss, and the general withdrawal of the sufferer as their senses decline.

Tangles are formed by hyperphosphorylation of a microtubule-associated protein known as TAU, causing it to aggregate in an insoluble form. (These aggregations of hyperphosphorylated TAU protein are also referred to as PHF, or “paired helical filaments”). The precise mechanism of tangle formation is not completely understood, and it is still controversial whether tangles are a primary causative factor in the disease or play a more peripheral role. AD is also classified as an amyloidosis because of the presence of senile plaques.

Other conditions in which neurofibrillary tangles are commonly observed include: Progressive supranuclear palsy, dementia pugilistica (chronic traumatic encephalopathy), frontotemporal dementia and parkinsonism linked to chromosome 17, Lytico-Bodig disease (Parkinson-dementia complex of Guam), tangle-predominant dementia with NFTs, similar to AD, but without plaques, ganglioglioma and gangliocytoma, meningioangiomatosis, subacute sclerosing panencephalitis, tuberous sclerosis, Hallervorden-Spatz disease, and lipofuscinosis.

The non-Alzheimer's tauopathies are sometimes grouped together as “Pick's complex”. In Pick's disease and corticobasal degeneration TAU proteins are deposited in the form of inclusion bodies within swollen or “ballooned” neurons. Argyrophilic grain disease (AGD), another type of dementia, is marked by the presence of abundant argyrophilic grains and coiled bodies on microscopic examination of brain tissue.

The compounds of the invention (including all embodiments thereof as described herein) are particularly useful for the prevention and/or treatment of diseases related to the biological function of dysfunction of TAU protein, which diseases can comprise Alzheimer's disease, Pick's disease, corticobasal degeneration, progressive supranuclear palsy, frontotemporal dementia and parkinsonism (linked to chromosome 17, FTDP-17). The preferred indication is Alzheimer's disease.

The present invention also encompasses a compound of the invention (including all embodiments thereof as described herein) for use in a method for the prevention and/or treatment the prevention and/or treatment of diseases related to the biological function of dysfunction of TAU protein, which diseases can comprise Alzheimer's disease, Pick's disease, corticobasal degeneration, progressive supranuclear palsy, frontotemporal dementia and parkinsonism (linked to chromosome 17, FTDP-17), which comprises administering to a patient in need thereof a therapeutically effective amount of a compound of the invention.

The present invention also encompasses a method for the prevention and/or treatment the prevention and/or treatment of diseases related to the biological function of dysfunction of TAU protein, which diseases can comprise Alzheimer's disease, Pick's disease, corticobasal degeneration, progressive supranuclear palsy, frontotemporal dementia and parkinsonism (linked to chromosome 17, FTDP-17), which comprises administering to a patient in need thereof a therapeutically effective amount of a compound of the invention (including all embodiments thereof as described herein).

The term “amyloidosis” as used herein, unless otherwise stated, refers to both systemic and localized amyloid-related diseases, and prion-related encephalopathies, and thereby includes, but is not limited to type II diabetes mellitus, Alzheimer's disease, Pick's disease, corticobasal degeneration, progressive supranuclear palsy, frontotemporal dementia, parkinsonism (linked to chromosome 17, FTDP-17), Parkinson's disease, diffuse Lewy body disease, traumatic brain injury, amyotrophic lateral sclerosis, Niemann-Pick disease, Hallervorden-Spatz syndrome, Down syndrome, neuroaxonal dystrophy, cataract, Creutzfeld-Jakob's disease, cystic fibrosis, phenylketonuria, Huntington's disease, Marfan syndrome, osteogenesis imperfect, sickle cell anemia, Tay-Sachs disease, α-antitrypsin deficiency, cerebral amyloid angiopathy, retinitis pigmentosa, amyloid A amyloidosis, AL amyloidosis, familial transthyretin amyloidosis, familial Mediterranean fever, amyloidosis associated with long term hemodialysis, amyloidosis associated with medullary carcinoma of the thyroid and multiple system atrophy. For example, the amyloidosis, may be a beta-amyloidopathy.

The term “α-synucleopathy” as used herein, unless otherwise stated, refers to a disease characterised by the presence of pathological deposition of insoluble α-synuclein polymers or aggregates intracellularly and/or extracellularly. Such diseases include, but are not limited to, Parkinson's disease, diffuse Lewy body disease, traumatic brain injury, amyotrophic lateral sclerosis, Niemann-Pick disease, Hallervorden-Spatz syndrome, Down syndrome, neuroaxonal dystrophy, and multiple system atrophy.

The present invention further also encompasses a compound of the invention for use in the prevention and/or treatment of a brain disease or disorder.

In particular embodiments, the brain disease or disorder may include or be characterized by dementia, such as by Alzheimer's disease-related dementia, vascular dementia, Lewy Body dementia, Frontotemporal dementia, Alcohol related dementia, Down syndrome-related dementia, HIV associated dementia and Chronic Traumatic Encephalopathy (CTE) dementia, but not being limited thereto.

In particular embodiments, the brain disease or disorder may include or be characterized by protein aggregation, such as aggregation of tau and/or amyloid β (Aβ), but not being limited thereto.

In particular embodiments, the brain disease or disorder may be a neurodegenerative condition and/or a vascular condition (e.g. neurovascular condition). For example, the brain disease or disorder may be a neurovascular condition including or being characterized by aggregation of amyloid β (AR), such as cerebral amyloid angiopathy.

In particular embodiments, the brain disease or disorder may be a tauopathy. For example, the brain disease or disorder may be Primary age-related tauopathy (PART) dementia, Chronic traumatic encephalopathy (CTE), Progressive supranuclear palsy (PSP), Corticobasal degeneration (CBD), Frontotemporal dementia and parkinsonism linked to chromosome 17 (FTDP-17), Vacuolar tauopathy, Lytico-bodig disease (Parkinson-dementia complex of Guam), Ganglioglioma and gangliocytoma, Meningioangiomatosis, or Postencephalitic parkinsonism.

In particular embodiments, the neurodegenerative disorder is selected from the group consisting of Alzheimer's disease, Pick's disease, corticobasal degeneration, progressive supranuclear palsy, vascular dementia, frontotemporal dementia, parkinsonism (linked to chromosome 17, FTDP-17), Parkinson's disease, diffuse Lewy body disease, traumatic brain injury, amyotrophic lateral sclerosis (ALS), Niemann-Pick disease, Hallervorden-Spatz syndrome, Down syndrome, neuroaxonal dystrophy, multiple system atrophy, Huntington's disease, frontotemporal lobar degeneration (FTLD), cystic fibrosis and Creutzfeld-Jacob's disease.

In further particular embodiments, the neurodegenerative disorder is selected from Parkinson's disease, Alzheimer's disease, diffuse Lewy body disease, amyotrophic lateral sclerosis, Niemann-Pick disease, Hallervorden-Spatz syndrome, Down syndrome, Pick's disease, progressive supranuclear palsy, vascular dementia, neuroaxonal dystrophy, Huntington's disease, frontotemporal lobar degeneration (FTLD), multiple system atrophy and Creutzfeld-Jacob's disease.

In particular embodiments, the neurodegenerative disorder is characterized by Ca²⁺ dyshomeostasis.

In particular embodiments, the neurodegenerative disorder is Alzheimer's disease.

The compounds of the invention as described herein (including all embodiments thereof as described herein) can be prepared while using a series of chemical reactions well known to those skilled in the art, altogether making up the process for preparing said compounds and exemplified further. The processes described further are only meant as examples and by no means are meant to limit the scope of the present invention.

Abbreviations used in the description, particularly in the Schemes and Examples, are as follows: Boc—ter-Butoxycarbonyl, CDI—carbonyldiimidazole, DCC—N,N-dicyclohexylcarbodiimide, CHCl₃—chloroform, DCE—Dichloroethane, DCM—Dichloromethane, DIPEA—Diisopropyl-ethyl amine, DMAc—Dimethylacetamide, DMF—N,N-Dimethylformamide, DMSO—Dimethylsulfoxide, DPPA—diphenylphosphoryl azide, Et₃N—Triethylamine, EtOAc—Ethyl acetate, EtOH—Ethanol, Eq.—Equivalent, Fmoc—Fluorenylmethyloxycarbonyl chloride, h—Hour, HATU—O-(7-Azabenzotriazol-1-yl)-N,N,N′,N′-tetramethyluronium hexafluorophosphate, HPLC—High performance liquid chromatography, KOtBu—potassium ter-butoxide, LCMS—Liquid chromatography-mass spectrometry, LiHDMS—Lithium bis(trimethylsilyl)amide, MeCN (CH₃CN)—ACN—Acetonitrile, MeOH—Methanol, min.—Minute, mL—ml—milliliter, MPLC—medium pressure liquid chromatography, NaBH₄—sodium borohydride, Na₂CO₃—Sodium carbonate, Na₂SO₄—Sodium sulfate, NH₄Cl—ammonium chloride, NH₃—ammonia, NMP—1-Methyl-2-pyrrolidinone, NMR—Nuclear Magnetic Resonance, 4-NPC—4-nitrophenyl chloroformate, PyBOP—benzotriazole-1-yl)oxytripyrrolidinophosphonium hexafluorophosphate, RP—Reverse phase, RT—Room temperature, sat.—Saturated, STAB—sodium triacetoxyborohydride, TEA—Triethylamine, TFA—Trifluoroacetic acid, THF—Tetrahydrofuran.

The preparation of compounds of the present invention as defined herein (including all embodiments thereof as described herein) may be carried out in sequential or convergent synthetic routes.

Embodiments for the syntheses of the compounds of the invention as defined herein (including all embodiments thereof as described herein) are shown in the following schemes.

The skills required for carrying out the reactions and purifications of the resulting products are known to those skilled in the art. The substituents and indices used in the following description of the processes have the significance given herein before unless indicated to the contrary.

In more detail, the compounds as defined herein (including all embodiments thereof as described herein) can be manufactured by the methods given below, by the methods given in the examples or by analogous methods. Appropriate reaction conditions for the individual reaction steps are known to a person skilled in the art. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

In some embodiments, compounds of formula B1A can be prepared according to the general procedure outlined in Scheme A.

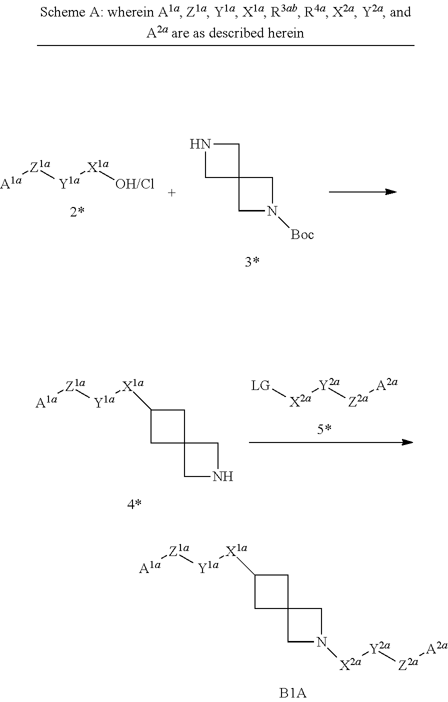

The commercially available amine derivative 3* can be coupled to derivatives *2. Other suitable protecting groups can be used instead of Boc, such as Ts and the like. After removal of the protecting group under standard conditions the intermediate 4* can be obtained. Compound B1A can be obtained upon alkylation or reductive amination of 4*, wherein LG is a suitable leaving group such as for example an aldehyde or a halogen.

In some embodiments, compounds of formula B1B can be prepared according to the general procedure outlined in Scheme B.

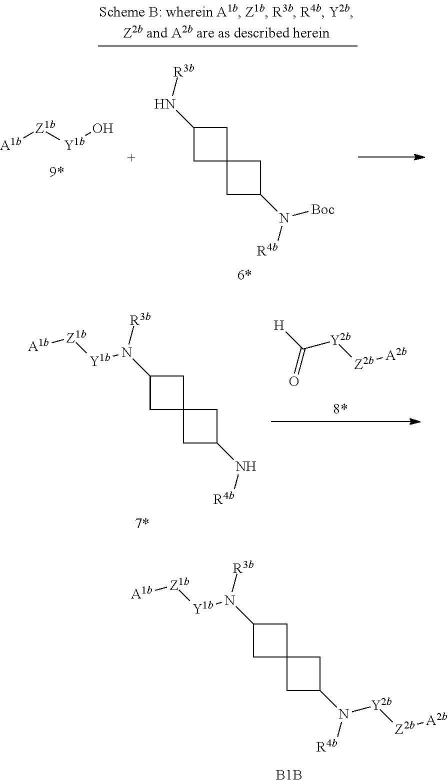

The commercially available amine derivative 6* can be coupled with derivatives 9*. Other suitable protecting groups can be used instead of Boc, such as Ts and the like. After removal of the Boc-protecting group under standard conditions the intermediate 7* can be obtained. Compound B1B can be obtained upon coupling of 7* with the appropriate arylaldehyde 8*. In some embodiments, R^{4b} is H in derivative 6* and a group R^{4b} different from H can be introduced by further reacting B1B with a suitable reagent when applicable.

In some embodiments, compounds of formula B1A1, B1A2, B1A3, B1A4, B1A5 can be prepared according to the general procedure outlined in Scheme A1.

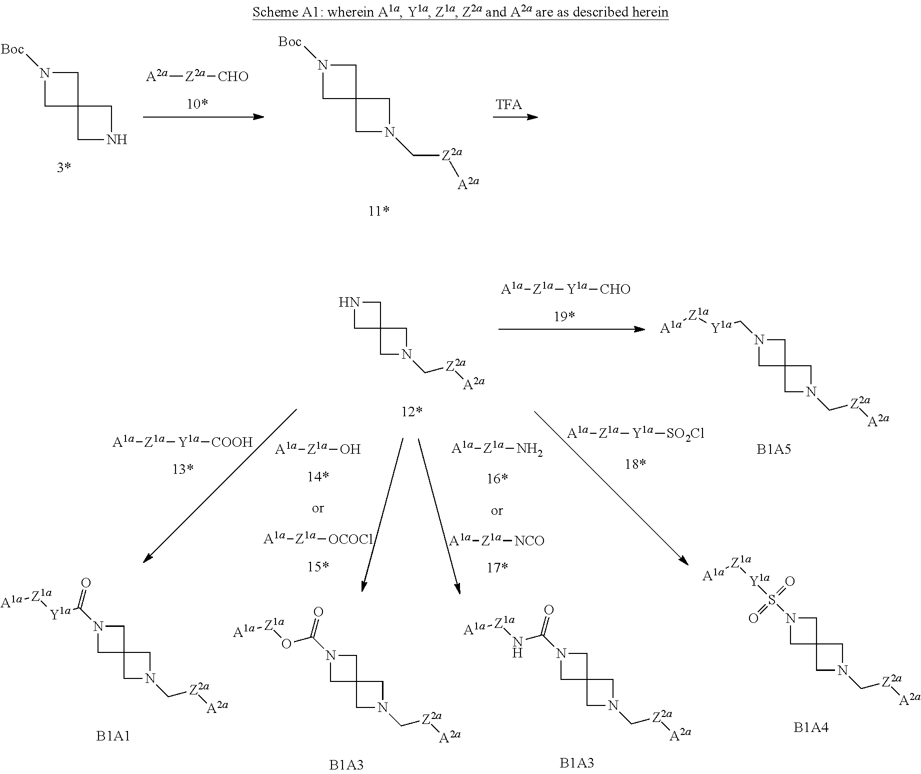

Compound 3*, commercially available, can be coupled with an aldehyde 10* (commercially available or synthesized by procedures known to the person skilled in the art) via reductive amination in presence of a suitable reductive reagent (e.g., STAB, NaBH₄, Pd/C H₂, and the like), for example in a suitable solvent or mixture of solvents (e.g., DCM, MeOH, DMF, DMAc, toluene, dioxane, water, and the like), for example at a temperature ranging from RT to 60° C., to provide intermediates of formula 11*. Other suitable protecting groups can be used instead of Boc, such as Fmoc, Ts and the like. After removal of the Boc-protecting group under standard conditions, the intermediate 12* can be obtained. Compound B1A1 can be obtained upon coupling of intermediate 12* with an acid 13* (commercially available or synthesized by procedures known to the person skilled in the art), for example in the presence of a suitable base (e.g., DIPEA and the like) and at least one suitable coupling agent (e.g., HATU, PyBOP, DCC, and the like), for example in a suitable solvent or mixture of solvents (e.g., DCM, MeCN, CHCl₃, DMF, DMAc, DMP, and the like), for example at a temperature ranging from 0° C. to 60° C.

Compound B1A2 can be obtained upon coupling of intermediate 12* with an alcohol 14* (commercially available or synthesized by procedures known to the person skilled in the art), for example in the presence of a suitable base (e.g., NaH, DIPEA, KOtBu, LiHMDS, and the like) and a suitable coupling reagent (e.g., bis(4-nitrophenyl) carbonate, 4-NPC, CDI, and the like), for example in a suitable solvent or mixture of solvents (e.g., DMF, THF, dioxane, toluene, and the like), for example at a temperature ranging from −20° C. to RT. Alternatively, the compound of general formula B1A2 can be obtained via a coupling between intermediate 12* and chloroformates 15* (commercially available or synthesized by procedures known to the person skilled in the art), for example in the presence of a suitable base (e.g., DIPEA and the like), for example in a suitable solvent or mixture of solvents (e.g., DCM, CHCl₃, THF, DMF, DMAc, dioxane, and the like), for example at a temperature ranging from −20° C. to 60° C.

Compound B1A3 can be obtained upon coupling of intermediate 12* with an amine 16* (commercially available or synthesized by procedures known to the person skilled in the art), for example in the presence of a suitable base (e.g., DIPEA, Et₃N, and the like) and a suitable coupling reagent (e.g. bis(4-nitrophenyl) carbonate, 4-NPC, CDI, and the like), for example in a suitable solvent or mixture of solvents (e.g., DCM, CHCl₃, THF, DMF, DMAc, dioxane, and the like), for example at a temperature ranging from 0° C. to 50° C. Alternatively, the compound of general formula B1A3 can be obtained via a coupling between intermediate 12* and isocyanates 17* (commercially available or synthesized by procedures known to the person skilled in the art), for example in the presence of a suitable base (e.g., DIPEA and the like), for example in a suitable solvent or mixture of solvents (e.g., DCM, CHCl₃, THF, DMF, DMAc, dioxane, and the like), for example at a temperature ranging from −20° C. to 60° C.

Compound B1A4 can be obtained upon coupling of intermediate 12* with a sulfonylchloride 18* (commercially available or synthesized by procedures known to the person skilled in the art), for example in a suitable solvent or mixture of solvents (e.g., DCM, CHCl₃, THF, DMF, DMAc, dioxane, and the like), for example at a temperature ranging from −20° C. to 60° C.

Compound B1A5 can be obtained upon reductive amination of intermediate 12* with an aldehyde 19* (commercially available or synthesized by procedures known to the person skilled in the art), for example in presence of a suitable reductive reagent (e.g. STAB, NaBH₄, Pd/C H₂, and the like), for example in a suitable solvent or mixture of solvents (e.g., DCM, MeOH, DMF, DMAc, toluene, dioxane, water, and the like), for example at a temperature ranging from RT to 60° C.

In some embodiments, compounds of formula B1A1 can be prepared according to the general procedure outlined in Scheme A2.

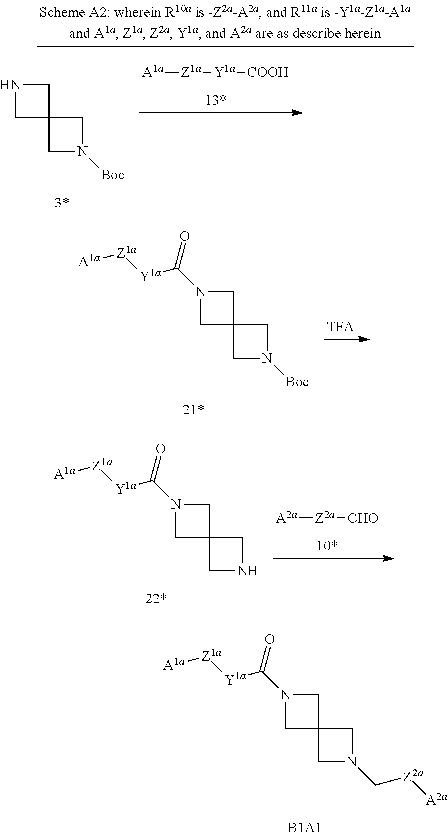

Compound 3*, commercially available, can be coupled with an acid 13* (commercially available or synthesized by procedures known to the person skilled in the art), for example in the presence of a suitable base (e.g., DIPEA and the like) and a suitable coupling agent (e.g. HATU, PyBOP, DCC, and the like), for example in a suitable solvent or solvent mixture (e.g., DCM, MeCN, CHCl₃, DMF, DMAc, DMP, and the like), for example at a temperature ranging from 0° C. to 60° C. to provide intermediates of formula 21*. Other suitable protecting groups can be used instead of Boc, such as Fmoc, Ts and the like. After removal of the Boc-protecting group under standard conditions, the intermediate 22* is obtained. Compound B1A1 can be obtained upon reductive amination of intermediate 22* with an aldehyde 10* (commercially available or synthesized by procedures known to the person skilled in the art), for example in presence of a suitable reductive reagent (e.g. STAB, NaBH₄, Pd/C H₂, and the like), for example in a suitable solvent or mixture of solvents (e.g., DCM, MeOH, DMF, DMAc, toluene, dioxane, water, and the like), for example at a temperature ranging from RT to 60° C.

In some embodiments, compounds of formula B1A2 can be prepared according to the general procedure outlined in Scheme A3.

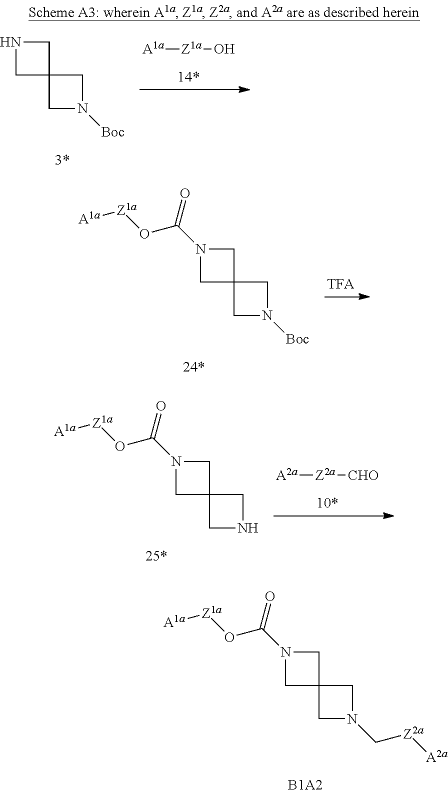

Compound 3*, commercially available, can be coupled with an alcohol 14* (commercially available or synthesized by procedures known to the person skilled in the art), for example in the presence of a suitable base (e.g., NaH, DIPEA, KOtBu, LiHMDS, and the like) and a suitable coupling reagent (e.g., bis(4-nitrophenyl) carbonate, 4-NPC, CDI, and the like), for example in a suitable solvent or solvent mixture (e.g., DMF, THF, toluene, dioxane, and the like), for example at a temperature ranging from −20° C. to RT to provide intermediates of formula 24*. Other suitable protecting groups can be used instead of Boc, such as Fmoc, Ts and the like. After removal of the Boc-protecting group under standard conditions, the intermediate 25* is obtained. Compound B1A2 can be obtained upon reductive amination of intermediate 25* with an aldehyde 10* (commercially available or synthesized by procedures known to the person skilled in the art), for example in presence of a reductive reagent (e.g. STAB, NaBH₄, Pd/C H₂, and the like), for example in a suitable solvent or mixture of solvents (e.g., DCM, MeOH, DMF, DMAc, toluene, dioxane, water, and the like), for example at a temperature ranging from RT to 60° C.

In some embodiments, compounds of formula B1A3 can be prepared according to the general procedure outlined in Scheme A4.

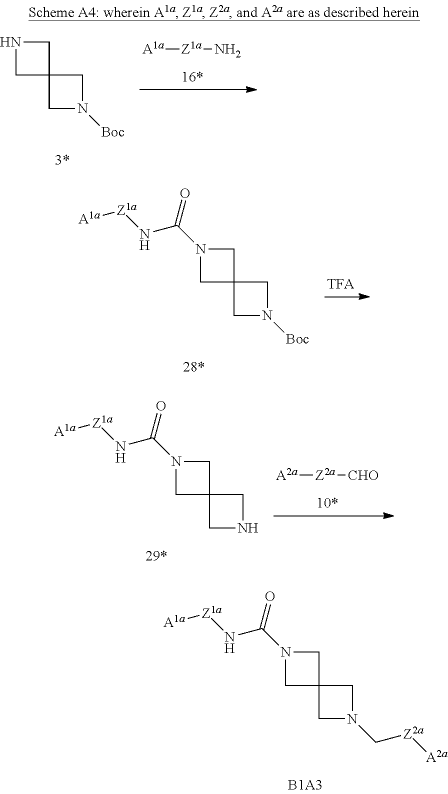

Compound 3*, commercially available, can be coupled with an amine 16* (commercially available or synthesized by procedures known to the person skilled in the art), for example in presence of a suitable base (e.g., DIPEA, Et₃N, and the like) and a coupling agent (e.g. bis(4-nitrophenyl) carbonate, 4-NPC, CDI, and the like), for example in a suitable solvent or mixture of solvents (e.g. DCM, CHCl₃, THF, DMF, DMAC, dioxane, and the like), for example at a temperature ranging from 0° C. to 50° C. to provide intermediates of formula 28*. Other suitable protecting groups can be used instead of Boc, such as Fmoc, Ts and the like. After removal of the Boc-protecting group under standard conditions, the intermediate 29* can be obtained. Compound B1A3 can be obtained upon reductive amination of intermediate 29* with an aldehyde 10* (commercially available or synthesized by procedures known to the person skilled in the art), for example in presence of a suitable reductive reagent (e.g., STAB, NaBH₄, Pd/C H₂, and the like), for example in a suitable solvent or mixture of solvents (e.g., DCM, MeOH, DMF, DMAc, toluene, dioxane, water, and the like), for example at a temperature ranging from RT to 60° C.

In some embodiments, compounds of formula B1A6 can be prepared according to the general procedure outlined in Scheme A5.

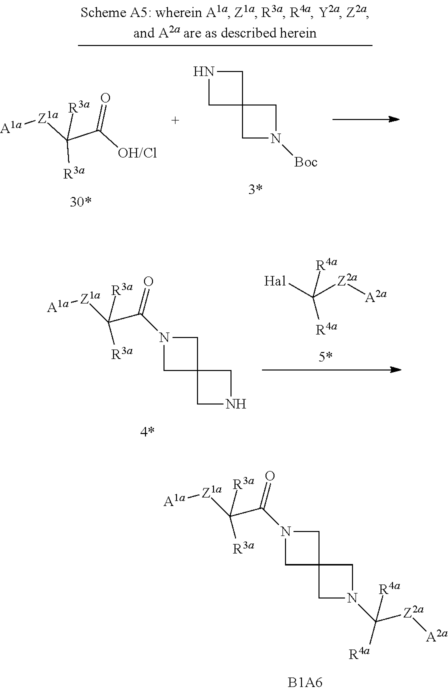

Compound 3* can be coupled to derivative 30*. Other suitable protecting groups can be used instead of Boc, such as Fmoc, Ts and the like. After removal of the Boc-protecting group under standard conditions the intermediate 31* can be obtained. Compound B1A6 can be obtained upon alkylation or reductive amination of 31*.

In some embodiments, compounds of formula B1B1 can be prepared according to the general procedure outlined in Scheme B1.

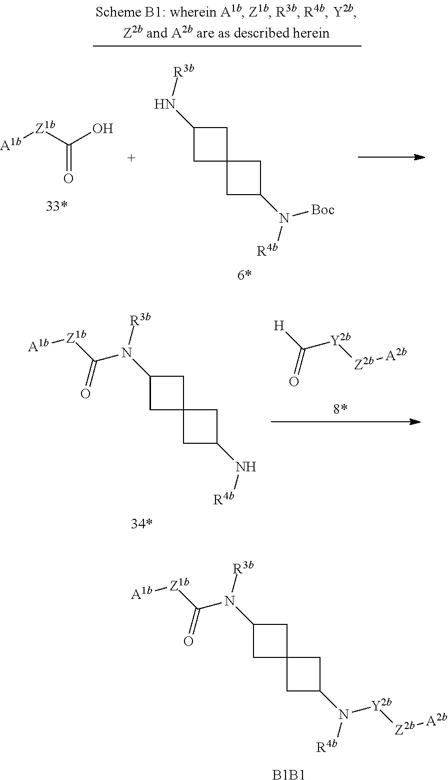

The commercially available amine derivative 6* can be coupled with derivatives 33*. After removal of the Boc-protecting group under standard conditions the intermediate 34* can be obtained. Final compound B1B1 can be obtained upon coupling of 34* with the appropriate arylaldehyde 8*. In some embodiments, R^{4b} is H in derivative 6* and a group R^{4b} different from H can be introduced by further reacting B1B1 with a suitable reagent when applicable.

In some embodiments, compounds of formula B1B2 can be prepared according to the general procedure outlined in Scheme B2.

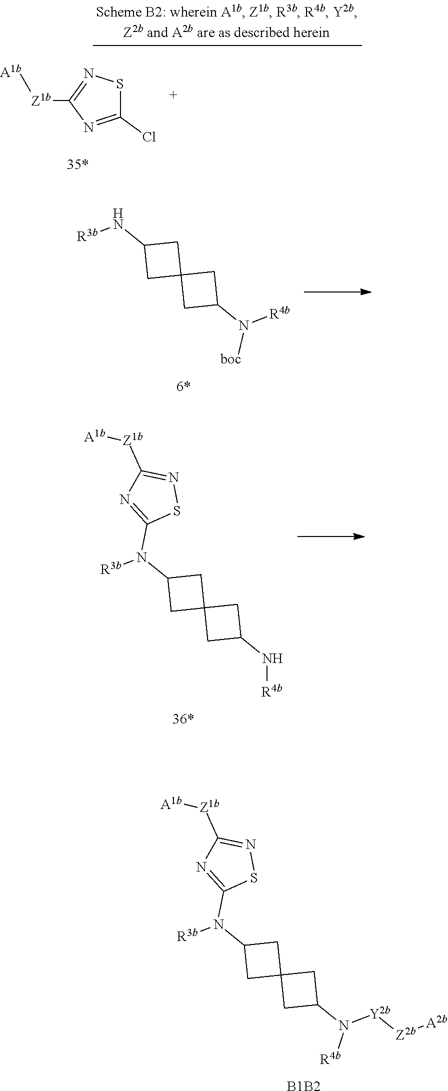

The commercially available amine derivative 6* can be coupled to compound 35*. After removal of the Boc-protecting group under standard conditions the intermediate 36* can be obtained. Final compound B1B2 can be obtained from amine 36* upon reductive amination an aldehyde or alkylation with an electrophile. In some embodiments, R^{4b} is H in derivative 6* and a group R^{4b}different from H can be introduced by further reacting B1B2 with a suitable reagent when applicable.

The general schemes depicted above should be considered as non-limiting examples. It will be understood that compounds of the invention may be obtained through other methods, which are known to people skilled in the art.

The following examples are provided for the purpose of illustrating the present invention and by no means should be interpreted to limit the scope of the present invention.

### EXAMPLES

TABLE 1B
Structures of the compounds of the invention and their respective codes

| Compound | Code |
|---|---|
| 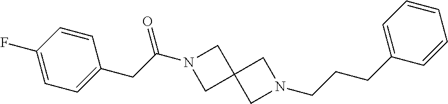 [SMILES: O=C(Cc1ccc(F)cc1)N1CC2(CN(CCCc3ccccc3)C2)C1] | Cpd B001 |
| 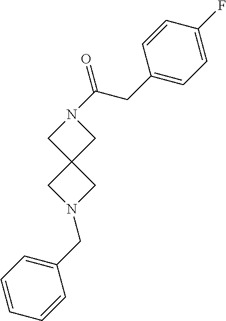 [SMILES: O=C(Cc1ccc(F)cc1)N1CC2(CN(Cc3ccccc3)C2)C1] | Cpd B002 |
| 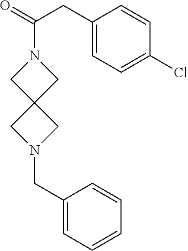 [SMILES: O=C(Cc1ccc(Cl)cc1)N1CC2(CN(Cc3ccccc3)C2)C1] | Cpd B003 |
| 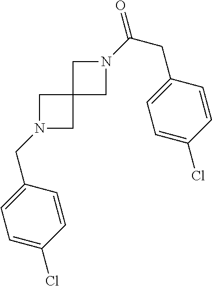 [SMILES: O=C(Cc1ccc(Cl)cc1)N1CC2(CN(Cc3ccc(Cl)cc3)C2)C1] | Cpd B004 |
| 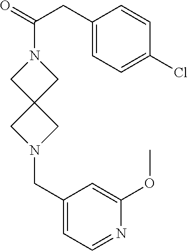 [SMILES: COc1cc(CN2CC3(C2)CN(C(=O)Cc2ccc(Cl)cc2)C3)ccn1] | Cpd B005 |
| 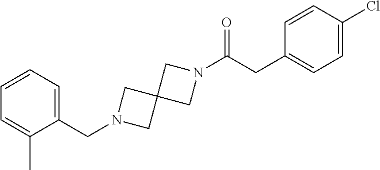 [SMILES: Cc1ccccc1CN1CC2(C1)CN(C(=O)Cc1ccc(Cl)cc1)C2] | Cpd B006 |
| 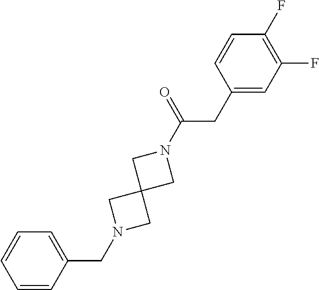 [SMILES: O=C(Cc1ccc(F)c(F)c1)N1CC2(CN(Cc3ccccc3)C2)C1] | Cpd B007 |
| 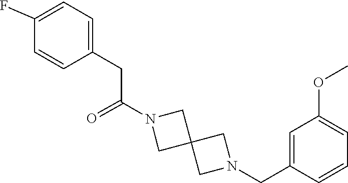 [SMILES: COc1cccc(CN2CC3(C2)CN(C(=O)Cc2ccc(F)cc2)C3)c1] | Cpd B008 |
| 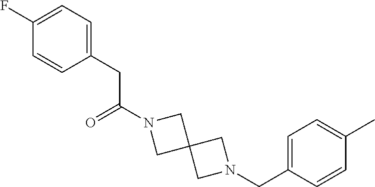 [SMILES: Cc1ccc(CN2CC3(C2)CN(C(=O)Cc2ccc(F)cc2)C3)cc1] | Cpd B009 |
| 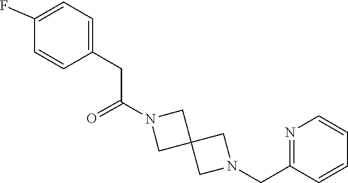 [SMILES: O=C(Cc1ccc(F)cc1)N1CC2(CN(Cc3ccccn3)C2)C1] | Cpd B010 |
| 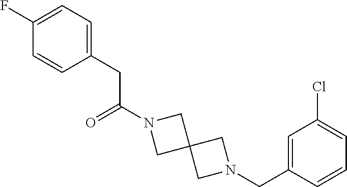 [SMILES: O=C(Cc1ccc(F)cc1)N1CC2(CN(Cc3cccc(Cl)c3)C2)C1] | Cpd B011 |
| 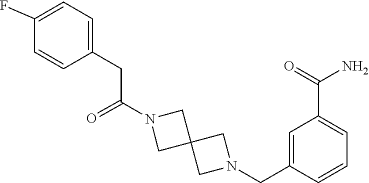 [SMILES: NC(=O)c1cccc(CN2CC3(C2)CN(C(=O)Cc2ccc(F)cc2)C3)c1] | Cpd B012 |
| 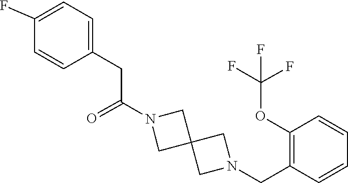 [SMILES: O=C(Cc1ccc(F)cc1)N1CC2(CN(Cc3ccccc3OC(F)(F)F)C2)C1] | Cpd B013 |
| 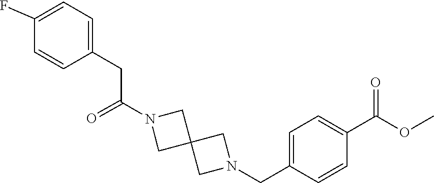 [SMILES: COC(=O)c1ccc(CN2CC3(C2)CN(C(=O)Cc2ccc(F)cc2)C3)cc1] | Cpd B014 |
| 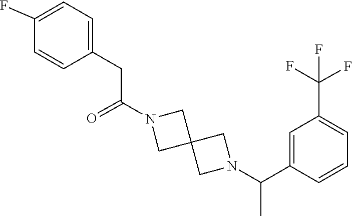 [SMILES: CC(c1cccc(C(F)(F)F)c1)N1CC2(CN(C(=O)Cc3ccc(F)cc3)C2)C1] | Cpd B015 |
| 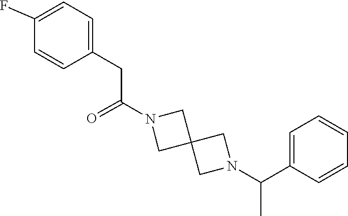 [SMILES: CC(c1ccccc1)N1CC2(CN(C(=O)Cc3ccc(F)cc3)C2)C1] | Cpd B016 |
| 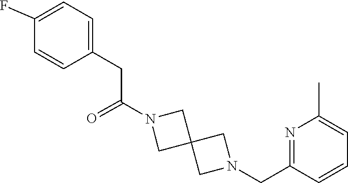 [SMILES: Cc1cccc(CN2CC3(C2)CN(C(=O)Cc2ccc(F)cc2)C3)n1] | Cpd B017 |
| 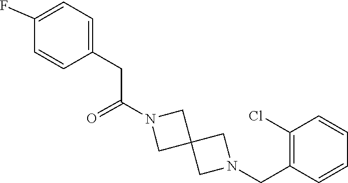 [SMILES: O=C(Cc1ccc(F)cc1)N1CC2(CN(Cc3ccccc3Cl)C2)C1] | Cpd B018 |
| 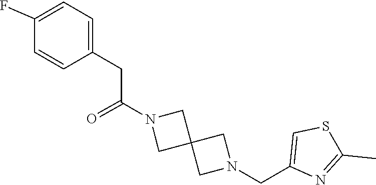 [SMILES: Cc1nc(CN2CC3(C2)CN(C(=O)Cc2ccc(F)cc2)C3)cs1] | Cpd B019 |
| 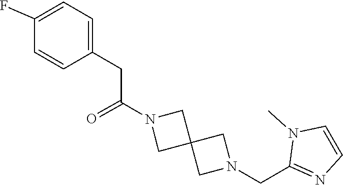 [SMILES: Cn1ccnc1CN1CC2(C1)CN(C(=O)Cc1ccc(F)cc1)C2] | Cpd B020 |
| 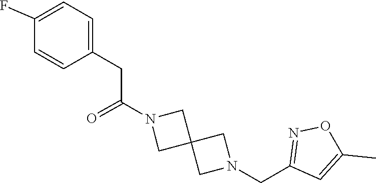 [SMILES: Cc1cc(CN2CC3(C2)CN(C(=O)Cc2ccc(F)cc2)C3)no1] | Cpd B021 |
| 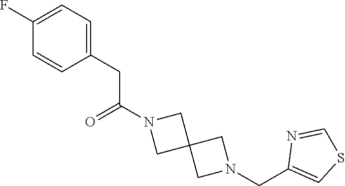 [SMILES: O=C(Cc1ccc(F)cc1)N1CC2(CN(Cc3cscn3)C2)C1] | Cpd B022 |
| 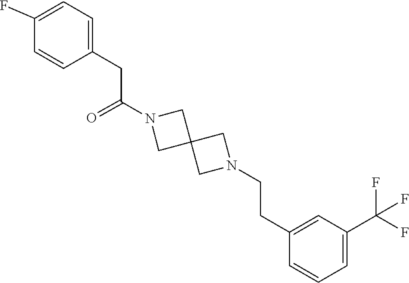 [SMILES: O=C(Cc1ccc(F)cc1)N1CC2(CN(CCc3cccc(C(F)(F)F)c3)C2)C1] | Cpd B023 |
| 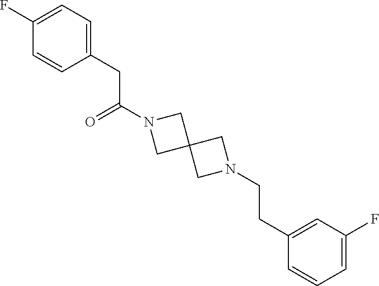 [SMILES: O=C(Cc1ccc(F)cc1)N1CC2(CN(CCc3cccc(F)c3)C2)C1] | Cpd B024 |
| 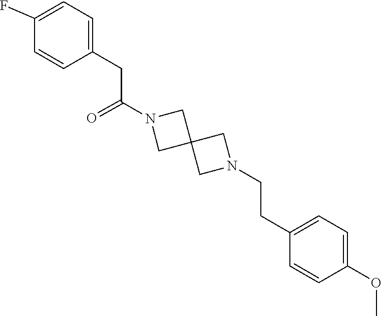 [SMILES: COc1ccc(CCN2CC3(C2)CN(C(=O)Cc2ccc(F)cc2)C3)cc1] | Cpd B025 |
| 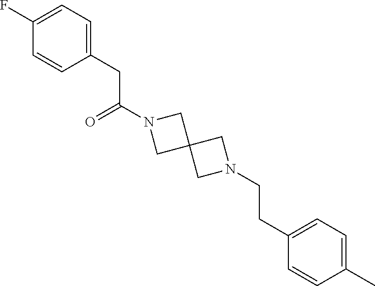 [SMILES: Cc1ccc(CCN2CC3(C2)CN(C(=O)Cc2ccc(F)cc2)C3)cc1] | Cpd B026 |
| 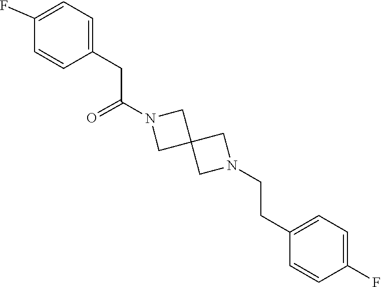 [SMILES: O=C(Cc1ccc(F)cc1)N1CC2(CN(CCc3ccc(F)cc3)C2)C1] | Cpd B027 |
| 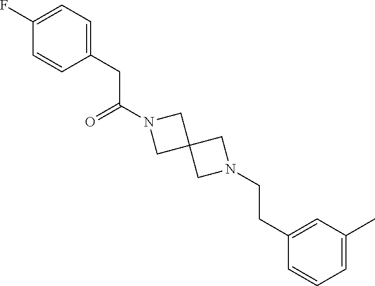 [SMILES: Cc1cccc(CCN2CC3(C2)CN(C(=O)Cc2ccc(F)cc2)C3)c1] | Cpd B028 |
| 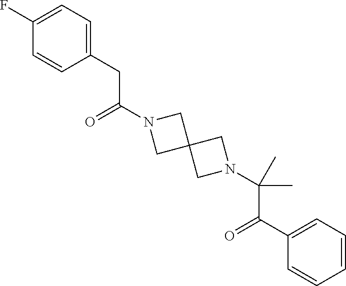 [SMILES: CC(C)(C(=O)c1ccccc1)N1CC2(CN(C(=O)Cc3ccc(F)cc3)C2)C1] | Cpd B029 |
| 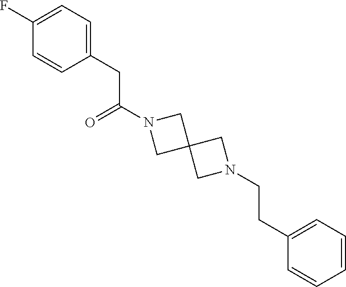 [SMILES: O=C(Cc1ccc(F)cc1)N1CC2(CN(CCc3ccccc3)C2)C1] | Cpd B030 |
| 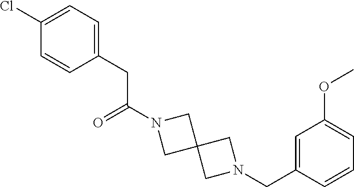 [SMILES: COc1cccc(CN2CC3(C2)CN(C(=O)Cc2ccc(Cl)cc2)C3)c1] | Cpd B031 |
| 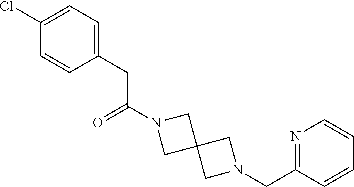 [SMILES: O=C(Cc1ccc(Cl)cc1)N1CC2(CN(Cc3ccccn3)C2)C1] | Cpd B032 |
| 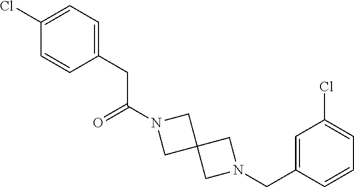 [SMILES: O=C(Cc1ccc(Cl)cc1)N1CC2(CN(Cc3cccc(Cl)c3)C2)C1] | Cpd B033 |
| 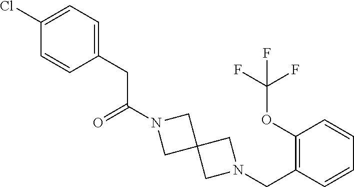 [SMILES: O=C(Cc1ccc(Cl)cc1)N1CC2(CN(Cc3ccccc3OC(F)(F)F)C2)C1] | Cpd B034 |
| 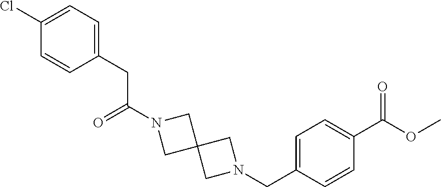 [SMILES: COC(=O)c1ccc(CN2CC3(C2)CN(C(=O)Cc2ccc(Cl)cc2)C3)cc1] | Cpd B035 |
| 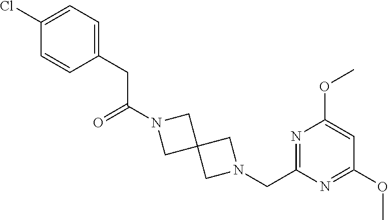 [SMILES: COc1cc(OC)nc(CN2CC3(C2)CN(C(=O)Cc2ccc(Cl)cc2)C3)n1] | Cpd B036 |
| 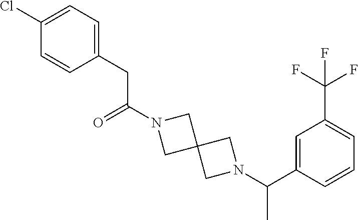 [SMILES: CC(c1cccc(C(F)(F)F)c1)N1CC2(CN(C(=O)Cc3ccc(Cl)cc3)C2)C1] | Cpd B037 |
| 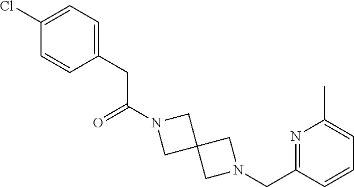 [SMILES: Cc1cccc(CN2CC3(C2)CN(C(=O)Cc2ccc(Cl)cc2)C3)n1] | Cpd B038 |
| 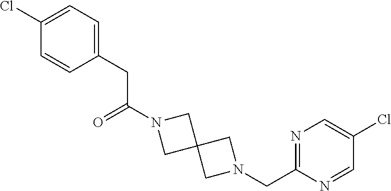 [SMILES: O=C(Cc1ccc(Cl)cc1)N1CC2(CN(Cc3ncc(Cl)cn3)C2)C1] | Cpd B039 |
| 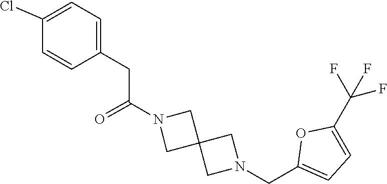 [SMILES: O=C(Cc1ccc(Cl)cc1)N1CC2(CN(Cc3ccc(C(F)(F)F)o3)C2)C1] | Cpd B040 |
| 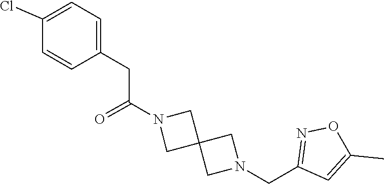 [SMILES: Cc1cc(CN2CC3(C2)CN(C(=O)Cc2ccc(Cl)cc2)C3)no1] | Cpd B041 |
| 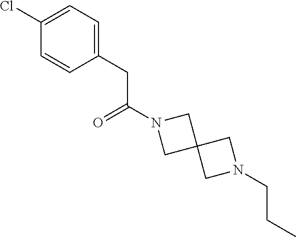 [SMILES: CCCN1CC2(C1)CN(C(=O)Cc1ccc(Cl)cc1)C2] | Cpd B042 |
| 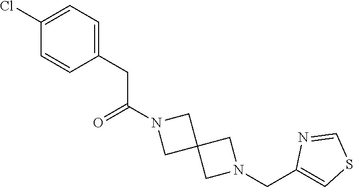 [SMILES: O=C(Cc1ccc(Cl)cc1)N1CC2(CN(Cc3cscn3)C2)C1] | Cpd B043 |
| 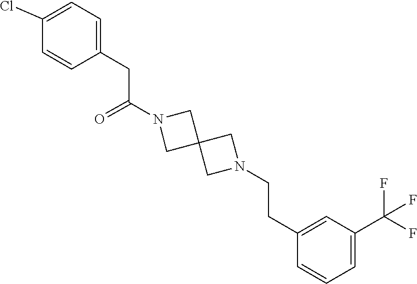 [SMILES: O=C(Cc1ccc(Cl)cc1)N1CC2(CN(CCc3cccc(C(F)(F)F)c3)C2)C1] | Cpd B044 |
| 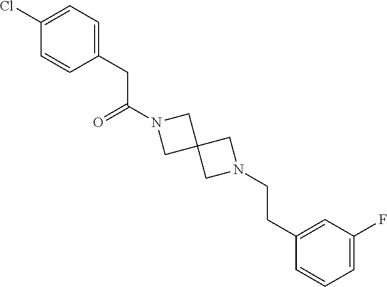 [SMILES: O=C(Cc1ccc(Cl)cc1)N1CC2(CN(CCc3cccc(F)c3)C2)C1] | Cpd B045 |
| 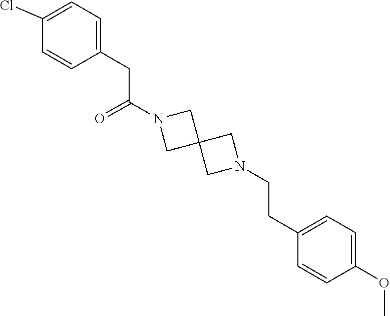 [SMILES: COc1ccc(CCN2CC3(C2)CN(C(=O)Cc2ccc(Cl)cc2)C3)cc1] | Cpd B046 |
| 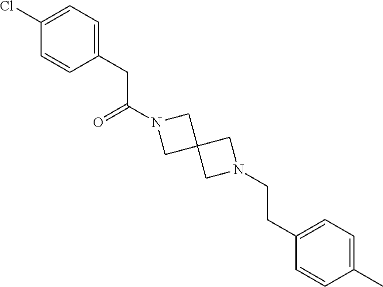 [SMILES: Cc1ccc(CCN2CC3(C2)CN(C(=O)Cc2ccc(Cl)cc2)C3)cc1] | Cpd B047 |
| 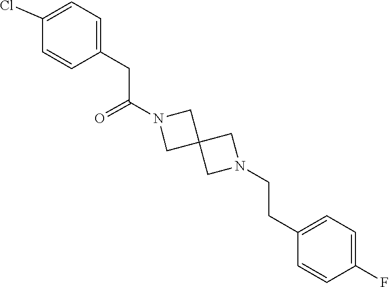 [SMILES: O=C(Cc1ccc(Cl)cc1)N1CC2(CN(CCc3ccc(F)cc3)C2)C1] | Cpd B048 |
| 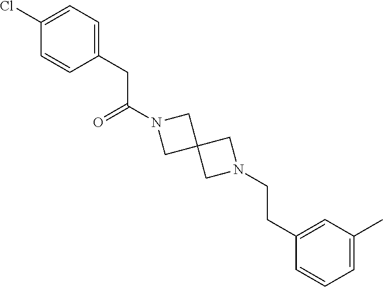 [SMILES: Cc1cccc(CCN2CC3(C2)CN(C(=O)Cc2ccc(Cl)cc2)C3)c1] | Cpd B049 |
| 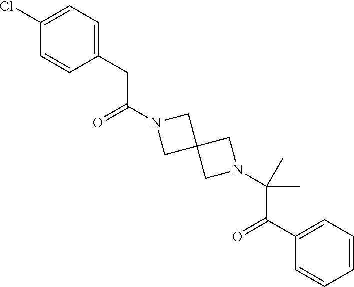 [SMILES: CC(C)(C(=O)c1ccccc1)N1CC2(CN(C(=O)Cc3ccc(Cl)cc3)C2)C1] | Cpd B050 |
| 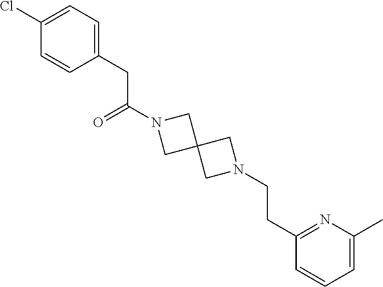 [SMILES: Cc1cccc(CCN2CC3(C2)CN(C(=O)Cc2ccc(Cl)cc2)C3)n1] | Cpd B051 |
| 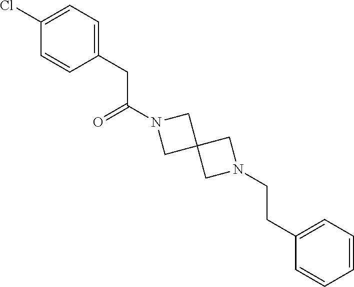 [SMILES: O=C(Cc1ccc(Cl)cc1)N1CC2(CN(CCc3ccccc3)C2)C1] | Cpd B052 |
| 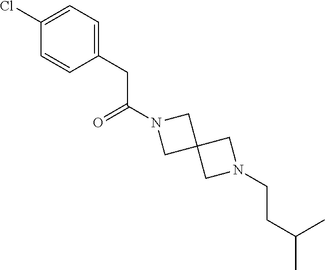 [SMILES: CC(C)CCN1CC2(C1)CN(C(=O)Cc1ccc(Cl)cc1)C2] | Cpd B053 |
| 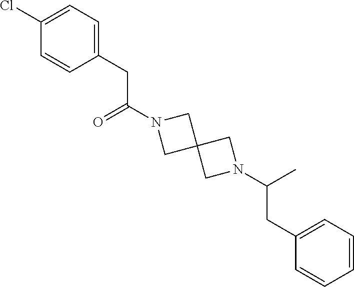 [SMILES: CC(Cc1ccccc1)N1CC2(CN(C(=O)Cc3ccc(Cl)cc3)C2)C1] | Cpd B054 |
| 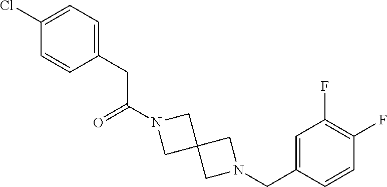 [SMILES: O=C(Cc1ccc(Cl)cc1)N1CC2(CN(Cc3ccc(F)c(F)c3)C2)C1] | Cpd B055 |
| 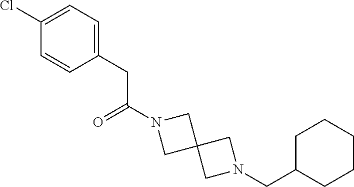 [SMILES: O=C(Cc1ccc(Cl)cc1)N1CC2(CN(CC3CCCCC3)C2)C1] | Cpd B056 |
| 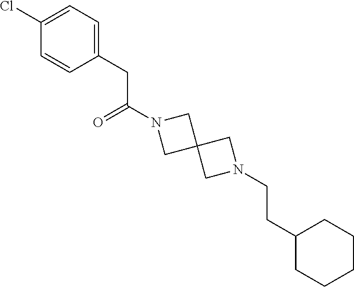 [SMILES: O=C(Cc1ccc(Cl)cc1)N1CC2(CN(CCC3CCCCC3)C2)C1] | Cpd B057 |
| 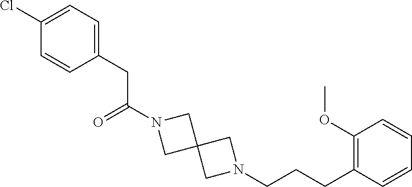 [SMILES: COc1ccccc1CCCN1CC2(C1)CN(C(=O)Cc1ccc(Cl)cc1)C2] | Cpd B058 |
| 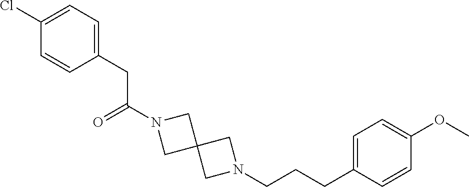 [SMILES: COc1ccc(CCCN2CC3(C2)CN(C(=O)Cc2ccc(Cl)cc2)C3)cc1] | Cpd B059 |
| 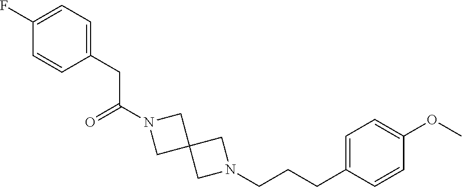 [SMILES: COc1ccc(CCCN2CC3(C2)CN(C(=O)Cc2ccc(F)cc2)C3)cc1] | Cpd B060 |
| 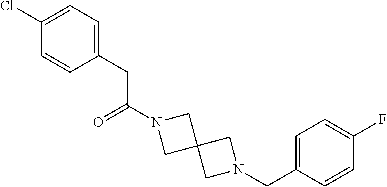 [SMILES: O=C(Cc1ccc(Cl)cc1)N1CC2(CN(Cc3ccc(F)cc3)C2)C1] | Cpd B061 |
| 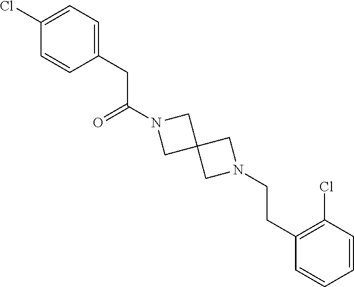 [SMILES: O=C(Cc1ccc(Cl)cc1)N1CC2(CN(CCc3ccccc3Cl)C2)C1] | Cpd B062 |
| 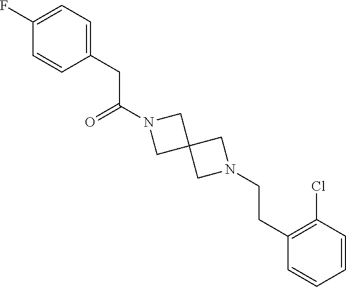 [SMILES: O=C(Cc1ccc(F)cc1)N1CC2(CN(CCc3ccccc3Cl)C2)C1] | Cpd B063 |
| 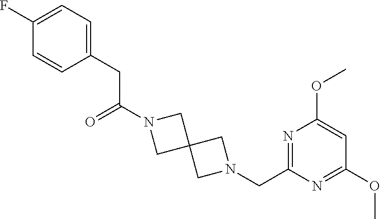 [SMILES: COc1cc(OC)nc(CN2CC3(C2)CN(C(=O)Cc2ccc(F)cc2)C3)n1] | Cpd B064 |
| 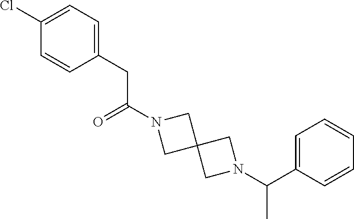 [SMILES: CC(c1ccccc1)N1CC2(CN(C(=O)Cc3ccc(Cl)cc3)C2)C1] | Cpd B065 |
| 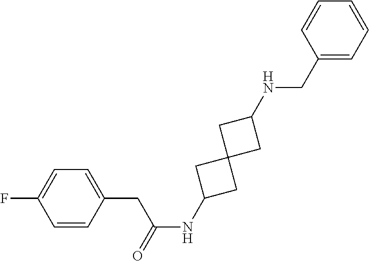 [SMILES: O=C(Cc1ccc(F)cc1)NC1CC2(CC(NCc3ccccc3)C2)C1] | Cpd B066 |
| 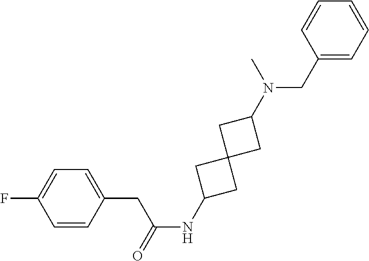 | Cpd B067 |
| 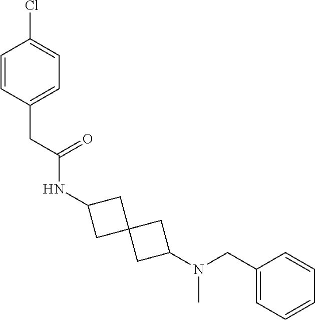 [SMILES: CN(Cc1ccccc1)C1CC2(CC(NC(=O)Cc3ccc(Cl)cc3)C2)C1] | Cpd B068 |
| 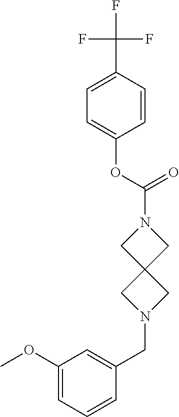 [SMILES: COc1cccc(CN2CC3(C2)CN(C(=O)Oc2ccc(C(F)(F)F)cc2)C3)c1] | Cpd B069 |
| 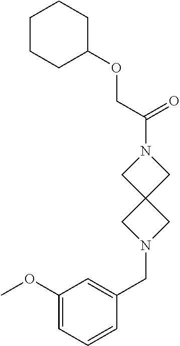 [SMILES: COc1cccc(CN2CC3(C2)CN(C(=O)COC2CCCCC2)C3)c1] | Cpd B070 |
| 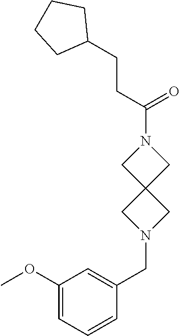 [SMILES: COc1cccc(CN2CC3(C2)CN(C(=O)CCC2CCCC2)C3)c1] | Cpd B071 |
| 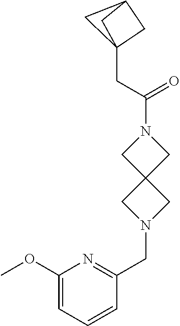 [SMILES: COc1cccc(CN2CC3(C2)CN(C(=O)CC24CC(C2)C4)C3)n1] | Cpd B072 |
| 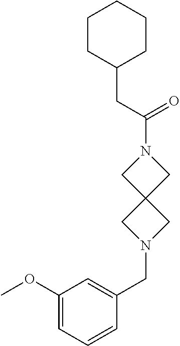 [SMILES: COc1cccc(CN2CC3(C2)CN(C(=O)CC2CCCCC2)C3)c1] | Cpd B073 |
| 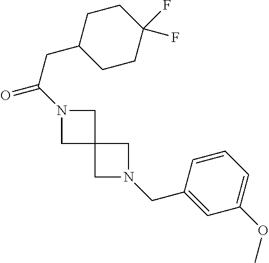 [SMILES: COc1cccc(CN2CC3(C2)CN(C(=O)CC2CCC(F)(F)CC2)C3)c1] | Cpd B074 |
| 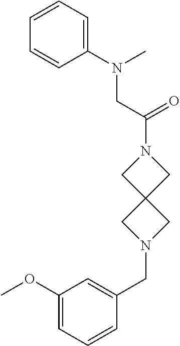 [SMILES: COc1cccc(CN2CC3(C2)CN(C(=O)CN(C)c2ccccc2)C3)c1] | Cpd B075 |
| 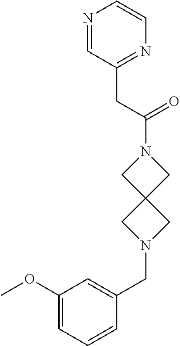 [SMILES: COc1cccc(CN2CC3(C2)CN(C(=O)Cc2cnccn2)C3)c1] | Cpd B076 |
| 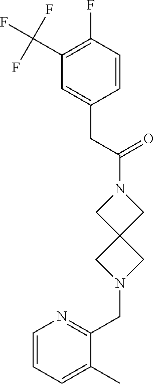 [SMILES: Cc1cccnc1CN1CC2(C1)CN(C(=O)Cc1ccc(F)c(C(F)(F)F)c1)C2] | Cpd B077 |
| 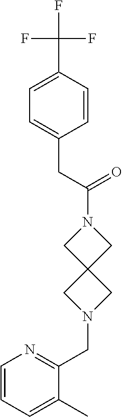 [SMILES: Cc1cccnc1CN1CC2(C1)CN(C(=O)Cc1ccc(C(F)(F)F)cc1)C2] | Cpd B078 |
| 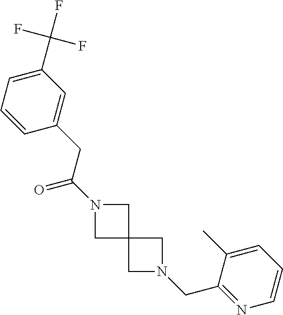 [SMILES: Cc1cccnc1CN1CC2(C1)CN(C(=O)Cc1cccc(C(F)(F)F)c1)C2] | Cpd B079 |
| 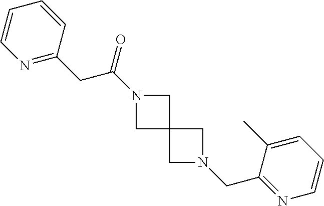 [SMILES: Cc1cccnc1CN1CC2(C1)CN(C(=O)Cc1ccccn1)C2] | Cpd B080 |
| 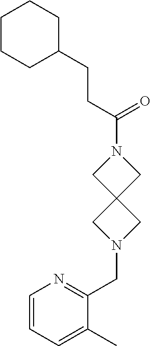 [SMILES: Cc1cccnc1CN1CC2(C1)CN(C(=O)CCC1CCCCC1)C2] | Cpd B081 |
| 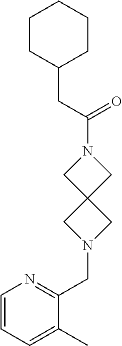 [SMILES: Cc1cccnc1CN1CC2(C1)CN(C(=O)CC1CCCCC1)C2] | Cpd B082 |
| 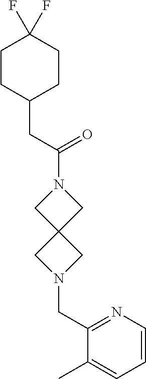 [SMILES: Cc1cccnc1CN1CC2(C1)CN(C(=O)CC1CCC(F)(F)CC1)C2] | Cpd B083 |
| 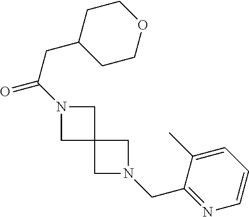 [SMILES: Cc1cccnc1CN1CC2(C1)CN(C(=O)CC1CCOCC1)C2] | Cpd B084 |
| 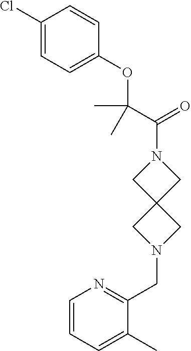 [SMILES: Cc1cccnc1CN1CC2(C1)CN(C(=O)C(C)(C)Oc1ccc(Cl)cc1)C2] | Cpd B085 |
| 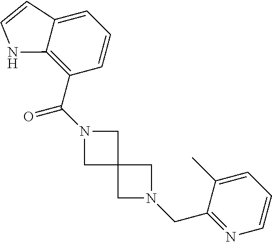 [SMILES: Cc1cccnc1CN1CC2(C1)CN(C(=O)c1cccc3cc[nH]c13)C2] | Cpd B086 |
| 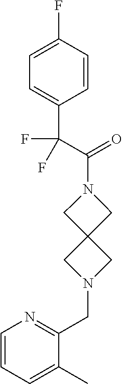 [SMILES: Cc1cccnc1CN1CC2(C1)CN(C(=O)C(F)(F)c1ccc(F)cc1)C2] | Cpd B087 |
| 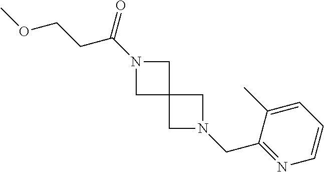 [SMILES: COCCC(=O)N1CC2(CN(Cc3ncccc3C)C2)C1] | Cpd B088 |
| 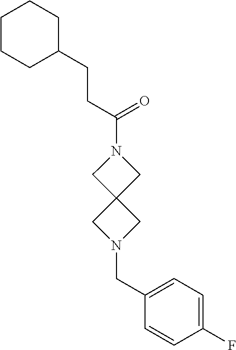 [SMILES: O=C(CCC1CCCCC1)N1CC2(CN(Cc3ccc(F)cc3)C2)C1] | Cpd B089 |
| 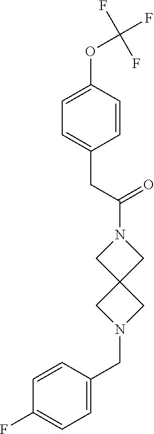 [SMILES: O=C(Cc1ccc(OC(F)(F)F)cc1)N1CC2(CN(Cc3ccc(F)cc3)C2)C1] | Cpd B090 |
| 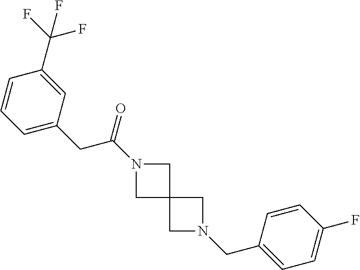 [SMILES: O=C(Cc1cccc(C(F)(F)F)c1)N1CC2(CN(Cc3ccc(F)cc3)C2)C1] | Cpd B091 |
| 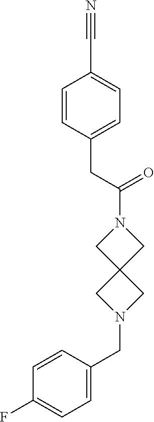 [SMILES: N#Cc1ccc(CC(=O)N2CC3(CN(Cc4ccc(F)cc4)C3)C2)cc1] | Cpd B092 |
| 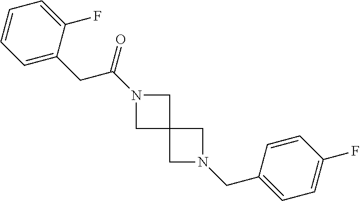 [SMILES: O=C(Cc1ccccc1F)N1CC2(CN(Cc3ccc(F)cc3)C2)C1] | Cpd B093 |
| 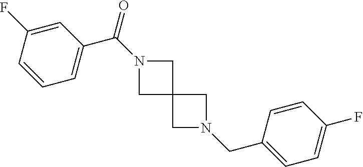 [SMILES: O=C(c1cccc(F)c1)N1CC2(CN(Cc3ccc(F)cc3)C2)C1] | Cpd B094 |
| 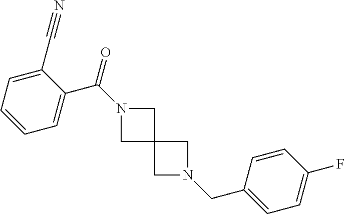 [SMILES: N#Cc1ccccc1C(=O)N1CC2(CN(Cc3ccc(F)cc3)C2)C1] | Cpd B095 |
| 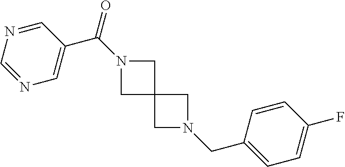 [SMILES: O=C(c1cncnc1)N1CC2(CN(Cc3ccc(F)cc3)C2)C1] | Cpd B096 |
| 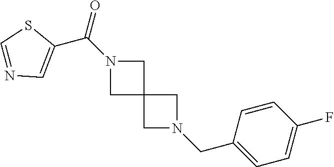 [SMILES: O=C(c1cncs1)N1CC2(CN(Cc3ccc(F)cc3)C2)C1] | Cpd B097 |
| 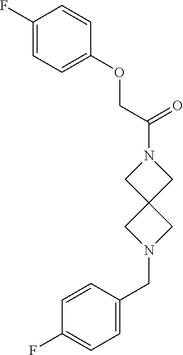 [SMILES: O=C(COc1ccc(F)cc1)N1CC2(CN(Cc3ccc(F)cc3)C2)C1] | Cpd B098 |
| 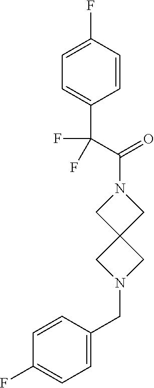 [SMILES: O=C(N1CC2(CN(Cc3ccc(F)cc3)C2)C1)C(F)(F)c1ccc(F)cc1] | Cpd B099 |
| 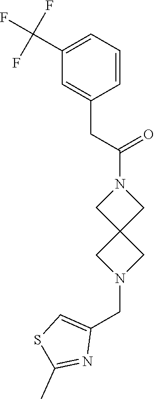 [SMILES: Cc1nc(CN2CC3(C2)CN(C(=O)Cc2cccc(C(F)(F)F)c2)C3)cs1] | Cpd B100 |
| 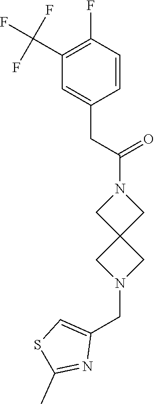 [SMILES: Cc1nc(CN2CC3(C2)CN(C(=O)Cc2ccc(F)c(C(F)(F)F)c2)C3)cs1] | Cpd B101 |
| 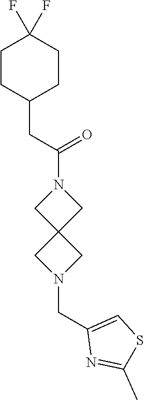 [SMILES: Cc1nc(CN2CC3(C2)CN(C(=O)CC2CCC(F)(F)CC2)C3)cs1] | Cpd B102 |
| 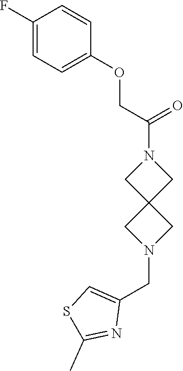 [SMILES: Cc1nc(CN2CC3(C2)CN(C(=O)COc2ccc(F)cc2)C3)cs1] | Cpd B103 |
| 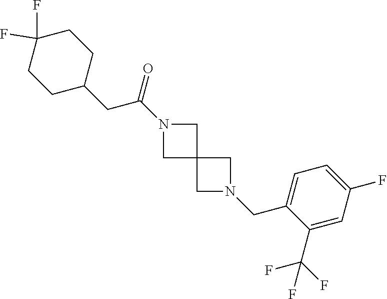 [SMILES: O=C(CC1CCC(F)(F)CC1)N1CC2(CN(Cc3ccc(F)cc3C(F)(F)F)C2)C1] | Cpd B104 |
| 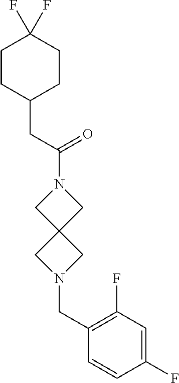 [SMILES: O=C(CC1CCC(F)(F)CC1)N1CC2(CN(Cc3ccc(F)cc3F)C2)C1] | Cpd B105 |
| 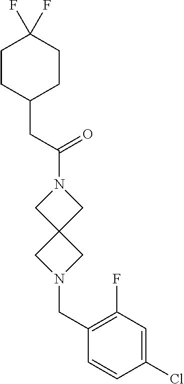 [SMILES: O=C(CC1CCC(F)(F)CC1)N1CC2(CN(Cc3ccc(Cl)cc3F)C2)C1] | Cpd B106 |
| 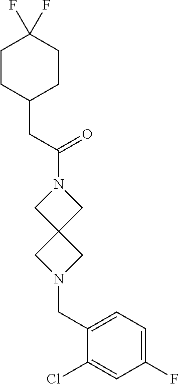 [SMILES: O=C(CC1CCC(F)(F)CC1)N1CC2(CN(Cc3ccc(F)cc3Cl)C2)C1] | Cpd B107 |
| 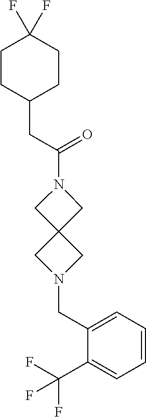 [SMILES: O=C(CC1CCC(F)(F)CC1)N1CC2(CN(Cc3ccccc3C(F)(F)F)C2)C1] | Cpd B108 |
| 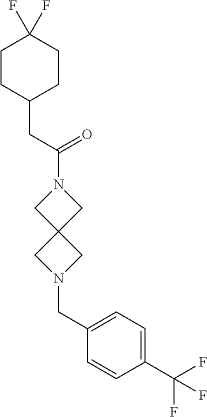 [SMILES: O=C(CC1CCC(F)(F)CC1)N1CC2(CN(Cc3ccc(C(F)(F)F)cc3)C2)C1] | Cpd B109 |
| 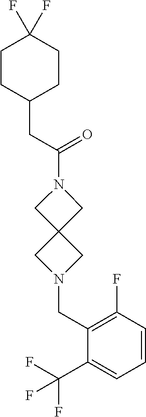 [SMILES: O=C(CC1CCC(F)(F)CC1)N1CC2(CN(Cc3c(F)cccc3C(F)(F)F)C2)C1] | Cpd B110 |
| 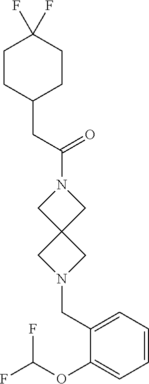 [SMILES: O=C(CC1CCC(F)(F)CC1)N1CC2(CN(Cc3ccccc3OC(F)F)C2)C1] | Cpd B111 |
| 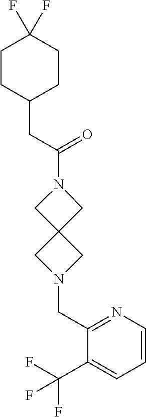 [SMILES: O=C(CC1CCC(F)(F)CC1)N1CC2(CN(Cc3ncccc3C(F)(F)F)C2)C1] | Cpd B112 |
| 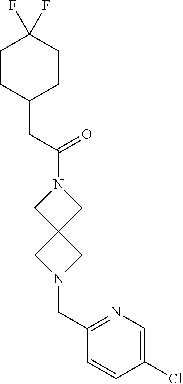 [SMILES: O=C(CC1CCC(F)(F)CC1)N1CC2(CN(Cc3ccc(Cl)cn3)C2)C1] | Cpd B113 |
| 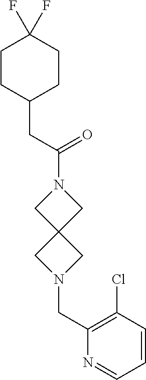 [SMILES: O=C(CC1CCC(F)(F)CC1)N1CC2(CN(Cc3ncccc3Cl)C2)C1] | Cpd B114 |
| 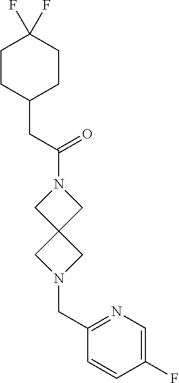 [SMILES: O=C(CC1CCC(F)(F)CC1)N1CC2(CN(Cc3ccc(F)cn3)C2)C1] | Cpd B115 |
| 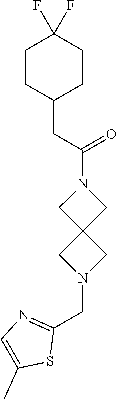 [SMILES: Cc1cnc(CN2CC3(C2)CN(C(=O)CC2CCC(F)(F)CC2)C3)s1] | Cpd B116 |
| 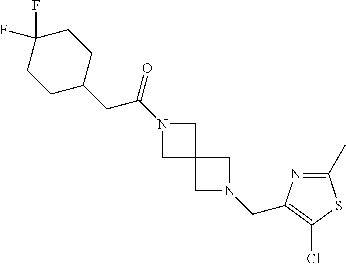 [SMILES: Cc1nc(CN2CC3(C2)CN(C(=O)CC2CCC(F)(F)CC2)C3)c(Cl)s1] | Cpd B117 |
| 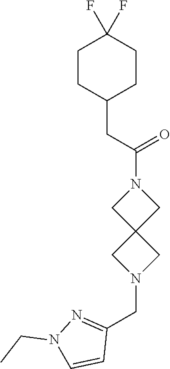 [SMILES: CCn1ccc(CN2CC3(C2)CN(C(=O)CC2CCC(F)(F)CC2)C3)n1] | Cpd B118 |
| 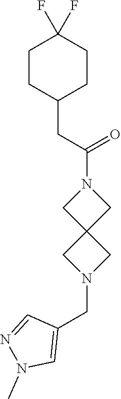 [SMILES: Cn1cc(CN2CC3(C2)CN(C(=O)CC2CCC(F)(F)CC2)C3)cn1] | Cpd B119 |
| 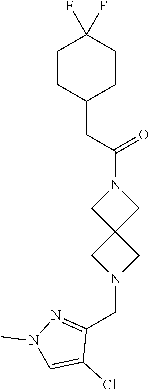 [SMILES: Cn1cc(Cl)c(CN2CC3(C2)CN(C(=O)CC2CCC(F)(F)CC2)C3)n1] | Cpd B120 |
| 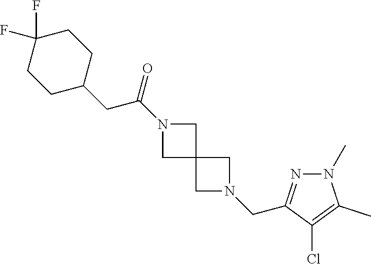 [SMILES: Cc1c(Cl)c(CN2CC3(C2)CN(C(=O)CC2CCC(F)(F)CC2)C3)nn1C] | Cpd B121 |
| 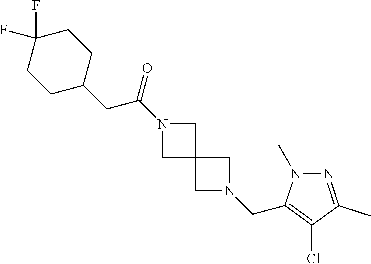 [SMILES: Cc1nn(C)c(CN2CC3(C2)CN(C(=O)CC2CCC(F)(F)CC2)C3)c1Cl] | Cpd B122 |
| 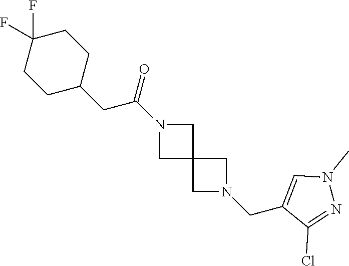 [SMILES: Cn1cc(CN2CC3(C2)CN(C(=O)CC2CCC(F)(F)CC2)C3)c(Cl)n1] | Cpd B123 |
| 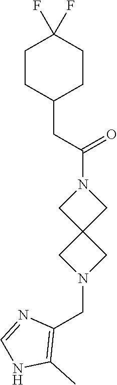 [SMILES: Cc1[nH]cnc1CN1CC2(C1)CN(C(=O)CC1CCC(F)(F)CC1)C2] | Cpd B124 |
| 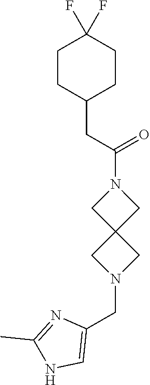 [SMILES: Cc1nc(CN2CC3(C2)CN(C(=O)CC2CCC(F)(F)CC2)C3)c[nH]1] | Cpd B125 |
| 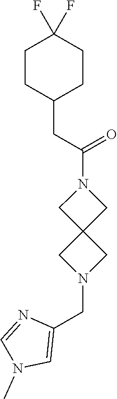 [SMILES: Cn1cnc(CN2CC3(C2)CN(C(=O)CC2CCC(F)(F)CC2)C3)c1] | Cpd B126 |
| 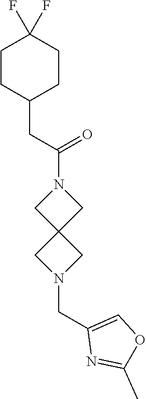 [SMILES: Cc1nc(CN2CC3(C2)CN(C(=O)CC2CCC(F)(F)CC2)C3)co1] | Cpd B127 |
| 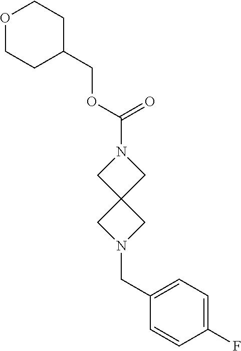 [SMILES: O=C(OCC1CCOCC1)N1CC2(CN(Cc3ccc(F)cc3)C2)C1] | Cpd B128 |
| 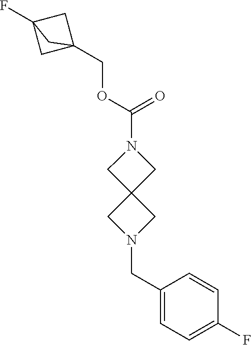 [SMILES: O=C(OCC12CC(F)(C1)C2)N1CC2(CN(Cc3ccc(F)cc3)C2)C1] | Cpd B129 |
| 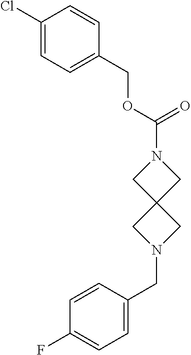 [SMILES: O=C(OCc1ccc(Cl)cc1)N1CC2(CN(Cc3ccc(F)cc3)C2)C1] | Cpd B130 |
| 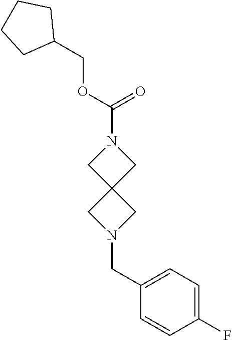 [SMILES: O=C(OCC1CCCC1)N1CC2(CN(Cc3ccc(F)cc3)C2)C1] | Cpd B131 |
| 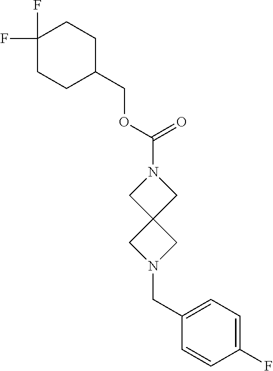 [SMILES: O=C(OCC1CCC(F)(F)CC1)N1CC2(CN(Cc3ccc(F)cc3)C2)C1] | Cpd B132 |
| 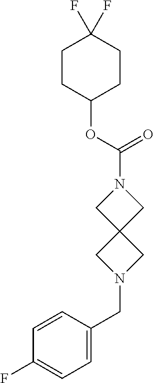 [SMILES: O=C(OC1CCC(F)(F)CC1)N1CC2(CN(Cc3ccc(F)cc3)C2)C1] | Cpd B133 |
| 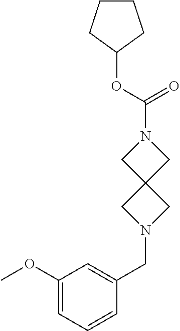 [SMILES: COc1cccc(CN2CC3(C2)CN(C(=O)OC2CCCC2)C3)c1] | Cpd B134 |
| 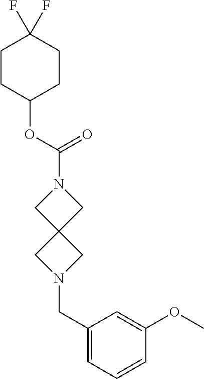 [SMILES: COc1cccc(CN2CC3(C2)CN(C(=O)OC2CCC(F)(F)CC2)C3)c1] | Cpd B135 |
| 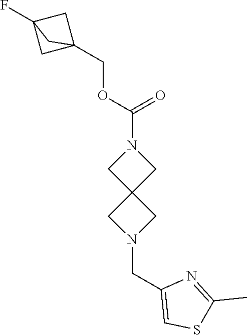 [SMILES: Cc1nc(CN2CC3(C2)CN(C(=O)OCC24CC(F)(C2)C4)C3)cs1] | Cpd B136 |
| 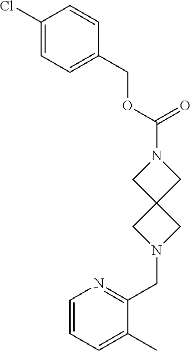 [SMILES: Cc1cccnc1CN1CC2(C1)CN(C(=O)OCc1ccc(Cl)cc1)C2] | Cpd B137 |
| 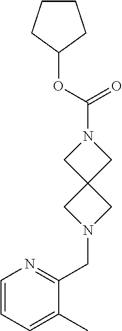 [SMILES: Cc1cccnc1CN1CC2(C1)CN(C(=O)OC1CCCC1)C2] | Cpd B138 |
| 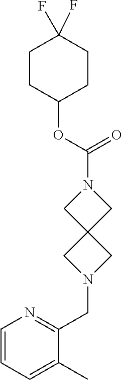 [SMILES: Cc1cccnc1CN1CC2(C1)CN(C(=O)OC1CCC(F)(F)CC1)C2] | Cpd B139 |
| 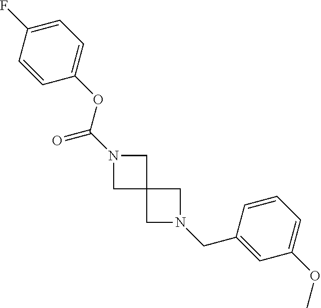 [SMILES: COc1cccc(CN2CC3(C2)CN(C(=O)Oc2ccc(F)cc2)C3)c1] | Cpd B140 |
| 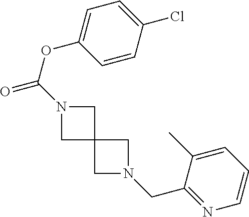 [SMILES: Cc1cccnc1CN1CC2(C1)CN(C(=O)Oc1ccc(Cl)cc1)C2] | Cpd B141 |
| 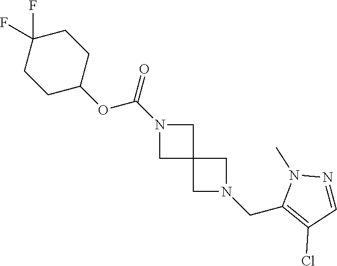 [SMILES: Cn1ncc(Cl)c1CN1CC2(C1)CN(C(=O)OC1CCC(F)(F)CC1)C2] | Cpd B142 |
| 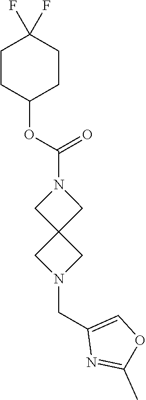 [SMILES: Cc1nc(CN2CC3(C2)CN(C(=O)OC2CCC(F)(F)CC2)C3)co1] | Cpd B143 |
| 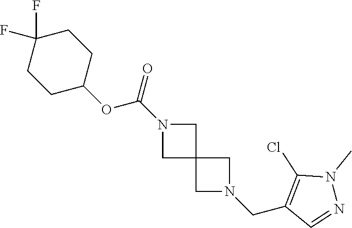 [SMILES: Cn1ncc(CN2CC3(C2)CN(C(=O)OC2CCC(F)(F)CC2)C3)c1Cl] | Cpd B144 |
| 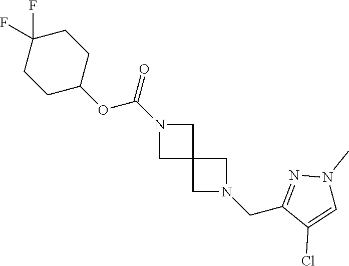 [SMILES: Cn1cc(Cl)c(CN2CC3(C2)CN(C(=O)OC2CCC(F)(F)CC2)C3)n1] | Cpd B145 |
| 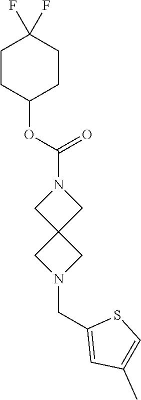 [SMILES: Cc1csc(CN2CC3(C2)CN(C(=O)OC2CCC(F)(F)CC2)C3)c1] | Cpd B146 |
| 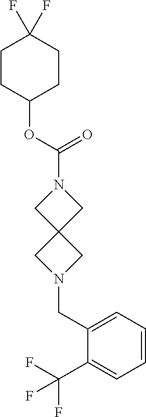 [SMILES: O=C(OC1CCC(F)(F)CC1)N1CC2(CN(Cc3ccccc3C(F)(F)F)C2)C1] | Cpd B147 |
| 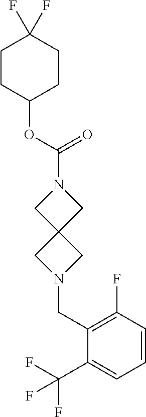 [SMILES: O=C(OC1CCC(F)(F)CC1)N1CC2(CN(Cc3c(F)cccc3C(F)(F)F)C2)C1] | Cpd B148 |
| 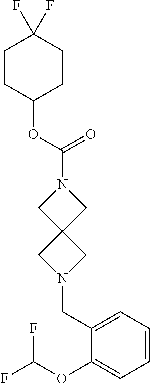 [SMILES: O=C(OC1CCC(F)(F)CC1)N1CC2(CN(Cc3ccccc3OC(F)F)C2)C1] | Cpd B149 |
| 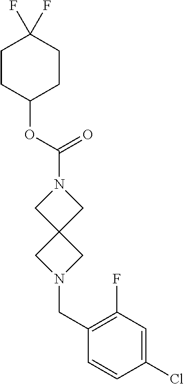 [SMILES: O=C(OC1CCC(F)(F)CC1)N1CC2(CN(Cc3ccc(Cl)cc3F)C2)C1] | Cpd B150 |
| 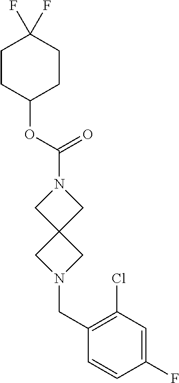 [SMILES: O=C(OC1CCC(F)(F)CC1)N1CC2(CN(Cc3ccc(F)cc3Cl)C2)C1] | Cpd B151 |
| 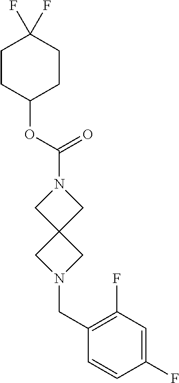 [SMILES: O=C(OC1CCC(F)(F)CC1)N1CC2(CN(Cc3ccc(F)cc3F)C2)C1] | Cpd B152 |
| 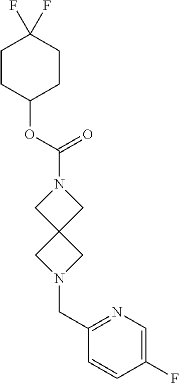 [SMILES: O=C(OC1CCC(F)(F)CC1)N1CC2(CN(Cc3ccc(F)cn3)C2)C1] | Cpd B153 |
| 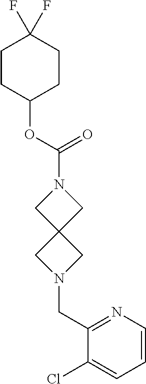 [SMILES: O=C(OC1CCC(F)(F)CC1)N1CC2(CN(Cc3ncccc3Cl)C2)C1] | Cpd B154 |
| 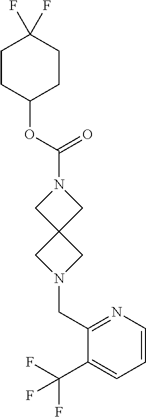 [SMILES: O=C(OC1CCC(F)(F)CC1)N1CC2(CN(Cc3ncccc3C(F)(F)F)C2)C1] | Cpd B155 |
| 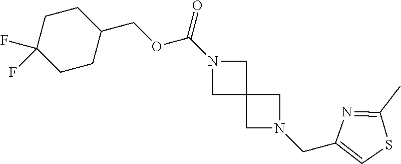 [SMILES: Cc1nc(CN2CC3(C2)CN(C(=O)OCC2CCC(F)(F)CC2)C3)cs1] | Cpd B156 |
| 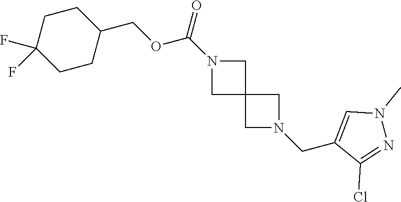 [SMILES: Cn1cc(CN2CC3(C2)CN(C(=O)OCC2CCC(F)(F)CC2)C3)c(Cl)n1] | Cpd B157 |
| 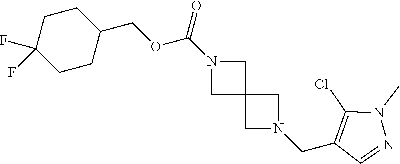 [SMILES: Cn1ncc(CN2CC3(C2)CN(C(=O)OCC2CCC(F)(F)CC2)C3)c1Cl] | Cpd B158 |
| 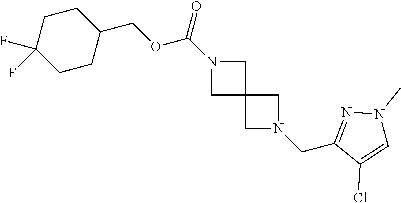 [SMILES: Cn1cc(Cl)c(CN2CC3(C2)CN(C(=O)OCC2CCC(F)(F)CC2)C3)n1] | Cpd B159 |
| 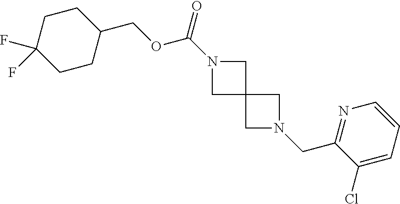 [SMILES: O=C(OCC1CCC(F)(F)CC1)N1CC2(CN(Cc3ncccc3Cl)C2)C1] | Cpd B160 |
| 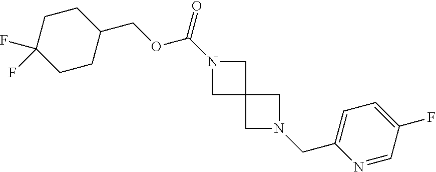 [SMILES: O=C(OCC1CCC(F)(F)CC1)N1CC2(CN(Cc3ccc(F)cn3)C2)C1] | Cpd B161 |
| 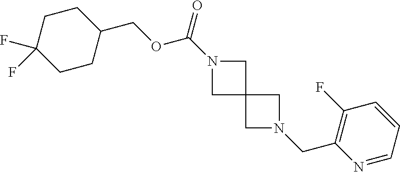 [SMILES: O=C(OCC1CCC(F)(F)CC1)N1CC2(CN(Cc3ncccc3F)C2)C1] | Cpd B162 |
| 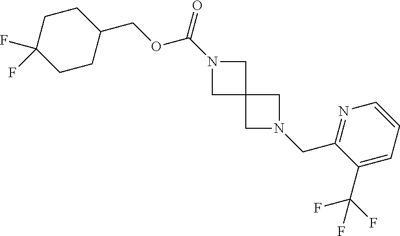 [SMILES: O=C(OCC1CCC(F)(F)CC1)N1CC2(CN(Cc3ncccc3C(F)(F)F)C2)C1] | Cpd B163 |
| 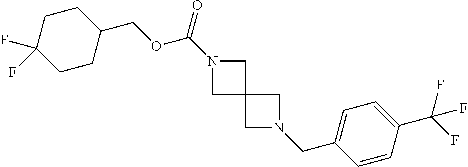 [SMILES: O=C(OCC1CCC(F)(F)CC1)N1CC2(CN(Cc3ccc(C(F)(F)F)cc3)C2)C1] | Cpd B164 |
| 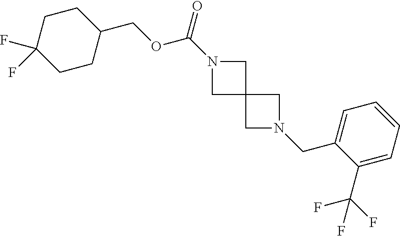 [SMILES: O=C(OCC1CCC(F)(F)CC1)N1CC2(CN(Cc3ccccc3C(F)(F)F)C2)C1] | Cpd B165 |
| 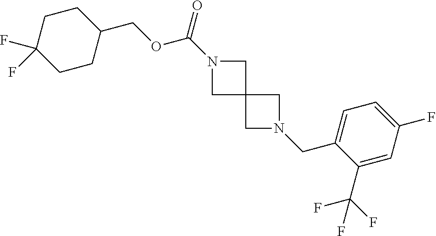 [SMILES: O=C(OCC1CCC(F)(F)CC1)N1CC2(CN(Cc3ccc(F)cc3C(F)(F)F)C2)C1] | Cpd B166 |
| 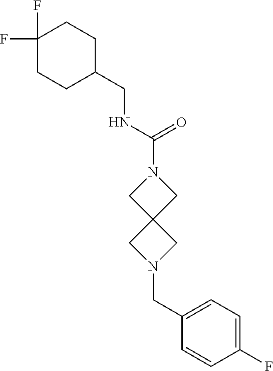 [SMILES: O=C(NCC1CCC(F)(F)CC1)N1CC2(CN(Cc3ccc(F)cc3)C2)C1] | Cpd B167 |
| 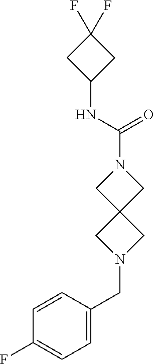 [SMILES: O=C(NC1CC(F)(F)C1)N1CC2(CN(Cc3ccc(F)cc3)C2)C1] | Cpd B168 |
| 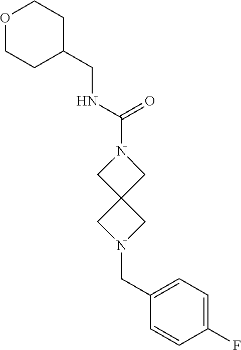 [SMILES: O=C(NCC1CCOCC1)N1CC2(CN(Cc3ccc(F)cc3)C2)C1] | Cpd B169 |
| 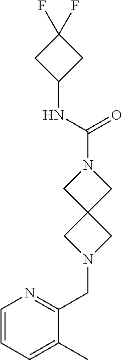 [SMILES: Cc1cccnc1CN1CC2(C1)CN(C(=O)NC1CC(F)(F)C1)C2] | Cpd B170 |
| 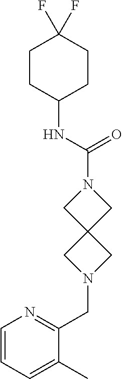 [SMILES: Cc1cccnc1CN1CC2(C1)CN(C(=O)NC1CCC(F)(F)CC1)C2] | Cpd B171 |
| 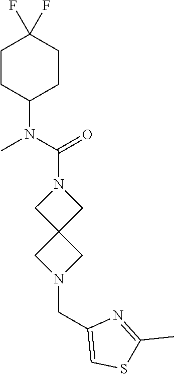 [SMILES: Cc1nc(CN2CC3(C2)CN(C(=O)N(C)C2CCC(F)(F)CC2)C3)cs1] | Cpd B172 |
| 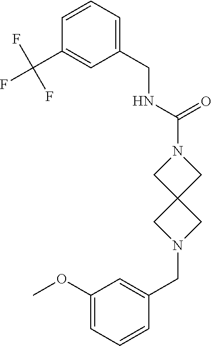 [SMILES: COc1cccc(CN2CC3(C2)CN(C(=O)NCc2cccc(C(F)(F)F)c2)C3)c1] | Cpd B173 |
| 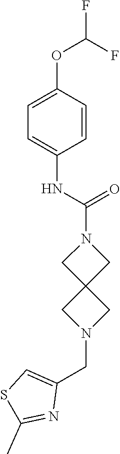 [SMILES: Cc1nc(CN2CC3(C2)CN(C(=O)Nc2ccc(OC(F)F)cc2)C3)cs1] | Cpd B174 |
| 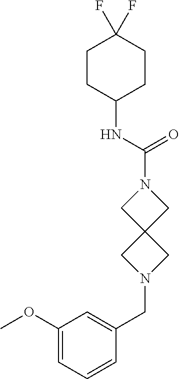 [SMILES: COc1cccc(CN2CC3(C2)CN(C(=O)NC2CCC(F)(F)CC2)C3)c1] | Cpd B175 |
| 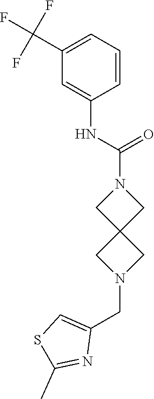 [SMILES: Cc1nc(CN2CC3(C2)CN(C(=O)Nc2cccc(C(F)(F)F)c2)C3)cs1] | Cpd B176 |
| 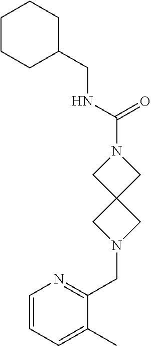 [SMILES: Cc1cccnc1CN1CC2(C1)CN(C(=O)NCC1CCCCC1)C2] | Cpd B177 |
| 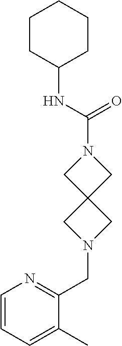 [SMILES: Cc1cccnc1CN1CC2(C1)CN(C(=O)NC1CCCCC1)C2] | Cpd B178 |
| 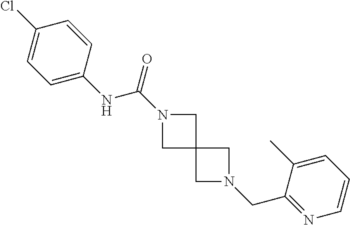 [SMILES: Cc1cccnc1CN1CC2(C1)CN(C(=O)Nc1ccc(Cl)cc1)C2] | Cpd B179 |
| 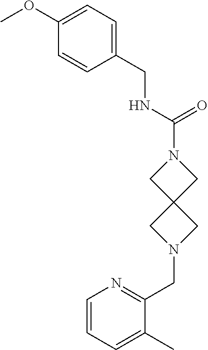 [SMILES: COc1ccc(CNC(=O)N2CC3(CN(Cc4ncccc4C)C3)C2)cc1] | Cpd B180 |
| 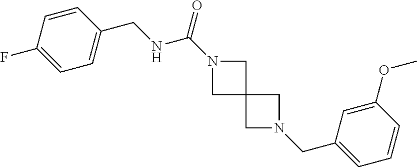 [SMILES: COc1cccc(CN2CC3(C2)CN(C(=O)NCc2ccc(F)cc2)C3)c1] | Cpd B181 |
| 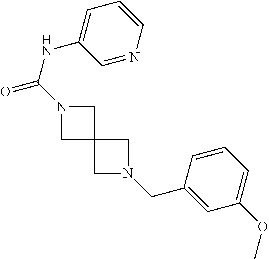 [SMILES: COc1cccc(CN2CC3(C2)CN(C(=O)Nc2cccnc2)C3)c1] | Cpd B182 |
| 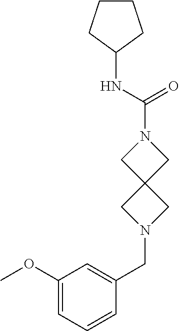 [SMILES: COc1cccc(CN2CC3(C2)CN(C(=O)NC2CCCC2)C3)c1] | Cpd B183 |
| 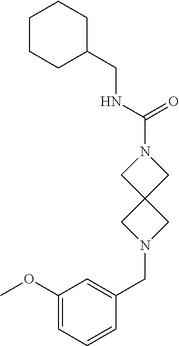 [SMILES: COc1cccc(CN2CC3(C2)CN(C(=O)NCC2CCCCC2)C3)c1] | Cpd B184 |
| 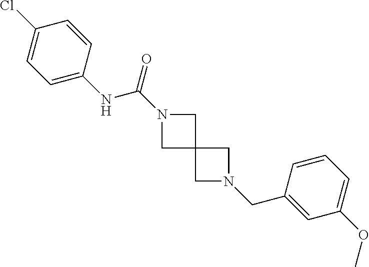 [SMILES: COc1cccc(CN2CC3(C2)CN(C(=O)Nc2ccc(Cl)cc2)C3)c1] | Cpd B185 |
| 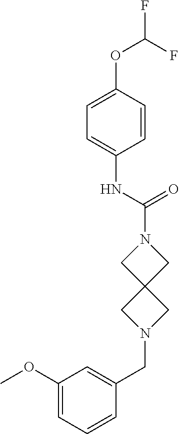 [SMILES: COc1cccc(CN2CC3(C2)CN(C(=O)Nc2ccc(OC(F)F)cc2)C3)c1] | Cpd B186 |
| 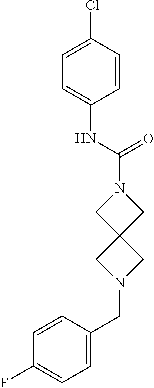 [SMILES: O=C(Nc1ccc(Cl)cc1)N1CC2(CN(Cc3ccc(F)cc3)C2)C1] | Cpd B187 |
| 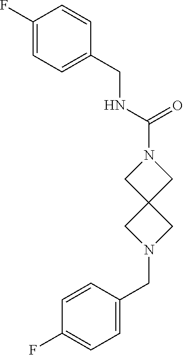 [SMILES: O=C(NCc1ccc(F)cc1)N1CC2(CN(Cc3ccc(F)cc3)C2)C1] | Cpd B188 |
| 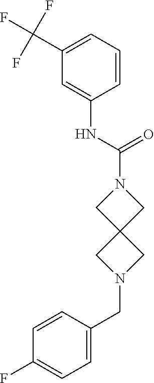 [SMILES: O=C(Nc1cccc(C(F)(F)F)c1)N1CC2(CN(Cc3ccc(F)cc3)C2)C1] | Cpd B189 |
| 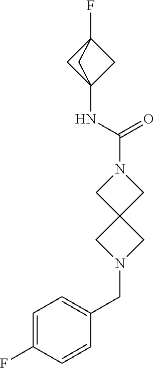 [SMILES: O=C(NC12CC(F)(C1)C2)N1CC2(CN(Cc3ccc(F)cc3)C2)C1] | Cpd B190 |
| 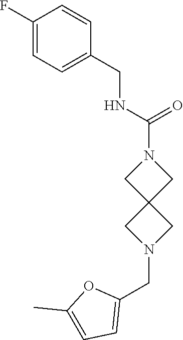 [SMILES: Cc1ccc(CN2CC3(C2)CN(C(=O)NCc2ccc(F)cc2)C3)o1] | Cpd B191 |
| 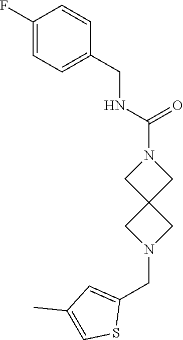 [SMILES: Cc1csc(CN2CC3(C2)CN(C(=O)NCc2ccc(F)cc2)C3)c1] | Cpd B192 |
| 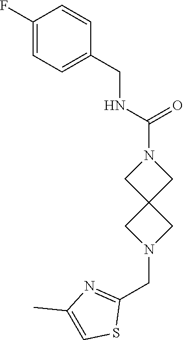 [SMILES: Cc1csc(CN2CC3(C2)CN(C(=O)NCc2ccc(F)cc2)C3)n1] | Cpd B193 |
| 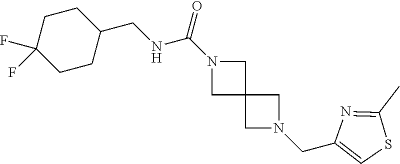 [SMILES: Cc1nc(CN2CC3(C2)CN(C(=O)NCC2CCC(F)(F)CC2)C3)cs1] | Cpd B194 |
| 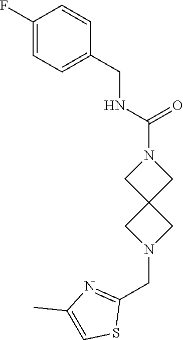 [SMILES: Cc1csc(CN2CC3(C2)CN(C(=O)NCc2ccc(F)cc2)C3)n1] | Cpd B193 |
| 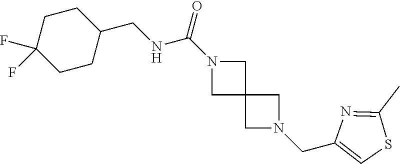 [SMILES: Cc1nc(CN2CC3(C2)CN(C(=O)NCC2CCC(F)(F)CC2)C3)cs1] | Cpd B194 |
| 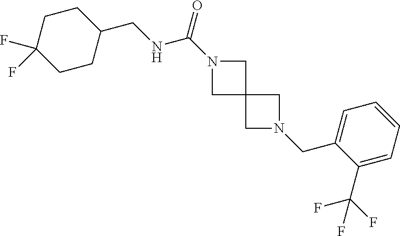 [SMILES: O=C(NCC1CCC(F)(F)CC1)N1CC2(CN(Cc3ccccc3C(F)(F)F)C2)C1] | Cpd B193 |
| 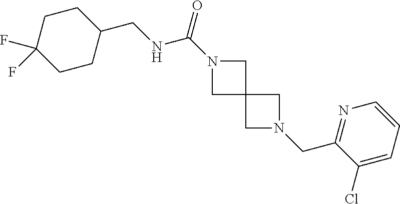 [SMILES: O=C(NCC1CCC(F)(F)CC1)N1CC2(CN(Cc3ncccc3Cl)C2)C1] | Cpd B196 |
| 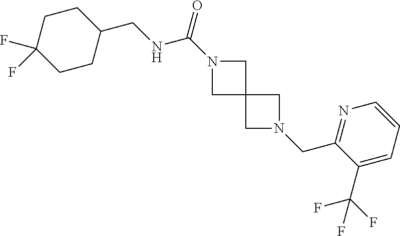 [SMILES: O=C(NCC1CCC(F)(F)CC1)N1CC2(CN(Cc3ncccc3C(F)(F)F)C2)C1] | Cpd B197 |
| 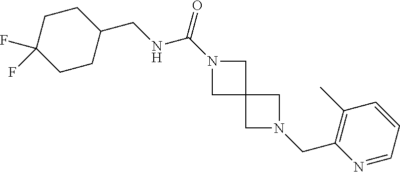 [SMILES: Cc1cccnc1CN1CC2(C1)CN(C(=O)NCC1CCC(F)(F)CC1)C2] | Cpd B198 |
| 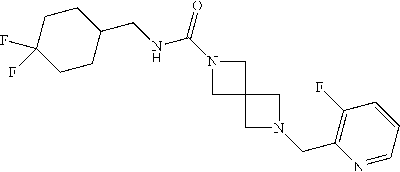 [SMILES: O=C(NCC1CCC(F)(F)CC1)N1CC2(CN(Cc3ncccc3F)C2)C1] | Cpd B199 |
| 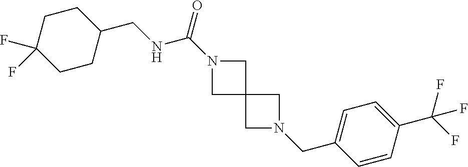 [SMILES: O=C(NCC1CCC(F)(F)CC1)N1CC2(CN(Cc3ccc(C(F)(F)F)cc3)C2)C1] | Cpd B200 |
| 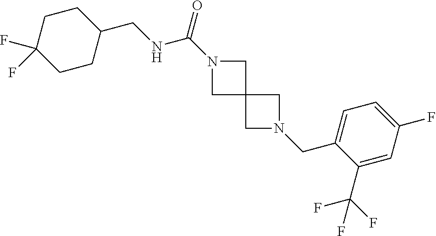 [SMILES: O=C(NCC1CCC(F)(F)CC1)N1CC2(CN(Cc3ccc(F)cc3C(F)(F)F)C2)C1] | Cpd B201 |
| 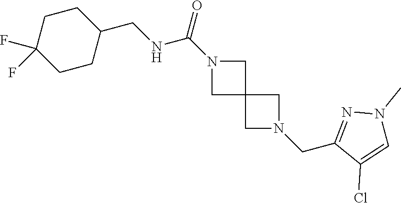 [SMILES: Cn1cc(Cl)c(CN2CC3(C2)CN(C(=O)NCC2CCC(F)(F)CC2)C3)n1] | Cpd B202 |
| 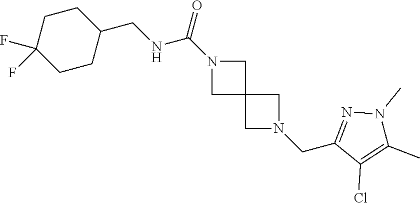 [SMILES: Cc1c(Cl)c(CN2CC3(C2)CN(C(=O)NCC2CCC(F)(F)CC2)C3)nn1C] | Cpd B203 |
| 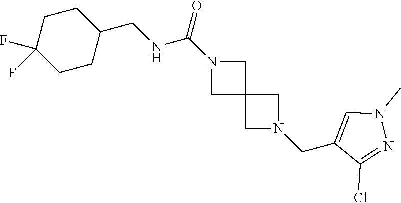 [SMILES: Cn1cc(CN2CC3(C2)CN(C(=O)NCC2CCC(F)(F)CC2)C3)c(Cl)n1] | Cpd B204 |
| 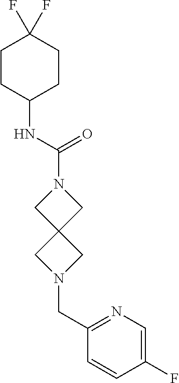 [SMILES: O=C(NC1CCC(F)(F)CC1)N1CC2(CN(Cc3ccc(F)cn3)C2)C1] | Cpd B205 |
| 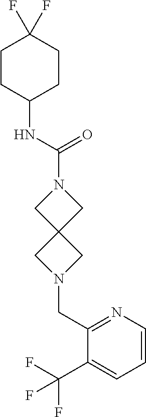 [SMILES: O=C(NC1CCC(F)(F)CC1)N1CC2(CN(Cc3ncccc3C(F)(F)F)C2)C1] | Cpd B206 |
| 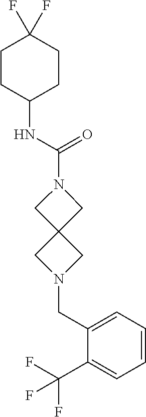 [SMILES: O=C(NC1CCC(F)(F)CC1)N1CC2(CN(Cc3ccccc3C(F)(F)F)C2)C1] | Cpd B207 |
| 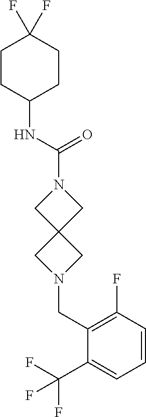 [SMILES: O=C(NC1CCC(F)(F)CC1)N1CC2(CN(Cc3c(F)cccc3C(F)(F)F)C2)C1] | Cpd B208 |
| 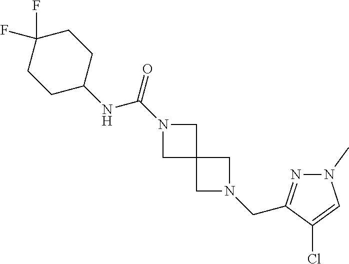 [SMILES: Cn1cc(Cl)c(CN2CC3(C2)CN(C(=O)NC2CCC(F)(F)CC2)C3)n1] | Cpd B209 |
| 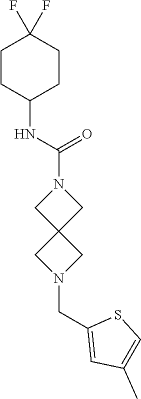 [SMILES: Cc1csc(CN2CC3(C2)CN(C(=O)NC2CCC(F)(F)CC2)C3)c1] | Cpd B210 |
| 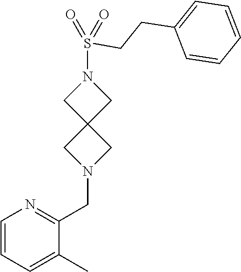 [SMILES: Cc1cccnc1CN1CC2(C1)CN(S(=O)(=O)CCc1ccccc1)C2] | Cpd B211 |
| 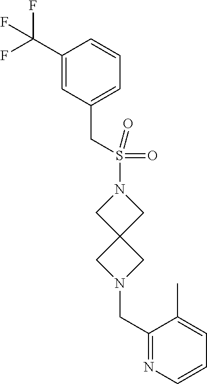 [SMILES: Cc1cccnc1CN1CC2(C1)CN(S(=O)(=O)Cc1cccc(C(F)(F)F)c1)C2] | Cpd B212 |
| 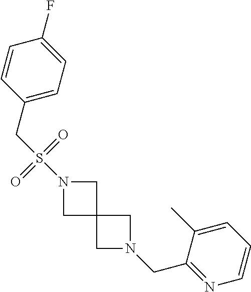 [SMILES: Cc1cccnc1CN1CC2(C1)CN(S(=O)(=O)Cc1ccc(F)cc1)C2] | Cpd B213 |
| 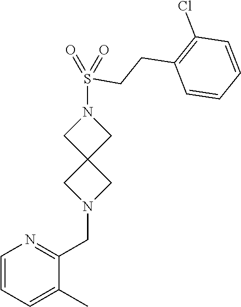 [SMILES: Cc1cccnc1CN1CC2(C1)CN(S(=O)(=O)CCc1ccccc1Cl)C2] | Cpd B214 |
| 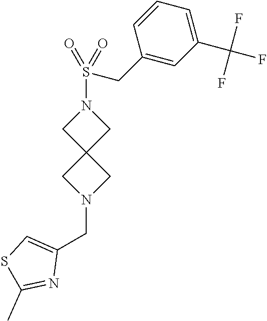 [SMILES: Cc1nc(CN2CC3(C2)CN(S(=O)(=O)Cc2cccc(C(F)(F)F)c2)C3)cs1] | Cpd B215 |
| 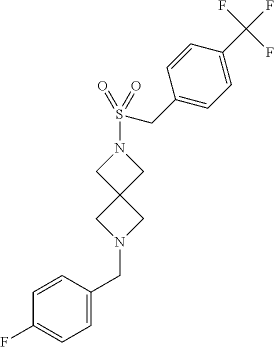 [SMILES: O=S(=O)(Cc1ccc(C(F)(F)F)cc1)N1CC2(CN(Cc3ccc(F)cc3)C2)C1] | Cpd B216 |
| 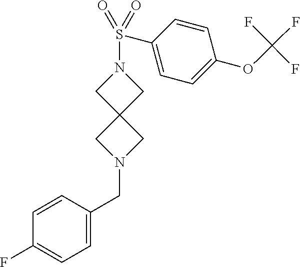 [SMILES: O=S(=O)(c1ccc(OC(F)(F)F)cc1)N1CC2(CN(Cc3ccc(F)cc3)C2)C1] | Cpd B217 |
| 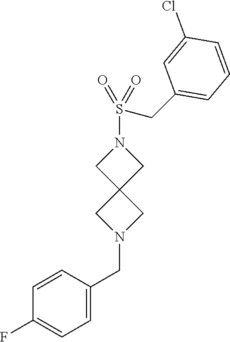 [SMILES: O=S(=O)(Cc1cccc(Cl)c1)N1CC2(CN(Cc3ccc(F)cc3)C2)C1] | Cpd B218 |
| 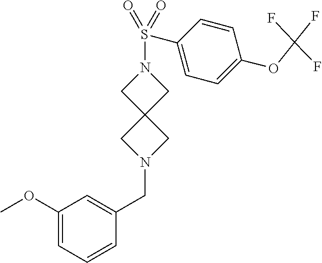 [SMILES: COc1cccc(CN2CC3(C2)CN(S(=O)(=O)c2ccc(OC(F)(F)F)cc2)C3)c1] | Cpd B219 |
| 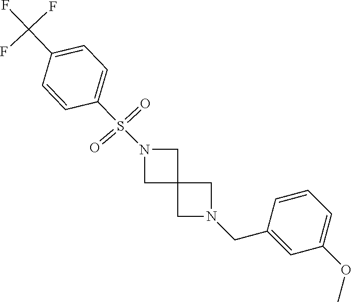 [SMILES: COc1cccc(CN2CC3(C2)CN(S(=O)(=O)c2ccc(C(F)(F)F)cc2)C3)c1] | Cpd B220 |
| 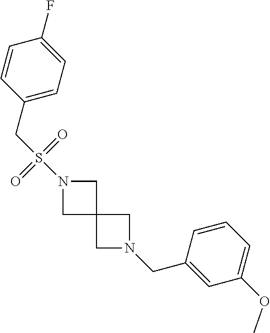 [SMILES: COc1cccc(CN2CC3(C2)CN(S(=O)(=O)Cc2ccc(F)cc2)C3)c1] | Cpd B221 |
| 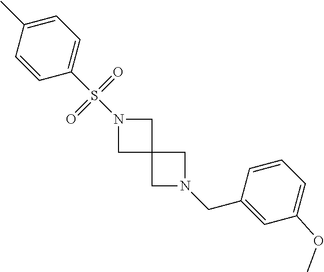 [SMILES: COc1cccc(CN2CC3(C2)CN(S(=O)(=O)c2ccc(C)cc2)C3)c1] | Cpd B222 |
| 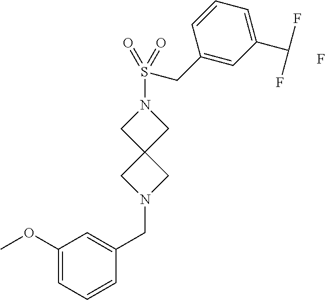 | Cpd B223 |
| 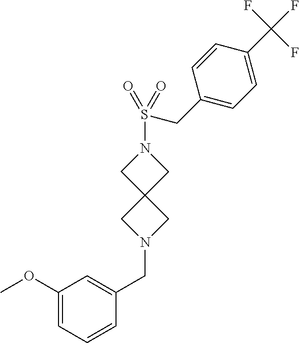 [SMILES: COc1cccc(CN2CC3(C2)CN(S(=O)(=O)Cc2ccc(C(F)(F)F)cc2)C3)c1] | Cpd B224 |
| 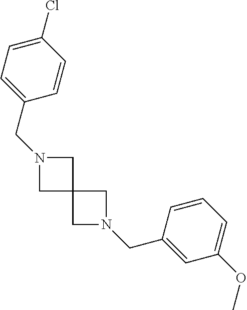 [SMILES: COc1cccc(CN2CC3(CN(Cc4ccc(Cl)cc4)C3)C2)c1] | Cpd B225 |
| 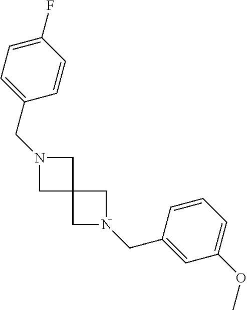 [SMILES: COc1cccc(CN2CC3(CN(Cc4ccc(F)cc4)C3)C2)c1] | Cpd B226 |
| 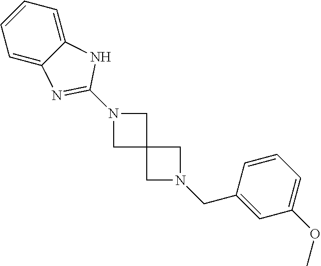 [SMILES: COc1cccc(CN2CC3(C2)CN(c2nc4ccccc4[nH]2)C3)c1] | Cpd B227 |
| 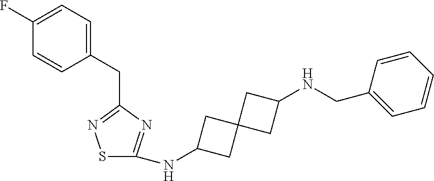 [SMILES: Fc1ccc(Cc2nsc(NC3CC4(CC(NCc5ccccc5)C4)C3)n2)cc1] | Cpd B301 |
| 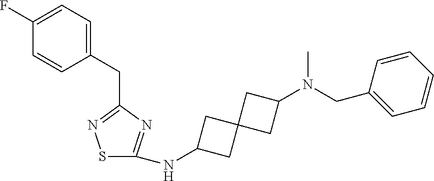 [SMILES: CN(Cc1ccccc1)C1CC2(CC(Nc3nc(Cc4ccc(F)cc4)ns3)C2)C1] | Cpd B302 |
| 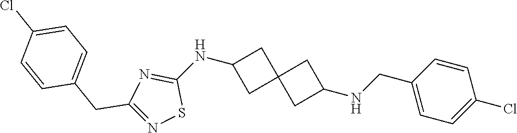 [SMILES: Clc1ccc(CNC2CC3(C2)CC(Nc2nc(Cc4ccc(Cl)cc4)ns2)C3)cc1] | Cpd B303 |
| 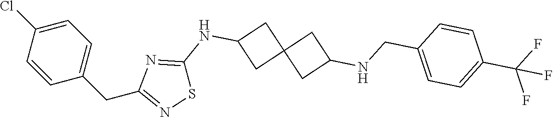 [SMILES: FC(F)(F)c1ccc(CNC2CC3(C2)CC(Nc2nc(Cc4ccc(Cl)cc4)ns2)C3)cc1] | Cpd B304 |
| 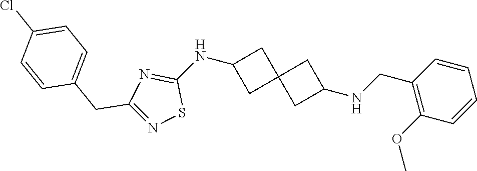 [SMILES: COc1ccccc1CNC1CC2(C1)CC(Nc1nc(Cc3ccc(Cl)cc3)ns1)C2] | Cpd B305 |
| 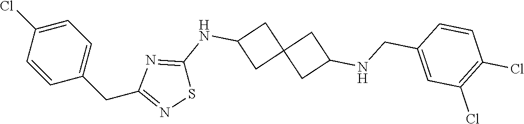 [SMILES: Clc1ccc(Cc2nsc(NC3CC4(CC(NCc5ccc(Cl)c(Cl)c5)C4)C3)n2)cc1] | Cpd B306 |
| 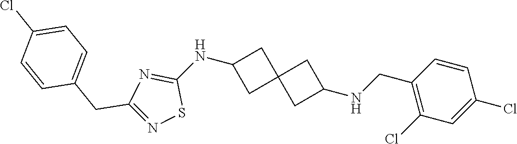 [SMILES: Clc1ccc(Cc2nsc(NC3CC4(CC(NCc5ccc(Cl)cc5Cl)C4)C3)n2)cc1] | Cpd B307 |
| 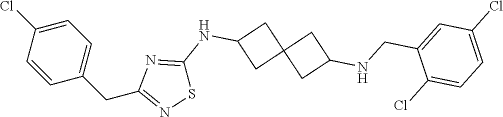 [SMILES: Clc1ccc(Cc2nsc(NC3CC4(CC(NCc5cc(Cl)ccc5Cl)C4)C3)n2)cc1] | Cpd B308 |
| 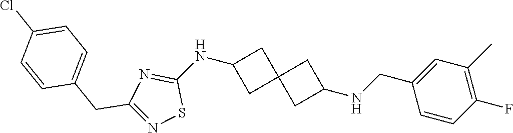 [SMILES: Cc1cc(CNC2CC3(C2)CC(Nc2nc(Cc4ccc(Cl)cc4)ns2)C3)ccc1F] | Cpd B309 |
| 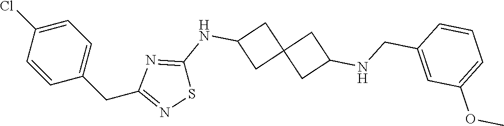 [SMILES: COc1cccc(CNC2CC3(C2)CC(Nc2nc(Cc4ccc(Cl)cc4)ns2)C3)c1] | Cpd B310 |
| 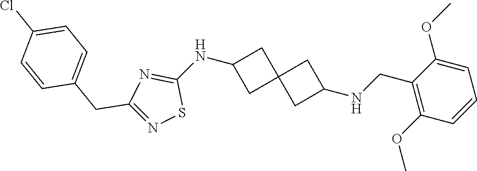 [SMILES: COc1cccc(OC)c1CNC1CC2(C1)CC(Nc1nc(Cc3ccc(Cl)cc3)ns1)C2] | Cpd B311 |
| 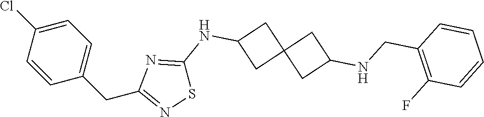 [SMILES: Fc1ccccc1CNC1CC2(C1)CC(Nc1nc(Cc3ccc(Cl)cc3)ns1)C2] | Cpd B312 |
| 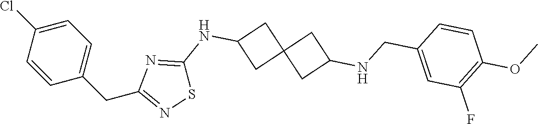 [SMILES: COc1ccc(CNC2CC3(C2)CC(Nc2nc(Cc4ccc(Cl)cc4)ns2)C3)cc1F] | Cpd B313 |
| 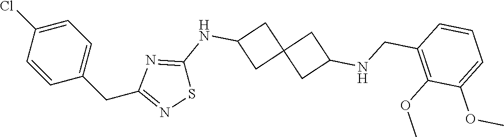 [SMILES: COc1cccc(CNC2CC3(C2)CC(Nc2nc(Cc4ccc(Cl)cc4)ns2)C3)c1OC] | Cpd B314 |
| 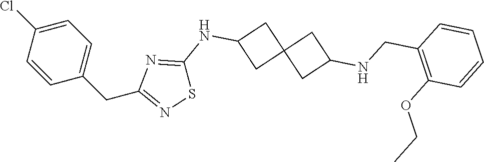 [SMILES: CCOc1ccccc1CNC1CC2(C1)CC(Nc1nc(Cc3ccc(Cl)cc3)ns1)C2] | Cpd B315 |
| 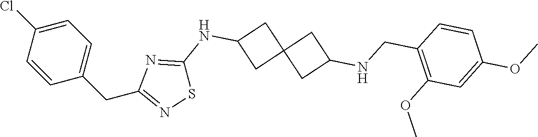 [SMILES: COc1ccc(CNC2CC3(C2)CC(Nc2nc(Cc4ccc(Cl)cc4)ns2)C3)c(OC)c1] | Cpd B316 |
| 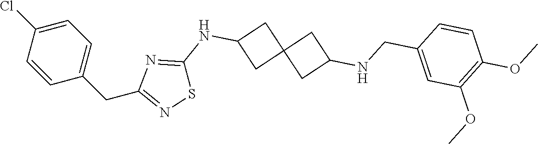 [SMILES: COc1ccc(CNC2CC3(C2)CC(Nc2nc(Cc4ccc(Cl)cc4)ns2)C3)cc1OC] | Cpd B317 |
| 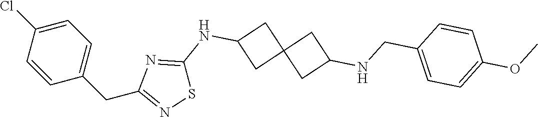 [SMILES: COc1ccc(CNC2CC3(C2)CC(Nc2nc(Cc4ccc(Cl)cc4)ns2)C3)cc1] | Cpd B318 |
| 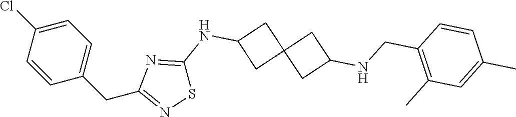 [SMILES: Cc1ccc(CNC2CC3(C2)CC(Nc2nc(Cc4ccc(Cl)cc4)ns2)C3)c(C)c1] | Cpd B319 |
| 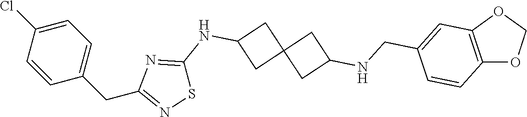 [SMILES: Clc1ccc(Cc2nsc(NC3CC4(CC(NCc5ccc6c(c5)OCO6)C4)C3)n2)cc1] | Cpd B320 |
| 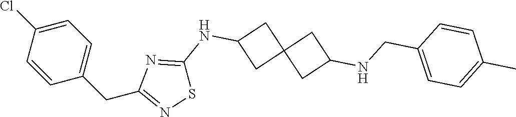 [SMILES: Cc1ccc(CNC2CC3(C2)CC(Nc2nc(Cc4ccc(Cl)cc4)ns2)C3)cc1] | Cpd B321 |
| 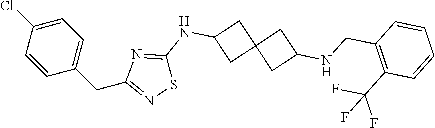 [SMILES: FC(F)(F)c1ccccc1CNC1CC2(C1)CC(Nc1nc(Cc3ccc(Cl)cc3)ns1)C2] | Cpd B322 |
| 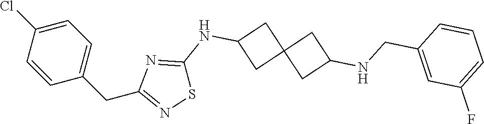 [SMILES: Fc1cccc(CNC2CC3(C2)CC(Nc2nc(Cc4ccc(Cl)cc4)ns2)C3)c1] | Cpd B323 |
| 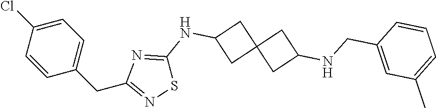 [SMILES: Cc1cccc(CNC2CC3(C2)CC(Nc2nc(Cc4ccc(Cl)cc4)ns2)C3)c1] | Cpd B324 |
| 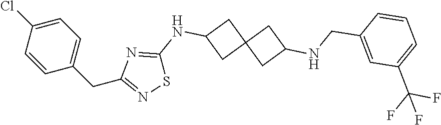 [SMILES: FC(F)(F)c1cccc(CNC2CC3(C2)CC(Nc2nc(Cc4ccc(Cl)cc4)ns2)C3)c1] | Cpd B325 |
| 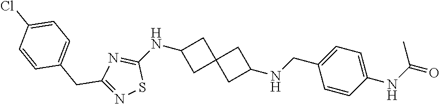 [SMILES: CC(=O)Nc1ccc(CNC2CC3(C2)CC(Nc2nc(Cc4ccc(Cl)cc4)ns2)C3)cc1] | Cpd B326 |
| 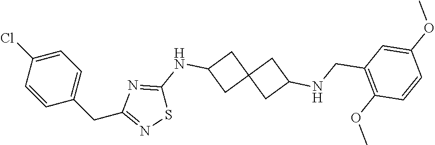 [SMILES: COc1ccc(OC)c(CNC2CC3(C2)CC(Nc2nc(Cc4ccc(Cl)cc4)ns2)C3)c1] | Cpd B327 |
| 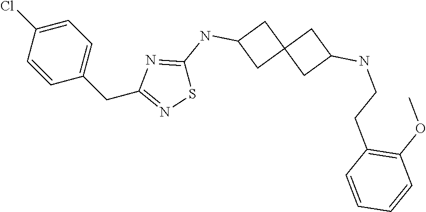 [SMILES: COc1ccccc1CCNC1CC2(C1)CC(Nc1nc(Cc3ccc(Cl)cc3)ns1)C2] | Cpd B328 |
| 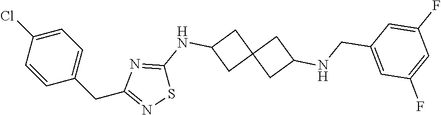 [SMILES: Fc1cc(F)cc(CNC2CC3(C2)CC(Nc2nc(Cc4ccc(Cl)cc4)ns2)C3)c1] | Cpd B329 |
| 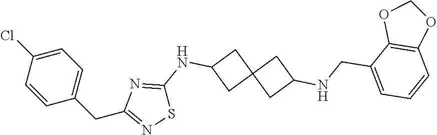 [SMILES: Clc1ccc(Cc2nsc(NC3CC4(CC(NCc5cccc6c5OCO6)C4)C3)n2)cc1] | Cpd B330 |
| 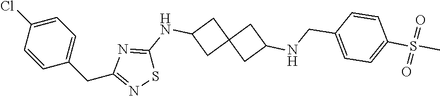 [SMILES: CS(=O)(=O)c1ccc(CNC2CC3(C2)CC(Nc2nc(Cc4ccc(Cl)cc4)ns2)C3)cc1] | Cpd B331 |
| 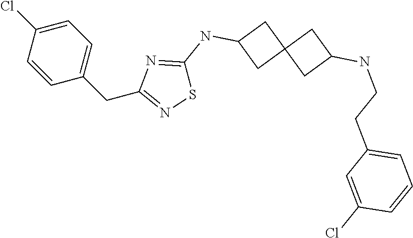 [SMILES: Clc1ccc(Cc2nsc(NC3CC4(CC(NCCc5cccc(Cl)c5)C4)C3)n2)cc1] | Cpd B332 |
| 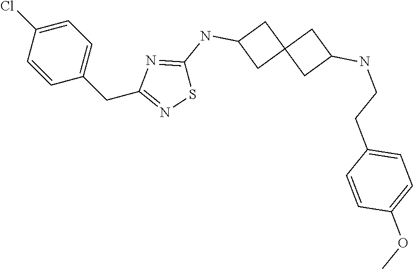 [SMILES: COc1ccc(CCNC2CC3(C2)CC(Nc2nc(Cc4ccc(Cl)cc4)ns2)C3)cc1] | Cpd B333 |
| 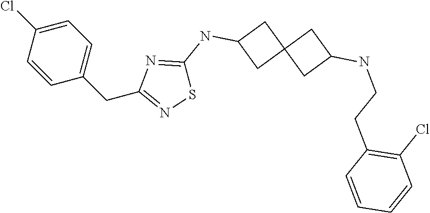 [SMILES: Clc1ccc(Cc2nsc(NC3CC4(CC(NCCc5ccccc5Cl)C4)C3)n2)cc1] | Cpd B334 |
| 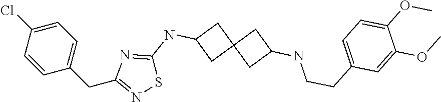 [SMILES: COc1ccc(CCNC2CC3(C2)CC(Nc2nc(Cc4ccc(Cl)cc4)ns2)C3)cc1OC] | Cpd B335 |
| 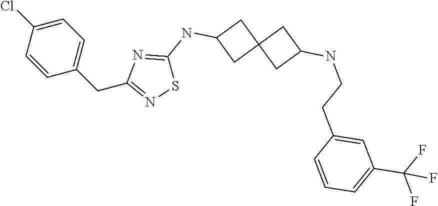 [SMILES: FC(F)(F)c1cccc(CCNC2CC3(C2)CC(Nc2nc(Cc4ccc(Cl)cc4)ns2)C3)c1] | Cpd B336 |
| 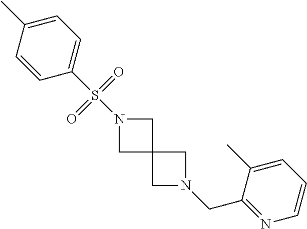 [SMILES: Cc1ccc(S(=O)(=O)N2CC3(CN(Cc4ncccc4C)C3)C2)cc1] | Cpd B401 |
| 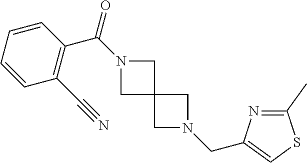 [SMILES: Cc1nc(CN2CC3(C2)CN(C(=O)c2ccccc2C#N)C3)cs1] | Cpd B402 |
| 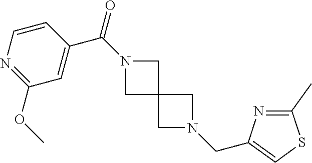 [SMILES: COc1cc(C(=O)N2CC3(CN(Cc4csc(C)n4)C3)C2)ccn1] | Cpd B403 |
| 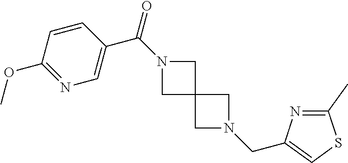 | Cpd B404 |
| 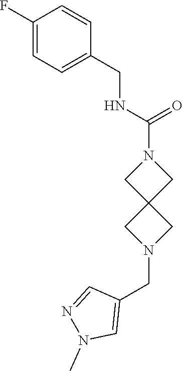 [SMILES: Cn1cc(CN2CC3(C2)CN(C(=O)NCc2ccc(F)cc2)C3)cn1] | Cpd B405 |
| 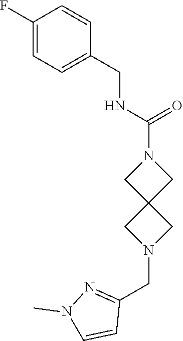 [SMILES: Cn1ccc(CN2CC3(C2)CN(C(=O)NCc2ccc(F)cc2)C3)n1] | Cpd B406 |
| 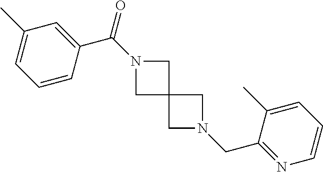 [SMILES: Cc1cccc(C(=O)N2CC3(CN(Cc4ncccc4C)C3)C2)c1] | Cpd B407 |
| 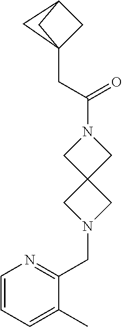 [SMILES: Cc1cccnc1CN1CC2(C1)CN(C(=O)CC13CC(C1)C3)C2] | Cpd B408 |
| 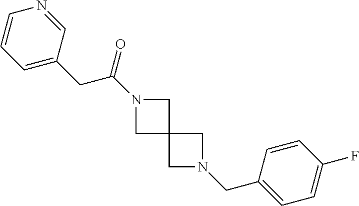 [SMILES: O=C(Cc1cccnc1)N1CC2(CN(Cc3ccc(F)cc3)C2)C1] | Cpd B409 |
| 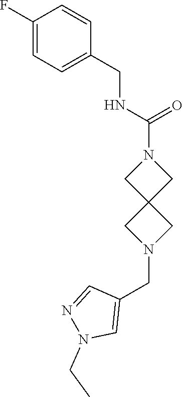 [SMILES: CCn1cc(CN2CC3(C2)CN(C(=O)NCc2ccc(F)cc2)C3)cn1] | Cpd B410 |
| 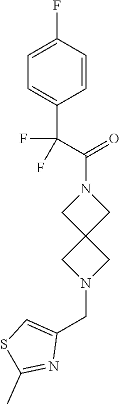 [SMILES: Cc1nc(CN2CC3(C2)CN(C(=O)C(F)(F)c2ccc(F)cc2)C3)cs1] | Cpd B411 |
| 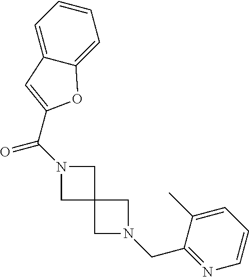 [SMILES: Cc1cccnc1CN1CC2(C1)CN(C(=O)c1cc3ccccc3o1)C2] | Cpd B412 |
| 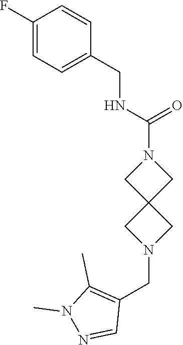 [SMILES: Cc1c(CN2CC3(C2)CN(C(=O)NCc2ccc(F)cc2)C3)cnn1C] | Cpd B413 |
| 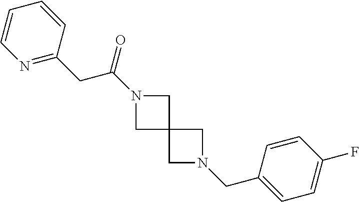 [SMILES: O=C(Cc1ccccn1)N1CC2(CN(Cc3ccc(F)cc3)C2)C1] | Cpd B414 |
| 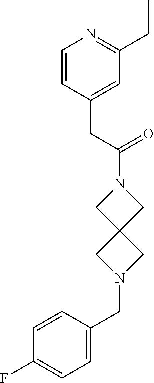 [SMILES: CCc1cc(CC(=O)N2CC3(CN(Cc4ccc(F)cc4)C3)C2)ccn1] | Cpd B415 |
| 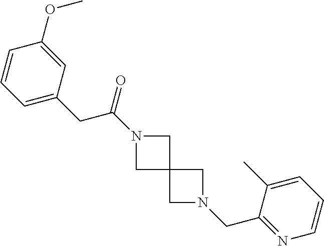 [SMILES: COc1cccc(CC(=O)N2CC3(CN(Cc4ncccc4C)C3)C2)c1] | Cpd B416 |
| 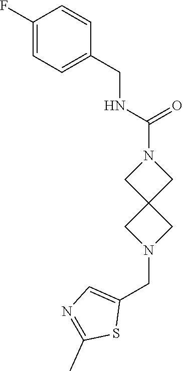 [SMILES: Cc1ncc(CN2CC3(C2)CN(C(=O)NCc2ccc(F)cc2)C3)s1] | Cpd B417 |
| 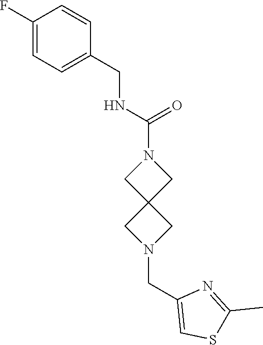 [SMILES: Cc1nc(CN2CC3(C2)CN(C(=O)NCc2ccc(F)cc2)C3)cs1] | Cpd B418 |
| 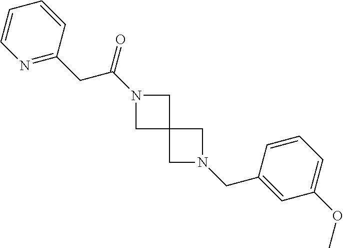 [SMILES: COc1cccc(CN2CC3(C2)CN(C(=O)Cc2ccccn2)C3)c1] | Cpd B419 |
| 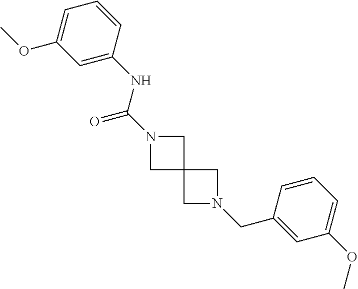 [SMILES: COc1cccc(CN2CC3(C2)CN(C(=O)Nc2cccc(OC)c2)C3)c1] | Cpd B420 |
| 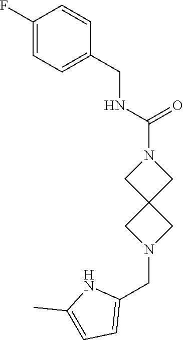 [SMILES: Cc1ccc(CN2CC3(C2)CN(C(=O)NCc2ccc(F)cc2)C3)[nH]1] | Cpd B421 |
| 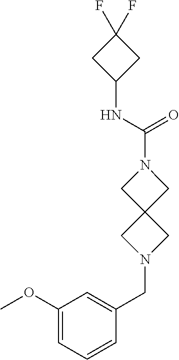 [SMILES: COc1cccc(CN2CC3(C2)CN(C(=O)NC2CC(F)(F)C2)C3)c1] | Cpd B422 |
| 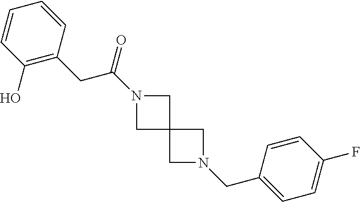 [SMILES: O=C(Cc1ccccc1O)N1CC2(CN(Cc3ccc(F)cc3)C2)C1] | Cpd B423 |
| 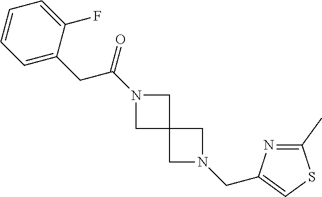 [SMILES: Cc1nc(CN2CC3(C2)CN(C(=O)Cc2ccccc2F)C3)cs1] | Cpd B424 |
| 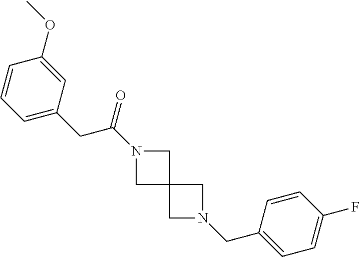 [SMILES: COc1cccc(CC(=O)N2CC3(CN(Cc4ccc(F)cc4)C3)C2)c1] | Cpd B425 |
| 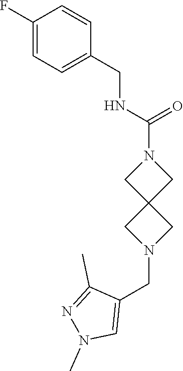 [SMILES: Cc1nn(C)cc1CN1CC2(C1)CN(C(=O)NCc1ccc(F)cc1)C2] | Cpd B426 |
| 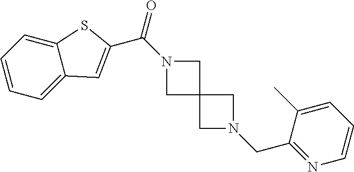 [SMILES: Cc1cccnc1CN1CC2(C1)CN(C(=O)c1cc3ccccc3s1)C2] | Cpd B427 |
| 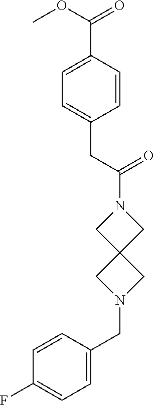 [SMILES: COC(=O)c1ccc(CC(=O)N2CC3(CN(Cc4ccc(F)cc4)C3)C2)cc1] | Cpd B428 |
| 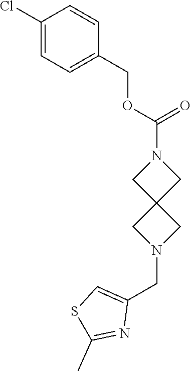 [SMILES: Cc1nc(CN2CC3(C2)CN(C(=O)OCc2ccc(Cl)cc2)C3)cs1] | Cpd B429 |
| 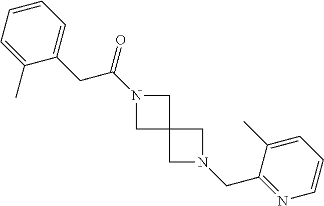 [SMILES: Cc1ccccc1CC(=O)N1CC2(CN(Cc3ncccc3C)C2)C1] | Cpd B430 |
| 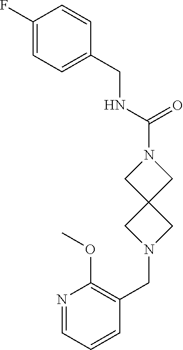 [SMILES: COc1ncccc1CN1CC2(C1)CN(C(=O)NCc1ccc(F)cc1)C2] | Cpd B431 |
| 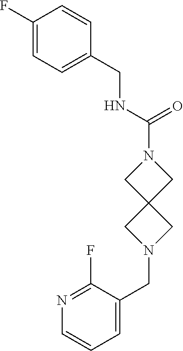 [SMILES: O=C(NCc1ccc(F)cc1)N1CC2(CN(Cc3cccnc3F)C2)C1] | Cpd B432 |
| 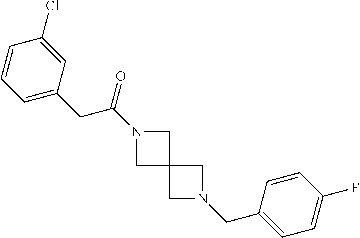 [SMILES: O=C(Cc1cccc(Cl)c1)N1CC2(CN(Cc3ccc(F)cc3)C2)C1] | Cpd B433 |
| 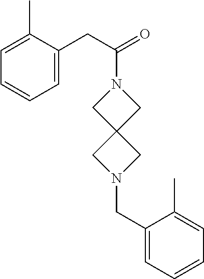 [SMILES: Cc1ccccc1CC(=O)N1CC2(CN(Cc3ccccc3C)C2)C1] | Cpd B434 |
| 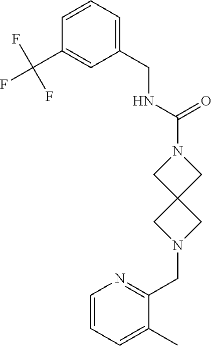 [SMILES: Cc1cccnc1CN1CC2(C1)CN(C(=O)NCc1cccc(C(F)(F)F)c1)C2] | Cpd B435 |
| 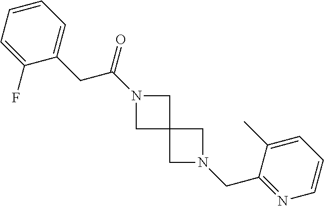 [SMILES: Cc1cccnc1CN1CC2(C1)CN(C(=O)Cc1ccccc1F)C2] | Cpd B436 |
| 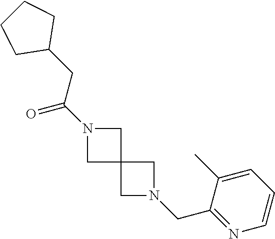 [SMILES: Cc1cccnc1CN1CC2(C1)CN(C(=O)CC1CCCC1)C2] | Cpd B437 |
| 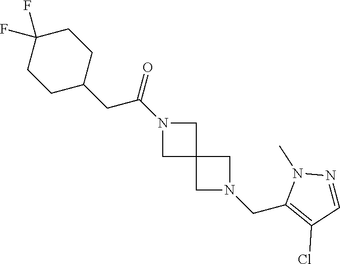 [SMILES: Cn1ncc(Cl)c1CN1CC2(C1)CN(C(=O)CC1CCC(F)(F)CC1)C2] | Cpd B438 |
| 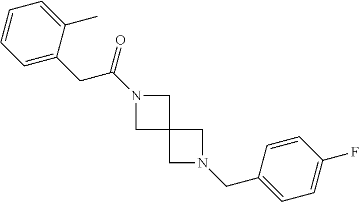 [SMILES: Cc1ccccc1CC(=O)N1CC2(CN(Cc3ccc(F)cc3)C2)C1] | Cpd B439 |
| 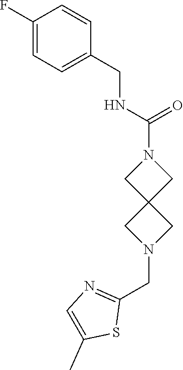 [SMILES: Cc1cnc(CN2CC3(C2)CN(C(=O)NCc2ccc(F)cc2)C3)s1] | Cpd B440 |
| 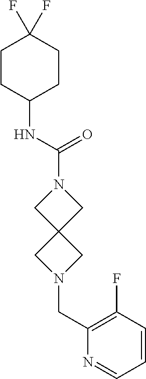 [SMILES: O=C(NC1CCC(F)(F)CC1)N1CC2(CN(Cc3ncccc3F)C2)C1] | Cpd B441 |
| 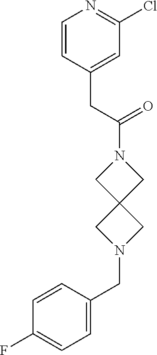 [SMILES: O=C(Cc1ccnc(Cl)c1)N1CC2(CN(Cc3ccc(F)cc3)C2)C1] | Cpd B442 |
| 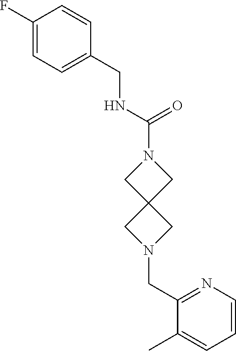 [SMILES: Cc1cccnc1CN1CC2(C1)CN(C(=O)NCc1ccc(F)cc1)C2] | Cpd B443 |
| 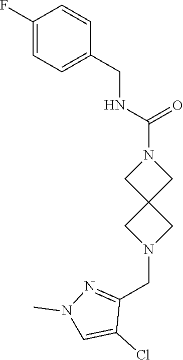 [SMILES: Cn1cc(Cl)c(CN2CC3(C2)CN(C(=O)NCc2ccc(F)cc2)C3)n1] | Cpd B444 |
| 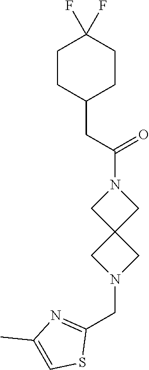 [SMILES: Cc1csc(CN2CC3(C2)CN(C(=O)CC2CCC(F)(F)CC2)C3)n1] | Cpd B445 |
| 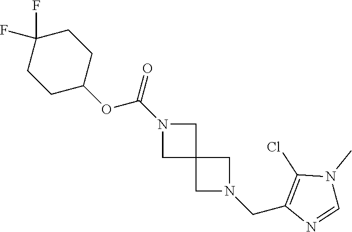 [SMILES: Cn1cnc(CN2CC3(C2)CN(C(=O)OC2CCC(F)(F)CC2)C3)c1Cl] | Cpd B446 |
| 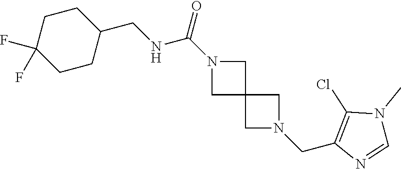 [SMILES: Cn1cnc(CN2CC3(C2)CN(C(=O)NCC2CCC(F)(F)CC2)C3)c1Cl] | Cpd B447 |
| 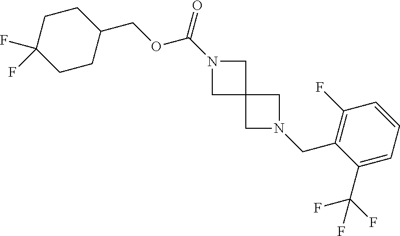 [SMILES: O=C(OCC1CCC(F)(F)CC1)N1CC2(CN(Cc3c(F)cccc3C(F)(F)F)C2)C1] | Cpd B448 |
| 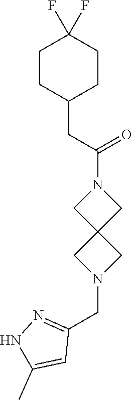 [SMILES: Cc1cc(CN2CC3(C2)CN(C(=O)CC2CCC(F)(F)CC2)C3)n[nH]1] | Cpd B449 |
| 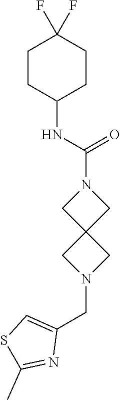 [SMILES: Cc1nc(CN2CC3(C2)CN(C(=O)NC2CCC(F)(F)CC2)C3)cs1] | Cpd B450 |
| 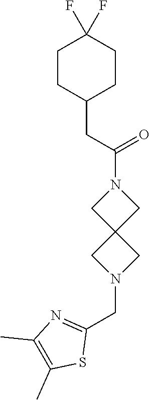 [SMILES: Cc1nc(CN2CC3(C2)CN(C(=O)CC2CCC(F)(F)CC2)C3)sc1C] | Cpd B451 |
| 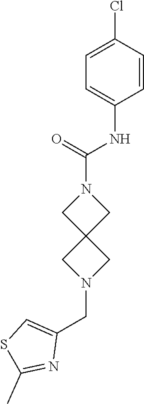 [SMILES: Cc1nc(CN2CC3(C2)CN(C(=O)Nc2ccc(Cl)cc2)C3)cs1] | Cpd B452 |
| 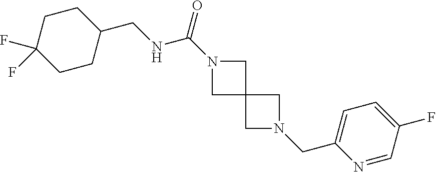 [SMILES: O=C(NCC1CCC(F)(F)CC1)N1CC2(CN(Cc3ccc(F)cn3)C2)C1] | Cpd B453 |
| 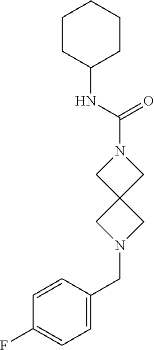 [SMILES: O=C(NC1CCCCC1)N1CC2(CN(Cc3ccc(F)cc3)C2)C1] | Cpd B454 |
| 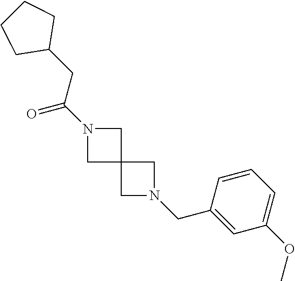 [SMILES: COc1cccc(CN2CC3(C2)CN(C(=O)CC2CCCC2)C3)c1] | Cpd B455 |
| 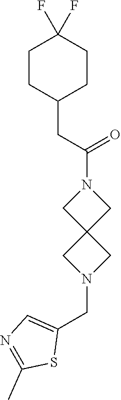 [SMILES: Cc1ncc(CN2CC3(C2)CN(C(=O)CC2CCC(F)(F)CC2)C3)s1] | Cpd B456 |
| 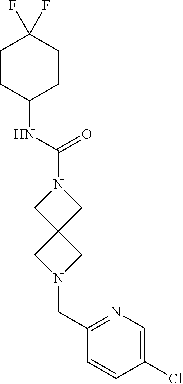 [SMILES: O=C(NC1CCC(F)(F)CC1)N1CC2(CN(Cc3ccc(Cl)cn3)C2)C1] | Cpd B457 |
| 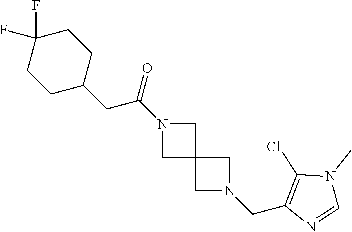 [SMILES: Cn1cnc(CN2CC3(C2)CN(C(=O)CC2CCC(F)(F)CC2)C3)c1Cl] | Cpd B458 |
| 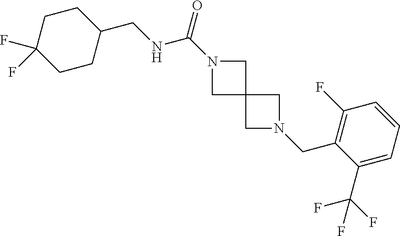 [SMILES: O=C(NCC1CCC(F)(F)CC1)N1CC2(CN(Cc3c(F)cccc3C(F)(F)F)C2)C1] | Cpd B459 |
| 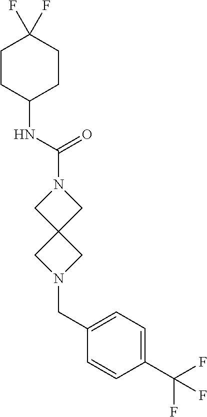 [SMILES: O=C(NC1CCC(F)(F)CC1)N1CC2(CN(Cc3ccc(C(F)(F)F)cc3)C2)C1] | Cpd B460 |
| 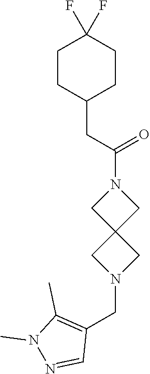 [SMILES: Cc1c(CN2CC3(C2)CN(C(=O)CC2CCC(F)(F)CC2)C3)cnn1C] | Cpd B461 |
| 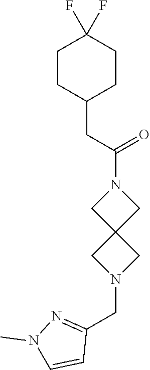 [SMILES: Cn1ccc(CN2CC3(C2)CN(C(=O)CC2CCC(F)(F)CC2)C3)n1] | Cpd B462 |
| 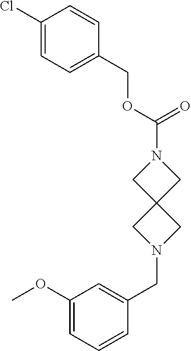 [SMILES: COc1cccc(CN2CC3(C2)CN(C(=O)OCc2ccc(Cl)cc2)C3)c1] | Cpd B463 |
| 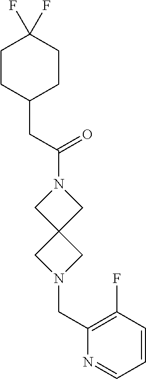 [SMILES: O=C(CC1CCC(F)(F)CC1)N1CC2(CN(Cc3ncccc3F)C2)C1] | Cpd B464 |
| 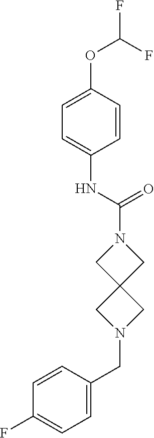 [SMILES: O=C(Nc1ccc(OC(F)F)cc1)N1CC2(CN(Cc3ccc(F)cc3)C2)C1] | Cpd B465 |
| 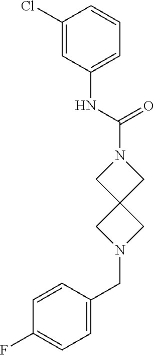 [SMILES: O=C(Nc1cccc(Cl)c1)N1CC2(CN(Cc3ccc(F)cc3)C2)C1] | Cpd B466 |
| 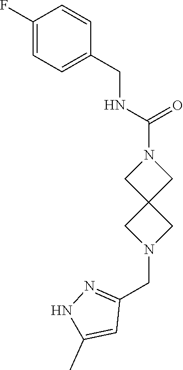 [SMILES: Cc1cc(CN2CC3(C2)CN(C(=O)NCc2ccc(F)cc2)C3)n[nH]1] | Cpd B467 |
| 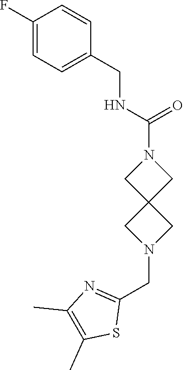 [SMILES: Cc1nc(CN2CC3(C2)CN(C(=O)NCc2ccc(F)cc2)C3)sc1C] | Cpd B468 |
| 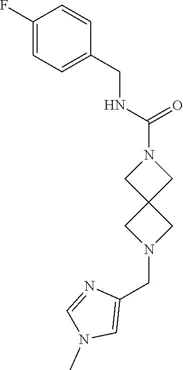 [SMILES: Cn1cnc(CN2CC3(C2)CN(C(=O)NCc2ccc(F)cc2)C3)c1] | Cpd B469 |
| 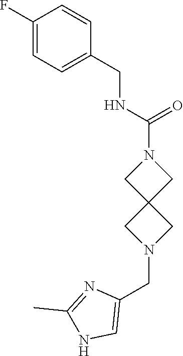 [SMILES: Cc1nc(CN2CC3(C2)CN(C(=O)NCc2ccc(F)cc2)C3)c[nH]1] | Cpd B470 |
| 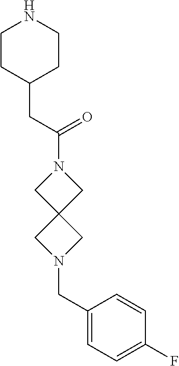 [SMILES: O=C(CC1CCNCC1)N1CC2(CN(Cc3ccc(F)cc3)C2)C1] | Cpd B471 |
| 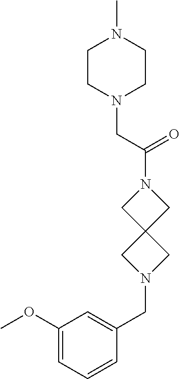 [SMILES: COc1cccc(CN2CC3(C2)CN(C(=O)CN2CCN(C)CC2)C3)c1] | Cpd B472 |
| 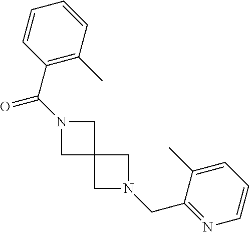 [SMILES: Cc1ccccc1C(=O)N1CC2(CN(Cc3ncccc3C)C2)C1] | Cpd B473 |
| 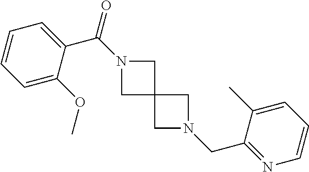 [SMILES: COc1ccccc1C(=O)N1CC2(CN(Cc3ncccc3C)C2)C1] | Cpd B474 |
| 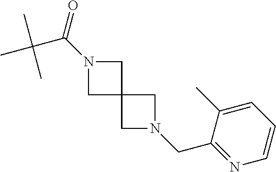 [SMILES: Cc1cccnc1CN1CC2(C1)CN(C(=O)C(C)(C)C)C2] | Cpd B475 |
| 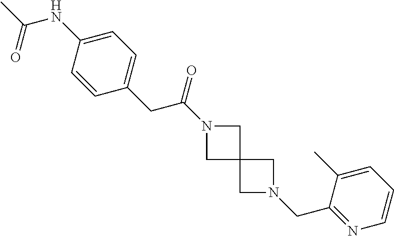 [SMILES: CC(=O)Nc1ccc(CC(=O)N2CC3(CN(Cc4ncccc4C)C3)C2)cc1] | Cpd B476 |
| 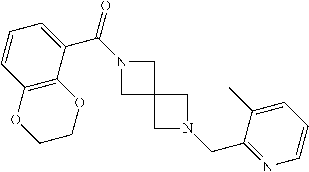 [SMILES: Cc1cccnc1CN1CC2(C1)CN(C(=O)c1cccc3c1OCCO3)C2] | Cpd B477 |
| 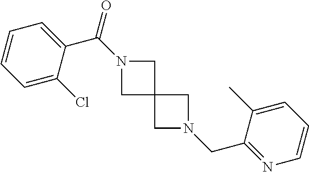 [SMILES: Cc1cccnc1CN1CC2(C1)CN(C(=O)c1ccccc1Cl)C2] | Cpd B478 |
| 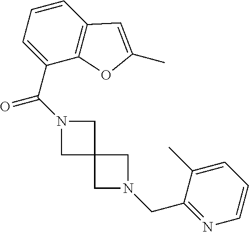 [SMILES: Cc1cc2cccc(C(=O)N3CC4(CN(Cc5ncccc5C)C4)C3)c2o1] | Cpd B479 |
| 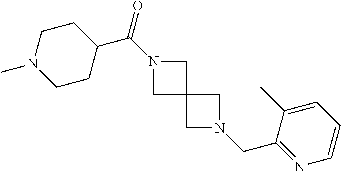 [SMILES: Cc1cccnc1CN1CC2(C1)CN(C(=O)C1CCN(C)CC1)C2] | Cpd B480 |
| 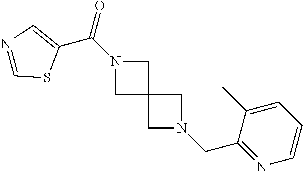 [SMILES: Cc1cccnc1CN1CC2(C1)CN(C(=O)c1cncs1)C2] | Cpd B481 |
| 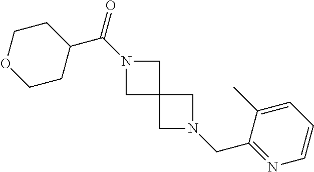 [SMILES: Cc1cccnc1CN1CC2(C1)CN(C(=O)C1CCOCC1)C2] | Cpd B482 |
| 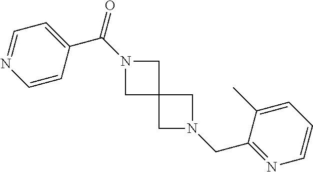 [SMILES: Cc1cccnc1CN1CC2(C1)CN(C(=O)c1ccncc1)C2] | Cpd B483 |
| 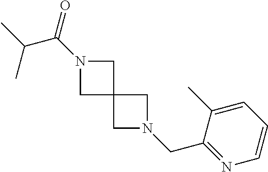 [SMILES: Cc1cccnc1CN1CC2(C1)CN(C(=O)C(C)C)C2] | Cpd B484 |
| 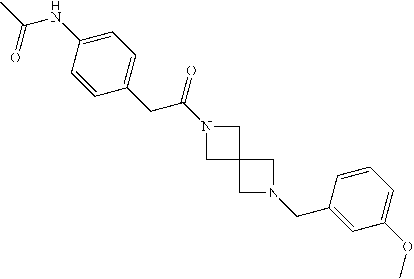 [SMILES: COc1cccc(CN2CC3(C2)CN(C(=O)Cc2ccc(NC(C)=O)cc2)C3)c1] | Cpd B485 |
| 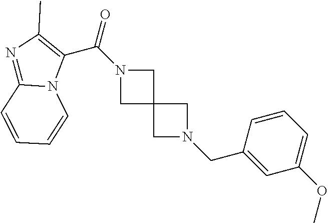 [SMILES: COc1cccc(CN2CC3(C2)CN(C(=O)c2c(C)nc4ccccn24)C3)c1] | Cpd B486 |
| 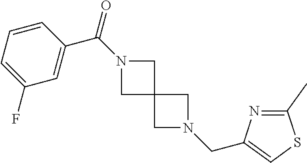 [SMILES: Cc1nc(CN2CC3(C2)CN(C(=O)c2cccc(F)c2)C3)cs1] | Cpd B487 |
| 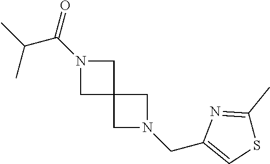 [SMILES: Cc1nc(CN2CC3(C2)CN(C(=O)C(C)C)C3)cs1] | Cpd B488 |
| 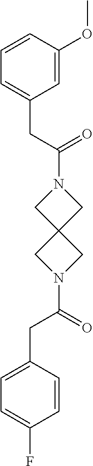 [SMILES: COc1cccc(CC(=O)N2CC3(CN(C(=O)Cc4ccc(F)cc4)C3)C2)c1] | Cpd B489 |
| 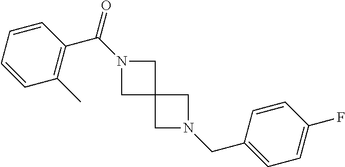 [SMILES: Cc1ccccc1C(=O)N1CC2(CN(Cc3ccc(F)cc3)C2)C1] | Cpd B490 |
| 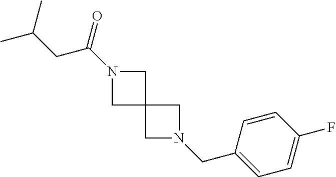 [SMILES: CC(C)CC(=O)N1CC2(CN(Cc3ccc(F)cc3)C2)C1] | Cpd B491 |
| 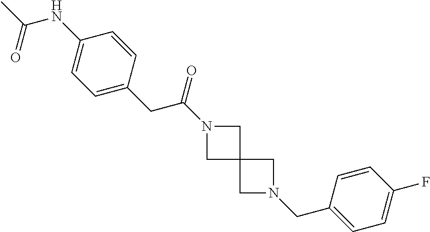 [SMILES: CC(=O)Nc1ccc(CC(=O)N2CC3(CN(Cc4ccc(F)cc4)C3)C2)cc1] | Cpd B492 |
| 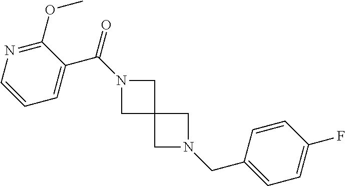 [SMILES: COc1ncccc1C(=O)N1CC2(CN(Cc3ccc(F)cc3)C2)C1] | Cpd B493 |
| 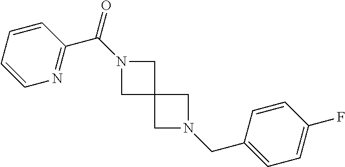 [SMILES: O=C(c1ccccn1)N1CC2(CN(Cc3ccc(F)cc3)C2)C1] | Cpd B494 |
| 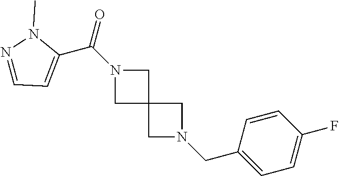 [SMILES: Cn1nccc1C(=O)N1CC2(CN(Cc3ccc(F)cc3)C2)C1] | Cpd B495 |
| 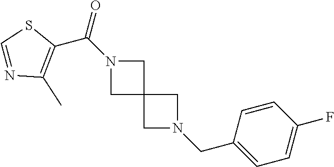 [SMILES: Cc1ncsc1C(=O)N1CC2(CN(Cc3ccc(F)cc3)C2)C1] | Cpd B496 |
| 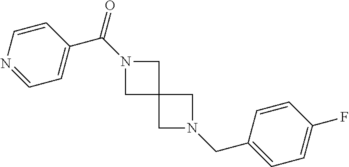 [SMILES: O=C(c1ccncc1)N1CC2(CN(Cc3ccc(F)cc3)C2)C1] | Cpd B497 |
| 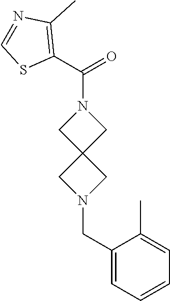 [SMILES: Cc1ccccc1CN1CC2(C1)CN(C(=O)c1scnc1C)C2] | Cpd B498 |
| 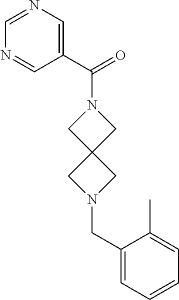 [SMILES: Cc1ccccc1CN1CC2(C1)CN(C(=O)c1cncnc1)C2] | Cpd B499 |
| 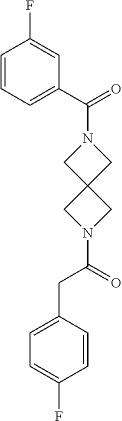 [SMILES: O=C(Cc1ccc(F)cc1)N1CC2(C1)CN(C(=O)c1cccc(F)c1)C2] | Cpd B500 |
| 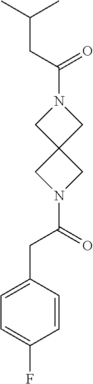 [SMILES: CC(C)CC(=O)N1CC2(CN(C(=O)Cc3ccc(F)cc3)C2)C1] | Cpd B501 |
| 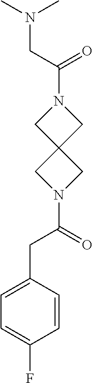 [SMILES: CN(C)CC(=O)N1CC2(CN(C(=O)Cc3ccc(F)cc3)C2)C1] | Cpd B502 |
| 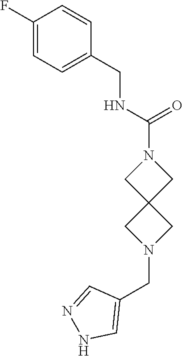 [SMILES: O=C(NCc1ccc(F)cc1)N1CC2(CN(Cc3cn[nH]c3)C2)C1] | Cpd B503 |
| 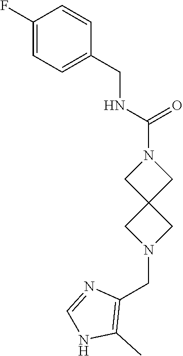 [SMILES: Cc1[nH]cnc1CN1CC2(C1)CN(C(=O)NCc1ccc(F)cc1)C2] | Cpd B504 |
| 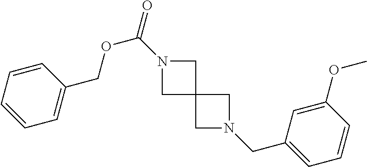 [SMILES: COc1cccc(CN2CC3(C2)CN(C(=O)OCc2ccccc2)C3)c1] | Cpd B505 |
| 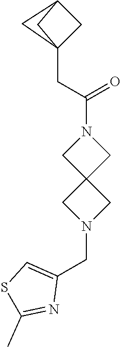 [SMILES: Cc1nc(CN2CC3(C2)CN(C(=O)CC24CC(C2)C4)C3)cs1] | Cpd B506 |
| 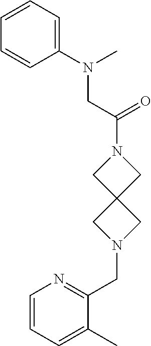 [SMILES: Cc1cccnc1CN1CC2(C1)CN(C(=O)CN(C)c1ccccc1)C2] | Cpd B507 |
| 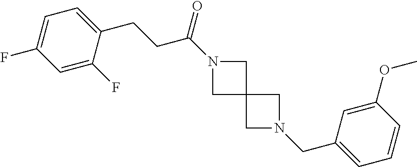 [SMILES: COc1cccc(CN2CC3(C2)CN(C(=O)CCc2ccc(F)cc2F)C3)c1] | Cpd B508 |
| 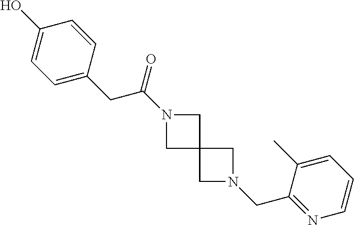 [SMILES: Cc1cccnc1CN1CC2(C1)CN(C(=O)Cc1ccc(O)cc1)C2] | Cpd B509 |
| 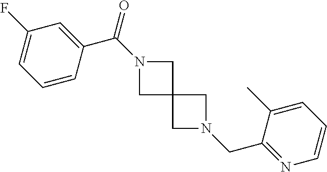 [SMILES: Cc1cccnc1CN1CC2(C1)CN(C(=O)c1cccc(F)c1)C2] | Cpd B510 |
| 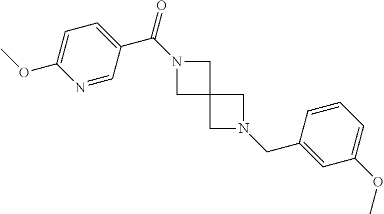 [SMILES: COc1cccc(CN2CC3(C2)CN(C(=O)c2ccc(OC)nc2)C3)c1] | Cpd B511 |
| 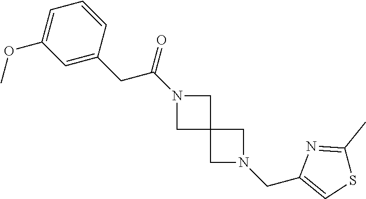 | Cpd B512 |
| 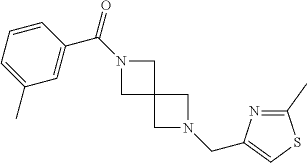 [SMILES: Cc1cccc(C(=O)N2CC3(CN(Cc4csc(C)n4)C3)C2)c1] | Cpd B513 |
| 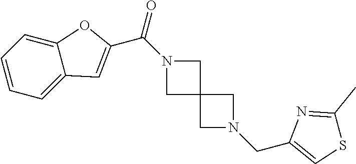 [SMILES: Cc1nc(CN2CC3(C2)CN(C(=O)c2cc4ccccc4o2)C3)cs1] | Cpd B514 |
| 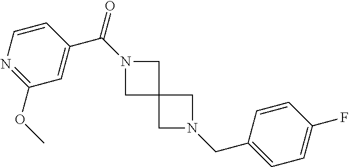 [SMILES: COc1cc(C(=O)N2CC3(CN(Cc4ccc(F)cc4)C3)C2)ccn1] | Cpd B515 |
| 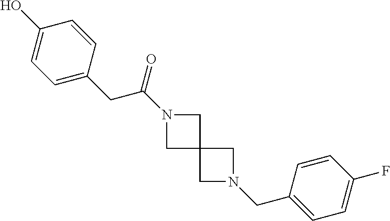 [SMILES: O=C(Cc1ccc(O)cc1)N1CC2(CN(Cc3ccc(F)cc3)C2)C1] | Cpd B516 |
| 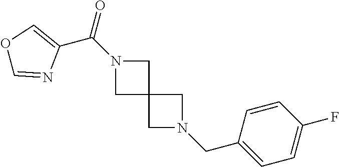 [SMILES: O=C(c1cocn1)N1CC2(CN(Cc3ccc(F)cc3)C2)C1] | Cpd B517 |
| 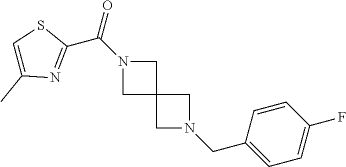 [SMILES: Cc1csc(C(=O)N2CC3(CN(Cc4ccc(F)cc4)C3)C2)n1] | Cpd B518 |
| 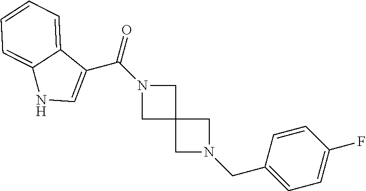 [SMILES: O=C(c1c[nH]c2ccccc12)N1CC2(CN(Cc3ccc(F)cc3)C2)C1] | Cpd B519 |
| 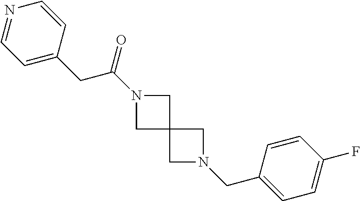 [SMILES: O=C(Cc1ccncc1)N1CC2(CN(Cc3ccc(F)cc3)C2)C1] | Cpd B520 |
| 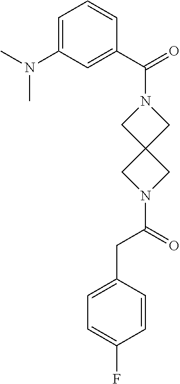 [SMILES: CN(C)c1cccc(C(=O)N2CC3(CN(C(=O)Cc4ccc(F)cc4)C3)C2)c1] | Cpd B521 |

Part A represents the preparation of the compounds (intermediates and final compounds) whereas Part B represents the pharmacological examples.

### Part A

All starting materials which are not explicitly described were either commercially available (the details of suppliers such as for example Aldrich, Combi-Blocks, Enamine, FluoroChem, MatrixScientific, Merck, TCI, etc. can be found in the SciFinder® Database for example) or the synthesis thereof has already been described precisely in the specialist literature (experimental guidelines can be found in the Reaxys® Database or the SciFinder® Database respectively, for example) or can be prepared using the conventional methods known to the person skilled in the art.

The reactions were, if necessary, carried out under an inert atmosphere (mostly argon and N₂). The number of equivalents of reagents and the amounts of solvents employed as well as the reaction temperatures and times can vary slightly between different reactions carried out by analogous methods. The work-up and purification methods were adapted according to the characteristic properties of each compound and can vary slightly for analogous methods. The yields of the compounds prepared are not optimized.

The indication “equivalents” (“eq.” or “eq” or “equiv.”) means molar equivalents, “RT” or “rt” means room temperature T (23±7° C.), “M” are indications of concentration in mol/1, “sol.” means solution, “conc.” means concentrated. The mixing ratios of solvents are usually stated in the volume /volume ratio.

Key analytical characterization was carried out by means of ¹H-NMR spectroscopy and/or mass spectrometry (MS, m/z for [M+H]⁺ and/or for [M−H]⁻) for all the exemplary compounds and selected intermediate products.

Analytical instruments employed were e.g., for NMR analysis a BRUKER 400 MHz (Software Topspin). For LC/MS analysis e.g., an Agilent 1260, Mass:6130 (ESI) (Software Open Lab Chemstation) was employed. Analytical UPLCs were measured e.g., on Waters IClass (Software MassLynx). For GCMS analysis e.g., an Agilent 6890N was employed.

Preparative HPLC were performed e.g., on an Agilent G6120 or Waters and reversed phase MPLC were performed e.g., on Reveleris.

Some structures of compounds that contain stereocentres are drawn and named with absolute stereochemistry, if known. In case of unknown absolute stereochemistry, the compounds can be either racemic, a mixture of diastereomers, a pure diastereomer of unknown stereochemistry, or a pure enantiomer of unknown stereochemistry.

The MS analyses mentioned in the experimental part were performed on a Waters system. In acidic conditions: Waters IClass; Bin. Pump: UPIBSM, SM: UPISMFTN with SO; UPCMA, PDA: UPPDATC, 210-320 nm, MS: QDa ESI, pos/neg 100-800; column: Waters XSelect CSH C18, 50×2.1 mm, 2.5μ, Temp: 40° C., Flow: 0.6 mL/min, Gradient: to =5% A, t_{2.0min}=98% A, t_{2.7min}=98% A, Posttime: 0.3 min, Eluent A: 0.1% formic acid in acetonitrile, Eluent B: 0.1% formic acid in water. In basic conditions: Apparatus: Waters IClass; Bin. Pump: UPIBSM, SM: UPISMFTN with SO; UPCMA, PDA: UPPDATC, 210-320 nm, MS: QDa ESI, pos/neg 100-800; column: Waters XSelect CSH C18, 50×2.1 mm, 2.5p, Temp: 25° C., Flow: 0.6 mL/min, Gradient: to =5% A, t_{2.0min}=98% A, t_{2.7min}=98% A, Posttime: 0.3 min, Eluent A: acetonitrile, Eluent B: 10 mM ammonium bicarbonate in water (pH=9.5) The GCMS analyses mentioned in the experimental part were performed on an Agilent 6890N G1530 gas chromatography system coupled with G2577A 5973 MSD detector (EI-positive, Det. temp.: 280° C.) Mass range 50-550, Column: Restek RXi-5MS (20 m ID 180 μm, df 0.18 μm), Average velocity: 50 cm/s, Injection vol: 1 μl, Injector temp: 250° C., Split ratio: 100/1, Carrier gas: He; Initial temp: 100° C., Initial time: 1.5 min, Solvent delay: 1.0 min, Rate 75° C./min, Final temp 250° C., Hold time 4.3 min.

Preparative HPLC purifications mentioned in this experimental part have been carried out with a Waters system in basic conditions: MS instrument type: ACQ-SQD2; HPLC instrument type: Waters Modular Preparative HPLC System; column: Waters XSelect (C18, 100×30 mm, 10 μm); flow: 55 ml/min prep pump; column temp: RT; eluent A: 10 mM ammonium bicarbonate in water pH=9.5, eluent B: 100% acetonitrile; Gradient: t=0 min 5% B, t=17 min 100% B; detection: DAD (220-320 nm); detection: MSD (ESI pos/neg) mass range: 100-800; fraction collection based on MS and DAD.

Preparative HPLC purifications mentioned in this experimental part in acidic conditions have been carried out with an Agilent system: MS instrument type: Agilent Technologies G6120AA Quadrupole; HPLC instrument type: Agilent Technologies 1200 preparative LC; Column: Waters XSelect CSH (C18, 100×30 mm, 10p); Flow: 55 ml/min; Column temp: RT; Eluent A: 0.1% formic acid in water; Eluent B: 100% acetonitrile; Gradient: t=0 min 5% B, t=17 min 100% B Detection: DAD (220-320 nm); Detection: MSD (ESI pos/neg) mass range: 100-1000; fraction collection based on MS and DAD.

Reversed phase MPLC purifications mentioned in this experimental part in acidic conditions have been carried out with Reveleris: Instrument type: Reveleris™ prep MPLC; Column: Phenomenex LUNA C18(3) (150×25 mm, 10p); Flow: 40 mL/min; Column temp: rt.; Eluent A: 0.1% (v/v) Formic acid in water, Eluent B: 0.1% (v/v) Formic acid in acetonitrile; Gradient: t=0 min 5% B, t=20 min 100% B.; Detection UV: 220, 254, 280 nm.

In basic conditions, reversed phase MPLC purifications mentioned in this experimental part in have been carried out with Reveleris: Instrument type: Reveleris™ prep MPLC; column: Waters XSelect CSH C18 (145×25 mm, 10p); Flow: 40 mL/min; Column temp: rt.; Eluent A: 10 mM ammoniumbicarbonate in water pH=9.0); Eluent B: 99% acetonitrile+1% 10 mM ammoniumbicarbonate in water; Gradient: t=0 min 5% B, t=20 min 100% B. Detection UV: 220, 254, 280 nm.

### Examples of the Preparation of Intermediates

### Synthesis of 2-(4-chlorophenyl)-1-(2,6-diazaspiro[3.3]heptan-2-yl)ethenone: (IB 4-1)

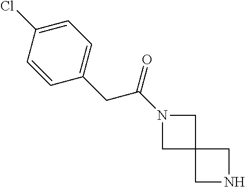 [SMILES: O=C(Cc1ccc(Cl)cc1)N1CC2(CNC2)C1]

### a) tert-butyl 6-(2-(4-chlorophenyl)acetyl)-2,6-diazaspiro[3.3]heptane-2-carboxylate

Tert-butyl 2,6-diazaspiro[3.3]heptane-2-carboxylate, 0.5 oxalic acid (1 g, 4.11 mmol, Eq: 1.00) and TEA (1.04 g, 1.42 ml, 10.3 mmol, Eq: 2.5) were combined in dry DCM (27.4 ml) to give a off-white suspension. Reaction mixture was cooled to 0° C. 2-(4-chlorophenyl)acetyl chloride (777 mg, 601 μl, 4.11 mmol, Eq: 1) was added slowly at 0° C. The reaction mixture was stirred at 0° C. for 1 h. LCMS showed 80% conversion. The crude reaction mixture was partially concentrated in vacuo and poured into EtOAc and extracted with H₂O. The aqueous layer was back-extracted with EtOAc. The crude material was purified by flash chromatography (silica gel, 100 g, 20% to 100% EtOAc in heptane) to give 686 mg (48% yield) of the title product. ESI [M+H]⁺: 351.1.

### b) 2-(4-chlorophenyl)-1-(2,6-diazaspiro[3.3]heptan-2-yl)ethanone

Tert-butyl 6-(2-(4-chlorophenyl)acetyl)-2,6-diazaspiro[3.3]heptane-2-carboxylate (686 mg, 1.96 mmol, Eq: 1.00) was combined in DCM (13.0 ml) to give a yellow solution. TFA (1.11 g, 749 μl, 9.78 mmol, Eq: 10) was added. After 2 h the crude reaction mixture was then poured into EtOAc and extracted with Na₂CO₃. Organic layer was dried on Na₂SO₄ and evaporated was concentrated in vacuo to give 1.10 g (92% yield) of the title product used directly as crude. ESI [M+H]⁺: 251.4.

### Synthesis of 2-(4-fluorophenyl)-1-(2,6-diazaspiro[3.3]heptan-2-yl)ethanone (IB 4-2)

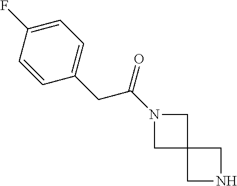 [SMILES: O=C(Cc1ccc(F)cc1)N1CC2(CNC2)C1]

### a) tert-butyl 6-(2-(4-fluorophenyl)acetyl)-2,6-diazaspiro[3.3]heptane-2-carboxylate

2-(4-fluorophenyl)acetic acid (488 mg, 3.17 mmol, Eq: 2.2), TEA (728 mg, 1.00 ml, 7.19 mmol, Eq: 5) and HATU (1.2 g, 3.17 mmol, Eq: 2.2) were combined in DCM (10 ml).

The reaction mixture was heated to 45° C. and stirred for 15 min. Tert-butyl 2,6-diazaspiro[3.3]heptane-2-carboxylate hemioxalate (700 mg, 1.44 mmol, Eq: 1.00) was added, and the reaction mixture was heated to 45° C. and stirred for 1 h. The reaction mixture was poured into DCM and extracted with sat NaHCO₃. The aqueous layer was back-extracted with DCM. Crude=1.59 g. The crude material was purified by flash chromatography (silica gel, 70 g, 0% to 10% MeOH in DCM) to give 451 mg (yield 47%) of the title product. ESI [M+H]⁺: 335.3.

### b) 2-(4-fluorophenyl)-1-(2,6-diazaspiro[3.3]heptan-2-yl)ethanone

Tert-butyl 6-(2-(4-fluorophenyl)acetyl)-2,6-diazaspiro[3.3]heptane-2-carboxylate (451 mg, 1.35 mmol, Eq: 1.00) was dissolved in DCM (8.99 ml). TFA (819 mg, 550 μl, 7.18 mmol, Eq: 5.32) was added. The reaction mixture was stirred for 2 h. Another 5 eq. TFA (550 μl) was added. The reaction mixture was stirred for 4 h. Another 5 eq TFA (550 μl) were added. The reaction mixture was stirred for 1.5 h. then the mixture was concentrated. The crude mixture was then poured into EtOAc and extracted with Na₂CO₃. Organic layer was dried on Na₂SO₄ and evaporated to afford 281 mg (yield 90%) of the title product. ESI [M+H]⁺: 235.1.

### Synthesis of 2-(3,4-difluorophenyl)-1-(2,6-diazaspiro[3.3]heptan-2-yl)ethanone (IB 4-3)

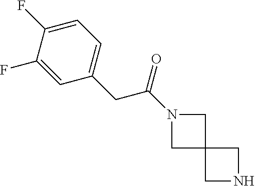 [SMILES: O=C(Cc1ccc(F)c(F)c1)N1CC2(CNC2)C1]

### a)tert-butyl 6-(2-(3,4-difluorophenyl)acetyl)-2,6-diazaspiro[3.3]heptane-2-carboxylate

To a solution of 2-(3,4-difluorophenyl)acetic acid (4.68 g, 27.2 mmol, 1.1 eq.) in DMF (200 mL) with DIPEA (8.63 mL, 49.4 mmol, 2 eq.) was added HATU (11.3 g, 29.7 mmol, 1.2 eq.). The reaction was stirred 20 min at RT, then a solution of tert-butyl 2,6-diazaspiro[3.3]heptane-2-carboxylate, 0.5 oxalic acid (6 g, 24.7 mmol, 1 eq.) in 50 mL with 2 eq. DIPEA was added. Reaction was stirred 4h at RT. LCMS showed SM and product. Another equivalent of 2-(3,4-difluorophenyl)acetic acid and HATU were added and the reaction stirred 30 min. at RT. LCMS showed the reaction was complete. After extraction with DCM/NaHCO₃ sat. aq. sol., the organic layer was dried on Na₂SO₄ and evaporated. Column on silica using with heptane/ethyl acetate (1/1 to 0/1) gave the desired product. ESI [M+H]⁺; 353.3.

### b) 2-(3,4-difluorophenyl)-1-(2,6-diazaspiro[3.3]heptan-2-yl)ethanone

To a solution of tert-butyl 6-(2-(3,4-difluorophenyl)acetyl)-2,6-diazaspiro[3.3]heptane-2-carboxylate (13.4 g, 30%, 11.4 mmol) was added TFA (13.2 mL, 171 mmol, 15 eq.) and the reaction was stirred at RT overnight. Extraction with ethylacetate/ether and Na₂CO₃ aq. sat. sol. Gave two layers where LCMS showed product was in the aqueous layer and impurities in the organic layer. Concentration of aqueous layer and extraction with isopropanol/ethyl acetate, gave the organic layer which was dried on Na₂SO₄ and evaporated, then azeotroped with heptane to remove the water and give the product (30%). [M+H]⁺; 253.2

### Synthesis of N-(6-Amino-spiro[3.3]hept-2-yl)-2-(4-fluoro-phenyl)-acetamide (HCl) (IB 7-1)

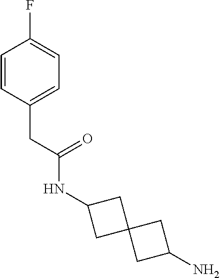 [SMILES: NC1CC2(C1)CC(NC(=O)Cc1ccc(F)cc1)C2]

### a) tert-butyl 6-(2-(4-fluorophenyl)acetamido)spiro[3.3]heptan-2-ylcarbamate

To a solution of 2-(4-fluorophenyl)acetic acid (1.12 g, 7.29 mmol, 1.1 eq.) in DCM (200 mL) with TEA (2.77 mL, 19.9 mmol, 3 eq.) was added HATU (3.02 g, 7.95 mmol, 1.2 eq.). The reaction was stirred 15 min at 45° C., then tert-butyl 6-aminospiro[3.3]heptan-2-ylcarbamate (1.5 g, 6.63 mmol, 1 eq.) was added. The reaction was stirred at 45° C. overnight. Extraction with DCM/water provided the organic layer which was dried on Na₂SO₄ and evaporated: the product was precipitated in the cold DCM and was filtered off. ESI [M+H-BOC]+=263.2.

### b) N-(6-Amino-spiro[3.3]hept-2-yl)-2-(4-fluoro-phenyl)-acetamide (HCl)

Tert-butyl 6-(2-(4-fluorophenyl)acetamido)spiro[3.3]heptan-2-ylcarbamate (110 mg, 0.3 mmol, Eq: 1.00) was dissolved in DCM and 4M HCl in dioxane (1 mL, 4 mmol, Eq: 13) was added. The reaction mixture was stirred at RT for 1 h. Evaporation of the solvent gave the desired product used directly as crude. ESI [M−H]⁻=297.1.

### Synthesis of N-(6-aminospiro[3.3]heptan-2-yl)-2-(4-chlorophenyl)acetamide (HCl) (IB 7-2)

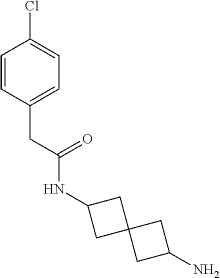 [SMILES: NC1CC2(C1)CC(NC(=O)Cc1ccc(Cl)cc1)C2]

### a) tert-butyl 6-(2-(4-chlorophenyl)acetamido)spiro[3.3]heptan-2-ylcarbamate

Tert-butyl 6-aminospiro[3.3]heptan-2-ylcarbamate (6 g, 26.5 mmol, Eq: 1.00) and TEA (5.37 g, 7.35 ml, 53.0 mmol, Eq: 2) were combined in DCM (177 ml) to give a white suspension. The solution was cooled to 0° C. 2-(4-chlorophenyl)acetyl chloride (7.52 g, 5.82 ml, 39.8 mmol, Eq: 1.5) was added at 0° C. to give an orange suspension. The reaction mixture was stirred for 1.5 h. and the reaction mixture was extracted with H₂O. The crude reaction mixture was concentrated in vacuo. Recrystallization from DCM and MeOH afforded a white solid. ESI [M+H-tBu]⁺=323.3.

### b) N-(6-aminospiro[3.3]heptan-2-yl)-2-(4-chlorophenyl)acetamide (HCl)

Tert-butyl 6-(2-(4-chlorophenyl)acetamido)spiro[3.3]heptan-2-ylcarbamate (7.682 g, 20.3 mmol, Eq: 1.00) was dissolved in 100 mL DCM and 4M HCl in dioxane (35.5 mL, 142 mmol, Eq: 7) was added. The reaction mixture was stirred overnight at RT. Evaporation of the solvent and recrystallization from DCM and MeOH afforded a white solid, 2.408 g. ESI [M+H]+=279.1.

### Synthesis of 2-(4-fluorobenzyl)-2,6-diazaspiro[3.3]heptane (IB12-1)

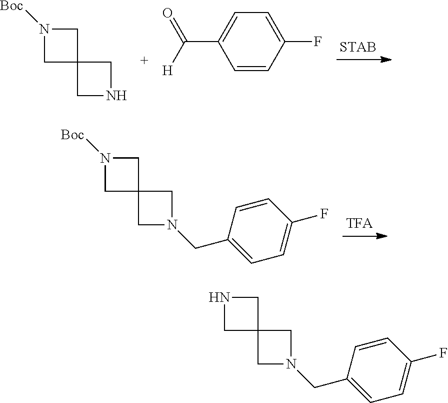

Step 1: To tert-butyl 2,6-diazaspiro[3.3]heptane-2-carboxylate hemioxalate (1 g, 2.055 mmol) in anhydrous DCM (24 ml)/MeOH (8 ml) were added acetic acid (0.471 ml, 8.22 mmol) and 4-fluorobenzaldehyde (0.665 ml, 6.17 mmol). The mixture was purged with nitrogen and titanium(IV) isopropoxide (2.409 ml, 8.22 mmol) was added. The mixture was stirred at rt for 2 hours. STAB (3.48 g, 16.44 mmol) was added and the mixture was stirred overnight at RT. The mixture was carefully quenched by addition of aq. sat. Na₂CO₃ and was stirred for 1 hour at RT. More DCM was added to the resulting suspension and the layers were separated. The aqueous layer was extracted with DCM (2×) and the extracted DCM layers were directly filtered over a path of celite. The combined and filtered extracts were collected and concentrated under reduced pressure. The crude product was purified by flash column chromatography to obtain tert-butyl 6-(4-fluorobenzyl)-2,6-diazaspiro[3.3]heptane-2-carboxylate (1.16 g, 3.79 mmol, 92% yield) as a colorless oil. MS (m/e): 307.2 (MH⁺).

Step 2: tert-butyl 6-(4-fluorobenzyl)-2,6-diazaspiro[3.3]heptane-2-carboxylate (1.16 g, 3.79 mmol) was dissolved in DCM (6 ml) and trifluoroacetic acid (3.50 ml, 45.4 mmol) was added. The mixture was stirred overnight at RT. The mixture was diluted with DCM and concentrated cold (above the water bath) under reduced pressure The crude mixture was dissolved in a few mL of DMSO and sat. aq. Na₂CO₃ was added until pH>7. The mixture was purified by RP chromatography (50 g column, gradient: 0% to 50% MeCN in 10 mM ammoniumbicarbonate in water) to obtain 2-(4-fluorobenzyl)-2,6-diazaspiro[3.3]heptane (695 mg, 3.37 mmol, 89% yield) as a slightly yellow oil. MS (m/e): 207.1 (MH⁺).

The following intermediates were prepared in a similar manner (use of appropriate reagents and purification methods known to the person skilled in the art) as described for IB 12-1: IB 12-2, IB 12-3, and IB 12-4.

| | |
|---|---|
| 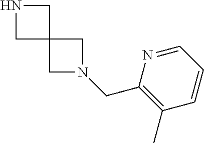 | IB 12-2 |
| MS (m/e): 204.2 (MH⁺) | |
| Yield: 89% | |
| 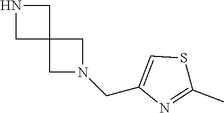 | IB 12-3 |
| MS (m/e): 210.1 (MH⁺) | |
| Yield: 91% | |
| 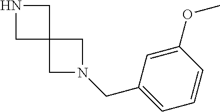 [SMILES: COc1cccc(CN2CC3(CNC3)C2)c1] | IB 12-4 |
| MS (m/e): 219.2 (MH⁺) | |
| Yield: 92% | |

### Synthesis of 2-(4,4-difluorocyclohexyl)-1-(2,6-diazaspiro[3.3]heptan-2-yl)ethan-1-one (IB 22-1)

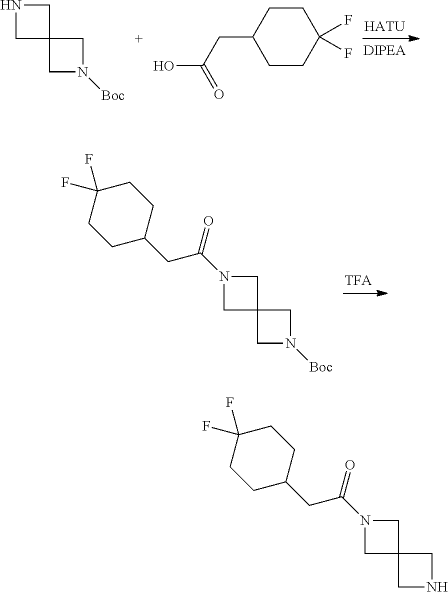

Step 1: 2-(4,4-difluorocyclohexyl)acetic acid (1.007 g, 5.65 mmol) was dissolved in DCM (25 ml), DIPEA (2.244 ml, 12.85 mmol) and HATU (2.051 g, 5.39 mmol) were added. tert-butyl 2,6-diazaspiro[3.3]heptane-2-carboxylate hemioxalate (1.25 g, 2.57 mmol) was added and the mixture was stirred overnight at RT. Water was added to the reaction mixture and the layers were separated. The aqueous layer was extracted with DCM twice. The combined organic fractions were dried over Na₂SO₄, filtered and concentrated, affording crude tert-butyl 6-(2-(4,4-difluorocyclohexyl)acetyl)-2,6-diazaspiro[3.3]heptane-2-carboxylate as a yellow oil. No further purification performed

Step 2: To tert-butyl 6-(2-(4,4-difluorocyclohexyl)acetyl)-2,6-diazaspiro[3.3]heptane-2-carboxylate (1.842 g, 5.14 mmol) in DCM (40 ml) was added trifluoroacetic acid (5.94 ml, 77 mmol). The mixture stirred at RT for 2 h and then concentrated cold on the rotovap. The crude mixture was dissolved in a few mL of DMSO and sat. aq. Na₂CO₃ was added until pH>7. The mixture was purified by RP chromatography (100 g column, gradient: 0% to 100% MeCN in 10 mM ammoniumbicarbonate in water), affording 2-(4,4-difluorocyclohexyl)-1-(2,6-diazaspiro[3.3]heptan-2-yl)ethan-1-one (1.08 g, 4.18 mmol, 81% yield) as an off-white foam. MS (m/e): 259.1 (MH⁺)

### Synthesis of 4,4-difluorocyclohexyl 2,6-diazaspiro[3.3]heptane-2-carboxylate (IB 25-1)

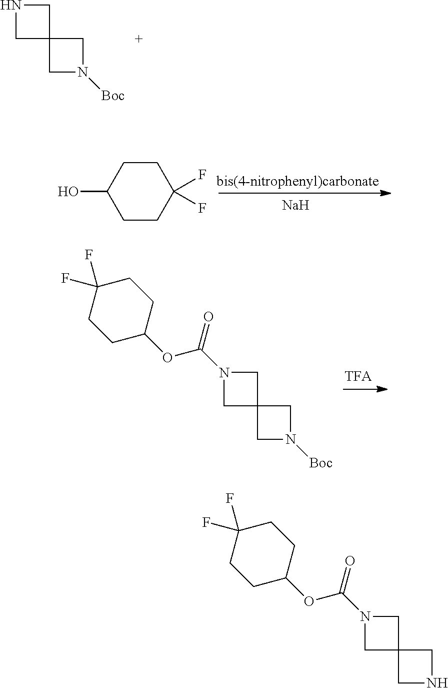

Step 1: To bis(4-nitrophenyl) carbonate (2.345 g, 7.71 mmol) in DMF (dry) (10 ml) were added 4,4-difluorocyclohexan-1-ol (1.119 g, 8.22 mmol) and NaH (60% w/w in mineral oil, 0.668 g, 16.70 mmol). The mixture was stirred at RT for 1.5h before addition of tert-butyl 2,6-diazaspiro[3.3]heptane-2-carboxylate hemioxalate (1.25 g, 2.57 mmol) in DMF (dry) (15 ml) with DIPEA (1.346 ml, 7.71 mmol). The mixture was stirred overnight at RT. The mixture was concentrated and to the residue was added EtOAc, little water and sat. aq. Na₂CO₃, and layers were separated. The aqueous layer was extracted twice more with EtOAc and the combined organics were washed with sat. aq. NH₄Cl, dried over Na₂SO₄, filtered and concentrated, affording crude 2-(tert-butyl) 6-(4,4-difluorocyclohexyl) 2,6-diazaspiro[3.3]heptane-2,6-dicarboxylate as an orange oil. No further purification was performed.

Step 2: To 2-(tert-butyl) 6-(4,4-difluorocyclohexyl) 2,6-diazaspiro[3.3]heptane-2,6-dicarboxylate (1.856 g, 5.15 mmol) in DCM (40 ml) was added trifluoroacetic acid (5.95 ml, 77 mmol). The mixture stirred overnight at RT and then concentrated cold. The crude mixture was dissolved in a few mL of DMSO and sat. aq. Na₂CO₃ was added until pH>7. The mixture was purified by RP chromatography (100 g column, gradient: 0% to 100% MeCN in 10 mM ammoniumbicarbonate in water), affording 4,4-difluorocyclohexyl 2,6-diazaspiro[3.3]heptane-2-carboxylate (873 mg, 3.35 mmol, 65.1% yield) as a yellow oil. MS (m/e): 261.2 (MH⁺)

The following intermediate was prepared in a similar manner (use of appropriate reagents and purification methods known to the person skilled in the art) as described for IB 25-1: IB25-2

| | |
|---|---|
| 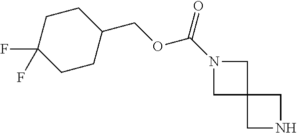 [SMILES: O=C(OCC1CCC(F)(F)CC1)N1CC2(CNC2)C1] | IB 25-2 |
| MS (m/e): 275.1 (MH⁺) | |
| Yield: 33.6% | |

### Synthesis of N-(4-fluorobenzyl)-2,6-diazaspiro[3.3]heptane-2-carboxamide (IB 29-1)

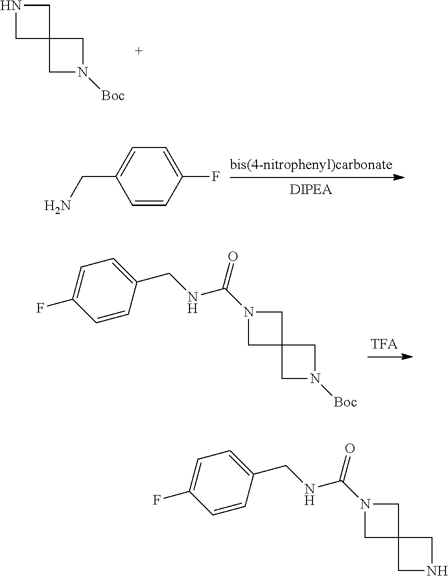 [SMILES: CC(C)(C)OC(=O)N1CC2(CNC2)C1 ; NCc1ccc(F)cc1 ; CC(C)(C)OC(=O)N1CC2(CN(C(=O)NCc3ccc(F)cc3)C2)C1 ; O=C(NCc1ccc(F)cc1)N1CC2(CNC2)C1]

Step 1: To bis(4-nitrophenyl) carbonate (1.876 g, 6.17 mmol) in DMF (dry) (40 ml) were added (4-fluorophenyl)methanamine (0.752 ml, 6.58 mmol) and DIPEA (2.154 ml, 12.33 mmol). The mixture was stirred at RT for 1.5h before addition of tert-butyl 2,6-diazaspiro[3.3]heptane-2-carboxylate hemioxalate (1 g, 2.055 mmol). The mixture was stirred overnight at RT. Saturated aq. Na₂CO₃ and EtOAc were added to the mixture and the layers were separated. The organic layer was washed with more sat. aq. Na₂CO₃ and sat. aq. NH₄Cl. The organic fractions were combined and concentrated under reduced pressure. The crude was purified by flash column chromatography (40 g silica, wet loading from DCM, heptane/EtOAc=35:65->0:1) to obtain tert-butyl 6-((4-fluorobenzyl)carbamoyl)-2,6-diazaspiro[3.3]heptane-2-carboxylate (1.11 g, 3.18 mmol, 77% yield) as a white solid. MS (m/e): 350.2 (MH⁺)

Step 2: To tert-butyl 6-((4-fluorobenzyl)carbamoyl)-2,6-diazaspiro[3.3]heptane-2-carboxylate (1.11 g, 3.18 mmol) in DCM (6 ml) was added trifluoroacetic acid (3.50 ml, 45.4 mmol). The mixture stirred overnight at RT and then concentrated cold. The crude mixture was dissolved in a few mL of DMSO and sat. aq. Na₂CO₃ was added until pH>7. The mixture was purified by RP chromatography (100 g column, gradient: 0% to 100% MeCN in 10 mM ammoniumbicarbonate in water), affording N-(4-fluorobenzyl)-2,6-diazaspiro[3.3]heptane-2-carboxamide (780 mg, 3.13 mmol, 98% yield) as a slightly yellow oil. MS (m/e): 250.1 (MH⁺)

The following intermediates were prepared in a similar manner (use of appropriate reagents and purification methods known to the person skilled in the art) as described for IB 29-1: IB 29-2, IB 29-3.

| | |
|---|---|
| 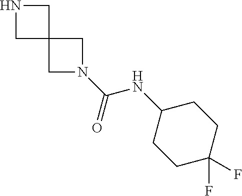 [SMILES: O=C(NC1CCC(F)(F)CC1)N1CC2(CNC2)C1] | IB 29-2 |
| MS (m/e): 260.2 (MH⁺) | |
| Yield: 30.6% | |
| 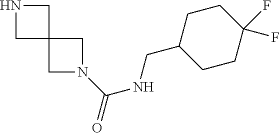 [SMILES: O=C(NCC1CCC(F)(F)CC1)N1CC2(CNC2)C1] | IB 29-3 |
| MS (m/e): 274.2 (MH⁺) | |
| Yield: 57.5% | |

### Synthesis of N-(3-(4-fluorobenzyl)-1,2,4-thiadiazol-5-yl)spiro[3.3]heptane-2,6-diamine hydrochloride (IB 30-1)

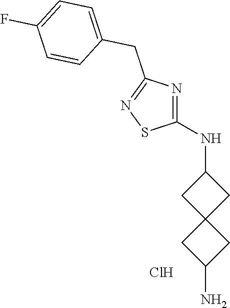 [SMILES: NC1CC2(C1)CC(Nc1nc(Cc3ccc(F)cc3)ns1)C2 ; Cl]

### a) tert-butyl 6-(3-(4-fluorobenzyl)-1,2,4-thiadiazol-5-ylamino)spiro[3.3]heptan-2-ylcarbamate

To a solution of commercially available tert-butyl 6-aminospiro[3.3]heptan-2-ylcarbamate (1.86 g, 8.2 mmol, Eq. 1.2) in dry DMF (45 ml) was added commercially available 3-chloro-5-(4-fluorobenzyl)-1,2,4-thiadiazole (1.5 g, 6.56 mmol, Eq. 1) and DIPEA (1.6 ml, 9.18 mmol, Eq. 1.4).

The reaction was stirred under argon at 140° C. for 30 min. Solvent was evaporated and the crude product redissolved in EtOAc and washed with water. Aqueous layer was re extracted with fresh EtOAc. Organic layer was dried over Na₂SO₄, concentrated and purified with flash chromatography using heptane and ethyl acetate providing 2.39 g (yield 87%) of the title product.

ESI [M−H]⁻; 417.7

### b) N-(3-(4-fluorobenzyl)-1,2,4-thiadiazol-5-yl)spiro[3.3]heptane-2,6-diamine hydrochloride

To a solution of tert-butyl 6-(3-(4-fluorobenzyl)-1,2,4-thiadiazol-5-ylamino)spiro[3.3]heptan-2-ylcarbamate (2.19 g, 5.23 mmol, Eq. 1) in dioxane (22 ml) was added HCl in dioxane (4 M, 4 ml, Eq. 7.6). Upon addition of HCl, the reaction mixture turned from a pale yellow suspension to clear solution. The reaction was stirred at room temp for 1 hour. LCMS showed no progression, so a further 6 ml of HCl in dioxane(4 M) was added. Still no progression after a further 3.5 hours. HCl in dioxane was added until reaction's completion. Then the reaction mixture was concentrated in vacuo and azeotroped 2 times with toluene. Product was dried overnight under high vacuum yielding the title product. ESI [M+H]⁺; 319.1

### Synthesis of N-(3-(4-chlorobenzyl)-1,2,4-thiadiazol-5-yl)spiro[3.3]heptane-2,6-diamine TFA salt (IB 30-2)

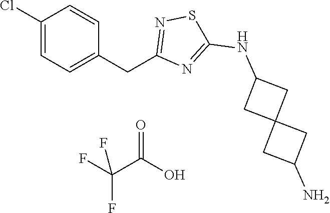 [SMILES: NC1CC2(C1)CC(Nc1nc(Cc3ccc(Cl)cc3)ns1)C2 ; O=C(O)C(F)(F)F]

### a) tert-butyl 6-(3-(4-chlorobenzyl)-1,2,4-thiadiazol-5-ylamino)spiro[3.3]heptan-2-ylcarbamate

Commercially available tert-butyl 6-aminospiro[3.3]heptan-2-ylcarbamate (3.84 g, 17.0 mmol, Eq: 1), commercially available 5-chloro-3-(4-chlorobenzyl)-1,2,4-thiadiazole (4.1637 g, 17.0 mmol, Eq: 1.00) and DIPEA (4.39 g, 5.93 ml, 34.0 mmol, Eq: 2) were combined with THF (113 ml) to give a yellow solution. The reaction mixture was heated to 110° C. and stirred for 2.5 h, followed by LCMS. The reaction mixture was poured into 400 mL EtOAc and extracted with H₂O (1×300 mL). The aqueous layer was back-extracted with EtOAc (1×100 mL). The organic layers were dried over Na₂SO₄, concentrated in vacuo, and the crude material was purified by flash chromatography on 330 g silica gel (0% to 50% EtOAc in heptane) to give 5.999 g of the title compound as an off-white solid (81% yield). ESI [M+H]⁺; 435.3

### b) N-(3-(4-chlorobenzyl)-1,2,4-thiadiazol-5-yl)spiro[3.3]heptane-2,6-diamine TFA salt

Tert-butyl 6-(3-(4-chlorobenzyl)-1,2,4-thiadiazol-5-ylamino)spiro[3.3]heptan-2-ylcarbamate (5.999 g, 13.8 mmol, Eq: 1.00) was combined with DCM (25 ml) to give a off-white suspension and TFA (7.86 g, 5.28 ml, 69.0 mmol, Eq: 5) was added giving an orange solution. The reaction mixture was stirred overnight. The crude reaction mixture was concentrated in vacuo to give an orange oil. Diethyl ether was added to the crude and evaporated 4 times, to give an orange solid which was dried at 40° C. under high vacuum yielding the title compound. ESI [M+H]⁺; 335.2

### SYNTHESIS OF EXAMPLES

### Final Compound of Formula B1A Following General Scheme A Starting from Intermediate 4* (for Example IB 4-1, IB 4-2 or IB 4-3)

i) Amine 4* was combined with aldehyde 5* (Eq: 1.00) in DCM. The reaction mixture was stirred for 30 min. Sodium triacetoxyborohydride (Eq: 1.5) was added. The reaction mixture was stirred at room temp. for 2 hours. The reaction was poured into DCM and extracted with Na₂CO₃. The aqueous layer was back-extracted with DCM and the organic layers were dried over Na₂SO₄ and concentrated in vacuo, then isolated after silica chromatography using DCM/MeOH. or:

ii) Amine 4* was combined with aldehyde 5* (Eq: 1.00) in EtOH with triethylamine (Eq: 1.5) and acetic acid (Eq: 2). The reaction mixture was stirred for 30 min. Sodium cyanoborohydride (Eq: 1.2) was added. The reaction mixture was stirred at room temp. for 2 hours. The reaction was poured into DCM and extracted with Na₂CO₃. The aqueous layer was back-extracted with DCM and the organic layers were dried over Na₂SO₄ and concentrated in vacuo then isolated by silica chromatography using DCM/MeOH. or:
iii) To a solution of amine 4* (0.15 mmol) with triethylamine (5 eq.) in DMF (1 mL) was added the appropriate halogenated electrophile 5*. Reaction was stirred overnight at room temperature and the resultant product was purified by prep. HPLC.

### Final Compound B1B Following General Scheme B Starting from Intermediate 7* (for Example IB 7-1, IB 7-2)

Amine 7* was combined with aldehyde 8* (Eq: 1.00) and TEA (Eq: 2) in DCE. The reaction mixture was stirred for 5 min. Sodium triacetoxyborohydride (Eq: 1.5) was added. The reaction mixture was stirred at RT 1 hour. The reaction was poured into DCM and extracted with NaHCO₃. The aqueous layer was back-extracted with DCM and the organic layers were dried over Na₂SO₄ and concentrated in vacuo. then isolated by silica chromatography using DCM/MeOH. iv) When R^{4b} in compound B1B is hydrogen, methyl can be introduced in that position by, for example, combining compound B1B wherein R^{4b} is hydrogen with formaldehyde (Eq: 1.5), TEA (Eq: 1) and acetic acid (Eq: 0.4) in DCE/MeOH:4/1. The reaction mixture was stirred for 5 min. Sodium triacetoxyborohydride (Eq: 2) was added. The reaction mixture was stirred at RT 1 hour. The reaction was poured into DCM and extracted with NaHCO₃. The aqueous layer was back-extracted with DCM and the organic layers were dried over Na₂SO₄ and concentrated in vacuo then isolated using silica chromatography using heptane/ethyl acetate.

### Synthesis of 2-(4-fluorophenyl)-1-(6-(3-phenylpropyl)-2,6-diazaspiro[3.3]heptan-2-yl)ethanone (Cpd B001)

Coupling between a building block described herein and aldehyde according to general procedure for preparation of B1A i):
Building block: 2-(4-fluorophenyl)-1-(2,6-diazaspiro[3.3]heptan-2-yl)ethanone (IB 4-2)
Aldehyde: 3-phenylpropanal (commercially available)

ESI [M+H]⁺; 353.4

### Synthesis of 1-(6-benzyl-2,6-diazaspiro[3.3]heptan-2-yl)-2-(4-fluorophenyl)ethanone (Cpd B002)

Coupling between a building block described herein and aldehyde according to general procedure for preparation of B1A i):
Building block: 2-(4-fluorophenyl)-1-(2,6-diazaspiro[3.3]heptan-2-yl)ethanone (IB 4-2)
Aldehyde: benzaldehyde (commercially available)

ESI [M+H]⁺; 325.2

### Synthesis of 1-(6-benzyl-2,6-diazaspiro[3.3]heptan-2-yl)-2-(4-chlorophenyl)ethanone(Cpd B003)

Coupling between a building block described herein and aldehyde according to general procedure for preparation of B1A i):
Building block: 2-(4-chlorophenyl)-1-(2,6-diazaspiro[3.3]heptan-2-yl)ethanone (IB 4-1)
Aldehyde: benzaldehyde (commercially available)

ESI [M+H]⁺; 341.0

### Synthesis of 1-(6-(4-chlorobenzyl)-2,6-diazaspiro[3.3]heptan-2-yl)-2-(4-chlorophenyl)ethanone (Cpd B004)

Coupling between a building block described herein and aldehyde according to general procedure for preparation of B1A i):
Building block: 2-(4-chlorophenyl)-1-(2,6-diazaspiro[3.3]heptan-2-yl)ethanone (IB 4-1)
Aldehyde: 4-chlorobenzaldehyde (commercially available)

ESI [M+H]⁺; 375.0

### Synthesis of 2-(4-chlorophenyl)-1-(6-((2-methoxypyridin-4-yl)methyl)-2,6-diazaspiro[3.3]heptan-2-yl)ethanone (Cpd B005)

Coupling between a building block described herein and aldehyde according to general procedure for preparation of B1A i):
Building block: 2-(4-chlorophenyl)-1-(2,6-diazaspiro[3.3]heptan-2-yl)ethanone (IB 4-1)
Aldehyde: 2-methoxyisonicotinaldehyde (commercially available)

ESI [M+H]⁺; 372.0

### Synthesis of 2-(4-chlorophenyl)-1-(6-(2-methylbenzyl)-2,6-diazaspiro[3.3]heptan-2-yl)ethanone (Cpd B006)

Coupling between a building block described herein and aldehyde according to general procedure for preparation of B1A i):
Building block: 2-(4-chlorophenyl)-1-(2,6-diazaspiro[3.3]heptan-2-yl)ethanone (IB 4-1)
Aldehyde: 2-methylbenzaldehyde (commercially available)

ESI [M+H]⁺; 355.1

### Synthesis of 1-(6-benzyl-2,6-diazaspiro[3.3]heptan-2-yl)-2-(3,4-difluorophenyl)ethanone (Cpd B007)

Coupling between a building block described herein and aldehyde according to general procedure for preparation of B1A ii):
Building block: 2-(3,4-difluorophenyl)-1-(2,6-diazaspiro[3.3]heptan-2-yl)ethanone (IB 4-3)
Aldehyde: benzaldehyde (commercially available)

ESI [M+H]⁺; 343.2

### Synthesis of 2-(4-Fluoro-phenyl)-1-[6-(3-methoxy-benzyl)-2,6-diaza-spiro[3.3]hept-2-yl]-ethanone (Cpd B008)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(bromomethyl)-3-methoxybenzene (commercially available)

ESI [M+H]⁺; 355.3

### Synthesis of 2-(4-Fluoro-phenyl)-1-[6-(4-methyl-benzyl)-2,6-diaza-spiro[3.3]hept-2-yl]-ethanone (Cpd B009)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(bromomethyl)-4-methylbenzene (commercially available)

ESI [M+H]⁺; 339.3

### Synthesis of 2-(4-Fluoro-phenyl)-1-(6-pyridin-2-ylmethyl-2,6-diaza-spiro[3.3]hept-2-yl)-ethanone (Cpd B010)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 2-(bromomethyl)pyridine (commercially available)

ESI [M+H]⁺; 326.3

### Synthesis of 1-[6-(3-Chloro-benzyl)-2,6-diaza-spiro[3.3]hept-2-yl]-2-(4-fluoro-phenyl)-ethanone (Cpd B011)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(bromomethyl)-3-chlorobenzene (commercially available)

ESI [M+H]⁺; 359.2

### Synthesis of 3-{6-[2-(4-Fluoro-phenyl)-acetyl]-2,6-diaza-spiro[3.3]hept-2-ylmethyl}-benzamide (Cpd B012)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 3-(chloromethyl)benzamide (commercially available)

ESI [M+H]⁺; 368.2

### Synthesis of 2-(4-Fluoro-phenyl)-1-[6-(2-trifluoromethoxy-benzyl)-2,6-diaza-spiro[3.3]hept-2-yl]-ethanone (Cpd B013)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(bromomethyl)-2-(trifluoromethoxy)benzene (commercially available)

ESI [M+H]⁺; 409.2

### Synthesis of 4-{6-[2-(4-Fluoro-phenyl)-acetyl]-2,6-diaza-spiro[3.3]hept-2-ylmethyl}-benzoic acid methyl ester (Cpd B014)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: methyl 4-(bromomethyl)benzoate (commercially available)

ESI [M+H]⁺; 383.2

### Synthesis of 2-(4-Fluoro-phenyl)-1-{6-[1-(3-trifluoromethyl-phenyl)-ethyl]-2,6-diaza-spiro[3.3]hept-2-yl}-ethanone (Cpd B015)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(1-bromoethyl)-3-(trifluoromethyl)benzene (commercially available)

ESI [M+H]⁺; 407.4

### Synthesis of 2-(4-Fluoro-phenyl)-1-[6-(1-phenyl-ethyl)-2,6-diaza-spiro[3.3]hept-2-yl]-ethanone (Cpd B016)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: (1-bromoethyl)benzene (commercially available)

ESI [M+H]⁺; 339.1

### Synthesis of 2-(4-Fluoro-phenyl)-1-[6-(6-methyl-pyridin-2-ylmethyl)-2,6-diaza-spiro[3.3]hept-2-yl]-ethanone (Cpd B017)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 2-(bromomethyl)-6-methylpyridine (commercially available)

ESI [M+H]⁺; 340.3

### Synthesis of 1-[6-(2-Chloro-benzyl)-2,6-diaza-spiro[3.3]hept-2-yl]-2-(4-fluoro-phenyl)-ethanone (Cpd B018)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(bromomethyl)-2-chlorobenzene (commercially available)

ESI [M+H]⁺; 359.0

### Synthesis of 2-(4-Fluoro-phenyl)-1-[6-(2-methyl-thiazol-4-ylmethyl)-2,6-diaza-spiro[3.3]hept-2-yl]-ethanone (Cpd B019)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 4-(chloromethyl)-2-methylthiazole hydrochloride (commercially available)

ESI [M+H]⁺; 346.0

### Synthesis of 2-(4-Fluoro-phenyl)-1-[6-(1-methyl-1H-imidazol-2-ylmethyl)-2,6-diaza-spiro[3.3]hept-2-yl]-ethanone (Cpd B020)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 2-(chloromethyl)-1-methyl-1H-imidazole hydrochloride (commercially available)

ESI [M+H]⁺; 329.4

### Synthesis of 2-(4-Fluoro-phenyl)-1-[6-(5-methyl-isoxazol-3-ylmethyl)-2,6-diaza-spiro[3.3]hept-2-yl]-ethanone (Cpd B021)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 3-(bromomethyl)-5-methylisoxazole (commercially available)

ESI [M+H]⁺; 330.0

### Synthesis of 2-(4-Fluoro-phenyl)-1-(6-thiazol-4-ylmethyl-2,6-diaza-spiro[3.3]hept-2-yl)-ethanone (Cpd B022)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 4-(chloromethyl)thiazole (commercially available)

ESI [M+H]⁺; 332.2

### Synthesis of 2-(4-Fluoro-phenyl)-1-{6-[2-(3-trifluoromethyl-phenyl)-ethyl]-2,6-diaza-spiro[3.3]hept-2-yl}-ethanone (Cpd B023)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(2-bromoethyl)-3-(trifluoromethyl)benzene (commercially available)

ESI [M+H]⁺; 407.4

### Synthesis of 2-(4-Fluoro-phenyl)-1-{6-[2-(3-fluoro-phenyl)-ethyl]-2,6-diaza-spiro[3.3]hept-2-yl}-ethanone (Cpd B024)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)-Halogenated electrophile: 1-(2-bromoethyl)-3-fluorobenzene (commercially available)

ESI [M+H]⁺; 357.2

### Synthesis of 2-(4-Fluoro-phenyl)-1-{6-[2-(4-methoxy-phenyl)-ethyl]-2,6-diaza-spiro[3.3]hept-2-yl}-ethanone (Cpd B025)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(2-bromoethyl)-4-methoxybenzene (commercially available)

ESI [M+H]⁺; 369.2

### Synthesis of 2-(4-Fluoro-phenyl)-1-[6-(2-p-tolyl-ethyl)-2,6-diaza-spiro[3.3]hept-2-yl]-ethanone (Cpd B026)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2) Halogenated electrophile: 1-(2-bromoethyl)-4-methylbenzene (commercially available)

ESI [M+H]⁺; 353.3

### Synthesis of 2-(4-Fluoro-phenyl)-1-{6-[2-(4-fluoro-phenyl)-ethyl]-2,6-diaza-spiro[3.3]hept-2-yl}-ethanone (Cpd B027)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(2-bromoethyl)-4-fluorobenzene (commercially available)

ESI [M+H]⁺; 357.2

### Synthesis of 2-(4-Fluoro-phenyl)-1-[6-(2-m-tolyl-ethyl)-2,6-diaza-spiro[3.3]hept-2-yl]-ethanone (Cpd B028)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(2-bromoethyl)-3-methylbenzene (commercially available)

ESI [M+H]⁺; 353.3

### Synthesis of 2-{6-[2-(4-Fluoro-phenyl)-acetyl]-2,6-diaza-spiro[3.3]hept-2-yl}-2-methyl-1-phenyl-propan-1-one (Cpd B029)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 2-bromo-2-methyl-1-phenylpropan-1-one (commercially available)

ESI [M+H]⁺; 381.3

### Synthesis of 2-(4-Fluoro-phenyl)-1-(6-phenethyl-2,6-diaza-spiro[3.3]hept-2-yl)-ethanone (Cpd B030)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: (2-bromoethyl)benzene (commercially available)

ESI [M+H]⁺; 339.2

### Synthesis of 2-(4-chlorophenyl)-1-(6-(3-methoxybenzyl)-2,6-diazaspiro[3.3]heptan-2-yl)ethanone (Cpd B031)

Coupling between a building block described herein above and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(bromomethyl)-3-methoxybenzene (commercially available)

ESI [M+H]⁺; 371.2

### Synthesis of 2-(4-chlorophenyl)-1-(6-(pyridin-2-ylmethyl)-2,6-diazaspiro[3.3]heptan-2-yl)ethanone (Cpd B032)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 2-(bromomethyl)pyridine (commercially available)

ESI [M+H]⁺; 342.1

### Synthesis of 1-(6-(3-chlorobenzyl)-2,6-diazaspiro[3.3]heptan-2-yl)-2-(4-chlorophenyl)ethanone (Cpd B033)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(bromomethyl)-3-chlorobenzene (commercially available)

ESI [M+H]⁺; 375.3

### Synthesis of 2-(4-chlorophenyl)-1-(6-(2-(trifluoromethoxy)benzyl)-2,6-diazaspiro[3.3]heptan-2-yl)ethanone (Cpd B034)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(bromomethyl)-2-(trifluoromethoxy)benzene (commercially available)

ESI [M+H]⁺; 424.9

### Synthesis of methyl 4-((6-(2-(4-chlorophenyl)acetyl)-2,6-diazaspiro[3.3]heptan-2-yl)methyl)benzoate (Cpd B035)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: methyl 4-(bromomethyl)benzoate (commercially available)

ESI [M+H]⁺; 399.0

### Synthesis of 2-(4-chlorophenyl)-1-(6-((4,6-dimethoxypyrimidin-2-yl)methyl)-2,6-diazaspiro[3.3]heptan-2-yl)ethanone (Cpd B036)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 2-(chloromethyl)-4,6-dimethoxypyrimidine (commercially available)

ESI [M+H]⁺; 403.1

### Synthesis of 2-(4-chlorophenyl)-1-(6-(1-(3-(trifluoromethyl)phenyl)ethyl)-2,6-diazaspiro[3.3]heptan-2-yl)ethanone (Cpd B037)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(1-bromoethyl)-3-(trifluoromethyl)benzene (commercially available)

ESI [M+H]⁺; 423.2

### Synthesis of 2-(4-chlorophenyl)-1-(6-((6-methylpyridin-2-yl)methyl)-2,6-diazaspiro[3.3]heptan-2-yl)ethanone (Cpd B038)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 2-(bromomethyl)-6-methylpyridine (commercially available)

ESI [M+H]⁺; 356.1

### Synthesis of 2-(4-chlorophenyl)-1-(6-((5-chloropyrimidin-2-yl)methyl)-2,6-diazaspiro[3.3]heptan-2-yl)ethanone (Cpd B039)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 5-chloro-2-(chloromethyl)pyrimidine (commercially available)

ESI [M+H]⁺; 377.3

### Synthesis of 2-(4-Chloro-phenyl)-1-[6-(5-trifluoromethyl-furan-2-ylmethyl)-2,6-diaza-spiro[3.3]hept-2-yl]-ethanone (Cpd B040)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 2-(bromomethyl)-5-(trifluoromethyl)furan (commercially available)

ESI [M+H]⁺; 399.0

### Synthesis of 2-(4-chlorophenyl)-1-(6-((5-methylisoxazol-3-yl)methyl)-2,6-diazaspiro[3.3]heptan-2-yl)ethanone (Cpd B041)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 3-(bromomethyl)-5-methylisoxazole (commercially available)

ESI [M+H]⁺; 346.1

### Synthesis of 2-(4-chlorophenyl)-1-(6-propyl-2,6-diazaspiro[3.3]heptan-2-yl)ethanone (Cpd B042)

Coupling between a building block described herein and halogenated electrophile according to general procedure for preparation of IB1A iii):
Building block: 2-(4-chlorophenyl)-1-(2,6-diazaspiro[3.3]heptan-2-yl)ethanone (IB 4-1)
Halogenated electrophile: 1-iodopropane (commercially available)

ESI [M+H]⁺; 293.1

### Synthesis of 2-(4-chlorophenyl)-1-(6-(thiazol-4-ylmethyl)-2,6-diazaspiro[3.3]heptan-2-yl)ethanone (Cpd B043)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1) Halogenated electrophile: 4-(chloromethyl)thiazole (commercially available)

ESI [M+H]⁺; 348.2

### Synthesis of 2-(4-chlorophenyl)-1-(6-(3-(trifluoromethyl)phenethyl)-2,6-diazaspiro[3.3]heptan-2-yl)ethanone (Cpd B044)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1) Halogenated electrophile: 1-(2-bromoethyl)-3-(trifluoromethyl)benzene (commercially available)

ESI [M+H]⁺; 423.2

### Synthesis of 2-(4-chlorophenyl)-1-(6-(3-fluorophenethyl)-2,6-diazaspiro[3.3]heptan-2-yl)ethanone (Cpd B045)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(2-bromoethyl)-3-fluorobenzene (commercially available)

ESI [M+H]⁺; 373.2

### Synthesis of 2-(4-chlorophenyl)-1-(6-(4-methoxyphenethyl)-2,6-diazaspiro[3.3]heptan-2-yl)ethanone (Cpd B046)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(2-bromoethyl)-4-methoxybenzene (commercially available)

ESI [M+H]⁺; 385.2

### Synthesis of 2-(4-chlorophenyl)-1-(6-(4-methylphenethyl)-2,6-diazaspiro[3.3]heptan-2-yl)ethanone (Cpd B047)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(2-bromoethyl)-4-methylbenzene (commercially available)

ESI [M+H]⁺; 369.0

### Synthesis of 2-(4-chlorophenyl)-1-(6-(4-fluorophenethyl)-2,6-diazaspiro[3.3]heptan-2-yl)ethanone (Cpd B048)

Coupling between a building block described herein and halogenated electrophile according to general procedure for preparation of B1A iii):
Building block: 2-(4-chlorophenyl)-1-(2,6-diazaspiro[3.3]heptan-2-yl)ethenone (IB 4-1)
Halogenated electrophile: 1-(2-bromoethyl)-4-fluorobenzene (commercially available)

ESI [M+H]⁺; 373.0

### Synthesis of 2-(4-chlorophenyl)-1-(6-(3-methylphenethyl)-2,6-diazaspiro[3.3]heptan-2-yl)ethanone (Cpd B049)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(2-bromoethyl)-3-methylbenzene (commercially available)

ESI [M+H]⁺; 369.2

### Synthesis of 2-(6-(2-(4-chlorophenyl)acetyl)-2,6-diazaspiro[3.3]heptan-2-yl)-2-methyl-1-phenylpropan-1-one (Cpd B050)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 2-bromo-2-methyl-1-phenylpropan-1-one (commercially available)

ESI [M+H]⁺; 397.3

### Synthesis of 2-(4-chlorophenyl)-1-(6-(2-(6-methylpyridin-2-yl)ethyl)-2,6-diazaspiro[3.3]heptan-2-yl)ethanone (Cpd B051)

Coupling between a building block described herein and halogenated electrophile according to general procedure for preparation of B1A iii):
Building block: 2-(4-chlorophenyl)-1-(2,6-diazaspiro[3.3]heptan-2-yl)ethenone; (IB 4-1)
Halogenated electrophile: 2-(2-bromoethyl)-6-methylpyridine (commercially available)

ESI [M+H]⁺; 370.3

### Synthesis of 2-(4-chlorophenyl)-1-(6-phenethyl-2,6-diazaspiro[3.3]heptan-2-yl)ethanone (Cpd B052)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: (2-bromoethyl)benzene (commercially available)

ESI [M+H]⁺; 355.3

### Synthesis of 2-(4-chlorophenyl)-1-(6-isopentyl-2,6-diazaspiro[3.3]heptan-2-yl)ethanone (Cpd B053)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-iodo-3-methylbutane (commercially available)

ESI [M+H]⁺; 321.3

### Synthesis of 2-(4-chlorophenyl)-1-(6-(1-phenylpropan-2-yl)-2,6-diazaspiro[3.3]heptan-2-yl)ethanone (Cpd B054)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: (2-bromopropyl)benzene (commercially available)

ESI [M+H]⁺; 369.2

### Synthesis of 2-(4-chlorophenyl)-1-(6-(3,4-difluorobenzyl)-2,6-diazaspiro[3.3]heptan-2-yl)ethanone (Cpd B055)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 4-(bromomethyl)-1,2-difluorobenzene (commercially available)

ESI [M+H]⁺; 377.3

### Synthesis of 2-(4-chlorophenyl)-1-(6-(cyclohexylmethyl)-2,6-diazaspiro[3.3]heptan-2-yl)ethanone (Cpd B056)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: (bromomethyl)cyclohexane (commercially available)

ESI [M+H]⁺; 347.2

### Synthesis of 2-(4-chlorophenyl)-1-(6-(2-cyclohexylethyl)-2,6-diazaspiro[3.3]heptan-2-yl)ethanone (Cpd B057)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: (2-bromoethyl)cyclohexane (commercially available)

ESI [M+H]⁺; 361.3

### Synthesis of 2-(4-chlorophenyl)-1-(6-(3-(2-methoxyphenyl)propyl)-2,6-diazaspiro[3.3]heptan-2-yl)ethanone (Cpd B058)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(3-bromopropyl)-2-methoxybenzene (commercially available)

ESI [M+H]⁺; 399.2

### Synthesis of 2-(4-chlorophenyl)-1-(6-(3-(4-methoxyphenyl)propyl)-2,6-diazaspiro[3.3]heptan-2-yl)ethanone (Cpd B059)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(3-bromopropyl)-4-methoxybenzene (commercially available)

ESI [M+H]⁺; 399.2

### Synthesis of 2-(4-Fluoro-phenyl)-1-{6-[3-(4-methoxy-phenyl)-propyl]-2,6-diaza-spiro[3.3]hept-2-yl}-ethanone (Cpd B060)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(3-bromopropyl)-4-methoxybenzene (commercially available)

ESI [M+H]⁺; 383.3

### Synthesis of 2-(4-chlorophenyl)-1-(6-(4-fluorobenzyl)-2,6-diazaspiro[3.3]heptan-2-yl)ethanone (Cpd B061)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(bromomethyl)-4-fluorobenzene (commercially available)

ESI [M+H]⁺; 359.2

### Synthesis of 1-(6-(2-chlorophenethyl)-2,6-diazaspiro[3.3]heptan-2-yl)-2-(4-chlorophenyl)ethanone (Cpd B062)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: 1-(2-bromoethyl)-2-chlorobenzene (commercially available)

ESI [M+H]⁺; 389.0

### Synthesis of 1-{6-[2-(2-Chloro-phenyl)-ethyl]-2,6-diaza-spiro[3.3]hept-2-yl}-2-(4-fluoro-phenyl)-ethanone (Cpd B063)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 1-(2-bromoethyl)-2-chlorobenzene (commercially available)

ESI [M+H]⁺; 373.2

### Synthesis of 1-[6-(4,6-Dimethoxy-pyrimidin-2-ylmethyl)-2,6-diaza-spiro[3.3]hept-2-yl]-2-(4-fluoro-phenyl)-ethanone (Cpd B064)

Coupling between a building block described herein and halogenated electrophile according to general procedure for preparation of B1A iii):
Building block: 2-(4-fluorophenyl)-1-(2,6-diazaspiro[3.3]heptan-2-yl)ethanone (IB 4-2)
Halogenated electrophile: 2-(chloromethyl)-4,6-dimethoxypyrimidine (commercially available)

ESI [M+H]⁺; 387.2

### Synthesis of 2-(4-chlorophenyl)-1-(6-(1-phenylethyl)-2,6-diazaspiro[3.3]heptan-2-yl)ethanone (Cpd B065)

Coupling between a building block described herein and halogenated electrophile according to general procedure for preparation of B1A iii):
Building block: 2-(4-chlorophenyl)-1-(2,6-diazaspiro[3.3]heptan-2-yl)ethanone (IB 4-1)
Halogenated electrophile: (1-bromoethyl)benzene (commercially available)

ESI [M+H]⁺; 355.2

### Synthesis of N-(6-(benzylamino)spiro[3.3]heptan-2-yl)-2-(4-fluorophenyl)acetamide (Cpd B066)

Coupling between a building block described herein and aldehyde according to general procedure for preparation of B1B:
Building block: N-(6-Amino-spiro[3.3]hept-2-yl)-2-(4-fluoro-phenyl)-acetamide (HCl) (IB 7-1)
Aldehyde: benzaldehyde (commercially available)

ESI [M+H]⁺; 353.2

### Synthesis of N-(6-(benzyl(methyl)amino)spiro[3.3]heptan-2-yl)-2-(4-fluorophenyl)acetamide (Cpd B067)

Coupling between a building block described herein and aldehyde according to general procedure for preparation of B1B:
Building block: (Cpd B066)
Aldehyde: formaldehyde (commercially available)

ESI [M+H]⁺; 367.2

### Synthesis of N-(6-(benzyl(methyl)amino)spiro[3.3]heptan-2-yl)-2-(4-chlorophenyl)acetamide (Cpd B068)

Coupling between a building block described herein and aldehyde according to general procedure for preparation of B1B:
Building block: N-(6-aminospiro[3.3]heptan-2-yl)-2-(4-chlorophenyl)acetamide, HCl (IB 7-2)
Aldehyde: benzaldehyde (commercially available)

giving N-(6-(benzylamino)spiro[3.3]heptan-2-yl)-2-(4-chlorophenyl)acetamide

(ESI [M+H]⁺; 369.2), followed by methylation according to general procedure for preparation B1B following further step iv)

ESI [M+H]⁺; 383.2

### Synthesis of 2-(4-(trifluoromethyl)phenyl)-1-(6-(3-methoxybenzyl)-2,6-diazaspiro[3.3]heptan-2-yl)ethan-1-one (Cpd B069)

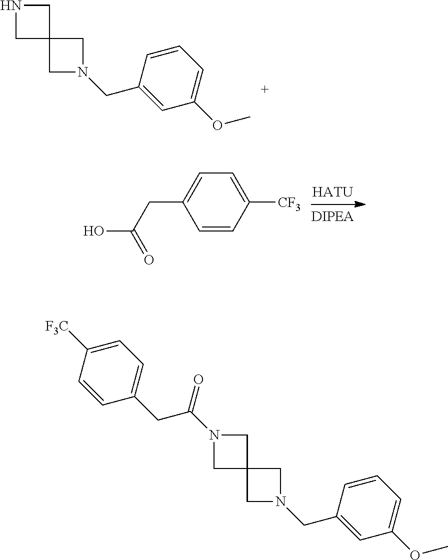

To 4-(trifluoromethyl)phenylacetic acid (0.030 g, 0.150 mmol) was added a stock solution of HATU (0.047 g, 0.125 mmol)/DIPEA (0.050 ml, 0.286 mmol) in DMF (dry) (1 ml). The mixture was stirred at RT for 30 min. Then, 2-(3-methoxybenzyl)-2,6-diazaspiro[3.3]heptane I12-4 (0.021 g, 0.1 mmol) in DMF (dry) (0.5 ml) was added and the mixture was stirred overnight at RT. The reaction was filtered (0.45 μm) and purified by basic preparative HPLC-MS. Product fractions were concentrated, combined with ACN, water was added and the product was lyophilized to afford 2-(4-(trifluoromethyl)phenyl)-1-(6-(3-methoxybenzyl)-2,6-diazaspiro[3.3]heptan-2-yl)ethan-1-one (Cpd B069). (Yield: 12%) MS (m/e): 405.2 (MH⁺) rt=1.51 min.

The following compounds were prepared in a similar manner (use of appropriate starting material, intermediates, reagents and purification methods known to the person skilled in the art or as described herein) as described for Cpd B069: Cpd B070, Cpd B071, Cpd B072, Cpd B073, Cpd B074, Cpd B075, Cpd B076, Cpd B077, Cpd B078, Cpd B079, Cpd B080, Cpd B081, Cpd B082, Cpd B083, Cpd B084, Cpd B085, Cpd B086, Cpd B087, Cpd B088, Cpd B089, Cpd B090, Cpd B091, Cpd B092, Cpd B093, Cpd B094, Cpd B095, Cpd B096, Cpd B097, Cpd B098, Cpd B099, Cpd B100, Cpd B101, Cpd B102 and Cpd B103.

| | [M + H]⁺ (m/e)/rt time | | [M + H]⁺ (m/e)/rt time | | [M + H]⁺ (m/e)/rt |
|---|---|---|---|---|---|
| Compounds | (min) | Compounds | (min) | Compounds | time mine |
| Cpd B070 | 359.2/1.43 | Cpd B082 | 328.3/0.79 | Cpd B093 | 343.1/1.36 |
| Cpd B071 | 343.2/1.53 | Cpd B083 | 364.2/1.19 | Cpd B094 | 329.1/1.36 |
| Cpd B072 | 327.2/1.32 | Cpd B084 | 330.3/0.88 | Cpd B095 | 336.2/1.29 |
| Cpd B073 | 343.2/1.50 | Cpd B085 | 400.2/1.48 | Cpd B096 | 313.1/1.01 |
| Cpd B074 | 379.2/1.38 | Cpd B086 | 347.2/1.27 | Cpd B097 | 318.0/1.09 |
| Cpd B075 | 366.2/1.40 | Cpd B087 | 376.2/1.33 | Cpd B098 | 359.1/1.40 |
| Cpd B076 | 339.2/0.99 | Cpd B088 | 290.2/0.83 | Cpd B099 | 379.1/1.57 |
| Cpd B077 | 408.2/1.33 | Cpd B089 | 345.2/1.67 | Cpd B100 | 396.2/1.25 |
| Cpd B078 | 390.2/1.30 | Cpd B090 | 409.2/1.56 | Cpd B101 | 414.1/1.29 |
| Cpd B079 | 390.1/1.32 | Cpd B091 | 393.2/1.54 | Cpd B102 | 370.2/1.27 |
| Cpd B080 | 323.2/0.88 | Cpd B092 | 350.1/1.28 | Cpd B103 | 362.1/1.13 |
| Cpd B081 | 342.2/0.92 | | | | |

### Synthesis of 2-(4,4-difluorocyclohexyl)-1-(6-(4-fluoro-2-(trifluoromethyl)benzyl)-2,6-diazaspiro[3.3]heptan-2-yl)ethan-1-one (Cpd B104)

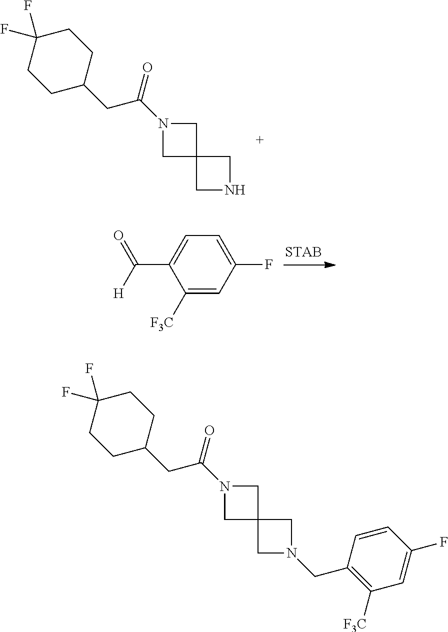 [SMILES: O=C(CC1CCC(F)(F)CC1)N1CC2(CNC2)C1 ; O=Cc1ccc(F)cc1C(F)(F)F ; O=C(CC1CCC(F)(F)CC1)N1CC2(CN(Cc3ccc(F)cc3C(F)(F)F)C2)C1]

2-(4,4-difluorocyclohexyl)-1-(2,6-diazaspiro[3.3]heptan-2-yl)ethan-1-one I22-1 (25.8 mg, 0.1 mmol) in MeOH (1 mL) and 4-fluoro-2-(trifluoromethyl)benzaldehyde (28.8 mg, 0.150 mmol) were mixed and the reaction was stirred at RT for 1.5h. STAB (0.053 g, 0.250 mmol) was added and the mixture was stirred overnight at RT. 7N NH₃ in MeOH (0.4 mL, -2.8 mmol) was added and mixed with the reaction. The reaction was filtered (0.45 μm) and purified by basic preparative HPLC-MS. Product fractions were concentrated, combined with ACN, water was added and the product was lyophilized to afford 2-(4,4-difluorocyclohexyl)-1-(6-(4-fluoro-2-(trifluoromethyl)benzyl)-2,6-diazaspiro[3.3]heptan-2-yl)ethan-1-one (Cpd B104). (12.8 mg, 0.029 mmol, 29%) MS (m/e): 435.2 (MH⁺) rt=1.68 min.

The following compounds were prepared in a similar manner (use of appropriate starting material, intermediates, reagents and purification methods (including chiral HPLC or chiral SFC) known to the person skilled in the art or as described herein) as described for Cpd B104: Cpd B105, Cpd B106, Cpd B107, Cpd B108, Cpd B109, Cpd B110, Cpd B111, Cpd B112, Cpd B113, Cpd B114, Cpd B115, Cpd B116, Cpd B117, Cpd B118, Cpd B119, Cpd B120, Cpd B121, Cpd B122, Cpd B123, Cpd B124, Cpd B125, Cpd B126 and Cpd B127.

| | [M + H]⁺ (m/e)/rt |
|---|---|
| Compounds | time (min) |
| Cpd B105 | 385.2/1.46 |
| Cpd B106 | 401.2/1.57 |
| Cpd B107 | 401.2/1.59 |
| Cpd B108 | 417.2/1.63 |
| Cpd B109 | 417.3/1.58 |
| Cpd B110 | 435.2/1.61 |
| Cpd B111 | 415.2/1.50 |
| Cpd B112 | 418.2/1.37 |
| Cpd B113 | 384.2/1.28 |
| Cpd B114 | 384.2/1.26 |
| Cpd B115 | 368.2/1.18 |
| Cpd B116 | 370.2/0.75 |
| Cpd B117 | 404.2/1.34 |
| Cpd B118 | 367.2/1.11 |
| Cpd B119 | 353.2/1.05 |
| Cpd B120 | 387.2/1.20 |
| Cpd B121 | 401.2/1.27 |
| Cpd B122 | 401.2/1.32 |
| Cpd B123 | 378.2/1.20 |
| Cpd B124 | 353.2/0.98 |
| Cpd B125 | 353.2/0.96 |
| Cpd B126 | 353.2/0.99 |
| Cpd B127 | 354.2/1.09 |

### Synthesis of (tetrahydro-2H-pyran-4-yl)methyl 6-(4-fluorobenzyl)-2,6-diazaspiro[3.3]heptane-2-carboxylate (Cpd B128)

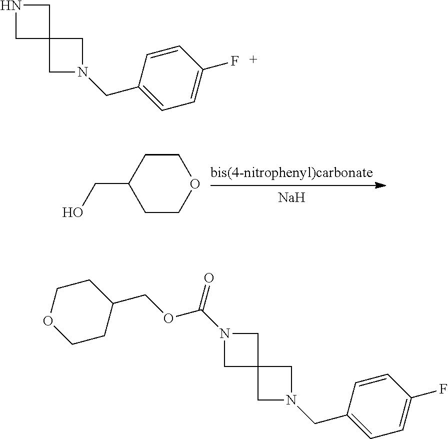

To bis(4-nitrophenyl) carbonate (35.0 mg, 0.115 mmol) in DMF (dry) (0.5 ml) were added (tetrahydro-2H-pyran-4-yl)methanol (17.42 mg, 0.150 mmol) and sodium hydride (8.00 mg, 0.200 mmol). The mixture was stirred at RT for 1.5h before addition of stock solution of 2-(4-fluorobenzyl)-2,6-diazaspiro[3.3]heptane I12-1 (20.63 mg, 0.1 mmol) in DMF (dry) (1 ml). The mixture was stirred overnight at RT. The product was filtered and purified by means of basic preparative HPLC (MeCN in 10 mM ammoniumbicarbonate in water). Purified fractions were combined, reformatted and analyzed to afford (tetrahydro-2H-pyran-4-yl)methyl 6-(4-fluorobenzyl)-2,6-diazaspiro[3.3]heptane-2-carboxylate (Cpd B128). (6.4 mg, 0.018 mmol, 18.4% yield) MS (m/e): 349.2 (MH⁺) rt=1.31 min.

The following compounds were prepared in a similar manner (use of appropriate starting material, intermediates, reagents and purification methods (including chiral HPLC or chiral SFC) known to the person skilled in the art or as described herein) as described for Cpd B128: Cpd B129, Cpd B130, Cpd B131, Cpd B132, Cpd B133, Cpd B134, Cpd B135, Cpd B136, Cpd B137, Cpd B138 and Cpd B139.

| | [M + H]+ (m/e)/rt |
|---|---|
| Compounds | time (min) |
| Cpd B129 | 349.1/1.05 |
| Cpd B130 | 375.1/1.71 |
| Cpd B131 | 333.2/1.74 |
| Cpd B132 | 383.2/1.63 |
| Cpd B133 | 369.2/1.58 |
| Cpd B134 | 372.1/1.56 |
| Cpd B135 | 381.4/1.68 |
| Cpd B136 | 352.2/1.38 |
| Cpd B137 | 372.1/0.92 |
| Cpd B138 | 316.4/1.49 |
| Cpd B139 | 366.2/0.81 |

### Synthesis of 4-fluorophenyl 6-(3-methoxybenzyl)-2,6-diazaspiro[3.3]heptane-2-carboxylate (Cpd B140)

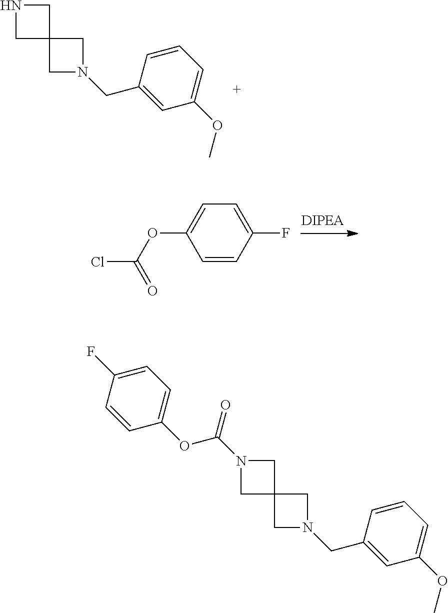 [SMILES: COc1cccc(CN2CC3(CNC3)C2)c1 ; O=C(Cl)Oc1ccc(F)cc1 ; COc1cccc(CN2CC3(C2)CN(C(=O)Oc2ccc(F)cc2)C3)c1]

To 4-fluorophenyl chloroformate (0.150 mmol) was added a stock solution of 2-(3-methoxybenzyl)-2,6-diazaspiro[3.3]heptane I12-4 (0.021 g, 0.1 mmol) in DMF (dry) (0.5 ml) and a stock solution of DIPEA (0.038 ml, 0.220 mmol) in DMF (dry) (0.5 ml). The mixture was stirred overnight at RT. The reaction mixture was filtered, purified via preparative basic LCMS. The purified fractions were combined and reformatted to afford 4-fluorophenyl 6-(3-methoxybenzyl)-2,6-diazaspiro[3.3]heptane-2-carboxylate (Cpd B140). (6.6 mg, 0.0185 mmol, 19% yield) MS (m/e): 357.2 (MH⁺) rt=1.53 min.

The compound Cpd B141 ([M+H]+(m/e) 358.2, rt=0.92 min) was prepared in a similar manner (use of appropriate starting material, intermediates, reagents and purification methods known to the person skilled in the art or as described herein) as described for Cpd B140.

### Synthesis of 4,4-difluorocyclohexyl 6-((4-chloro-1-methylpyrazol-5-yl)methyl)-2,6-diazaspiro[3.3]heptane-2-carboxylate (Cpd B142)

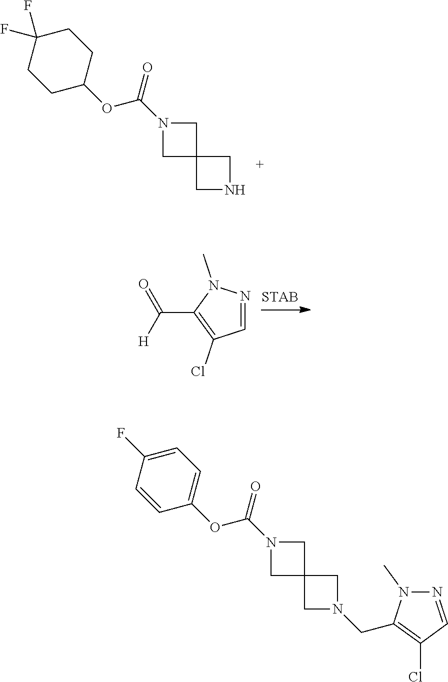

4,4-difluorocyclohexyl 2,6-diazaspiro[3.3]heptan-2-carboxylate I25-1 (26.0 mg, 0.1 mmol) in MeOH (1 mL) and 4-chloro-1-methyl-1H-pyrazole-5-carboxaldehyde (21.7 mg, 0.150 mmol) were mixed and the reaction was stirred at RT for 1.5h. STAB (0.053 g, 0.250 mmol) was added and the mixture was stirred overnight at RT. 7N NH₃ in MeOH (0.4 mL, -2.8 mmol) was added and mixed with the reaction. The reaction was filtered (0.45 μm) and purified by basic preparative HPLCMS. Product fractions were concentrated, combined with ACN, water was added and the product was lyophilized to afford 4,4-difluorocyclohexyl 6-((4-chloro-1-methylpyrazol-5-yl)methyl)-2,6-diazaspiro[3.3]heptane-2-carboxylate_(Cpd B142). (3.7 mg, 0.010 mmol, 10% yield) MS (m/e): 389.2 (MH⁺) rt=1.44 min.

The following compounds were prepared in a similar manner (use of appropriate starting material, intermediates, reagents and purification methods (including chiral HPLC or chiral SFC) known to the person skilled in the art or as described herein) as described for Cpd B142: Cpd B143, Cpd B144, Cpd B145, Cpd B146, Cpd B147, Cpd B148, Cpd B149, Cpd B150, Cpd B151, Cpd B152, Cpd B153, Cpd B154, Cpd B155, Cpd B156, Cpd B157, Cpd B158, Cpd B159, Cpd B160, Cpd B161,162, Cpd B163, Cpd B164, Cpd B165 and Cpd B166.

| | [M + H]+ (m/e)/rt |
|---|---|
| Compounds | time (min) |
| Cpd B143 | 356.1/1.21 |
| Cpd B144 | 389.2/1.32 |
| Cpd B145 | 389.2/1.36 |
| Cpd B146 | 371.1/1.65 |
| Cpd B147 | 419.2/1.83 |
| Cpd B148 | 437.2/1.80 |
| Cpd B149 | 417.2/1.67 |
| Cpd B150 | 403.1/1.76 |
| Cpd B151 | 403.2/1.79 |
| Cpd B152 | 387.2/1.64 |
| Cpd B153 | 370.2/1.32 |
| Cpd B154 | 386.2/1.43 |
| Cpd B155 | 420.2/1.54 |
| Cpd B156 | 386.2/1.35 |
| Cpd B157 | 403.2/1.38 |
| Cpd B158 | 403.2/1.38 |
| Cpd B159 | 403.2/1.41 |
| Cpd B160 | 400.0/1.49 |
| Cpd B161 | 384.2/1.38 |
| Cpd B162 | 384.2/1.36 |
| Cpd B163 | 434.2/1.59 |
| Cpd B164 | 433.2/1.80 |
| Cpd B165 | 433.2/1.87 |
| Cpd B166 | 451.2/1.91 |

### Synthesis of N-((4,4-difluorocyclohexyl)methyl)-6-(4-fluorobenzyl)-2,6-diazaspiro[3.3]heptane-2-carboxamide (Cpd B167)

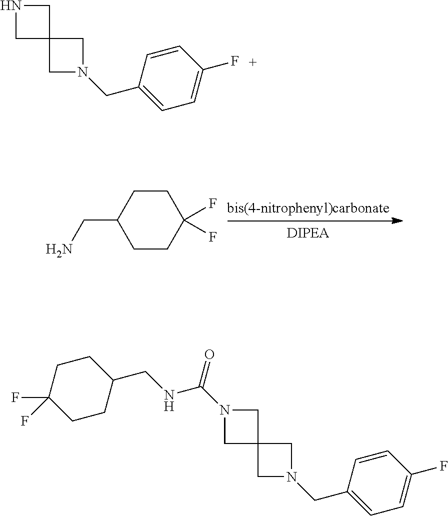 [SMILES: Fc1ccc(CN2CC3(CNC3)C2)cc1 ; NCC1CCC(F)(F)CC1 ; O=C(NCC1CCC(F)(F)CC1)N1CC2(CN(Cc3ccc(F)cc3)C2)C1]

To bis(4-nitrophenyl) carbonate (35.0 mg, 0.115 mmol) in DMF (dry) (0.5 ml) was added 4,4-difluorocyclohexyl)methanamine (22.38 mg, 0.150 mmol) and DIPEA (0.050 ml, 0.286 mmol). The reaction was stirred at RT for 1.5h before addition of a solution of 2-(4-fluorobenzyl)-2,6-diazaspiro[3.3]heptane I12-1 (20.63 mg, 0.1 mmol) in DMF (dry) (1 ml). The reaction was shaken overnight at RT. The reaction was filtered (0.45 μm) and purified by basic preparative HPLC-MS. Product fractions were concentrated, combined with ACN, water was added and the product was lyophilized to afford N-((4,4-difluorocyclohexyl)methyl)-6-(4-fluorobenzyl)-2,6-diazaspiro[3.3]heptane-2-carboxamide (Cpd B167). (22.4 mg, 0.059 mmol, 58.7% yield) MS (m/e): 382.2 (MH⁺) rt=1.35 min.

The following compounds were prepared in a similar manner (use of appropriate starting material, intermediates, reagents and purification methods (including chiral HPLC or chiral SFC) known to the person skilled in the art or as described herein) as described for Cpd B167: Cpd B168, Cpd B169, Cpd B170, Cpd B171, Cpd B172, Cpd B173 and Cpd B174.

| | [M + H]+ (m/e)/rt |
|---|---|
| Compounds | time (min) |
| Cpd B168 | 340.1/1.21 |
| Cpd B169 | 348.2/1.08 |
| Cpd B170 | 337.1/0.99 |
| Cpd B171 | 365.2/1.08 |
| Cpd B172 | 385.2/1.18 |
| Cpd B173 | 420.2/1.45 |
| Cpd B174 | 380.2/1.28 |

### Synthesis of N-(4-difluoromethoxybenzene)-6-((2-methylthiazol-4-yl)methyl)-2,6-diazaspiro[3.3]heptane-2-carboxamide (Cpd B175)

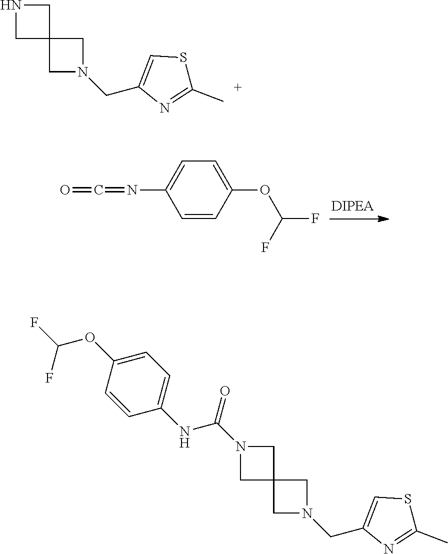 [SMILES: Cc1nc(CN2CC3(CNC3)C2)cs1 ; O=C=Nc1ccc(OC(F)F)cc1 ; Cc1nc(CN2CC3(C2)CN(C(=O)Nc2ccc(OC(F)F)cc2)C3)cs1]

To 1-(Difluoromethoxy)-4-isocyanatobenzene (27.8 mg, 0.150 mmol) (1.5 eq) was added DMAc, (0.5 mL), DIPEA (0.050 mL, 0.286 mmol) and 4-((2,6-diazaspiro[3.3]heptan-2-yl)methyl)-2-methylthiazole I12-3 (20.9 mg, 0.1 mmol) in DMAc (0.5 mL) and the reaction was shaken overnight at RT. The reaction mixture was filtered and purified via preparative basic LCMS. Purified fractions were combined and reformatted to afford N-(4-difluoromethoxybenzene)-6-((2-methylthiazol-4-yl)methyl)-2,6-diazaspiro[3.3]heptane-2-carboxamide (Cpd B175). (1.9 mg, 0.005 mmol, 4.8% yield) MS (m/e): 395.1 (MH⁺) rt=1.17 min.

The following compounds were prepared in a similar manner (use of appropriate starting material, intermediates, reagents and purification methods (including chiral HPLC or chiral SFC) known to the person skilled in the art or as described herein) as described for Cpd B175: Cpd B176, Cpd B177, Cpd B178, Cpd B179, Cpd B180, Cpd B181, Cpd B182, Cpd B183, Cpd B184, Cpd B185, Cpd B186, Cpd B187, Cpd B188 and Cpd B189.

| | [M + H]+ (m/e)/rt |
|---|---|
| Compounds | time (min) |
| Cpd B176 | 397.1/1.28 |
| Cpd B177 | 343.2/1.21 |
| Cpd B178 | 329.2/1.10 |
| Cpd B179 | 357.1/1.24 |
| Cpd B180 | 367.2/1.07 |
| Cpd B181 | 370.2/1.31 |
| Cpd B182 | 339.2/1.06 |
| Cpd B183 | 330.2/1.23 |
| Cpd B184 | 358.2/1.42 |
| Cpd B185 | 372.1/1.43 |
| Cpd B186 | 404.2/1.41 |
| Cpd B187 | 360.1/1.45 |
| Cpd B188 | 358.2/1.31 |
| Cpd B189 | 394.2/1.54 |

### Synthesis of 6-(4-fluorobenzyl)-N-(3-fluorobicyclo[1.1.1]pentan-1-yl)-2,6-diazaspiro[3.3]heptane-2-carboxamide (Cpd B190)

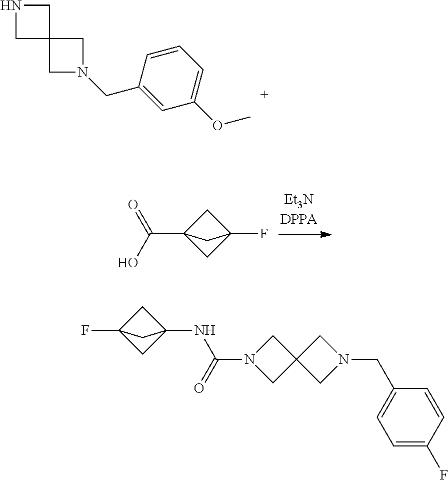

3-fluorobicyclo[1.1.1]pentane-1-carboxylic acid (19.52 mg, 0.150 mmol) was dissolved in toluene (dry) (1 ml), and Et₃N (0.024 ml, 0.175 mmol) and DPPA (0.035 ml, 0.163 mmol) were subsequently added. The resulting mixture was stirred for 3 hours at 80° C. The reaction mixture was cooled to RT. 2-(4-fluorobenzyl)-2,6-diazaspiro[3.3]heptane I12-1 (20.63 mg, 0.10 mmol) was added and the resulting mixture was stirred overnight at RT. The reaction mixture was concentrated under reduced pressure and purified by RP chromatography to obtain 6-(4-fluorobenzyl)-N-(3-fluorobicyclo[1.1.1]pentan-1-yl)-2,6-diazaspiro[3.3]heptane-2-carboxamide (Cpd B190) (15.9 mg, 0.048 mmol, 47.7% yield) as a white solid. MS (m/e): 334.2 (MH⁺) rt=1.53 min.

### Synthesis of N-(4-fluorobenzyl)-6-((2-methyl-furan-5-yl)methyl)-2,6-diazaspiro[3.3]heptane-2-carboxamide (Cpd B191)

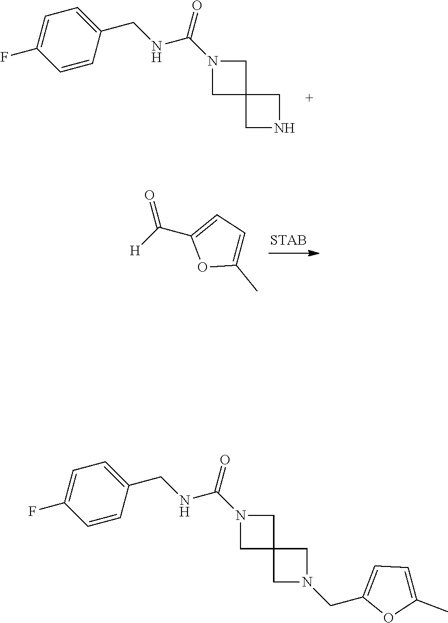

N-(4-fluorobenzyl)-2,6-diazaspiro[3.3]heptane-2-carboxamide I29-1 (24.9 mg, 0.1 mmol) in MeOH (1 mL) and 2-Methyl-5-furaldehyde (14.9 mg, 0.150 mmol) were mixed and the reaction was stirred at RT for 1.5h. STAB (0.053 g, 0.250 mmol) was added and the mixture was stirred overnight at RT. 7N NH₃ in MeOH (0.4 mL, ˜2.8 mmol) was added and mixed with the reaction. The reaction was filtered (0.45 μm) and purified by basic preparative HPLC-MS. Product fractions were concentrated, combined with ACN, water was added and the product was lyophilized to afford N-(4-fluorobenzyl)-6-((2-methyl-furan-5-yl)methyl)-2,6-diazaspiro[3.3]heptane-2-carboxamide_(Cpd B191). (21.9 mg, 0.064 mmol, 64% yield) MS (m/e): 344.2 (MH⁺) rt=1.26 min.

The following compounds were prepared in a similar manner (use of appropriate starting material, intermediates, reagents and purification methods (including chiral HPLC or chiral SFC) known to the person skilled in the art or as described herein) as described for Cpd B191: Cpd B192, Cpd B193, Cpd B194, Cpd B195, Cpd B196, Cpd B197, Cpd B198, Cpd B199, Cpd B200, Cpd B201, Cpd B202, Cpd B203, Cpd B204, Cpd B205, Cpd B206, Cpd B207, Cpd B208, Cpd B209 and Cpd B210.

| | [M + H]+ (m/e)/rt |
|---|---|
| Compounds | time (min) |
| Cpd B192 | 360.2/1.35 |
| Cpd B193 | 361.2/1.10 |
| Cpd B194 | 385.2/1.12 |
| Cpd B195 | 432.2/1.55 |
| Cpd B196 | 399.2/1.21 |
| Cpd B197 | 433.2/1.32 |
| Cpd B198 | 379.2/1.15 |
| Cpd B199 | 383.2/1.12 |
| Cpd B200 | 432.2/1.51 |
| Cpd B201 | 450.2/1.59 |
| Cpd B202 | 402.2/1.18 |
| Cpd B203 | 416.2/1.22 |
| Cpd B204 | 402.3/1.14 |
| Cpd B205 | 369.2/1.09 |
| Cpd B206 | 419.2/1.27 |
| Cpd B207 | 418.2/1.51 |
| Cpd B208 | 436.2/1.49 |
| Cpd B209 | 388.1/1.13 |
| Cpd B210 | 370.2/1.35 |

### Synthesis of 2-((3-methylpyridin-2-yl)methyl)-6-((phenethyl)sulfonyl)-2,6-diazaspiro[3.3]heptane (Cpd B211)

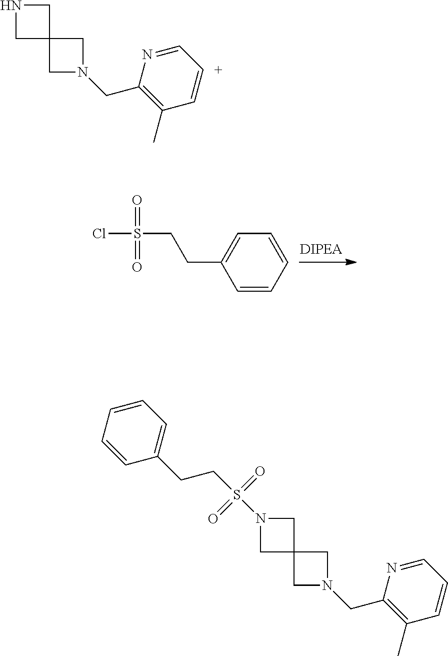 [SMILES: CC1=C(CN2CC3(CNC3)C2)N=C[C+]=C1 ; O=S(=O)(Cl)CCc1ccccc1 ; Cc1cccnc1CN1CC2(C1)CN(S(=O)(=O)CCc1ccccc1)C2]

To phenethylsulfonyl chloride (22.5 mg, 0.110 mmol) was added DMAc, (0.5 mL), DIPEA (0.050 mL, 0.286 mmol) and 2-((3-methylpyridin-2-yl)methyl)-2,6-diazaspiro[3.3]heptane I12-2 (20.3 mg, 0.1 mmol) in DMAc (0.5 mL) and the reaction was shaken overnight at RT. The reaction mixture was filtered, purified via preparative basic LCMS. Purified fractions were combined and concentrated, combined with ACN, water was added and the product was lyophilized to afford 2-((3-methylpyridin-2-yl)methyl)-6-((phenethyl)sulfonyl)-2,6-diazaspiro[3.3]heptane (Cpd B211). (19.0 mg, 0.051 mmol, 54% yield) MS (m/e): 372.2 (MH⁺) rt=0.84 min.

The following compounds were prepared in a similar manner (use of appropriate starting material, intermediates, reagents and purification methods known to the person skilled in the art or as described herein) as described for Cpd B211: Cpd B212, Cpd B213, Cpd B214, Cpd B215, Cpd B216, Cpd B217, Cpd B218, Cpd B219, Cpd B220, Cpd B221, Cpd B222, Cpd B223 and Cpd B224.

| | [M + H]+ (m/e)/rt |
|---|---|
| Compounds | time (min) |
| Cpd B212 | 426.2/1.58 |
| Cpd B213 | 376.1/1.29 |
| Cpd B214 | 392.0/1.34 |
| Cpd B215 | 432.1/1.40 |
| Cpd B216 | 429.1/1.67 |
| Cpd B217 | 431.1/1.72 |
| Cpd B218 | 395.1/1.62 |
| Cpd B219 | 443.1/1.70 |
| Cpd B220 | 427.1/1.65 |
| Cpd B221 | 390.1/1.49 |
| Cpd B222 | 373.1/1.54 |
| Cpd B223 | 441.1/1.64 |
| Cpd B224 | 441.1/1.66 |

### Synthesis of 2-(4-chlorobenzyl)-6-(3-methoxybenzyl)-2,6-diazaspiro[3.3]heptane (Cpd B225)

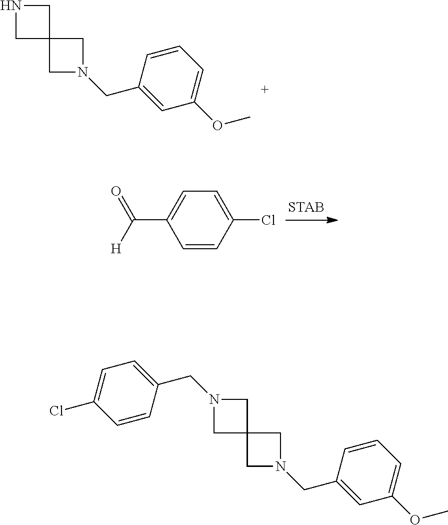 [SMILES: COc1cccc(CN2CC3(CNC3)C2)c1 ; O=Cc1ccc(Cl)cc1 ; COc1cccc(CN2CC3(CN(Cc4ccc(Cl)cc4)C3)C2)c1]

4-chlorobenzaldehyde (29.6 mg, 0.211 mmol) was added to a stirred solution of 2-(3-methoxybenzyl)-2,6-diazaspiro[3.3]heptane I12-4 (23 mg, 0.105 mmol) in DCM (2 ml). STAB (60 mg, 0.283 mmol) was added. The resulting mixture was stirred over night at RT. The reaction mixture was evaporated and to the crude material was added 150 μl NH₃ 35%, 250 μl MeCN and 550 μl DMSO (total ˜1 ml), the mixture was shortly heated by heat gun, filtered, and purified by basic preparative HPLC to afford 2-(4-chlorobenzyl)-6-(3-methoxybenzyl)-2,6-diazaspiro[3.3]heptane (Cpd B225). (17.4 mg, 0.051 mmol, 48% yield) MS (m/e): 343.0 (MH⁺) rt=1.61 min.

The compound Cpd B226 (MS (m/e): 327.1 (MH⁺), rt=1.49 min) was prepared in a similar manner (use of appropriate starting material, intermediates, reagents and purification methods known to the person skilled in the art or as described herein) as described for Cpd B225.

### Synthesis of 2-(4-fluorobenzyl)-6-(3-methoxybenzyl)-2,6-diazaspiro[3.3]heptane (Cpd B227)

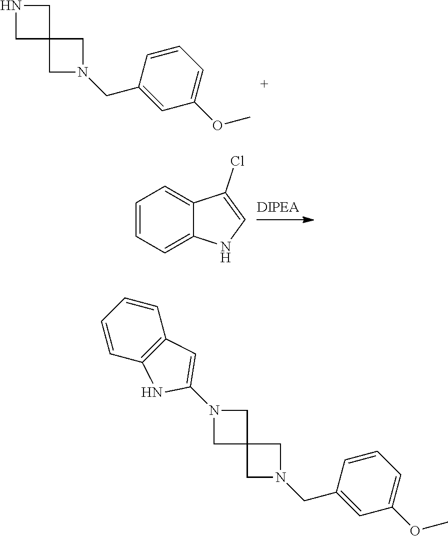

2-(3-methoxybenzyl)-2,6-diazaspiro[3.3]heptane I12-4 (53.3 mg, 0.244 mmol) was dissolved in NMP (dry) (1 ml). DIPEA (0.170 ml, 0.977 mmol) and 2-chlorobenzimidazole (48.4 mg, 0.317 mmol) were added. The resulting solution was stirred at 100° C. for 20 hours. The mixture was purified by basic preparative HPLC to afford 2-(4-fluorobenzyl)-6-(3-methoxybenzyl)-2,6-diazaspiro[3.3]heptane (Cpd B227) (20.7 mg, 0.62 mmol, 25% yield). MS (m/e): 335.2 (MH⁺) rt=1.20 min.

TABLE 2B
Analytical data (¹H-NMR, 400 MHz) of example compounds

| Cpd | Frequency | | |
|---|---|---|---|
| number | [MHz] | Solvent | δ [ppm] |
| Cpd | 400 | DMSO-d6 | 8.27-8.29 (m, 1H), 7.51-7.63 (m, 3H), 7.40-7.45 (m, 1H), 7.16-7.19 (m, 1H), 4.26 |
| B077 | | | (s, 2H), 3.90 (s, 2H), 3.62 (s, 2H), 3.50 (s, 2H), 3.31 (m, 4H), 2.30 (s, 3H) |
| Cpd | 400 | DMSO-d6 | 7-33-7-53 (m, 4H), 7.26-7.32 (2H, m),7.09-7.14 (m, 2H), 4.39 (s, 2H), 4.11 (s, |
| B094 | | | 2H), 3.47 (s, 2H), 3.24-3.34 (m, 4H) |
| Cpd | 400 | DMSO-d6 | 9.30 (s, 1H), 9.00 (s, 2H), 7.26-7.29 (m, 2H), 7.10-7.14 (m, 2H), 4.47 (s, 2H), |
| B096 | | | 4.16 (s, 2H), 3.48 (s, 2H), 3.24-3.31 (m, 4H) |
| Cpd | 400 | DMSO-d6 | 7.16 (s, 1H), 4.14 (s, 2H), 3.88 (s, 2H), 3.54 (s, 2H), 3.29 (m, 4H), 2.60 (s, 3H), |
| B102 | | | 1.91-1.96 (m, 4H), 1.68-1.84 (m, 5H), 1.12-1.21 (m, 2H) |
| Cpd | 400 | DMSO-d6 | 7.68-7.71 (m, 1H), 7.49-7.59 (m, 2H), 4.17 (s, 2H), 3.91 (s, 2H), 3.66 (s, 2H) 3.32 |
| B104 | | | (m, 4H), 1.69-1.97 (m, 9H), 1.12-1.22 (m, 2H) |
| Cpd | 400 | DMSO-d6 | 8.78 (d, 1H), 8.13 (d, 1H), 7.52 (dd, 1H), 4.14 (s, 2H), 3.87 (s, 2H), 3.74 (s, 2H), |
| B112 | | | 3.39 (s, 4H), 2.03 − 1.63 (m, 9H), 1.27 − 1.06 (m, 2H). |
| Cpd | 400 | DMSO-d6 | 8.46 (s, 1H), 7.64-7.70 (m, 1H), 7.37-7.41 (m, 1H), 4.16(s, 2H), 3.89 (s, 2H), 3.62 |
| B115 | | | (s, 2H), 3.32 (m, 4H), 1.95-1.96 (m, 4H), 1.69-1.84 (m, 5H), 1.12-1.21 (2H) |
| Cpd | 400 | DMSO-d6 | 4.13 (s, 2H), 3.87 (s, 2H), 3.48 (s, 2H), 3.31 (m, 4H), 2.58 (s, 3H), (1.94-1.97 (m, |
| B117 | | | 4H), 1.69-1.81 (m, 5H), 1.11-1.21 (m, 2H) |
| Cpd | 400 | DMSO-d6 | 4.11 (s, 2H), 3.85 (s, 2H), 3.69 (s, 3H), 3.36 (s, 2H), 3.23 (m, 4H), 2.18 (s, 3H), |
| B121 | | | 1.91-1.96 (m, 4H), 1.68-1.84 (m, 5H), 1.11-1.21 (m, 2H) |
| Cpd | 400 | DMSO-d6 | 11.61-11.68 (d, 1H), 7.35-7.36 (m, 1H), 4.10-4.11 (m, 2H), 3.83-3.86 (m, 2H), |
| B124 | | | 3.39 (s, 1H), 3.28 (s, 1H), 3.18 (s, 4H), 2.05-2.11 (d, 3H), 1.94-1.95 (m, 4H), |
| | | | 1.68-1.77 (m, 5H), 1.11-1.20 (m, 2H) |
| Cpd | 400 | DMSO-d6 | 7.25-7.29 (m, 2H), 7.08-7.14 (m, 2H), 4.19 (s, 2H), 3.96 (s, 4H), 3.46 (s, 2H), |
| B129 | | | 3.22 (s, 4H), 1.96 (s, 6H) |
| Cpd | 400 | DMSO-d6 | 7.18-7.22 (m, 1H), 6.78-6.80 (m, 3H), 4.88-4.92 (m, 1H), 3.92 (s, 4H), 3.72 (s, |
| B134 | | | 3H), 3.45 (s, 2H), 3.22 (s, 4H), 1.71-1.79 (m, 2H), 1.48-1.62 (m, 6H) |
| Cpd | 400 | DMSO-d6 | 7.18-7.22 (m, 1H), 6.77-6.80 (m, 4H), 4.66-4.70 (m, 1H), 3.97 (s, 4H), 3.72 (s, |
| B135 | | | 3H), 3.46 (s, 2H), 3.23 (s, 4H), 1.89-2.00 (m, 4H), 1.75-1.82 (m 2H), 1.63-1.71 (m |
| | | | 2H) |
| Cpd | 400 | DMSO-d6 | 7.15 (s, 1H), 4.19 (s, 2H), 3.96 (s, 4H), 3.53 (s, 2H), 3.30 (s, 4H), 2.60 (s, 3H), |
| B136 | | | 1.97 (s, 6H) |
| Cpd | 400 | DMSO-d6 | 8.27-8.28 (m, 1H), 7.51-7.52 (m, 1H), 7.15-7.18 (m, 1H), 4.66-4.69 (m, 1H), 3.95 |
| B139 | | | (s, 4H), 3.60 (s, 2H), 3.29 (s, 4H), 2.29 (s, 3H), 1.89-1.98 (m, 4H), 1.76-1.79 (m, |
| | | | 2H), 1.64-1.69 (m, 2H) |
| Cpd | 400 | CDCl₃ | 7.21-7.26 (m, 1H), 7.00-7.07 (m, 4H), 6.80-6.85 (m, 3H), 4.15-4.25 (m, 4H), 3.82 |
| B140 | | | (s, 3H), 3.56 (s, 2H), 3.39 (s, 4H) |
| Cpd | 400 | DMSO-d6 | 7.43 (s, 1H), 4.66-4.70 (m, 1H), 3.94 (s, 4H), 3.76 (s, 3H), 3.27 (s, 2H), 3.19 (s, |
| B144 | | | 4H), 1.89-1.99 (m, 4H), 1.64-1.82 (m, 4H) |
| Cpd | 400 | DMSO-d6 | 7.84 (s, 1H), 4.67 (m, 1H), 3.93 (s, 4H), 3.76 (s, 3H), 3.37 (s, 2H), 3.24 (s, 4H), |
| B145 | | | 2.03-1.86 (m, 4H), 1.84-1.60 (m, 4H). |
| Cpd | 400 | DMSO-d6 | 7.52-7.59 (m, 3H), 4.65-4.70 (m, 1H), 3.93 (s, 4H), 3.65 (s, 2H), 3.33 (s, 4H), |
| B148 | | | 1.88-1.97 (m, 4H), 1.64-1.80 (m, 4H) |
| Cpd | 400 | DMSO-d6 | 7.65 (s, 1H), 3.93 (s, 4H), 3.81-3.82 (d, 2H), 3.75 (s, 3H), 3.25 (s, 2H), 3.20 (s, |
| B157 | | | 4H), 1.95-2.00 (m, 2H), 1.39-1.86 (m, 5H), 1.16-1.23 (m, 2H) |
| Cpd | 400 | DMSO-d6 | 7.62-7.69 (m, 3H), 7.42-7.46 (m, 1H), 3.98 (s, 4H), 3.81-3.83 (d, 2H), 3.69 (s, |
| B165 | | | 2H), 3.31 (s, 4H), 1.99 (m, 2H), 1.72-1.86 (m, 5H), 1.15-1.28 (m, 2H) |
| Cpd | 400 | DMSO-d6 | 7.67-7.71 (m, 1H), 7.49-7.58 (m, 2H), 3.98 (s, 4H), 3.81-3.83 (d, 2H), 3.65 (s, |
| B166 | | | 2H), 3.32 (s, 4H), 1.96-2.01 (m, 2H), 1.68-1.90 (m, 5H), 1.14-1.24 (m, 2H) |
| Cpd | 400 | DMSO-d6 | 7.25-7.29 (m, 2H), 7.09-7.14 (m, 2H), 6.33-6.36 (m, 1H), 3.81 (s, 4H), 3.62 (s, |
| B167 | | | 2H), 3.20 (s, 4H), 2.83-2.86 (m, 2H), 1.95-1.98 (m, 2H), 1.64-1.79 (m, 4H), 1.48 |
| | | | (s, 1H), 1.03-1.13 (m, 2H) |
| Cpd | 400 | DMSO-d6 | 8.27-8.28 (m, 1H), 7.51-7.53 (m, 1H), 7.15-7.18 (m, 1H), 6.66-6.86 (m, 1H), 3.86- |
| B170 | | | 3.93 (m, 1H), 3.82 (s, 4H), 3.59 (s, 2H), 3.28 (s, 4H), 2.73-2.83 (m, 2H), 2.55- |
| | | | 2.61 (m, 2H), 1.23 (s, 3H) |
| Cpd | 400 | DMSO-d6 | 7.15 (s, 1H), 3.93 (s, 4H), 3.76-3.82 (m, 1H), 3.53 (s, 2H), 3.28 (s, 4H), 2.60 (s, |
| B172 | | | 3H), 2.58 (s, 3H), 1.88-2.03 (m, 4H), 1.23-1.72 (m, 4H) |
| Cpd | 400 | DMSO-d6 | 8.77 (s, 1H), 7.94-7.95 (m, 1H), 7.73-7.76 (m, 1H), 7.43-7.47 (m, 1H), 7.24-7.26 |
| B176 | | | (m, 1H), 7.18 (s, 1H), 4.03 (s, 4H), 3.56 (s, 2H), 3.33 (s, 4H), 2.61 (s, 3H) |
| Cpd | 400 | CDCl₃ | 8.37-8.39 (m, 1H), 7.42-7.44 (m, 1H), 7.31-7.35 (m, 2H), 7.21-7.26 (m 2H), 7.08- |
| B179 | | | 7.11 (m, 1H), 5.92 (s, 1H), 4.12 (s, 4H), 3.72 (s, 2H), 3.48 (s, 4H), 2.33 (s, 3H) |
| Cpd | 400 | CDCl₃ | 7.18-7.28 (m, 3H), 7.11-7.13 (m, 2H), 6.86-6.89 (m, 1H), 6.78-6.82 (m, 3H), 4.13- |
| B181 | | | 4.15 (d, 2H), 3.86 (s, 4H), 3.73 (s, 3H), 3.46 (s, 2H), 3.22 (s, 4H) |
| Cpd | 400 | CDCl₃ | 8.42-8.43 (m, 1H), 8.26-8.27 (m, 1H), 8.00-8.04 (m, 1H), 7.21-7.27 (m, 2H), 6.79- |
| B182 | | | 6.85 (m, 3H), 6.04 (s, 1H), 4.15 (s, 4H), 3.81 (s, 3H), 3.56 (s, 2H), 3.38 (s, 4H) |
| Cpd | 400 | DMSO-d6 | 7.25-7.28 (m, 2H), 7.08-7.13 (m, 2H), 7.05 (s, 1H), 3.81 (s, 4H), 3.46 (s, 2H), |
| B190 | | | 3.19 (s, 4H), 2.01 (6H) |
| Cpd | 400 | DMSO-d6 | 7.24-7.28 (m, 2H), 7.09-7.14 (m, 2H), 6.85-6.88 (m, 1H), 6.06 (d, 1H), 5.94 (s, |
| B191 | | | 1H), 4.13-4.15 (d, 2H), 3.84 (s, 4H), 3.38 (s, 2H), 3.22 (s, 4H), 2.20 (s, 3H) |
| Cpd | 400 | DMSO-d6 | 8.34-8.36 (m, 1H), 7.64-7.69 (m, 1H), 7.37-7.41 (m, 1H), 6.32-6.35 (m, 1H), 3.79 |
| B199 | | | (s, 4H), 3.64 (s, 2H), 3.31 (s, 4H), 2.83-2.86 (m, 2H), 1.95-1.98 (m, 2H), 1.64- |
| | | | 1.79 (m, 4H), 1.47 (m, 1H), 1.03-1.13 (m, 2H) |
| Cpd | 400 | DMSO-d6 | 7.68-7.71 (m, 1H), 7.49-7.58 (m, 2H), 6.34-6.37 (m, 1H), 3.84 (s, 4H), 3.66 (s, |
| B201 | | | 2H), 3.33 (s, 4H), 2.84-2.89 (m, 2H), 1.96-1.98 (m, 2H), 1.65-1.78 (m, 4H), 1.48 |
| | | | (m, 1H), 1.04-1.13 (m, 2H) |
| Cpd | 400 | DMSO-d6 | 7.84 (1H), 6.32-6.35 (m, 1H), 3.76-3.78 (m, 7H), 3.38 (s, 2H), 3.22 (s, 4H), 2.83- |
| B202 | | | 2.86 (m, 2H), 1.95-1.98 (m, 2H), 1.64-1.79 (m, 4H), 1.48 (m, 1H), 1.03-1.13 (2H) |
| Cpd | 400 | DMSO-d6 | 8.77-8.79 (m, 1H), 8.12-8.14 (m, 1H), 7.50-7.54 (m, 1H), 6.10-6.12 (d, 1H), 3.80 |
| B206 | | | (s, 4H), 3.74 (s, 2H), 3.51-3.54 (m, 1H), 3.37 (s, 4H), 1.71-1.99 (m, 6H), 1.40- |
| | | | 1.50 (m, 2H) |
| Cpd | 400 | DMSO-d6 | 8.27-8.29 (m, 1H), 7.79 (s, 1H), 7.71-7.75 (m, 1H), 7.61-7.65 (m, 1H), 7.51-7.53 |
| B212 | | | (m, 1H), 7.16-7.19 (m, 1H), 4.62 (s, 2H), 3.94 (s, 4H), 3.61 (s, 2H), 3.29 (s, 4H), |
| | | | 2.29 (s, 3H) |
| Cpd | 400 | CDCl₃ | 8.36 (s, 1H), 7.35-7.42 (m, 3H), 7.04-7.10 (m, 3H), 4.14 (s, 2H), 3.83 (s, 4H), |
| B213 | | | 3.66 (s, 2H), 3.38 (s, 4H), 2.30 (s, 3H) |
| Cpd | 400 | DMSO-d6 | 7.76-7.78 (d, 2H), 7.62-7.64 (d, 2H), 7.25-7.29 (m, 2H), 7.10-7.14 (m, 2H), 4.64 |
| B216 | | | (s, 2H), 3.96 (s, 4H), 3.47 (s, 2H), 3.23 (s, 4H) |
| Cpd | 400 | DMSO-d6 | 7.24-7.28 (m, 2H), 7.17-7.21 (m, 1H), 7.08-7.13 (m, 2H), 6.76-6.80 (m, 3H), 3.72 |
| B226 | | | (s, 3H), 3.45-3.46 (m, 4H), 3.17 (m, 8H). |
| Cpd | 400 | DMSO-d6 | 11.34 (br s, 1H), 7.17-7.23 (m, 3H), 6.91 (m, 2H), 6.84-6.78 (m, 3H), 4.41 (s, 4H), |
| B227 | | | 3.73 (s, 3H), 3.50 (m, 2H), 3.31 (m, 4H). |

General Procedure for Preparation of B1B2 via amine 36*

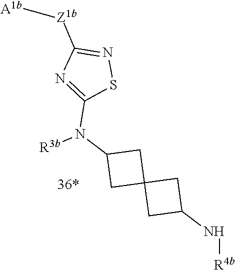

i) Amine 36* was combined with the aldehyde (Eq: 1.00) in EtOH with DIPEA (Eq: 3) and acetic acid (Eq: 3). The reaction was stirred for 30 min. Sodium cyanoborohydride (Eq: 1.5) was added and the reaction mixture was stirred at room temperature for 2 hours. Solvent was evaporated and the product purified using prep. HPLC. or:
ii) Amine 36* was combined with the aldehyde (Eq: 1.00) and TEA (Eq: 2) in DCE. The reaction mixture was stirred for 5 min. Sodium triacetoxyborohydride (Eq: 1.5) was added. The reaction mixture was stirred at RT 1 hour then poured into DCM and extracted with NaHCO₃. The aqueous layer was back-extracted with DCM. The organic phase was dried over Na₂SO₄, concentrated in vacuo and the product purified by silica chromatography using heptane/ethyl acetate. or:
iii) To a solution of Amine 36* (0.15 mmol) with triethylamine (5 eq.) in DMF (1 mL) was added the halogenated electrophile (1.1 eq). Reaction was stirred overnight at room temperature. Product was purified by prep. HPLC.
   General Procedure for Preparation of B1B2 Wherein R^{4b} is Different from H
iv) Compound B1B2 wherein R^{4b} is H was combined with formaldehyde (Eq: 1.2) in DCE, sodium triacetoxyborohydride (Eq: 1.5) was added and the reaction mixture was stirred at RT overnight. The reaction was poured into EtOAc, extracted with NaHCO₃ and the organic layers dried over Na₂SO₄ and concentrated in vacuo. The crude product was purified by silica chromatography using heptane/ethyl acetate giving the desired product.

### Synthesis of N-benzyl-N′-(3-(4-fluorobenzyl)-1,2,4-thiadiazol-5-yl) spiro[3.3]heptane-2,6-diamine (Cpd B301)

Coupling between a building block described herein above and aldehyde according to general procedure for preparation of B1 B2 step ii):
Building block: N-(3-(4-fluorobenzyl)-1,2,4-thiadiazol-5-yl)spiro[3.3]heptane-2,6-diamine hydrochloride (IB 30-1)
Aldehyde: benzaldehyde (commercially available)

ESI [M+H]⁺; 409.1

### Synthesis of N-benzyl-N′-(3-(4-fluorobenzyl)-1,2,4-thiadiazol-5-yl)-N-methyl spiro[3.3]heptane-2,6-diamine (Cpd B302)

Coupling between a building block described herein above and aldehyde according to general procedure for preparation of B2B2 step iv):
Building block: N-(3-(4-fluorobenzyl)-1,2,4-thiadiazol-5-yl)spiro[3.3]heptane-2,6-diamine hydrochloride (IB 30-1)
Aldehyde: formaldehyde (commercially available)

ESI [M+H]⁺; 423.1

### Synthesis of N-(4-chlorobenzyl)-N′-(3-(4-chlorobenzyl)-1,2,4-thiadiazol-5-yl) spiro[3.3]heptane-2,6-diamine (Cpd B303)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 4-chlorobenzaldehyde (commercially available)

ESI [M+H]⁺; 459.4

### Synthesis of N-(3-(4-chlorobenzyl)-1,2,4-thiadiazol-5-yl)-N′-(4-(trifluoromethyl)benzyl) spiro[3.3]heptane-2,6-diamine (Cpd B304)

Coupling between a building block described herein above and 4-(trifluoromethyl)benzaldehyde (commercially available) according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 493.4

### Synthesis of N-(3-(4-chlorobenzyl)-1,2,4-thiadiazol-5-yl)-N′-(2-methoxybenzyl) spiro[3.3]heptane-2,6-diamine (Cpd B305)

Coupling between a building block described herein above and 2-methoxybenzaldehyde (commercially available) according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 455.4

### Synthesis of N-(3-(4-chlorobenzyl)-1,2,4-thiadiazol-5-yl)-N′-(3,4-dichlorobenzyl) spiro[3.3]heptane-2,6-diamine (Cpd B306)

Coupling between a building block described herein above and 3,4-dichlorobenzaldehyde (commercially available) according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 493.3

### Synthesis of N-(3-(4-chlorobenzyl)-1,2,4-thiadiazol-5-yl)-N′-(2,4-dichlorobenzyl) spiro[3.3]heptane-2,6-diamine (Cpd B307)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2,4-dichlorobenzaldehyde (commercially available)

ESI [M+H]⁺; 493.3

### Synthesis of N-(3-(4-chlorobenzyl)-1,2,4-thiadiazol-5-yl)-N′-(2,5-dichlorobenzyl) spiro[3.3]heptane-2,6-diamine (Cpd B308)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2,5-dichlorobenzaldehyde (commercially available)

ESI [M+H]⁺; 493.3

### Synthesis of N-(3-(4-chlorobenzyl)-1,2,4-thiadiazol-5-yl)-N′-(4-fluoro-3-methylbenzyl) spiro[3.3]heptane-2,6-diamine (Cpd B309)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 4-fluoro-3-methylbenzaldehyde (commercially available)

ESI [M+H]⁺; 457.4

### Synthesis of N-(3-(4-chlorobenzyl)-1,2,4-thiadiazol-5-yl)-N′-(3-methoxybenzyl) spiro[3.3]heptane-2,6-diamine (Cpd B310)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 3-methoxybenzaldehyde (commercially available)

ESI [M+H]⁺; 455.4

### Synthesis of N-(3-(4-chlorobenzyl)-1,2,4-thiadiazol-5-yl)-N′-(2,6-dimethoxybenzyl) spiro[3.3]heptane-2,6-diamine (Cpd B311)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2,6-dimethoxybenzaldehyde (commercially available)

ESI [M+H]⁺; 485.4

### Synthesis of N-(3-(4-chlorobenzyl)-1,2,4-thiadiazol-5-yl)-N′-(2-fluorobenzyl) spiro[3.3]heptane-2,6-diamine (Cpd B312)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2-fluorobenzaldehyde (commercially available)

ESI [M+H]⁺; 443.4

### Synthesis of N-(3-(4-chlorobenzyl)-1,2,4-thiadiazol-5-yl)-N′-(3-fluoro-4-methoxybenzyl) spiro[3.3]heptane-2,6-diamine (Cpd B313)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 3-fluoro-4-methoxybenzaldehyde (commercially available)

ESI [M+H]⁺; 473.4

### Synthesis of N-(3-(4-chlorobenzyl)-1,2,4-thiadiazol-5-yl)-N′-(2,3-dimethoxybenzyl) spiro[3.3]heptane-2,6-diamine (Cpd B314)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2,3-dimethoxybenzaldehyde (commercially available)

ESI [M+H]⁺; 485.4

### Synthesis of N-(3-(4-chlorobenzyl)-1,2,4-thiadiazol-5-yl)-N′-(2-ethoxybenzyl) spiro[3.3]heptane-2,6-diamine (Cpd B315)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2-ethoxybenzaldehyde (commercially available)

ESI [M+H]⁺; 469.4

### Synthesis of N-(3-(4-chlorobenzyl)-1,2,4-thiadiazol-5-yl)-N′-(2,4-dimethoxybenzyl) spiro[3.3]heptane-2,6-diamine (Cpd B316)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2,4-dimethoxybenzaldehyde (commercially available)

ESI [M+H]⁺; 485.4

### Synthesis of N-(3-(4-chlorobenzyl)-1,2,4-thiadiazol-5-yl)-N′-(3,4-dimethoxybenzyl) spiro[3.3]heptane-2,6-diamine (Cpd B317)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 3,4-dimethoxybenzaldehyde (commercially available)

ESI [M+H]⁺; 485.4

### Synthesis of N-(3-(4-chlorobenzyl)-1,2,4-thiadiazol-5-yl)-N′-(4-methoxybenzyl) spiro[3.3]heptane-2,6-diamine (Cpd B318)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 4-methoxybenzaldehyde (commercially available)

ESI [M+H]⁺; 455.4

### Synthesis of N-(3-(4-chlorobenzyl)-1,2,4-thiadiazol-5-yl)-N′-(2,4-dimethylbenzyl) spiro[3.3]heptane-2,6-diamine (Cpd B319)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 2,4-dimethylbenzaldehyde (commercially available)

ESI [M+H]⁺; 453.4

### Synthesis of N-(benzo[d][1,3]dioxol-5-ylmethyl)-N′-(3-(4-chlorobenzyl)-1,2,4-thiadiazol-5-yl) spiro[3.3]heptane-2,6-diamine (Cpd B320)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: benzo[d][1,3]dioxole-5-carbaldehyde (commercially available)

ESI [M+H]⁺; 469.4

### Synthesis of N-(3-(4-chlorobenzyl)-1,2,4-thiadiazol-5-yl)-N′-(4-methylbenzyl) spiro[3.3]heptane-2,6-diamine (Cpd B321)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 4-methylbenzaldehyde (commercially available)

ESI [M+H]⁺; 439.4

### Synthesis of N-(3-(4-chlorobenzyl)-1,2,4-thiadiazol-5-yl)-N′-(2-(trifluoromethyl)benzyl) spiro[3.3]heptane-2,6-diamine (Cpd B322)

Coupling between a building block described herein above and aldehyde according to general procedure for preparation of B2B2 step i):

Building block: N-(3-(4-chlorobenzyl)-1,2,4-thiadiazol-5-yl)spiro[3.3]heptane-2,6-diamine TFA salt (IB 30-2)
Aldehyde: 2-(trifluoromethyl)benzaldehyde (commercially available)

ESI [M+H]⁺; 493.4

### Synthesis of N-(3-(4-chlorobenzyl)-1,2,4-thiadiazol-5-yl)-N′-(3-fluorobenzyl)spiro[3.3]heptane-2,6-diamine (Cpd B323)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 3-fluorobenzaldehyde (commercially available)

ESI [M+H]⁺; 443.4

### Synthesis of N-(3-(4-chlorobenzyl)-1,2,4-thiadiazol-5-yl)-N′-(3-methylbenzyl) spiro[3.3]heptane-2,6-diamine (Cpd B324)

Coupling between a building block described herein above and aldehyde according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)
Aldehyde: 3-methylbenzaldehyde (commercially available)

ESI [M+H]⁺; 439.4

### Synthesis of N-(3-(4-chlorobenzyl)-1,2,4-thiadiazol-5-yl)-N′-(3-(trifluoromethyl)benzyl) spiro[3.3]heptane-2,6-diamine (Cpd B325)

Coupling between a building block described herein above and aldehyde: 3-(trifluoromethyl)benzaldehyde (commercially available) according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 493.4

### Synthesis of N-(4-((6-(3-(4-chlorobenzyl)-1,2,4-thiadiazol-5-ylamino) spiro[3.3]heptan-2-ylamino)methyl)phenyl) acetamide (Cpd B326)

Coupling between a building block described herein above and aldehyde: N-(4-formylphenyl)acetamide (commercially available) according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 482.4

### Synthesis of N-(3-(4-chlorobenzyl)-1,2,4-thiadiazol-5-yl)-N′-(2,5-dimethoxybenzyl) spiro[3.3]heptane-2,6-diamine (Cpd B327)

Coupling between a building block described herein above and aldehyde: 2,5-dimethoxybenzaldehyde (commercially available) according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 485.4

### Synthesis of N-(3-(4-chlorobenzyl)-1,2,4-thiadiazol-5-yl)-N′-(2-methoxyphenethyl) spiro[3.3]heptane-2,6-diamine (Cpd B328)

Coupling between a building block described herein above and halogenated electrophile: 1-(2-bromoethyl)-2-methoxybenzene (commercially available) according to general procedure B2B2 step iii):
Building block: N-(3-(4-chlorobenzyl)-1,2,4-thiadiazol-5-yl)spiro[3.3]heptane-2,6-diamine TFA salt (IB 30-2)

ESI [M+H]⁺; 469.4

### Synthesis of N-(3-(4-chlorobenzyl)-1,2,4-thiadiazol-5-yl)-N′-(3,5-difluorobenzyl) spiro[3.3]heptane-2,6-diamine (Cpd B329)

Coupling between a building block described herein above and aldehyde: 3,5-difluorobenzaldehyde (commercially available) according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 461.4

### Synthesis of N-(benzo[d][1,3]dioxol-4-ylmethyl)-N′-(3-(4-chlorobenzyl)-1,2,4-thiadiazol-5-yl) spiro[3.3]heptane-2,6-diamine (Cpd B330)

Coupling between a building block described herein above and aldehyde: benzo[d][1,3]dioxole-4-carbaldehyde (commercially available), according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 469.4

### Synthesis of N-(3-(4-chlorobenzyl)-1,2,4-thiadiazol-5-yl)-N′-(4-(methylsulfonyl)benzyl) spiro[3.3]heptane-2,6-diamine (Cpd B331)

Coupling between a building block described herein above and aldehyde: benzo 4-(methylsulfonyl)benzaldehyde (commercially available), according to general procedure for preparation of B2B2 step i):
Building block: N-(3-(4-chlorobenzyl)-1,2,4-thiadiazol-5-yl)spiro[3.3]heptane-2,6-diamine TFA salt (IB 30-2)

ESI [M+H]⁺; 503.4

### Synthesis of N-(3-(4-chlorobenzyl)-1,2,4-thiadiazol-5-yl)-N′-(3-chlorophenethyl) spiro[3.3]heptane-2,6-diamine (Cpd B332)

Coupling between a building block described herein above and halogenated electrophile: 1-(2-bromoethyl)-3-chlorobenzene (commercially available), according to general procedure B2B2 step iii):
Building block: N-(3-(4-chlorobenzyl)-1,2,4-thiadiazol-5-yl)spiro[3.3]heptane-2,6-diamine TFA salt (IB 30-2)

ESI [M+H]⁺; 473.4

### Synthesis of N-(3-(4-chlorobenzyl)-1,2,4-thiadiazol-5-yl)-N′-(4-methoxyphenethyl) spiro[3.3]heptane-2,6-diamine (Cpd B333)

Coupling between a building block described herein above and halogenated electrophile: 1-(2-bromoethyl)-4-methoxybenzene (commercially available), according to general procedure B2B2 step iii):
Building block: N-(3-(4-chlorobenzyl)-1,2,4-thiadiazol-5-yl)spiro[3.3]heptane-2,6-diamine TFA salt (IB 30-2)

ESI [M+H]⁺; 469.4

### Synthesis of N-(3-(4-chlorobenzyl)-1,2,4-thiadiazol-5-yl)-N′-(2-chlorophenethyl) spiro[3.3]heptane-2,6-diamine (Cpd B334)

Coupling between a building block described herein above and halogenated electrophile: 1-(2-bromoethyl)-2-chlorobenzene (commercially available), according to general procedure B2B2 step iii):
Building block: N-(3-(4-chlorobenzyl)-1,2,4-thiadiazol-5-yl)spiro[3.3]heptane-2,6-diamine TFA salt (IB 30-2)

ESI [M+H]⁺; 473.4

### Synthesis of N-(3-(4-chlorobenzyl)-1,2,4-thiadiazol-5-yl)-N′-(3,4-dimethoxyphenethyl) spiro[3.3]heptane-2,6-diamine (Cpd B335)

Coupling between a building block described herein above and halogenated electrophile: 4-(2-bromoethyl)-1,2-dimethoxybenzene (commercially available), according to general procedure B2B2 step iii):
Building block: N-(3-(4-chlorobenzyl)-1,2,4-thiadiazol-5-yl)spiro[3.3]heptane-2,6-diamine TFA salt (IB 30-2)

ESI [M+H]⁺; 499.4

### Synthesis of N-(3-(4-chlorobenzyl)-1,2,4-thiadiazol-5-yl)-N′-(3-(trifluoromethyl)phenethyl) spiro[3.3]heptane-2,6-diamine (Cpd B336)

Coupling between a building block described herein above and halogenated electrophile: 1-(2-bromoethyl)-3-(trifluoromethyl)benzene (commercially available), according to general procedure for B2B2 step iii):
Building block: N-(3-(4-chlorobenzyl)-1,2,4-thiadiazol-5-yl)spiro[3.3]heptane-2,6-diamine TFA salt (IB 30-2)

ESI [M+H]⁺; 507.4

### Compounds Cpd B401 to Compound Cpd B521

These compounds were prepared following the procedures as indicated in the below Table:

| Cpd number | Procedure |
|---|---|
| Cpd B401 | As described for Compound B1A4 in Scheme A1 |
| Cpd B402 | As described for Compound B1A1 in Scheme A1 |
| Cpd B403 | As described for Compound B1A1 in Scheme A1 |
| Cpd B404 | As described for Compound B1A1 in Scheme A1 |
| Cpd B405 | As described for Compound B1A3 in Scheme A4 |
| Cpd B406 | As described for Compound B1A3 in Scheme A4 |
| Cpd B407 | As described for Compound B1A1 in Scheme A1 |
| Cpd B408 | As described for Compound B1A1 in Scheme A1 |
| Cpd B409 | As described for Compound B1A1 in Scheme A1 |
| Cpd B410 | As described for Compound B1A3 in Scheme A4 |
| Cpd B411 | As described for Compound B1A1 in Scheme A1 |
| Cpd B412 | As described for Compound B1A1 in Scheme A1 |
| Cpd B413 | As described for Compound B1A3 in Scheme A4 |
| Cpd B414 | As described for Compound B1A1 in Scheme A1 |
| Cpd B415 | As described for Compound B1A1 in Scheme A1 |
| Cpd B416 | As described for Compound B1A1 in Scheme A1 |
| Cpd B417 | As described for Compound B1A3 in Scheme A4 |
| Cpd B418 | As described for Compound B1A3 in Scheme A1 |
| Cpd B419 | As described for Compound B1A1 in Scheme A1 |
| Cpd B420 | As described for Compound B1A3 in Scheme A1 |
| Cpd B421 | As described for Compound B1A3 in Scheme A4 |
| Cpd B422 | As described for Compound B1A3 in Scheme A1 |
| Cpd B423 | As described for Compound B1A1 in Scheme A1 |
| Cpd B424 | As described for Compound B1A1 in Scheme A1 |
| Cpd B425 | As described for Compound B1A1 in Scheme A1 |
| Cpd B426 | As described for Compound B1A3 in Scheme A4 |
| Cpd B427 | As described for Compound B1A1 in Scheme A1 |
| Cpd B428 | As described for Compound B1A1 in Scheme A1 |
| Cpd B429 | As described for Compound B1A2 in Scheme A1 |
| Cpd B430 | As described for Compound B1A1 in Scheme A1 |
| Cpd B431 | As described for Compound B1A3 in Scheme A4 |
| Cpd B432 | As described for Compound B1A3 in Scheme A4 |
| Cpd B433 | As described for Compound B1A1 in Scheme A1 |
| Cpd B434 | As described for Compound B1A1 in Scheme A1 |
| Cpd B435 | As described for Compound B1A3 in Scheme A1 |
| Cpd B436 | As described for Compound B1A1 in Scheme A1 |
| Cpd B437 | As described for Compound B1A1 in Scheme A1 |
| Cpd B438 | As described for Compound B1A1 in Scheme A2 |
| Cpd B439 | As described for Compound B1A1 in Scheme A1 |
| Cpd B440 | As described for Compound B1A3 in Scheme A4 |
| Cpd B441 | As described for Compound B1A3 in Scheme A4 |
| Cpd B442 | As described for Compound B1A1 in Scheme A1 |
| Cpd B443 | As described for Compound B1A3 in Scheme A1 |
| Cpd B444 | As described for Compound B1A3 in Scheme A4 |
| Cpd B445 | As described for Compound B1A1 in Scheme A2 |
| Cpd B446 | As described for Compound B1A2 in Scheme A3 |
| Cpd B447 | As described for Compound B1A3 in Scheme A4 |
| Cpd B448 | As described for Compound B1A2 in Scheme A3 |
| Cpd B449 | As described for Compound B1A1 in Scheme A2 |
| Cpd B450 | As described for Compound B1A3 in Scheme A1 |
| Cpd B451 | As described for Compound B1A1 in Scheme A2 |
| Cpd B452 | As described for Compound B1A3 in Scheme A1 |
| Cpd B453 | As described for Compound B1A3 in Scheme A4 |
| Cpd B454 | As described for Compound B1A3 in Scheme A1 |
| Cpd B455 | As described for Compound B1A1 in Scheme A1 |
| Cpd B456 | As described for Compound B1A1 in Scheme A2 |
| Cpd B457 | As described for Compound B1A3 in Scheme A4 |
| Cpd B458 | As described for Compound B1A1 in Scheme A2 |
| Cpd B459 | As described for Compound B1A3 in Scheme A4 |
| Cpd B460 | As described for Compound B1A3 in Scheme A4 |
| Cpd B461 | As described for Compound B1A1 in Scheme A2 |
| Cpd B462 | As described for Compound B1A1 in Scheme A2 |
| Cpd B463 | As described for Compound B1A2 in Scheme A1 |
| Cpd B464 | As described for Compound B1A2 in Scheme A3 |
| Cpd B465 | As described for Compound B1A3 in Scheme A1 |
| Cpd B466 | As described for Compound B1A3 in Scheme A1 |
| Cpd B467 | As described for Compound B1A3 in Scheme A4 |
| Cpd B468 | As described for Compound B1A3 in Scheme A4 |
| Cpd B469 | As described for Compound B1A3 in Scheme A4 |
| Cpd B470 | As described for Compound B1A3 in Scheme A4 |
| Cpd B471 | As described for Compound B1A1 in Scheme A1 |
| Cpd B472 | As described for Compound B1A1 in Scheme A1 |
| Cpd B473 | As described for Compound B1A1 in Scheme A1 |
| Cpd B474 | As described for Compound B1A1 in Scheme A1 |
| Cpd B475 | As described for Compound B1A1 in Scheme A1 |
| Cpd B476 | As described for Compound B1A1 in Scheme A1 |
| Cpd B477 | As described for Compound B1A1 in Scheme A1 |
| Cpd B478 | As described for Compound B1A1 in Scheme A1 |
| Cpd B479 | As described for Compound B1A1 in Scheme A1 |
| Cpd B480 | As described for Compound B1A1 in Scheme A1 |
| Cpd B481 | As described for Compound B1A1 in Scheme A1 |
| Cpd B482 | As described for Compound B1A1 in Scheme A1 |
| Cpd B483 | As described for Compound B1A1 in Scheme A1 |
| Cpd B484 | As described for Compound B1A1 in Scheme A1 |
| Cpd B485 | As described for Compound B1A1 in Scheme A1 |
| Cpd B486 | As described for Compound B1A1 in Scheme A1 |
| Cpd B487 | As described for Compound B1A1 in Scheme A1 |
| Cpd B488 | As described for Compound B1A1 in Scheme A1 |
| Cpd B489 | As described for Compound B1A1 in Scheme A1 |
| Cpd B490 | As described for Compound B1A1 in Scheme A1 |
| Cpd B491 | As described for Compound B1A1 in Scheme A1 |
| Cpd B492 | As described for Compound B1A1 in Scheme A1 |
| Cpd B493 | As described for Compound B1A1 in Scheme A1 |
| Cpd B494 | As described for Compound B1A1 in Scheme A1 |
| Cpd B495 | As described for Compound B1A1 in Scheme A1 |
| Cpd B496 | As described for Compound B1A1in Scheme A1 |
| Cpd B497 | As described for Compound B1A1 in Scheme A1 |
| Cpd B498 | As described for Compound B1A1 in Scheme A1 |
| Cpd B499 | As described for Compound B1A1 in Scheme A1 |
| Cpd B500 | As described for Compound B1A1in Scheme A1 |
| Cpd B501 | As described for Compound B1A1 in Scheme A1 |
| Cpd B502 | As described for Compound B1A1 in Scheme A1 |
| Cpd B503 | As described for Compound B1A3 in Scheme A4 |
| Cpd B504 | As described for Compound B1A3 in Scheme A4 |
| Cpd B505 | As described for Compound B1A2 in Scheme A1 |
| Cpd B506 | As described for Compound B1A1 in Scheme A1 |
| Cpd B507 | As described for Compound B1A1 in Scheme A1 |
| Cpd B508 | As described for Compound B1A1 in Scheme A1 |
| Cpd B509 | As described for Compound B1A1 in Scheme A1 |
| Cpd B510 | As described for Compound B1A1 in Scheme A1 |
| Cpd B511 | As described for Compound B1A1 in Scheme A1 |
| Cpd B512 | As described for Compound B1A1 in Scheme A1 |
| Cpd B513 | As described for Compound B1A1 in Scheme A1 |
| Cpd B514 | As described for Compound B1A1 in Scheme A1 |
| Cpd B515 | As described for Compound B1A1 in Scheme A1 |
| Cpd B516 | As described for Compound B1A1 in Scheme A1 |
| Cpd B517 | As described for Compound B1A1 in Scheme A1 |
| Cpd B518 | As described for Compound B1A1 in Scheme A1 |
| Cpd B519 | As described for Compound B1A1 in Scheme A1 |
| Cpd B520 | As described for Compound B1A1 in Scheme A1 |
| Cpd B521 | As described for Compound B1A1 in Scheme A1 |

### Part B

The compounds were investigated in accordance with the test given hereinafter.

### Construction of a TAU Gene Over-Expressing Cell Line

A TAU expression plasmid was constructed by sub-cloning the cDNA encoding for human TAU-P301L protein, wherein proline at position 301 is substituted by a leucine residue, into mammalian expression vector pcDNA3.1 resulting in the plasmid pcDNA3.1-TAUP301L. Plasmids pcDNA3.1 and pcDNA3.1-TAU P301L were transfected into human neuroblastoma cells (BE-M17; ATCC No. CRL-2267™) using lipofectamine reagent and subsequently, independent clonal cell lines with the plasmids stably integrated into the genome were selected by antibiotic resistance selection (Geneticin (G418)), resulting in cell lines M17.pcDNA3 and M17_3TAUP301L. Expression of the TAUP301 L gene in the M17_3TAUP301 L cells was confirmed by Western blot analysis.

### Use of TAU Expressing Cells as a Model of Neuronal Degeneration

The expression of TAU P301L in M17_3TAU(P301L) cells was found to confer increased toxicity relative to control cells expressing no TAU after 7 days of cell differentiation using retinoic acid (RA). Differentiation of the cells with RA leads to phosphorylation and subsequent aggregation of TAU, inducing a tauopathy in these cells. Cytotoxicity of cells was measured by quantification of lactate dehydrogenase (LDH) levels. In dead cells LDH is leaked out of the cells into the medium due to a loss of plasma-membrane integrity.

Briefly, 3 days preceding the experiment pre-cultures of M17.pcDNA3 and M17_3TAU(P301L) cells were prepared, starting from a stock culture, at a density of 50.000-100.000 cells/cm2 in detection medium (Optimem Reduced Serum without phenol red (Gibco, Cat. 31985-047) supplemented with 1% fetal calf serum (FCS), 1 mM sodium pyruvate, 1×non-essential amino acids (NEAA), 500 μg/ml G418 and 0.5×antibiotic/antimycotic (ABAM)). At the day of the experiment these precultures were diluted to ˜10≡cells/ml in detection medium without FCS and 60 μL of this suspension is dispensed per well into a 96-well microtiter plate. After 3 hours of incubation at 37° C./5% C02 an equal volume of detection medium containing 2.5 μM RA was added and subsequently incubated for 7 days at 37° C./5% CO₂. After 7 days, LDH activity was determined using the Promega Cytotox 96 Non-Radioactive cytotoxicity assay (Cat. G1780), according the manufacturer's instructions. Cytotoxicity is measured as the ratio of LDH increase in the supernatant divided by the LDH increase in the total cell suspension (sum of the LDH measured in cells and supernatant). Toxicity was determined after 7 days of differentiation with retinoic acid in M17_3TAU(P301 L) cells compared to M17.pcDNA3 cells. Toxicity is clearly higher in the M17_3TAU(P301L) cells demonstrating that it is specifically provoked by the presence of the mutant TAU P301 protein.

### Use of the Neuroblastoma Tauopathy Model to Screen Compounds

The M17_3TAU(P301L) cell line makes it possible to assess the ability of novel compounds to inhibit TAU-induced cytotoxicity. Active inhibitors of Tauopathy in these cells were found to inhibit cytotoxicity or LDH increase in the medium of M17_3TAU(P301L) cells treated as described in Example above. Compounds were tested for their ability to hamper TAU-induced toxicity at different concentrations, ranging from low non-effective concentrations to high potent concentrations. Afterwards, the dose-dependent inhibition curve was used to calculate their EC₅₀ (Table 3B).

Although the pharmacological properties of the compounds disclosed in this invention vary with structural change, active compounds most particularly possess EC₅₀ in a cell-based assay in a range from about <0.00019 to 5.0 μM.

The tested compounds show a EC₅₀ value (μM) as shown in the Table 3B below.

TABLE 3B
EC₅₀ values (μM)

| Com- | | Com- | | Com- | |
|---|---|---|---|---|---|
| pounds | EC₅₀ | pounds | EC₅₀ | pounds | EC₅₀ |
| Cpd B001 | 0.01073 | Cpd B024 | 0.02667 | Cpd B047 | 0.0136 |
| Cpd B002 | 0.00952 | Cpd B025 | 0.01786 | Cpd B048 | 0.00885 |
| Cpd B003 | 0.00697 | Cpd B026 | 0.02613 | Cpd B049 | 0.0124 |
| Cpd B004 | 0.00286 | Cpd B027 | 0.0217 | Cpd B050 | 0.07276 |
| Cpd B005 | 0.1617 | Cpd B028 | 0.01293 | Cpd B051 | 0.03069 |
| Cpd B006 | 0.00291 | Cpd B029 | 0.0083 | Cpd B052 | 0.01595 |
| Cpd B007 | 0.00416 | Cpd B030 | 0.0377 | Cpd B053 | 0.00507 |
| Cpd B008 | 0.004 | Cpd B031 | 0.00201 | Cpd B054 | 0.01922 |
| Cpd B009 | 0.00106 | Cpd B032 | 0.04853 | Cpd B055 | 0.00513 |
| Cpd B010 | 0.04747 | Cpd B033 | 0.00246 | Cpd B056 | 0.00144 |
| Cpd B011 | 0.00207 | Cpd B034 | 0.00604 | Cpd B057 | 0.00158 |
| Cpd B012 | 0.04517 | Cpd B035 | 0.00606 | Cpd B058 | 0.00172 |
| Cpd B013 | 0.00627 | Cpd B036 | 0.01756 | Cpd B059 | 0.00123 |
| Cpd B014 | 0.00092 | Cpd B037 | 0.00541 | Cpd B060 | 0.00081 |
| Cpd B015 | 0.00954 | Cpd B038 | 0.01386 | Cpd B061 | 0.00246 |
| Cpd B016 | 0.00551 | Cpd B039 | 0.05635 | Cpd B062 | 0.00108 |
| Cpd B017 | 0.02666 | Cpd B040 | 0.04044 | Cpd B063 | 0.00312 |
| Cpd B018 | 0.00097 | Cpd B041 | 0.1413 | Cpd B064 | 0.03283 |
| Cpd B019 | 0.07175 | Cpd B042 | 0.03285 | Cpd B065 | 0.00323 |
| Cpd B020 | 0.422 | Cpd B043 | 0.07951 | Cpd B066 | 0.1302 |
| Cpd B021 | 0.1667 | Cpd B044 | 0.01612 | Cpd B067 | 0.00354 |
| Cpd B022 | 0.1488 | Cpd B045 | 0.01156 | Cpd B068 | 0.01344 |
| Cpd B023 | 0.01856 | Cpd B046 | 0.00411 | | |
| Cpd B069 | 0.00485 | Cpd B127 | 0.06762 | Cpd B177 | 0.00539 |
| Cpd B070 | 0.05397 | Cpd B128 | 0.126 | Cpd B178 | 0.12773 |
| Cpd B071 | 0.00296 | Cpd B129 | 0.09698 | Cpd B179 | 0.01613 |
| Cpd B072 | 1.297 | Cpd B130 | 0.0133 | Cpd B180 | <0.00019 |
| Cpd B073 | 0.005 | Cpd B131 | 0.00438 | Cpd B181 | 0.00498 |
| Cpd B074 | 0.00361 | Cpd B132 | 0.00316 | Cpd B183 | <0.00019 |
| Cpd B075 | 0.06131 | Cpd B133 | 0.0029 | Cpd B184 | 0.00512 |
| Cpd B077 | 0.00368 | Cpd B134 | 0.0485 | Cpd B185 | 0.01778 |
| Cpd B078 | 0.01263 | Cpd B135 | <0.00019 | Cpd B186 | 0.03399 |
| Cpd B079 | 0.01338 | Cpd B136 | 0.6712 | Cpd B187 | 0.00915 |
| Cpd B081 | 0.00969 | Cpd B137 | 0.04932 | Cpd B188 | 0.05301 |
| Cpd B082 | 0.02791 | Cpd B138 | 0.8875 | Cpd B189 | 0.01061 |
| Cpd B083 | 0.01891 | Cpd B139 | 0.02056 | Cpd B190 | 0.4907 |
| Cpd B085 | 3.762 | Cpd B140 | 0.0015 | Cpd B191 | 0.1439 |
| Cpd B086 | 0.4368 | Cpd B141 | 0.00257 | Cpd B192 | 0.1406 |
| Cpd B087 | 1.019 | Cpd B142 | 0.8635 | Cpd B193 | 0.03623 |
| Cpd B089 | 0.00293 | Cpd B143 | 0.1088 | Cpd B194 | 0.2641 |
| Cpd B090 | 0.00915 | Cpd B144 | 0.08651 | Cpd B193 | 0.00341 |
| Cpd B091 | 0.00456 | Cpd B145 | 0.00278 | Cpd B196 | 0.01252 |
| Cpd B092 | 0.01392 | Cpd B146 | <0.00019 | Cpd B197 | 0.01282 |
| Cpd B093 | 0.07465 | Cpd B147 | 0.00325 | Cpd B198 | 0.01646 |
| Cpd B098 | 0.0715 | Cpd B148 | 0.3955 | Cpd B199 | 0.05387 |
| Cpd B099 | 0.08596 | Cpd B149 | 0.00758 | Cpd B200 | 0.02931 |
| Cpd B100 | 0.2609 | Cpd B150 | <0.00019 | Cpd B201 | 0.00355 |
| Cpd B101 | 0.0766 | Cpd B151 | 0.00052 | Cpd B202 | 0.01409 |
| Cpd B102 | 0.02641 | Cpd B152 | 0.00276 | Cpd B203 | 0.01554 |
| Cpd B103 | 1.998 | Cpd B153 | 0.03725 | Cpd B204 | 0.05488 |
| Cpd B104 | 0.00163 | Cpd B154 | 0.00781 | Cpd B205 | 0.263 |
| Cpd B105 | 0.0026 | Cpd B155 | 0.00467 | Cpd B206 | 0.03098 |
| Cpd B106 | <0.00019 | Cpd B156 | 0.08334 | Cpd B207 | 0.01779 |
| Cpd B107 | <0.00019 | Cpd B157 | 0.00933 | Cpd B208 | 0.2516 |
| Cpd B108 | 0.00258 | Cpd B158 | 0.06528 | Cpd B209 | 0.01524 |
| Cpd B109 | 0.01206 | Cpd B159 | 0.00229 | Cpd B210 | 0.01455 |
| Cpd B110 | 0.1796 | Cpd B160 | 0.00368 | Cpd B211 | 0.09479 |
| Cpd B111 | 0.00355 | Cpd B161 | 0.04928 | Cpd B212 | 0.00494 |
| Cpd B112 | 0.01219 | Cpd B162 | 0.05156 | Cpd B213 | 0.01456 |
| Cpd B113 | 0.01227 | Cpd B163 | 0.00367 | Cpd B214 | 0.03115 |
| Cpd B114 | 0.01357 | Cpd B164 | 0.0191 | Cpd B215 | 0.00373 |
| Cpd B115 | 0.04126 | Cpd B165 | 0.00249 | Cpd B216 | 0.00569 |
| Cpd B116 | 0.07222 | Cpd B166 | 0.00074 | Cpd B217 | 0.00046 |
| Cpd B117 | 0.00381 | Cpd B167 | 0.00417 | Cpd B218 | 0.01681 |
| Cpd B118 | 0.02783 | Cpd B168 | 0.7797 | Cpd B219 | 0.00834 |
| Cpd B119 | 0.04603 | Cpd B169 | 0.8806 | Cpd B220 | 0.03391 |
| Cpd B120 | 0.00427 | Cpd B170 | 3.445 | Cpd B221 | 0.01859 |
| Cpd B121 | 0.01248 | Cpd B171 | 0.07782 | Cpd B223 | 0.00399 |
| Cpd B122 | 3.725 | Cpd B172 | 0.1023 | Cpd B224 | 0.01216 |
| Cpd B123 | 0.01466 | Cpd B173 | 0.03031 | Cpd B225 | 0.00527 |
| Cpd B124 | 0.05445 | Cpd B174 | 0.01468 | Cpd B226 | 0.00116 |
| Cpd B125 | 0.7046 | Cpd B175 | <0.00019 | Cpd B227 | 0.01289 |
| Cpd B126 | 0.1924 | Cpd B176 | 0.0734 | | |
| Cpd B301 | 0.0112 | Cpd B313 | 0.0029 | Cpd B325 | 0.0362 |
| Cpd B302 | 0.0009 | Cpd B314 | 0.003 | Cpd B326 | 0.1452 |
| Cpd B303 | 0.0118 | Cpd B315 | 0.004 | Cpd B327 | 0.0099 |
| Cpd B304 | 0.0315 | Cpd B316 | 0.0031 | Cpd B328 | 0.002 |
| Cpd B305 | 0.0022 | Cpd B317 | 0.041 | Cpd B329 | 0.0023 |
| Cpd B306 | 0.0357 | Cpd B318 | 0.0101 | Cpd B330 | 0.008 |
| Cpd B307 | 0.0013 | Cpd B319 | 0.0021 | Cpd B331 | 0.0733 |
| Cpd B308 | 0.0038 | Cpd B320 | 0.0014 | Cpd B332 | 0.002 |
| Cpd B309 | 0.012 | Cpd B321 | 0.0086 | Cpd B333 | 0.002 |
| Cpd B310 | 0.0046 | Cpd B322 | 0.0028 | Cpd B334 | 0.002 |
| Cpd B311 | 0.004 | Cpd B323 | 0.002 | Cpd B335 | 0.003 |
| Cpd B312 | 0.0022 | Cpd B324 | 0.0026 | Cpd B336 | 0.007 |
| Cpd B405 | 4.819 | Cpd B426 | 0.8499 | Cpd B447 | 0.148 |
| Cpd B406 | 4.616 | Cpd B427 | 0.8456 | Cpd B448 | 0.12 |
| Cpd B407 | 4.309 | Cpd B428 | 0.7784 | Cpd B449 | 0.1194 |
| Cpd B408 | 4.002 | Cpd B429 | 0.7461 | Cpd B450 | 0.1164 |
| Cpd B409 | 3.725 | Cpd B430 | 0.6905 | Cpd B451 | 0.1055 |
| Cpd B410 | 3.321 | Cpd B431 | 0.4919 | Cpd B452 | 0.09446 |
| Cpd B411 | 3.008 | Cpd B432 | 0.482 | Cpd B453 | 0.08364 |
| Cpd B412 | 2.851 | Cpd B433 | 0.4692 | Cpd B454 | 0.07663 |
| Cpd B413 | 2.77 | Cpd B434 | 0.396 | Cpd B455 | 0.07448 |
| Cpd B414 | 2.709 | Cpd B435 | 0.3904 | Cpd B456 | 0.07394 |
| Cpd B415 | 2.413 | Cpd B436 | 0.3891 | Cpd B457 | 0.07149 |
| Cpd B416 | 2.217 | Cpd B437 | 0.3538 | Cpd B458 | 0.06551 |
| Cpd B417 | 1.688 | Cpd B438 | 0.3167 | Cpd B459 | 0.0648 |
| Cpd B418 | 1.329 | Cpd B439 | 0.2953 | Cpd B460 | 0.06302 |
| Cpd B419 | 1.263 | Cpd B440 | 0.2601 | Cpd B461 | 0.0565 |
| Cpd B420 | 1.211 | Cpd B441 | 0.253 | Cpd B462 | 0.05311 |
| Cpd B421 | 0.9808 | Cpd B442 | 0.1998 | Cpd B463 | 0.04171 |
| Cpd B422 | 0.9776 | Cpd B443 | 0.1708 | Cpd B464 | 0.04009 |
| Cpd B423 | 0.9715 | Cpd B444 | 0.1674 | Cpd B465 | 0.03809 |
| Cpd B424 | 0.9303 | Cpd B445 | 0.1567 | Cpd B466 | 0.01861 |
| Cpd B425 | 0.8543 | Cpd B446 | 0.1503 | | |

### Evaluation of Compounds Cpd B061 and Cpd B112, on Hippocampal Long-Term Potentiation in Human Tau Transgenic Mice Model of Alzheimer Disease:

The Tau-P301S line of mice was transgenic for the 383 aa isoform of human tau with the P301S mutation (Allen et al., 2002). In these mice, a clinical mutant of the human Tau (P301S) was expressed under control of the murine Thy1 promoter (neuron-specific expression) in C57Bl/6JxCBA/ca background. The homozygous hTauP301S-Tg model used consisted of the progeny from a cross between two homozygous hTauP301S-Tg parental mice in C57Bl/6JxCBA/ca background, bred in SPF breeding conditions.

Electrophysiological assessment of hippocampal long-term potentiation (LTP) using high-frequency stimulation (HFS) of the Schaffer collaterals demonstrated a statistically significant reduction of CA3-CA1 LTP around the age of 3 months, indicating that overexpression of mutant human TAU.P301S in mouse caused a deficit in synaptic plasticity.

Six mice (3,5 months of age) per group were treated orally once daily for 7 days with vehicle or 0.4, 2 or 10 mg/kg cpd B061, or twice daily for 7 days with vehicle or 25 mg/kg cpd B112, formulated in labrasol/capryol (70/30% W/V). A wild type vehicle group was included as well. The mice in study were sacrificed after a minimum of 7 days of acclimatization and 7 days of dosing. LTP measurements occurred between 90 and 120 minutes after excision of the brain.

Mice were anaesthetized with isoflurane and then decapitated. Heads were immediately immersed in ice-cold freshly prepared artificial CSF (cerebrospinal fluid) (aCSF) for at least 2 min before brain extraction. Acute slices (400 μm thick) were prepared with a vibratome (VT 1000S; Leica Microsystems, Bannockburn, IL) in ice-cold gassed aCSF. Sections were incubated in aCSF at 34° C. for 20 min and then kept at room temperature for at least 1 h before recording. aCSF was composed of the following (in mM): 119 NaCl, 11 d-glucose, 1.3 MgCl₂·6H₂O, 1.3 NaH₂PO₄, 2.5 KCl, 2.5 CaCl₂, 26 NaHCO₃, gassed with O₂/CO₂ (95/5%) at least 20 min before use and throughout the experiment.

Recordings were performed in a submerged chamber continuously flowed with aCSF at 2 ml/min. Extracellular field excitatory postsynaptic potentials (fEPSPs) were recorded in the CA1 stratum radiatum using a glass micropipette filled with aCSF. fEPSPs were evoked by the electrical

TABLE 4B
Averaged fEPSP slopes (% of baseline) of the 41-60 minute post-induction interval
during long term potentiation (LTP) in cpd B061 and vehicle treated hTAU mice and
wild type mice. Statistics: Two-way ANOVA test, Dunnett's multiple comparisons

| | Average fEPSP slope | |
|---|---|---|
| Treatment group | (% of baseline) | p-value (vs TAU-P301S_Vehicle) |
| WT_Vehicle | 165.3 | 0.003 |
| Tau-P301S_Vehicle | 129.3 | |
| Tau-P301S_Cpd B061 0.4 mpk | 129.0 | >0.999 |
| Tau-P301S_Cpd B061 2 mpk | 152.2 | 0.140 |
| Tau-P301S_Cpd B061 10 mpk | 169.3 | 0.002 |

TABLE 5B
Averaged fEPSP slopes (% of baseline) of the 41-60 minute post-induction interval
during long term potentiation (LTP) in cpd B112 and vehicle treated hTAU mice and
wild type mice. Statistics: One-way ANOVA test, Dunnett multiple comparisons

| Treatment group | Average fEPSP slope (% of baseline) | p-value (vs TAU-P301S_Vehicle) |
|---|---|---|
| WT_Vehicle | 151.2 | 0.0304 |
| Tau-P301S_Vehicle | 121.1 | |
| Tau-P301S_Cpd B112 25 mpk | 144.4 | 0.0966 |

The compounds as defined herein (including all embodiments thereof as described herein) can be used as medicaments, e.g., in the form of pharmaceutical preparations. The pharmaceutical preparations can be administered orally, e.g., in the form of tablets, coated tablets, dragees, hard and soft gelatine capsules, solutions, emulsions or suspensions. The administration can, however, also be effected rectally, e.g., in the form of suppositories, or parenterally, e.g., in the form of injection solutions.

The compounds as defined herein (including all embodiments thereof as described herein) can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragees and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound as defined herein (including all embodiments thereof as described herein) and a therapeutically inert carrier are also an object of the present invention, as is a process for their production, which comprises bringing one or more compounds as defined herein (including all embodiments thereof as described herein) and, if desired, one or more other therapeutically valuable substances into a galenical administration form together with one or more therapeutically inert carriers.

The most preferred indications in accordance with the present invention are those, which include disorders of the central nervous system, for example the treatment or prevention of Alzheimer's disease, Pick's disease, corticobasal degeneration, progressive supranuclear palsy, frontotemporal dementia and parkinsonism (linked to chromosome 17, FTDP-17).

The dosage can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

1-18. (canceled)

19. A compound of formula (B1A), or a salt, a solvate, a hydrate, a polymorph, a tautomer, a racemate or a stereoisomer thereof, or a prodrug thereof, wherein 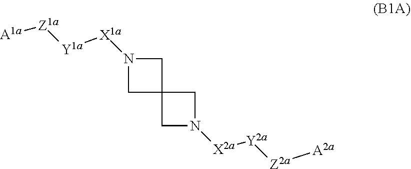
A^{1a} is selected from the group comprising C₆₋₁₀aryl, C₄₋₆cycloalkyl, and 5-6 membered heteroaryl containing at least one N, O and/or S; wherein each of said group can be unsubstituted or substituted with one or more Ria, wherein each R^{1a} is independently selected from the group comprising halo, hydrogen, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C1.6alkoxycarbonyl, C₃₋₆ cycloalkyl, (R^{5a})₂N-carbonyl, and C₁₋₆alkylcarbonylamino; and/or two R^{1a} together with the atom(s) to which they are attached can form a C₃₋₆cycloalkyl, or a 5-10 membered saturated or partially saturated heterocyclyl;
X^{1a} is selected from —CO—, or —SO₂—;
Y^{1a} is selected from —C(R^{3a})₂—, —NR^{5a}— or —O—; or Y^{1a} is a single bond;
Z^{1a} is a single bond, or is selected from —C(R^{3a})₂—, —NR^{5a}—, or —O—;
each R^{3a} is independently selected from hydrogen, halo or C₁₋₆alkyl;
each R^{5a} is independently selected from hydrogen, or C₁₋₆alkyl;
X^{2a} is a single bond or —CO—;
Y^{2a} is —C(R^{4a})₂—,
Z^{2a} is a single bond, or is —(C(R^{4a})₂)ₙ—; wherein n is an integer selected from 1 or 2;
each R^{4a} is independently selected from hydrogen, halo or C₁₋₆alkyl; or two R^{4a} together with the atom(s) to which they are attached can form a C₃₋₆cycloalkyl;
A^{2a} is selected from the group comprising phenyl, 5-6 membered heteroaryl containing at least one N, O and/or S, and C₅₋₆cycloalkyl; wherein each of said group can be unsubstituted or substituted with one or more R^{2a}; wherein each R^{2a} is independently selected from the group comprising halo, hydrogen, C₁₋₆alkyl, C₂₋₆alkenyl, C₂₋₆alkynyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a 5-10 membered saturated or partially saturated heterocyclyl;
with the proviso that said compound is not:
2-Benzyl-6-(toluene-4-sulfonyl)-2,6-diazaspiro[3.3]heptane;
2-(1,3-benzodioxol-5-ylmethyl)-6-[(4-methylphenyl)sulfonyl-2,6-Diazaspiro[3.3]heptane.

20. The compound according to claim 19, wherein
A^{1a} is selected from the group comprising C₆₋₁₀aryl, C₄₋₆cycloalkyl, and 5-6 membered heteroaryl containing at least one N, O and/or S; each of said group can be unsubstituted or substituted with one, or more Ria, for example 1, 2, 3 or 4 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C1.6alkoxycarbonyl, C₃₋₆ cycloalkyl, (R^{5a})₂N-carbonyl, and C₁₋₆alkylcarbonylamino; and/or two R^{1a} together with the atom(s) to which they are attached can form a C₃₋₆cycloalkyl, or a 5-9 membered saturated or partially saturated heterocyclyl;
X^{1a} is selected from —CO—, or —SO₂;
Y^{1a} is a single bond, or is selected from —C(R^{3a})₂—, —NR^{5a}— or —O—;
Zia is a single bond, or is selected from —C(R^{3a})₂—, —NR^{5a}— or —O—;
each R^{3a} is hydrogen, halo or C₁₋₄alkyl;
each R^{5a} is hydrogen, or C₁₋₄alkyl;
X^{2a} is a single bond or —CO—;
Y^{2a} is —C(R^{4a})₂—,
Z^{2a} is a single bond, or is —(C(R^{4a})₂)ₙ—; wherein n is an integer selected from 1 or 2;
each R^{4a} is independently selected from hydrogen, halo or C₁₋₄alkyl; or two R^{4a} together with the atom(s) to which they are attached can form a C₃₋₅cycloalkyl; and
A^{2a} is selected from the group comprising phenyl, 5-6 membered heteroaryl containing at least one N, O and/or S, and C₅₋₆cycloalkyl; wherein each of said group can be unsubstituted or substituted with one or more R^{2a}, for example 1, 2, 3 or 4 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a 5-10 membered saturated or partially saturated heterocyclyl.

21. The compound according to claim 19, having structural formula (B1A1), (B1A2), (B1A3), (B1A4), or (B1A6),

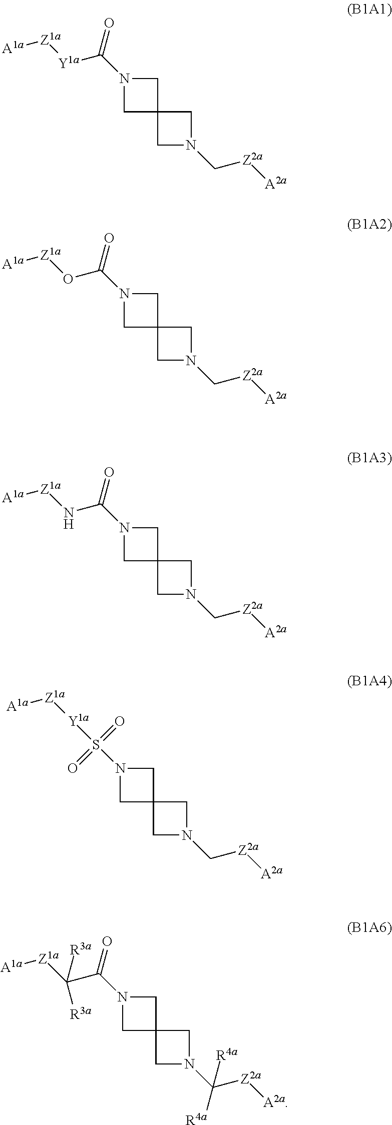

22. The compound according to claim 19, wherein
A^{1a} is selected from the group comprising C₆₋₁₀aryl, C₅₋₆cycloalkyl, and 5-6 membered heteroaryl containing at least one N, O and/or S; each of said group can be unsubstituted or substituted with one, or more Ria, for example 1, 2, or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, and C₃₋₆cycloalkyl; and/or two R^{1a} together with the atom(s) to which they are attached can form a C₃₋₆cycloalkyl, or a 5-9 membered saturated or partially saturated heterocyclyl; and
A^{2a} is selected from the group comprising phenyl, 5-6 membered heteroaryl containing at least one N, O and/or S and C₅₋₆cycloalkyl; wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a 5-10 membered saturated or partially saturated heterocyclyl.

23. The compound according to claim 19, having structural formula (B1A8), (B1A9), (B1A10), (B1A11), (B1A13),

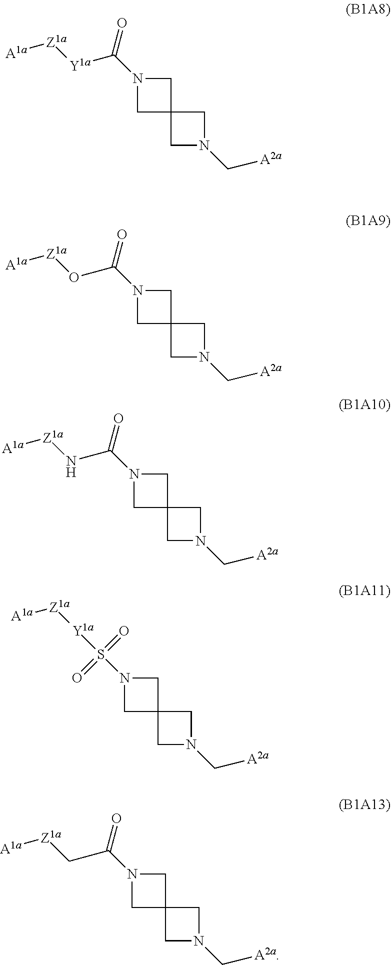

24. The compound according to claim 19, having structural formula (B1A14), (B1A15), (B1A16), (B1A17), (B1A18), (B1A19), (B1A20), (B1A21), (B1A22), (B1A23), (B1A24),

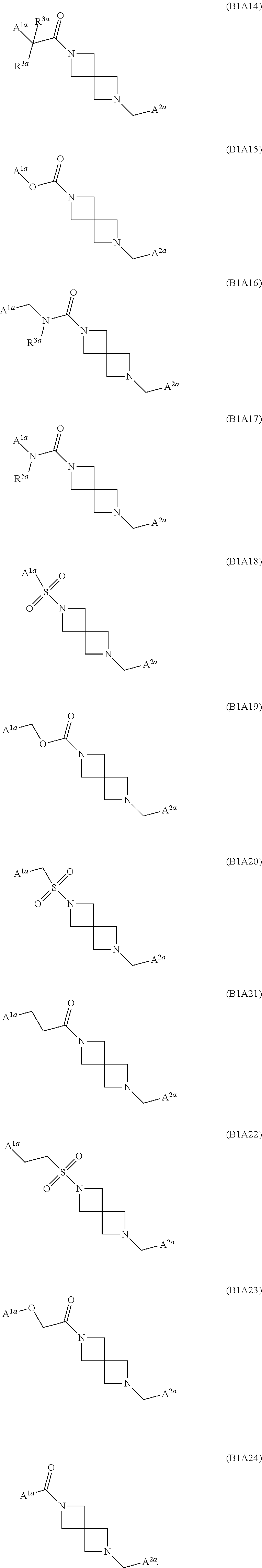

25. The compound according to claim 19, wherein
A^{1a} is selected from the group comprising phenyl, C₅₋₆cycloalkyl, and 5-6 membered heteroaryl containing at least one N; each of said group can be unsubstituted or substituted with one, or more R^{1a}, for example 1, 2, or 3 R^{1a}, wherein each R^{1a} is independently selected from the group comprising hydrogen, halo, C₁₋₆alkyl, C₁₋₆alkoxy, haloC₁₋₆alkyl, haloC₁₋₆alkoxy, cyano, hydroxy; and/or two R^{1a} together with the atom(s) to which they are attached can form a C₃₋₆cycloalkyl, or a 5-9 membered saturated or partially saturated heterocyclyl; and
A^{2a} is selected from the group comprising phenyl, 5-6 membered heteroaryl containing at least one N, O and/or S and C₅₋₆cycloalkyl; wherein each of said group can be unsubstituted or substituted with one or more R^{2a}, for example 1, 2, or 3 R^{2a}; wherein each R^{2a} is independently selected from the group comprising hydrogen, halo, C₁₋₆alkyl, C₁₋₆alkoxy, haloC₁₋₆alkyl, haloC₁₋₆alkoxy, cyano, hydroxy, carboxyl, C₁₋₆alkoxycarbonyl, (R^{5a})₂N-carbonyl, —SO₂—R^{5a}, C₃₋₆cycloalkyl, and C₁₋₆alkylcarbonylamino; and/or two R^{2a} together with the atom(s) to which they are attached can form a 5-10 membered saturated or partially saturated heterocyclyl.

26. The compound according to claim 19, having structural formula (B1A25), (B1A26), (B1A27), (B1A28), (B1A29), (B1A30), (B1A31), (B1A32), (B1A33), (B1A34), (B1A35), (B1A36), (B1A37), (B1A38), (B1A39), (B1A40), (B1A41), (B1A42), (B1A43), (B1A44), (B1A45), (B1A46), (B1A47), (B1A48), (B1A49), (B1A50), (B1A51), (B1A52), (B1A53), (B1A54), (B1A55), (B1A56), (B1A57), (B1A58), (B1A59), (B1A60), (B1A61), (B1A62), (B1A63), (B1A64), (B1A65), (B1A66), (B1A67), (B1A68), (B1A69), (B1A70), (B1A71), (B1A72), (B1A73), (B1A74), (B1A75), (B1A76), 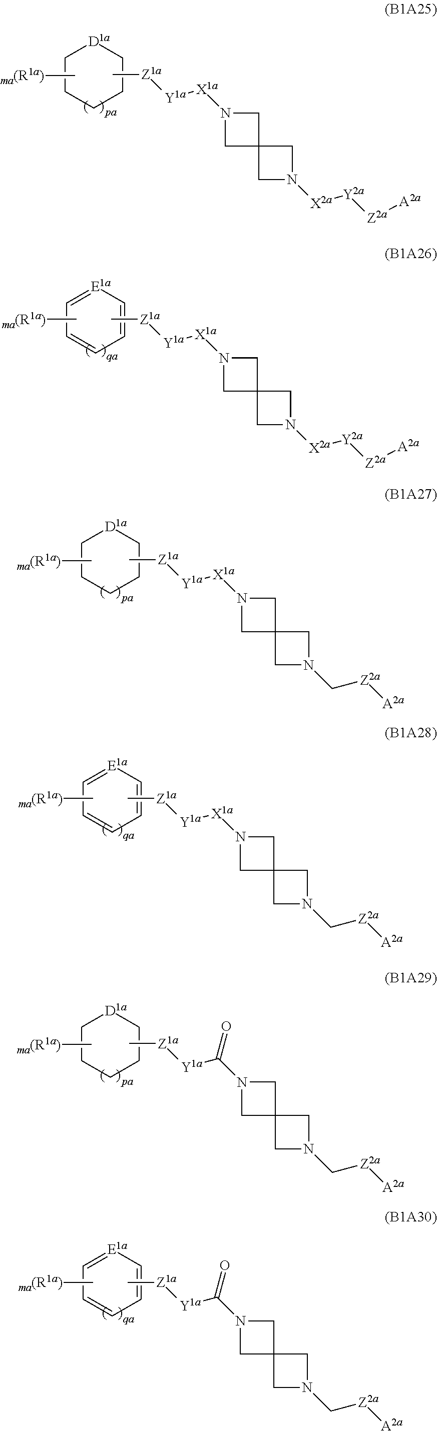 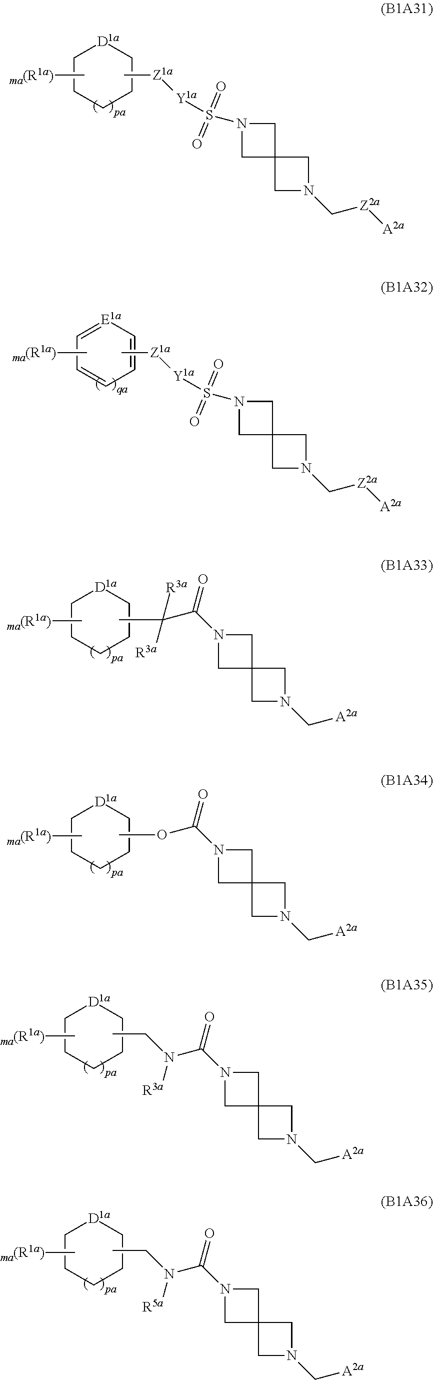 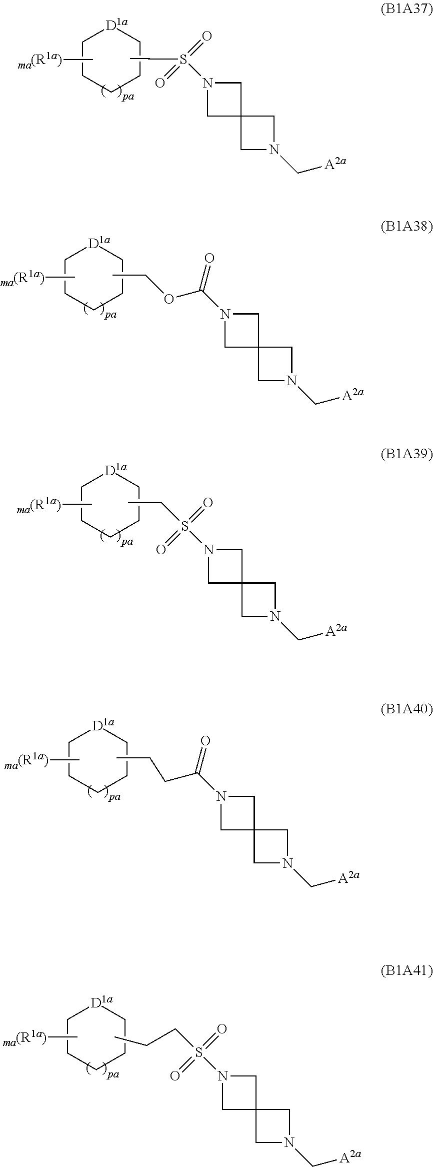 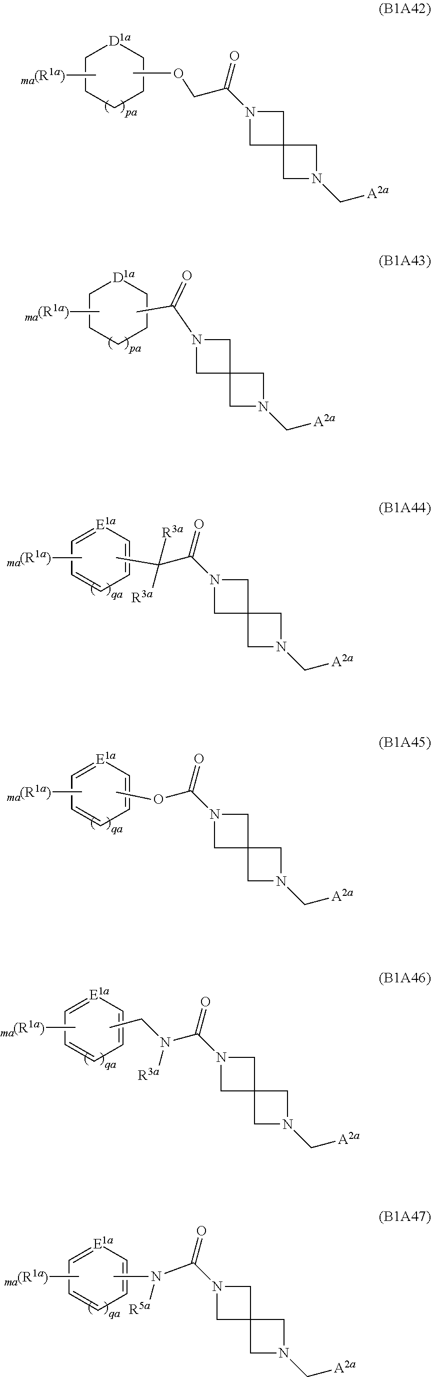 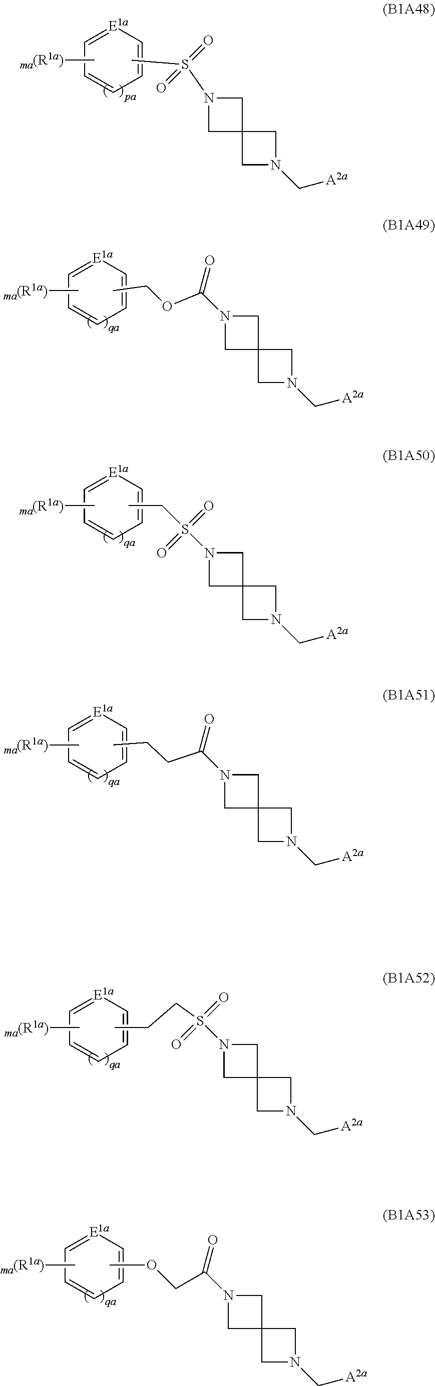 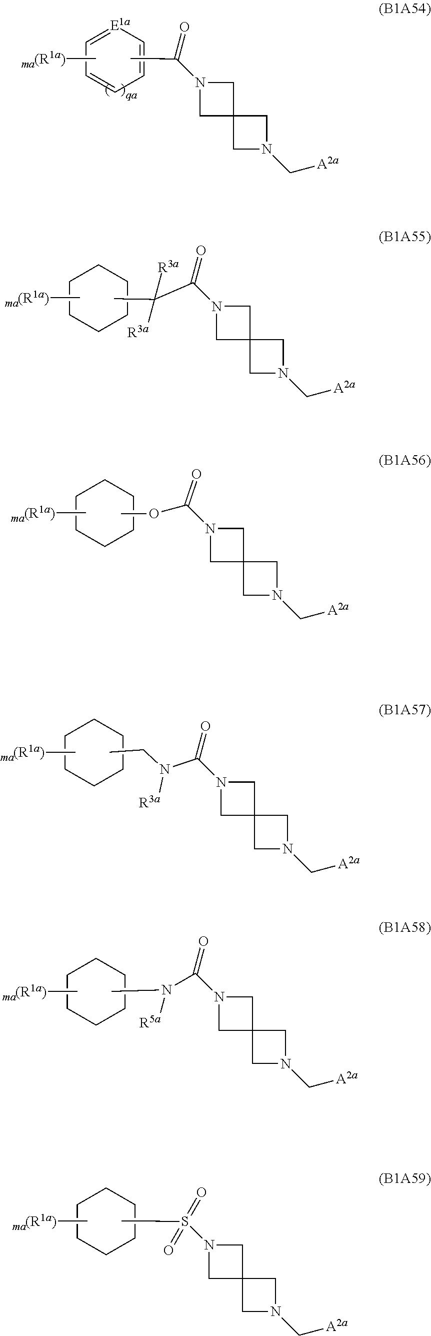 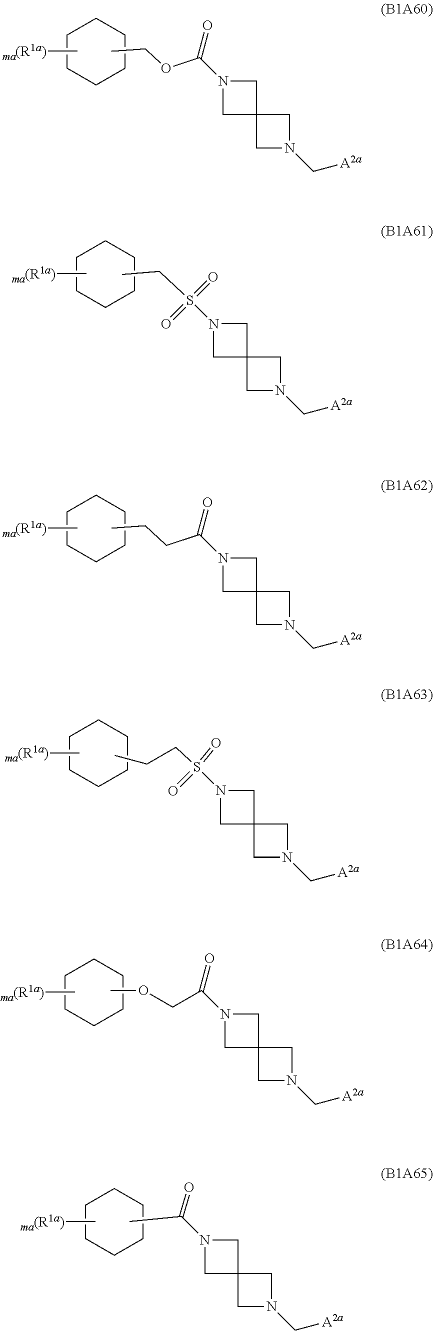 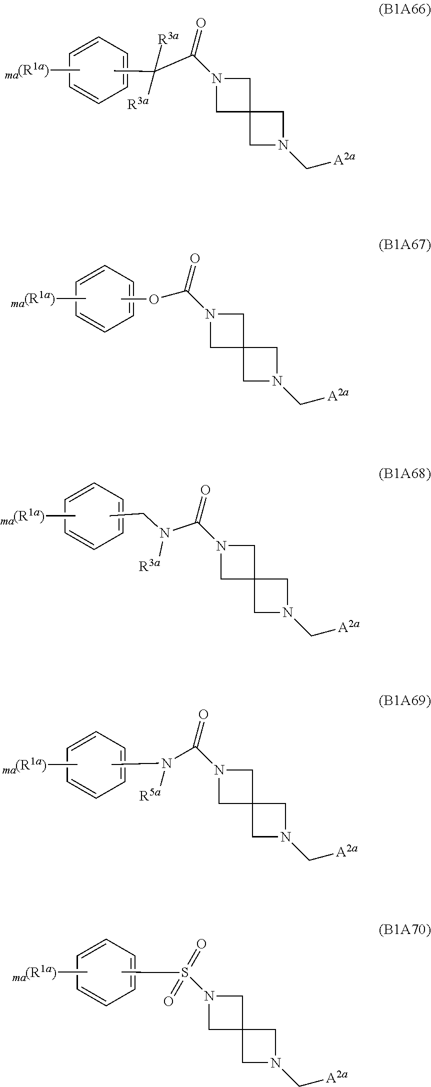 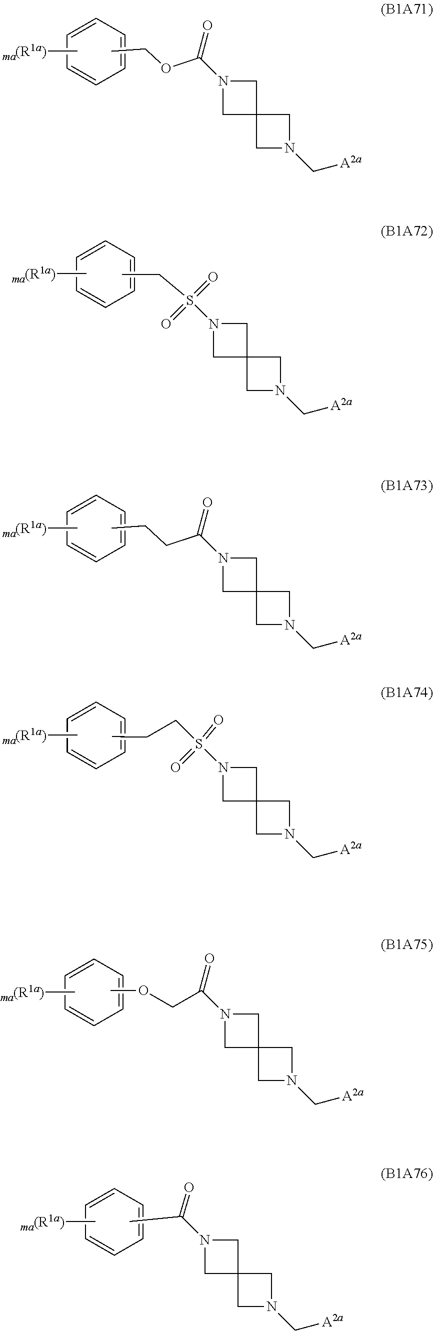
wherein ma is an integer selected from 1, 2, 3 or 4, pa is an integer selected from 0 or 1, D^{1a} is CH₂, qa is 0, and E^{1a} is NH, or qa is 1 and E^{1a} is CH, or N.

27. The compound according to claim 19, having structural formula (B1A77), (B1A78), 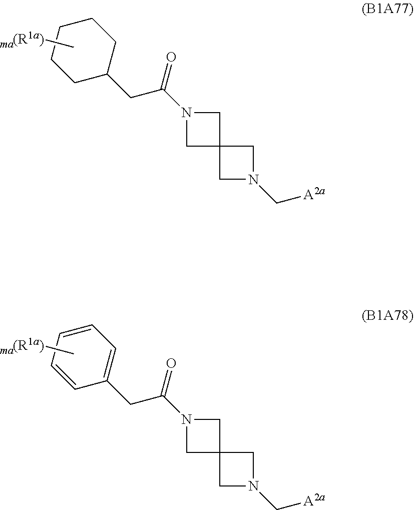
wherein ma is an integer selected from 1, 2, 3 or 4.

28. The compound according to claim 19, having structural formula (B1A79), (B1A80), 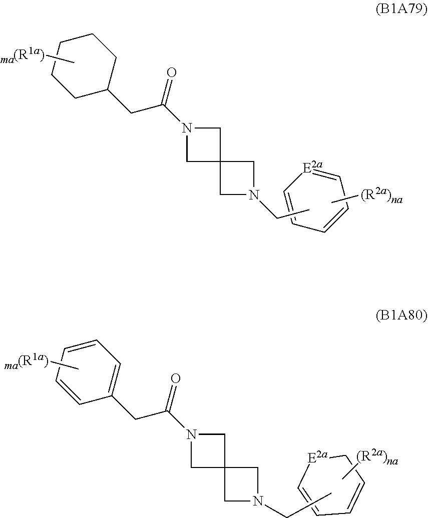
wherein ma is an integer selected from 1, 2, 3 or 4, na is an integer selected from 1, 2, 3 or 4, E^{2a} is selected from CH or N.

29. The compound according to claim 19, wherein said compound is selected from the group consisting of Cpd B001 to Cpd B041, Cpd B043 to Cpd B052, Cpd B054 to Cpd B065, Cpd B069 to Cpd B087, Cpd B089 to Cpd B227, Cpd B401 to Cpd B474, Cpd B476 to Cpd B483, Cpd B485 to Cpd B487, Cpd B489 to Cpd B490, Cpd B492 to Cpd B500, and Cpd B503 to Cpd B521 as listed in Table 1B.

30. A pharmaceutical composition comprising the compound according to claim 19, and at least one pharmaceutical acceptable carrier.

31. A medicine comprising the compound according to claim 19, or a pharmaceutical composition thereof.

32. A method of treating or preventing a neurodegenerative disorder in a subject in need thereof comprising administering to the subject a therapeutic amount of the compound according to claim 19, or a pharmaceutical composition thereof.

33. The method according to claim 32, wherein the neurodegenerative disorder is selected from the group consisting of Parkinson's disease, Alzheimer's disease, diffuse Lewy body disease, amyotrophic lateral sclerosis, Niemann Pick disease, Hallervorden Spatz syndrome, Down syndrome, Pick's disease, progressive supranuclear palsy, vascular dementia, neuroaxonal dystrophy, Huntington's disease, frontotemporal lobar degeneration (FTLD), multiple system atrophy and Creutzfeld-Jacob's disease.

34. The method according to claim 32, wherein the neurodegenerative disorder is Alzheimer's disease.
